SEQUENCE LISTING (44 sequences)

SEQ ID NO: 1-8 - skipped (8 entries)

SEQ ID NO: 9 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - RNA, 4283 nt, synthetic construct
  source 1..4283: mol_type = other RNA
agaataaactagtattcttctggtccccacagactcagagagaacccgccaccatgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccctcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 11 - RNA, 47 nt, synthetic construct
  source 1..47: mol_type = other RNA
atagtaaactagtattcttctggtccccacagactcagagagaaccc

SEQ ID NO: 12 - RNA, 42 nt, synthetic construct
  source 1..42: mol_type = other RNA
aaactagtattcttctggtccccacagactcagagagaaccc

SEQ ID NO: 13 - RNA, 278 nt, synthetic construct
  source 1..278: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 14 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 15-20 - skipped (6 entries)

SEQ ID NO: 21 - protein, 266 aa, Severe acute respiratory syndrome coronavirus 2
MFVFLVLLPLVSSQCVVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGRSLEVLFQGPG

SEQ ID NO: 22-27 - skipped (6 entries)

SEQ ID NO: 28 - RNA, 4282 nt, Severe acute respiratory syndrome coronavirus 2
  source 1..4282: mol_type = genomic RNA
gggcgaactagtattcttctggtccccacagactcagagagaacccgccaccatgtttgtgtttcttgtgctgctgcctcttgtgtcttctcagtgtgtgaatttgacaacaagaacacagctgccaccagcttatacaaattcttttaccagaggagtgtattatcctgataaagtgtttagatcttctgtgctgcacagcacacaggacctgtttctgccattttttagcaatgtgacatggtttcatgcaattcatgtgtctggaacaaatggaacaaaaagatttgataatcctgtgctgccttttaatgatggagtgtattttgcttcaacagaaaagtcaaatattattagaggatggatttttggaacaacactggattctaaaacacagtctctgctgattgtgaataatgcaacaaatgtggtgattaaagtgtgtgaatttcagttttgtaatgatccttttctgggagtgtattatcacaaaaataataaatcttggatggaatctgaatttagagtgtattcctctgcaaataattgtacatttgaatatgtgtctcagccttttctgatggatctggaaggaaaacagggcaattttaaaaatctgagagaatttgtgtttaaaaatattgatggatattttaaaatttattctaaacacacaccaattaatttagtgagagatctgcctcagggattttctgctctggaacctctggtggatctgccaattggcattaatattacaagatttcagacactgctggctctgcacagatcttatctgacacctggagattcttcttctggatggacagccggagctgcagcttattatgtgggctatctgcagccaagaacatttctgctgaaatataatgaaaatggaacaattacagatgctgtggattgtgctctggatcctctgtctgaaacaaaatgtacattaaaatcttttacagtggaaaaaggcatttatcagacatctaattttagagtgcagccaacagaatctattgtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaaaatctacaaatttagtgaaaaataaatgtgtgaattttaattttaatggattaacaggaacaggagtgctgacagaatctaataaaaaatttctgccttttcagcagtttggcagagatattgcagataccacagatgcagtgagagatcctcagacattagaaattctggatattacaccttgttcttttgggggtgtgtctgtgattacacctggaacaaatacatctaatcaggtggctgtgctgtatcaggatgtgaattgtacagaagtgccagtggcaattcatgcagatcagctgacaccaacatggagagtgtattctacaggatctaatgtgtttcagacaagagcaggatgtctgattggagcagaacatgtgaataattcttatgaatgtgatattccaattggagcaggcatttgtgcatcttatcagacacagacaaattccccaaggagagcaagatctgtggcatctcagtctattattgcatacaccatgtctctgggagcagaaaattctgtggcatattctaataattctattgctattccaacaaattttaccatttctgtgacaacagaaattttacctgtgtctatgacaaaaacatctgtggattgtaccatgtacatttgtggagattctacagaatgttctaatctgctgctgcagtatggatctttttgtacacagctgaatagagctttaacaggaattgctgtggaacaggataaaaatacacaggaagtgtttgctcaggtgaaacagatttacaaaacaccaccaattaaagattttggaggatttaattttagccagattctgcctgatccttctaaaccttctaaaagatcttttattgaagatctgctgtttaataaagtgacactggcagatgcaggatttattaaacagtatggagattgcctgggtgatattgctgcaagagatctgatttgtgctcagaaatttaatggactgacagtgctgcctcctctgctgacagatgaaatgattgctcagtacacatctgctttactggctggaacaattacaagcggatggacatttggagctggagctgctctgcagattccttttgcaatgcagatggcttacagatttaatggaattggagtgacacagaatgtgttatatgaaaatcagaaactgattgcaaatcagtttaattctgcaattggcaaaattcaggattctctgtcttctacagcttctgctctgggaaaactgcaggatgtggtgaatcagaatgcacaggcactgaatactctggtgaaacagctgtctagcaattttggggcaatttcttctgtgctgaatgatattctgtctagactggatcctcctgaagctgaagtgcagattgatagactgatcacaggaagactgcagtctctgcagacttatgtgacacagcagctgattagagctgctgaaattagagcttctgctaatctggctgctacaaaaatgtctgaatgtgtgctgggacagtcaaaaagagtggatttttgtggaaaaggatatcatctgatgtcttttccacagtctgctccacatggagtggtgtttttacatgtgacatatgtgccagcacaggaaaagaattttaccacagcaccagcaatttgtcatgatggaaaagcacattttccaagagaaggagtgtttgtgtctaatggaacacattggtttgtgacacagagaaatttttatgaacctcagattattacaacagataatacatttgtgtcaggaaattgtgatgtggtgattggaattgtgaataatacagtgtatgatccactgcagccagaactggattcttttaaagaagaactggataaatattttaaaaatcacacatctcctgatgtggatttaggagatatttctggaatcaatgcatctgtggtgaatattcagaaagaaattgatagactgaatgaagtggccaaaaatctgaatgaatctctgattgatctgcaggaacttggaaaatatgaacagtacattaaatggccttggtacatttggcttggatttattgcaggattaattgcaattgtgatggtgacaattatgttatgttgtatgacatcatgttgttcttgtttaaaaggatgttgttcttgtggaagctgttgtaaatttgatgaagatgattctgaacctgtgttaaaaggagtgaaattgcattacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 29 - RNA, 4282 nt, Severe acute respiratory syndrome coronavirus 2
  source 1..4282: mol_type = genomic RNA
gggcgaactagtattcttctggtccccacagactcagagagaacccgccaccatgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccctcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 30 - RNA, 1261 nt, Severe acute respiratory syndrome coronavirus 2
  source 1..1261: mol_type = genomic RNA
gggcgaactagtattcttctggtccccacagactcagagagaacccgccaccatgtttgtgtttcttgtgctgctgcctcttgtgtcttctcagtgtgtggtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaggctcccccggctccggctccggatctggttatattcctgaagctccaagagatgggcaagcttacgttcgtaaagatggcgaatgggtattactttctacctttttaggccggtccctggaggtgctgttccagggccccggctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 31 - RNA, 4283 nt, Severe acute respiratory syndrome coronavirus 2
  source 1..4283: mol_type = genomic RNA
agaataaactagtattcttctggtccccacagactcagagagaacccgccaccatgtttgtgtttcttgtgctgctgcctcttgtgtcttctcagtgtgtgaatttgacaacaagaacacagctgccaccagcttatacaaattcttttaccagaggagtgtattatcctgataaagtgtttagatcttctgtgctgcacagcacacaggacctgtttctgccattttttagcaatgtgacatggtttcatgcaattcatgtgtctggaacaaatggaacaaaaagatttgataatcctgtgctgccttttaatgatggagtgtattttgcttcaacagaaaagtcaaatattattagaggatggatttttggaacaacactggattctaaaacacagtctctgctgattgtgaataatgcaacaaatgtggtgattaaagtgtgtgaatttcagttttgtaatgatccttttctgggagtgtattatcacaaaaataataaatcttggatggaatctgaatttagagtgtattcctctgcaaataattgtacatttgaatatgtgtctcagccttttctgatggatctggaaggaaaacagggcaattttaaaaatctgagagaatttgtgtttaaaaatattgatggatattttaaaatttattctaaacacacaccaattaatttagtgagagatctgcctcagggattttctgctctggaacctctggtggatctgccaattggcattaatattacaagatttcagacactgctggctctgcacagatcttatctgacacctggagattcttcttctggatggacagccggagctgcagcttattatgtgggctatctgcagccaagaacatttctgctgaaatataatgaaaatggaacaattacagatgctgtggattgtgctctggatcctctgtctgaaacaaaatgtacattaaaatcttttacagtggaaaaaggcatttatcagacatctaattttagagtgcagccaacagaatctattgtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaaaatctacaaatttagtgaaaaataaatgtgtgaattttaattttaatggattaacaggaacaggagtgctgacagaatctaataaaaaatttctgccttttcagcagtttggcagagatattgcagataccacagatgcagtgagagatcctcagacattagaaattctggatattacaccttgttcttttgggggtgtgtctgtgattacacctggaacaaatacatctaatcaggtggctgtgctgtatcaggatgtgaattgtacagaagtgccagtggcaattcatgcagatcagctgacaccaacatggagagtgtattctacaggatctaatgtgtttcagacaagagcaggatgtctgattggagcagaacatgtgaataattcttatgaatgtgatattccaattggagcaggcatttgtgcatcttatcagacacagacaaattccccaaggagagcaagatctgtggcatctcagtctattattgcatacaccatgtctctgggagcagaaaattctgtggcatattctaataattctattgctattccaacaaattttaccatttctgtgacaacagaaattttacctgtgtctatgacaaaaacatctgtggattgtaccatgtacatttgtggagattctacagaatgttctaatctgctgctgcagtatggatctttttgtacacagctgaatagagctttaacaggaattgctgtggaacaggataaaaatacacaggaagtgtttgctcaggtgaaacagatttacaaaacaccaccaattaaagattttggaggatttaattttagccagattctgcctgatccttctaaaccttctaaaagatcttttattgaagatctgctgtttaataaagtgacactggcagatgcaggatttattaaacagtatggagattgcctgggtgatattgctgcaagagatctgatttgtgctcagaaatttaatggactgacagtgctgcctcctctgctgacagatgaaatgattgctcagtacacatctgctttactggctggaacaattacaagcggatggacatttggagctggagctgctctgcagattccttttgcaatgcagatggcttacagatttaatggaattggagtgacacagaatgtgttatatgaaaatcagaaactgattgcaaatcagtttaattctgcaattggcaaaattcaggattctctgtcttctacagcttctgctctgggaaaactgcaggatgtggtgaatcagaatgcacaggcactgaatactctggtgaaacagctgtctagcaattttggggcaatttcttctgtgctgaatgatattctgtctagactggatcctcctgaagctgaagtgcagattgatagactgatcacaggaagactgcagtctctgcagacttatgtgacacagcagctgattagagctgctgaaattagagcttctgctaatctggctgctacaaaaatgtctgaatgtgtgctgggacagtcaaaaagagtggatttttgtggaaaaggatatcatctgatgtcttttccacagtctgctccacatggagtggtgtttttacatgtgacatatgtgccagcacaggaaaagaattttaccacagcaccagcaatttgtcatgatggaaaagcacattttccaagagaaggagtgtttgtgtctaatggaacacattggtttgtgacacagagaaatttttatgaacctcagattattacaacagataatacatttgtgtcaggaaattgtgatgtggtgattggaattgtgaataatacagtgtatgatccactgcagccagaactggattcttttaaagaagaactggataaatattttaaaaatcacacatctcctgatgtggatttaggagatatttctggaatcaatgcatctgtggtgaatattcagaaagaaattgatagactgaatgaagtggccaaaaatctgaatgaatctctgattgatctgcaggaacttggaaaatatgaacagtacattaaatggccttggtacatttggcttggatttattgcaggattaattgcaattgtgatggtgacaattatgttatgttgtatgacatcatgttgttcttgtttaaaaggatgttgttcttgtggaagctgttgtaaatttgatgaagatgattctgaacctgtgttaaaaggagtgaaattgcattacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 32 - RNA, 1262 nt, Severe acute respiratory syndrome coronavirus 2
  source 1..1262: mol_type = genomic RNA
agaataaactagtattcttctggtccccacagactcagagagaacccgccaccatgtttgtgtttcttgtgctgctgcctcttgtgtcttctcagtgtgtggtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaggctcccccggctccggctccggatctggttatattcctgaagctccaagagatgggcaagcttacgttcgtaaagatggcgaatgggtattactttctacctttttaggccggtccctggaggtgctgttccagggccccggctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 33 - RNA, 11917 nt, Severe acute respiratory syndrome coronavirus 2
  source 1..11917: mol_type = genomic RNA
gatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgaaaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagagctagtcactgggctagggctcacaggcgagctggtcgatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaggcttttgcttgtcatgcaggtactctcagagcgctcatagccattataagacctaaaaaggcagtgctctgcggagatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggtagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagctaagcatgttgaccaagaaagcatgtctgcatctgaatcccggcggaacctgtgtcagcataggttatggttacgctgacagggccagcgaaagcatcattggtgctatagcgcggcagttcaagttttcccgagtatgcaaaccgaaatcctcacttgaggagacggaagttctgtttgtattcattgggtacgatcgcaaggcccgtacgcacaatccttacaagctatcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagtttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgattcttcagtgacagaacctgatgcagagctggtgagggtgcatcccaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaggagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaagaagaagaagatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctccagaaccagcctagtttccaccccgccaggcgtgaatagggtgatcactagagaggagctcgaagcgcttaccccgtcacgcactcctagcaggtcggtctccagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagctaggcaaacctctggcagcagacgatgaacatgatgatgacaggagaagggcattgcatgaggagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagactagtatgtttgtgtttcttgtgctgctgcctcttgtgtcttctcagtgtgtgaatttgacaacaagaacacagctgccaccagcttatacaaattcttttaccagaggagtgtattatcctgataaagtgtttagatcttctgtgctgcacagcacacaggacctgtttctgccattttttagcaatgtgacatggtttcatgcaattcatgtgtctggaacaaatggaacaaaaagatttgataatcctgtgctgccttttaatgatggagtgtattttgcttcaacagaaaagtcaaatattattagaggatggatttttggaacaacactggattctaaaacacagtctctgctgattgtgaataatgcaacaaatgtggtgattaaagtgtgtgaatttcagttttgtaatgatccttttctgggagtgtattatcacaaaaataataaatcttggatggaatctgaatttagagtgtattcctctgcaaataattgtacatttgaatatgtgtctcagccttttctgatggatctggaaggaaaacagggcaattttaaaaatctgagagaatttgtgtttaaaaatattgatggatattttaaaatttattctaaacacacaccaattaatttagtgagagatctgcctcagggattttctgctctggaacctctggtggatctgccaattggcattaatattacaagatttcagacactgctggctctgcacagatcttatctgacacctggagattcttcttctggatggacagccggagctgcagcttattatgtgggctatctgcagccaagaacatttctgctgaaatataatgaaaatggaacaattacagatgctgtggattgtgctctggatcctctgtctgaaacaaaatgtacattaaaatcttttacagtggaaaaaggcatttatcagacatctaattttagagtgcagccaacagaatctattgtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaaaatctacaaatttagtgaaaaataaatgtgtgaattttaattttaatggattaacaggaacaggagtgctgacagaatctaataaaaaatttctgccttttcagcagtttggcagagatattgcagataccacagatgcagtgagagatcctcagacattagaaattctggatattacaccttgttcttttgggggtgtgtctgtgattacacctggaacaaatacatctaatcaggtggctgtgctgtatcaggatgtgaattgtacagaagtgccagtggcaattcatgcagatcagctgacaccaacatggagagtgtattctacaggatctaatgtgtttcagacaagagcaggatgtctgattggagcagaacatgtgaataattcttatgaatgtgatattccaattggagcaggcatttgtgcatcttatcagacacagacaaattccccaaggagagcaagatctgtggcatctcagtctattattgcatacaccatgtctctgggagcagaaaattctgtggcatattctaataattctattgctattccaacaaattttaccatttctgtgacaacagaaattttacctgtgtctatgacaaaaacatctgtggattgtaccatgtacatttgtggagattctacagaatgttctaatctgctgctgcagtatggatctttttgtacacagctgaatagagctttaacaggaattgctgtggaacaggataaaaatacacaggaagtgtttgctcaggtgaaacagatttacaaaacaccaccaattaaagattttggaggatttaattttagccagattctgcctgatccttctaaaccttctaaaagatcttttattgaagatctgctgtttaataaagtgacactggcagatgcaggatttattaaacagtatggagattgcctgggtgatattgctgcaagagatctgatttgtgctcagaaatttaatggactgacagtgctgcctcctctgctgacagatgaaatgattgctcagtacacatctgctttactggctggaacaattacaagcggatggacatttggagctggagctgctctgcagattccttttgcaatgcagatggcttacagatttaatggaattggagtgacacagaatgtgttatatgaaaatcagaaactgattgcaaatcagtttaattctgcaattggcaaaattcaggattctctgtcttctacagcttctgctctgggaaaactgcaggatgtggtgaatcagaatgcacaggcactgaatactctggtgaaacagctgtctagcaattttggggcaatttcttctgtgctgaatgatattctgtctagactggatcctcctgaagctgaagtgcagattgatagactgatcacaggaagactgcagtctctgcagacttatgtgacacagcagctgattagagctgctgaaattagagcttctgctaatctggctgctacaaaaatgtctgaatgtgtgctgggacagtcaaaaagagtggatttttgtggaaaaggatatcatctgatgtcttttccacagtctgctccacatggagtggtgtttttacatgtgacatatgtgccagcacaggaaaagaattttaccacagcaccagcaatttgtcatgatggaaaagcacattttccaagagaaggagtgtttgtgtctaatggaacacattggtttgtgacacagagaaatttttatgaacctcagattattacaacagataatacatttgtgtcaggaaattgtgatgtggtgattggaattgtgaataatacagtgtatgatccactgcagccagaactggattcttttaaagaagaactggataaatattttaaaaatcacacatctcctgatgtggatttaggagatatttctggaatcaatgcatctgtggtgaatattcagaaagaaattgatagactgaatgaagtggccaaaaatctgaatgaatctctgattgatctgcaggaacttggaaaatatgaacagtacattaaatggccttggtacatttggcttggatttattgcaggattaattgcaattgtgatggtgacaattatgttatgttgtatgacatcatgttgttcttgtttaaaaggatgttgttcttgtggaagctgttgtaaatttgatgaagatgattctgaacctgtgttaaaaggagtgaaattgcattacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgcggccgcatgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 34 - RNA, 11917 nt, Severe acute respiratory syndrome coronavirus 2
  source 1..11917: mol_type = genomic RNA
gatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgaaaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagagctagtcactgggctagggctcacaggcgagctggtcgatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaggcttttgcttgtcatgcaggtactctcagagcgctcatagccattataagacctaaaaaggcagtgctctgcggagatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggtagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagctaagcatgttgaccaagaaagcatgtctgcatctgaatcccggcggaacctgtgtcagcataggttatggttacgctgacagggccagcgaaagcatcattggtgctatagcgcggcagttcaagttttcccgagtatgcaaaccgaaatcctcacttgaggagacggaagttctgtttgtattcattgggtacgatcgcaaggcccgtacgcacaatccttacaagctatcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagtttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgattcttcagtgacagaacctgatgcagagctggtgagggtgcatcccaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaggagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaagaagaagaagatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctccagaaccagcctagtttccaccccgccaggcgtgaatagggtgatcactagagaggagctcgaagcgcttaccccgtcacgcactcctagcaggtcggtctccagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagctaggcaaacctctggcagcagacgatgaacatgatgatgacaggagaagggcattgcatgaggagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagactagtatgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccctcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgcggccgcatgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 35 - RNA, 8896 nt, Severe acute respiratory syndrome coronavirus 2
  source 1..8896: mol_type = genomic RNA
gatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgaaaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagagctagtcactgggctagggctcacaggcgagctggtcgatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaggcttttgcttgtcatgcaggtactctcagagcgctcatagccattataagacctaaaaaggcagtgctctgcggagatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggtagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagctaagcatgttgaccaagaaagcatgtctgcatctgaatcccggcggaacctgtgtcagcataggttatggttacgctgacagggccagcgaaagcatcattggtgctatagcgcggcagttcaagttttcccgagtatgcaaaccgaaatcctcacttgaggagacggaagttctgtttgtattcattgggtacgatcgcaaggcccgtacgcacaatccttacaagctatcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagtttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgattcttcagtgacagaacctgatgcagagctggtgagggtgcatcccaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaggagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaagaagaagaagatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctccagaaccagcctagtttccaccccgccaggcgtgaatagggtgatcactagagaggagctcgaagcgcttaccccgtcacgcactcctagcaggtcggtctccagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagctaggcaaacctctggcagcagacgatgaacatgatgatgacaggagaagggcattgcatgaggagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagactagtatgtttgtgtttcttgtgctgctgcctcttgtgtcttctcagtgtgtggtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaggctcccccggctccggctccggatctggttatattcctgaagctccaagagatgggcaagcttacgttcgtaaagatggcgaatgggtattactttctacctttttaggccggtccctggaggtgctgttccagggccccggctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgcggccgcatgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 36 - RNA, 9079 nt, Severe acute respiratory syndrome coronavirus 2
  source 1..9079: mol_type = genomic RNA
gatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgaaaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagagctagtcactgggctagggctcacaggcgagctggtcgatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaggcttttgcttgtcatgcaggtactctcagagcgctcatagccattataagacctaaaaaggcagtgctctgcggagatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggtagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagctaagcatgttgaccaagaaagcatgtctgcatctgaatcccggcggaacctgtgtcagcataggttatggttacgctgacagggccagcgaaagcatcattggtgctatagcgcggcagttcaagttttcccgagtatgcaaaccgaaatcctcacttgaggagacggaagttctgtttgtattcattgggtacgatcgcaaggcccgtacgcacaatccttacaagctatcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagtttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgattcttcagtgacagaacctgatgcagagctggtgagggtgcatcccaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaggagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaagaagaagaagatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctccagaaccagcctagtttccaccccgccaggcgtgaatagggtgatcactagagaggagctcgaagcgcttaccccgtcacgcactcctagcaggtcggtctccagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagctaggcaaacctctggcagcagacgatgaacatgatgatgacaggagaagggcattgcatgaggagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagactagtatgtttgtgtttcttgtgctgctgcctcttgtgtcttctcagtgtgtggtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaggctcccccggctccggctccggatctggttatattcctgaagctccaagagatgggcaagcttacgttcgtaaagatggcgaatgggtattactttctacctttttaggaagcggcagcggatctgaacagtacattaaatggccttggtacatttggcttggatttattgcaggattaattgcaattgtgatggtgacaattatgttatgttgtatgacatcatgttgttcttgtttaaaaggatgttgttcttgtggaagctgttgtaaatttgatgaagatgattctgaacctgtgttaaaaggagtgaaattgcattacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgcggccgcatgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 37 - protein, 327 aa, Severe acute respiratory syndrome coronavirus 2
MFVFLVLLPLVSSQCVVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGSGSGSEQYIKWPWYIWLGFIAGLIAIVMVTIMLCCMTSCCSCLKGCCSCGSCCKFDEDDSEPVLKGVKLHYT

SEQ ID NO: 38 - protein, 311 aa, Severe acute respiratory syndrome coronavirus 2
MFVFLVLLPLVSSQCVNLTVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGSGSGSEQYIKWPWYIWLGFIAGLIAIVMVTIMLCCMTSCCSCLKGCCSCGSCC

SEQ ID NO: 39 - RNA, 1397 nt, Severe acute respiratory syndrome coronavirus 2
  source 1..1397: mol_type = genomic RNA
agaataaactagtattcttctggtccccacagactcagagagaacccgccaccatgtttgtgtttcttgtgctgctgcctcttgtgtcttctcagtgtgtgaatttgacagtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaggctcccccggctccggctccggatctggttatattcctgaagctccaagagatgggcaagcttacgttcgtaaagatggcgaatgggtattactttctacctttttaggaagcggcagcggatctgaacagtacattaaatggccttggtacatttggcttggatttattgcaggattaattgcaattgtgatggtgacaattatgttatgttgtatgacatcatgttgttcttgtttaaaaggatgttgttcttgtggaagctgttgttgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 40 - protein, 314 aa, Severe acute respiratory syndrome coronavirus 2
MDWIWRILFLVGAATGAHSQMQVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGSGSGSEQYIKWPWYIWLGFIAGLIAIVMVTIMLCCMTSCCSCLKGCCSCGSCC

SEQ ID NO: 41 - RNA, 1406 nt, Severe acute respiratory syndrome coronavirus 2
  source 1..1406: mol_type = genomic RNA
agaataaactagtattcttctggtccccacagactcagagagaacccgccaccatggattggatttggagaatcctgttcctcgtgggagccgctacaggagcccactcccagatgcaggtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaggctcccccggctccggctccggatctggttatattcctgaagctccaagagatgggcaagcttacgttcgtaaagatggcgaatgggtattactttctacctttttaggaagcggcagcggatctgaacagtacattaaatggccttggtacatttggcttggatttattgcaggattaattgcaattgtgatggtgacaattatgttatgttgtatgacatcatgttgttcttgtttaaaaggatgttgttcttgtggaagctgttgttgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 42 - RNA, 20 nt, synthetic construct
  source 1..20: mol_type = other RNA
gagtcgctagccgcgtcgct

SEQ ID NO: 43 - RNA, 458 nt, synthetic construct
  source 1..458: mol_type = other RNA
atagtaaactagtattcttctggtccccacagactcagagagaacccgccaccctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 44 - RNA, 39 nt, synthetic construct
  source 1..39: mol_type = other RNA
tgtgggctgatgaggccgtgaggccgaaaccagaagaat

(19) United States
(12) Patent Application Publication
(10) Pub. No.: US 2026/0109727 A1
(43) Pub. Date: Apr. 23, 2026

(54) RNA CONSTRUCTS AND USES THEREOF

(71) Applicant: BioNTech SE, Mainz (DE)

(72) Inventors: Gábor Boros, Mainz (DE); Surjendu Dey, Munich (DE); Irena Vlatkovic, Mainz (DE); Christophe Henry, Munich (DE); Frank Eggert, Mainz (DE); Christoph Kröner, Mainz (DE); Andreas Kuhn, Mainz (DE)

(21) Appl. No.: 19/117,603

(22) PCT Filed: Oct. 3, 2023
(86) PCT No.: PCT/IB2023/059937

§ 371 Date: Apr. 1, 2025

Related U.S. Application Data
(60) Provisional application No. 63/413,207, filed on Oct. 4, 2022.

(51) Int. Cl. C07H 21/02; A61K 31/7088

(52) U.S. Cl. CPC C07H 21/02; A61K 31/7088

(57) ABSTRACT

Disclosed herein are RNA polynucleotides comprising a 5′ Cap, a 5′ UTR comprising a cap proximal sequence disclosed herein, and a sequence encoding a payload. Also disclosed herein are compositions and medical preparations comprising the same, and compositions and methods of making and using the same.

### BACKGROUND

Use of RNA polynucleotides as therapeutics is a new and emerging field.

### SUMMARY

The present disclosure identifies certain challenges that can be associated with in vitro production of RNA, for example of RNA therapeutics.

For example, in some embodiments, the present disclosure identifies the source of certain problems that can be encountered with expression of polypeptides encoded by RNA therapeutics. Among other things, the present disclosure provides technologies for improving capping efficiency (e.g., percentage of capped transcripts in an in vitro transcription reaction), quality of an RNA preparation (e.g., of an in vitro transcribed RNA, such as, e.g., the amount of short polynucleotide byproducts produced), translation efficiency of an RNA encoding a payload, and/or expression of a polypeptide payload encoded by an RNA. In some embodiments, translation efficiency and/or expression of an RNA-encoded payload can be improved with an RNA polynucleotide comprising: a 5′ cap as defined and described herein; a 5′ UTR comprising a cap proximal sequence as defined and described herein, and a sequence encoding a payload. Without wishing to be bound by a particular theory, the present disclosure proposes that improved RNA transcription, capping efficiency, translation efficiency, and/or polypeptide payload expression and/or reduced transcription byproduct formation can be achieved through use of a 5′ cap structure as described herein in combination with certain transcription start site sequences of a template DNA.

In some embodiments, the present disclosure recognizes that certain caps provide improved RNA transcription, capping efficiency, translation efficiency, and/or polypeptide payload expression and/or reduced byproduct formation. In some embodiments, the present disclosure recognizes that certain caps when utilized with particular transcription start sites provide improved RNA transcription, capping efficiency, translation efficiency, and/or polypeptide payload expression and/or reduced byproduct formation.

T7 RNA polymerase most commonly utilizes a GGG transcriptional start site (e.g., generating an RNA whose first three residues are each “G”), and, moreover, has been reported to prefer “G” as an initiating residue (e.g., generating an RNA whose first residue is “G”). Conrad, et al. (2020) Communications Biology 3:439. Studies comparing T7 transcription of templates with different initiating residues report levels of transcripts beginning with “A” are only 25% of those observed for transcripts beginning with “G”. Milligan, et al. (1987) Nucleic Acids Research 15:8783-8798.

The 3′ end of commonly used dinucleotide cap analogs also employ “G” (e.g., m^{7,2′-O}GppSpG “β-S-ARCA” or “D1”). Grudzien-Nogalska, et al. RNA 13:1745-1755. Indeed, certain such caps, e.g., β-S-ARCA, provide advantages including, e.g., being more resistant to human decapping enzymes (Kowalska et al. (2008) RNA 14:1119-1131) and interferon-induced proteins with tetratricopeptide repeats (IFITs), which inhibit Cap0-dependent translation (Diamond et al. (2014) Cytokine & Growth Factor Reviews 25:543-550; and Miedziak et al. (2019) RNA 25:58-68). However, poor capping efficiency is sometimes observed. Without wishing to be bound by any particular theory, the present disclosure proposes that competition with GTP in the transcription reaction may contribute to such poor capping efficiency.

In some embodiments, cap1 analogs (including, e.g., ones that are commercially available) can be incorporated into synthetic RNAs (e.g., RNAs produced by in vitro transcription (IVT)) in the correct orientation to produce cap1 RNA with a high capping efficiency, e.g., all in a rapid co-transcriptional reaction. For example, a cap analog for a synthetic self-amplifying RNA (saRNA) may be or comprise CleanCap AU, TriLink (#N7-114). For example, a cap analog for a synthetic mRNA may be or comprise CleanCap AG, Trilink, #N7-413. See Henderson, J. M., et al. (2021) Current protocols, 1, e39. An appealing feature of these trinucleotide cap1 analogs is that they require an A initiator, which may avoid potential slippage of RNA polymerases on the DNA template strand as opposed to those containing a G triplet as a transcriptional start site. See Imburgio, et al. (2000) Biochemistry, 39, 10419-10430.

Furthermore, anti reverse cap analog (ARCA)-capped mRNA may possess higher translation efficiency compared to conventional cap analogs. See Stepinski, J., et al. (2001) RNA (New York, N.Y.), 7, 1486-1495; and Kuhn, A. N., et al. (2010) Gene therapy, 17, 961-971. For example, a variant of the CleanCap AG with a modification at the C3′ position of 7-methylguanosine (CleanCap AG 3′ OMe) may play an important role in the progress of immunotherapeutic vaccination strategy against SARS-CoV-2. See Sahin, U. et al. (2021) Nature, 595, 572-577. In some embodiments, without wishing to be bound to a particular theory, the present disclosure provides the recognition that an ARCA cap1 analog may exhibit better translational efficiency and/or biological activity as compared to those capped with its non-ARCA version (See FIG. 1). Additionally or alternatively, cap analogs paired with particular start sequences have been described to attempt to address one or more of these problems, e.g., WO 2021/214204A1.

Additionally or alternatively, incorporation of nucleoside modifications (e.g., modified uridines (including, e.g., N1-methylpseudouridine (m1Ψ)) and/or modified adenosines (including, e.g., N6-methyladenine (m6A)) into synthetic RNA (e.g., in some embodiments IVTmRNAs) may increase biological stability and thereby enhance the durability of the encoded protein compared to unmodified RNAs. See Karikó, K., et al. (2008) Molecular therapy: the journal of the American Society of Gene Therapy, 16, 1833-1840; and Gao, Y., et al. (2020) Immunity, 52, 1007-1021.e8. However, without wishing to be bound to a particular theory, because certain saRNA cannot contain modified nucleosides, use of such modified nucleosides has primarily been limited to preventive vaccines against infectious diseases in contrast to non-replicating mRNAs. See Bloom, K., et al. (2021) Gene therapy, 28, 117-129. Additionally or alternatively, non-replicating mRNAs also have great potential in research areas such as gene editing, protein replacement therapy, where the reduction and elimination of immunomodulation is important for reaching the appropriate therapeutic goal.

While potential advantages of using various modified nucleosides have been contemplated, the effects of cap analogs containing modified nucleosides on quality, translational efficiency, and biological activity or immunogenicity of mRNA that encodes a potentially therapeutic protein are not understood. In some embodiments, the present disclosure also provides the recognition that 5′ caps comprising modified nucleoside(s) may be a promising alternative to current capping strategies in mRNA vaccines and especially in RNA-based therapeutics.

In some embodiments, the present disclosure recognizes that certain 5′ cap structures (e.g., trinucleotide caps comprising N₁pN₂, wherein N₁ is A or an analog thereof, and N₂ is U or an analog thereof), e.g., when paired with certain transcription start sites (e.g., AUN, such as AUA), provide improved RNA transcription, improved translation efficiency, and/or improved and/or prolonged polypeptide payload expression as compared to transcripts comprising other 5′ cap structures (such as, e.g., CC114 or CC413 caps used). Additionally or alternatively, in some embodiments, the present disclosure recognizes that certain 5′ cap structures (e.g., trinucleotide caps comprising N₁pN₂, wherein N₁ is A or an analog thereof, and N₂ is U or an analog thereof), e.g., when paired with certain transcription start sites (e.g., AUN, such as AUA), result in higher capping efficiency, reduced amounts of short contaminants, and reduced toxicity due to cytokine/chemokine secretion as compared to transcripts comprising other 5′ cap structures (such as, e.g., CC114 or CC413 caps used). Additionally or alternatively, in some embodiments, the present disclosure recognizes that the demonstrated effects of certain 5′ cap structures (e.g., trinucleotide caps comprising N₁pN₂, wherein N₁ is A or an analog thereof, and N₂ is U or an analog thereof), e.g., when paired with certain transcription start sites (e.g., AUN, such as AUA), can be adapted not only to replicative mRNA, but also to non-replicative mRNA.

Additionally or alternatively, in some embodiments, the present disclosure recognizes that disclosed 5′ cap structures where N₂ is a modified U (e.g., pseudouridine, i.e., Ψ, and analogs thereof, such as 1-methylpseudouridine ((m¹)Ψ)) display improved RNA transcription, improved translation efficiency, and/or improved and/or prolonged polypeptide payload expression as compared to transcripts comprising other 5′ cap structures (such as, e.g., CC114 or CC413 caps, or caps comprising unmodified U). Additionally or alternatively, in some embodiments, the present disclosure recognizes that disclosed 5′ cap structures where N₂ is a modified U (e.g., pseudouridine, i.e., Ψ, and analogs thereof, such as (m¹)Ψ) result in higher capping efficiency, lesser amounts of short contaminants, and reduced toxicity due to cytokine/chemokine secretion as compared to transcripts comprising other 5′ cap structures (such as, e.g., CC114 or CC413 caps, or caps comprising unmodified U). Additionally or alternatively, in some embodiments, the present disclosure recognizes that the demonstrated effects of disclosed 5′ cap structures where N₂ is a modified U (e.g., pseudouridine, i.e., Ψ, and analogs thereof, such as (m¹)Ψ) display can be adapted not only to replicative mRNA, but also to non-replicative mRNA.

Accordingly, in some embodiments, the present disclosure provides, inter alia, a composition or medical preparation comprising an RNA polynucleotide, comprising: (i) a 5′ cap, e.g., as disclosed herein; (ii) a cap proximal sequence, e.g., as disclosed herein; and (iii) a sequence encoding a payload. Also disclosed herein are methods of making and using the same to, e.g., induce an immune response in a subject.

In some embodiments, the present disclosure also provides trinucleotide caps G*N₁pN₂, or a salt thereof, wherein:
G* comprises a structure of formula I′:

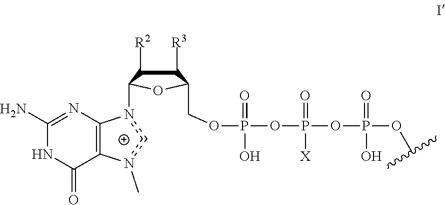

wherein:
   each R² and R³ is independently —OH or —OCH₃; and
   X is OH or SH;
N₁ is A or an analog thereof;
N₂ is U or an analog thereof; and
p is a group selected from phosphate (e.g., —P(═O)(OH)— or —P(═O)(O⁻)—) or thiophosphate (e.g., —P(═S)(OH)— or —P(═S)(O⁻)—).

In some embodiments, it will be appreciated that trinucleotide caps having the structure of formula I′ (e.g., trinucleotide caps comprising a N₂ nucleotide of formula II″ or formula II′″) demonstrate surprising advantages such as, for example, improved translation efficiencies, as discussed in greater detail herein.

### BRIEF DESCRIPTION OF THE DRAWING

FIG. 1 shows a comparison of levels of murine EPO and hematocrit %. CC113 corresponds to (m⁷)Gppp(m^{2′-O})ApG; CC413 corresponds to (m₂^{7,3′-O})Gppp(m^{2′-O})ApG. Translational efficiency as well as biological activity of EPO mRNA capped with CC413 is significantly better than CC113.

FIG. 2A shows a comparison of RNA quality after in vitro transcription using (m₂^{7,3′-O})Gppp(m^{2′-O})ApU cap (i.e., compound I′-1) and various start sites. The highest yield was observed with AUAGU start site. FIG. 2B shows a comparison of capping efficiency by 21% Urea-PAGE. A high yield was observed when (m₂^{7,3′-O})Gppp(m^{2′-O})ApU cap (i.e., compound I′-1) was used in the range of 3-6 mM concentration. Capping efficiency is close to 100% regardless of the concentration used.

FIG. 3 shows a comparison of capping efficiency by 21% Urea-PAGE. Cap 1 corresponds to compound I′-1 ((m₂^{7,3′-O})Gppp(m^{2′-O})ApU); Cap 2 corresponds to compound I′-6 ((m₂^{7,3′-O})Gppp(m^{2′-O})Ap(m¹)Ψ); CC114 corresponds to (m⁷)Gppp(m^{2′-O})ApU; CC413 corresponds to (m₂^{7,3′-O})Gppp(m^{2′-O})ApG. The capping efficiency of compound I′-1 and compound I′-6 is close to 100% and is comparable to CC114 and CC413.

FIG. 4 shows a comparison of amounts of short contaminants for certain caps and start sites. Cap 1 corresponds to compound I′-1 ((m₂^{7,3′-O})Gppp(m^{2′-O})ApU); Cap 2 corresponds to compound I′-6 ((m₂^{7,3′-O})Gppp(m^{2′-O})Ap(m¹)Ψ); CC114 corresponds to (m⁷)Gppp(m^{2′-O})ApU; CC413 corresponds to (m₂^{7,3′-O})Gppp(m^{2′-O})ApG. A minimal amount of short contaminants was observed for compound I′-6 and CC413 mRNA, while significant amount was observed for other unmodified mRNAs tested, independent of the cap.

FIG. 5 shows a comparison of XTT assay of viable PMBCs at 24 hours. Cap 1 corresponds to compound I′-1 ((m₂^{7,3′-O})Gppp(m^{2′-O})ApU); Cap 2 corresponds to compound I′-6 ((m₂^{7,3′-O})Gppp(m^{2′-O})Ap(m¹)Ψ); CC114 corresponds to (m⁷)Gppp(m^{2′-O})ApU; CC413 corresponds to (m₂^{7,3′-O})Gppp(m^{2′-O})ApG. No toxic effect on cell viability of PMBCs up to 1 μg/well mRNA originating from compound I′-1 or compound I′-6 is observed. Transfection of unmodified mRNAs leads to decrease in cellular viability starting at dose 0.333 μg/well, however this effect does not depend on the cap used, but on the mRNA modification.

FIGS. 6A, 6B, 6C, 6D, 6E, 6F, and 6G show a comparison of cytokine/chemokine secretion in human PBMCs. Cap 1 corresponds to compound I′-1 ((m₂^{7,3′-O})Gppp(m^{2′-O})ApU); Cap 2 corresponds to compound I′-6 ((m₂^{7,3′-O})Gppp(m^{2′-O})Ap(m¹)Ψ); CC114 corresponds to (m⁷)Gppp(m^{2′-O})ApU; CC413 corresponds to (m₂^{7,3′-O})Gppp(m^{2′-O})ApG. Compound I′-6 is comparable to CC413 in regard to the amount of cytokines/chemokines secreted by human PBMCs after transfection of m1Ψ-modified mRNA. In regard to cytokines/chemokines of unmodified mRNA, compound I′-1 is comparable to CC114 and leads to significantly higher cytokines/chemokines in comparison to CC413.

FIG. 7 shows a comparison of EPO secretion in human hepatocytes at day 1 after transfection of 0.1 μg/well TransIT-EPO mRNA (IV187). Cap 1 corresponds to compound I′-1 ((m₂^{7,3′-O})Gppp(m^{2′-O})ApU); Cap 2 corresponds to compound I′-6 ((m₂^{7,3′-O})Gppp(m^{2′-O})Ap(m¹)Ψ); CC114 is (m⁷)Gppp(m^{2′-O})ApU; CC413 is (m₂^{7,3′-O})Gppp(m^{2′-O})ApG. Compound I′-1 and compound I′-6 show higher translation compared to CC413 in human hepatocytes at 24 hrs.

FIG. 8A shows a comparison of plasma EPO mice IV injected with 3 μg TransIT-formulated somEPO mRNA (JR81). Cap 1 corresponds to compound I′-1 ((m₂^{7,3′-O})Gppp(m^{2′-O})ApU); Cap 2 corresponds to compound I′-6 ((m₂^{7,3′-O})Gppp(m^{2′-O})Ap(m¹)Ψ); CC114 corresponds to (m⁷)Gppp(m^{2′-O})ApU; CC413 corresponds to (m₂^{7,3′-O})Gppp(m^{2′-O})ApG. EPO mRNA capped with compound I′-6 translated 2- to 3-fold greater at later time points compared to those capped with CC413, demonstrating that compound I′-6 has a strong beneficial effect on translational capacity and biological activity of mRNA. FIG. 8B shows hematocrit level in mice IV injected with 3 μg TransIT-complexed somEPO mRNA (hAg) capped with certain caps. Cap 1 corresponds to compound I′-1 ((m₂^{7,3′-O})Gppp(m^{2′-O})ApU); Cap 2 corresponds to compound I′-6 ((m₂^{7,3′-O})Gppp(m^{2′-O})Ap(m¹)Ψ); CC114 corresponds to (m⁷)Gppp(m^{2′-O})ApU; CC413 corresponds to (m₂^{7,3′-O})Gppp(m^{2′-O})ApG. Hematocrit values in mice injected with EPO mRNA capped with compound I′-6 are very high and further increased after day 14 after injection.

FIG. 9 depicts a comparison of plasma EPO mice IV injected with 3 μg TransIT-formulated somEPO mRNA capped with cap analogs of formula I′. CC114 corresponds to (m⁷)Gppp(m^{2′-O})ApU; CC413 corresponds to (m₂^{7,3′-O})Gppp(m^{2′-O})ApG. I′-1 corresponds to (m₂^{7,3′-O})Gppp(m^{2′-O})ApU. I′-2 corresponds to (m₂^{7,2′-O})Gppp(m^{2′-O})ApU. I′-13 corresponds to m7Gppp(m^{2′-O})Ap(m1)Ψ. I′-5 corresponds to (m₂^{7,2′-O})Gppp(m^{2′-O})Ap(m1)Ψ. I′-6 corresponds to (m₂^{7,3′-O})Gppp(m^{2′-O})Ap(m¹)Ψ. ARCA analogs I′-1 and I′-6 translated significantly better compared to non-ARCA caps CC114 and I′-13 regardless of the RNA modification. m1-mRNA capped with non-ARCA cap I′-13 containing m1Ψ-modified RNA translated 8 and 25-fold more than U-mRNA capped with non-ARCA CC114 without nucleoside modification at 6 and 24 h after injection, respectively.

FIG. 10 shows a comparison of EPO levels in mice injected with 3 μg TransIT-formulated with U-containing mRNA capped with I′-I and m1Ψ-modified mRNA capped with I′-6. I′-6 (i.e., mRNA with the combination of m1Ψ-m1Ψ) performed the best and translated 2-3-fold more than I′-1 at each time points after administration. U-containing mRNA capped with I′-1 showed translational capacity which is significantly lower in each time point than that observed for m1Ψ modification is present both in the cap analog (I′-6) and in the mRNA.

FIG. 11 shows a comparison of EPO levels in mice injected with 3 ug TransIT-formulated m1Ψ-modified mRNA with caps comprising unmodified uridine (I′-1) and unmodified pseudouridine (I′-3) and modified uridine (U) or pseudouridine (1) (N5-methyluridine (I′-9), N5-methoxyuridine (I′-12), N1-methylpseudouridine (I′-6), and N1-propargylpseudouridine (I′-16). At 48 and 72 hours after injection. EPO level in mice injected with mRNA capped with Ψ-containing cap analog (I′-3—(m₂^{7,3′-O})G(5′)ppp(5′)(m^{2′-O})ApΨ) is equal or slightly less to those bearing m1Ψ (I′-6—(m₂^{7,3′-O})G(5′)ppp(5′)(m^{2′-O})Apm1Ψ) (FIG. 11). Neither uridine (U) and its derivatives (5-methylU, 5-methoxyU) nor pseudouridine derivative 1-propargylΨ could improve the potency of N₁-methylpseudouridine (1-methylΨ)-containing cap (I′-6).

FIG. 12 depicts the effect on levels of cytokines and chemokines after application of Lipoplex (LPX)-formulated EPO mRNAs. FIG. 12A depicts the effect CC413 and I′-6 on IL-6 levels. FIG. 12B depicts the effect CC413 and I′-6 on TNF-α levels. FIG. 12C depicts the effect CC413 and I′-6 on IL-1 levels. FIG. 12D depicts the effect CC413 and I′-6 on IFN-γ levels. FIG. 12E depicts the effect CC413 and I′-6 on IP-1β levels. I′-6 demonstrated less of an increase in the levels of proinflammatory cytokines and chemokines (i.e., I′-6 showed less immunogenicity) as compared to CC413 across the concentrations tested.

FIG. 13 shows a comparison of EPO levels in primary human hepatocytes transfected with 0.1 μg/well TransIT-formulated somEPO mRNA. EPO level was measured from supernatants transfected using I′-6 or CC114-capped uRNA. Increased secretion of EPO in human primary cells was detected at all three tested time points: 24 h, 48 h and 144 h. These results suggested that cap1 analogs such as I′-6 are suitable for translation of the encoded protein and can be used for synthesizing non-replicating functional mRNAs.

FIG. 14 shows a comparison of EPO uRNA capped with I′-6 and I′-1. In this case both mRNAs had the same TAGT 5′end. I′-6 showed benefit leading to significantly lower cytokines (IL-6 (FIG. 14A), TNF-α (FIG. 14B), IL-1β (FIG. 14C) and IFN-γ (FIG. 14D)) 24 h after application to human PBMCs. Thus, I′-6 results in lower immunogenicity.

FIG. 15 shows EPO secretion after application of EPO-encoding Ψ-mRNA capped with uridine (U) or pseudouridine (Ψ) derivatives CC413 and I′-3, respectively. The level of EPO was higher at 24 h and 48 h when I′-3 was used compared to CC413.

FIG. 16 shows a comparison of EPO-encoding mRNA capped with cap1 analogs bearing N5-methyluridine (I′-9), N5-methoxyuridine (I′-12), N1-methylpseudouridine (I′-6) and N1-propargylpseudouridine (I′-16). I′-6 showed increased level of secreted EPO at 24 h as compared to other caps. In addition, mRNAs with modified caps led to increase in EPO secretion at 24 h and 48 h when compared to the unmodified I′-1.

### CERTAIN DEFINITIONS

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present disclosure will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

Compounds of the present disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms are within the scope of the disclosure. Additionally, unless otherwise stated, the present disclosure also includes compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure. In some embodiments, compounds of this disclosure comprise one or more deuterium atoms.

In the following, the elements of the present disclosure will be described. These elements are listed with specific embodiments, however, it should be understood that they may be combined in any manner and in any number to create additional embodiments. The variously described examples and embodiments should not be construed to limit the present disclosure to only the explicitly described embodiments. This description should be understood to disclose and encompass embodiments which combine the explicitly described embodiments with any number of the disclosed elements. Furthermore, any permutations and combinations of all described elements should be considered disclosed by this description unless the context indicates otherwise. The term “about” means approximately or nearly, and in the context of a numerical value or range set forth herein in some embodiments means ±20%, ±10%, ±5%, or ±3% of the numerical value or range recited or claimed.

The terms “a” and “an” and “the” and similar reference used in the context of describing the disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the disclosure and does not pose a limitation on the scope of the claims. No language in the specification should be construed as indicating any non-claimed element essential to the practice of the disclosure.

Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of” or “consisting essentially of”.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

In the following, definitions will be provided which apply to all aspects of the present disclosure. The following terms have the following meanings unless otherwise indicated. Any undefined terms have their art recognized meanings.

Agent: As used herein, the term “agent”, may refer to a physical entity or phenomenon. In some embodiments, an agent may be characterized by a particular feature and/or effect. In some embodiments, an agent may be a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Aliphatic or aliphatic group: as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle”, “carbocyclic”, “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Unsaturated: as used herein, means that a moiety has one or more units of unsaturation.

Partially unsaturated: as used herein, refers to a ring moiety that includes at least one double or triple bond. The term “partially unsaturated”, as used herein, is intended to encompass rings having multiple sites of unsaturation, but is not intended to include aryl or heteroaryl moieties, as herein defined.

Amino acid: in its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Antibody agent: As used herein, the term “antibody agent” refers to an agent that specifically binds to a particular antigen. In some embodiments, the term encompasses a polypeptide or polypeptide complex that includes immunoglobulin structural elements sufficient to confer specific binding. For example, in some embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent in or comprises a polypeptide whose amino acid sequence includes structural elements recognized by those skilled in the art to correspond to CDRs1, 2, and 3 of an antibody variable domain; in some such embodiments, an antibody agent in or comprises a polypeptide or set of polypeptides whose amino acid sequence(s) together include structural elements recognized by those skilled in the art to correspond to both heavy chain and light chain variable region CDRs, e.g., heavy chain CDRs 1, 2, and/or 3 and light chain CDRs 1, 2, and/or 3. In some embodiments, an antibody agent is a polypeptide protein having a binding domain which is homologous or largely homologous to an immunoglobulin-binding domain. In some embodiments, an antibody agent may be or comprise a polyclonal antibody preparation. In some embodiments, an antibody agent may be or comprise a monoclonal antibody preparation.

In some embodiments, an antibody agent may include one or more constant region sequences that are characteristic of a particular organism, such as a camel, human, mouse, primate, rabbit, rat; in many embodiments, an antibody agent may include one or more constant region sequences that are characteristic of a human. In some embodiments, an antibody agent may include one or more sequence elements that would be recognized by one skilled in the art as a humanized sequence, a primatized sequence, a chimeric sequence, etc. In some embodiments, an antibody agent may be a canonical antibody (e.g., may comprise two heavy chains and two light chains). In some embodiments, an antibody agent may be in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)₂ fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies, Adnectins®; Affilins®; Trans-Bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.].

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of, susceptibility to, severity of, stage of, etc the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell). Binding between two entities may be considered “specific” if, under the conditions assessed, the relevant entities are more likely to associate with one another than with other available binding partners.

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Complementary: As used herein, the term “complementary” is used in reference to oligonucleotide hybridization related by base-pairing rules. For example, the sequence “C-A-G-T” is complementary to the sequence “G-T-C-A.” Complementarity can be partial or total. Thus, any degree of partial complementarity is intended to be included within the scope of the term “complementary” provided that the partial complementarity permits oligonucleotide hybridization. Partial complementarity is where one or more nucleic acid bases is not matched according to the base pairing rules. Total or complete complementarity between nucleic acids is where each and every nucleic acid base is matched with another base under the base pairing rules.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein, the term “corresponding to” refers to a relationship between two or more entities. For example, the term “corresponding to” may be used to designate the position/identity of a structural element in a compound or composition relative to another compound or composition (e.g., to an appropriate reference compound or composition). For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure. Those of skill in the art will also appreciate that, in some instances, the term “corresponding to” may be used to describe an event or entity that shares a relevant similarity with another event or entity (e.g., an appropriate reference event or entity). To give but one example, a gene or protein in one organism may be described as “corresponding to” a gene or protein from another organism in order to indicate, in some embodiments, that it plays an analogous role or performs an analogous function and/or that it shows a particular degree of sequence identity or homology, or shares a particular characteristic sequence element.

Designed: As used herein, the term “designed” refers to an agent (i) whose structure is or was selected by the hand of man; (ii) that is produced by a process requiring the hand of man; and/or (iii) that is distinct from natural substances and other known agents.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Encode: As used herein, the term “encode” or “encoding” refers to sequence information of a first molecule that guides production of a second molecule having a defined sequence of nucleotides (e.g., mRNA) or a defined sequence of amino acids. For example, a DNA molecule can encode an RNA molecule (e.g., by a transcription process that includes a DNA-dependent RNA polymerase enzyme). An RNA molecule can encode a polypeptide (e.g., by a translation process). Thus, a gene, a cDNA, or a single-stranded RNA (e.g., an mRNA) encodes a polypeptide if transcription and translation of mRNA corresponding to that gene produces the polypeptide in a cell or other biological system. In some embodiments, a coding region of a single-stranded RNA encoding a target polypeptide agent refers to a coding strand, the nucleotide sequence of which is identical to the mRNA sequence of such a target polypeptide agent. In some embodiments, a coding region of a single-stranded RNA encoding a target polypeptide agent refers to a non-coding strand of such a target polypeptide agent, which may be used as a template for transcription of a gene or cDNA.

Engineered: In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature.

Epitope: as used herein, the term “epitope” refers to a moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Expression: As used herein, the term “expression” of a nucleic acid sequence refers to the generation of any gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc); (3) translation of an RNA into a polypeptide or protein; and/or (4) post-translational modification of a polypeptide or protein.

Improved, increased or reduced: As used herein, these terms, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

In vitro: The term “in vitro” as used herein refers to events that occur in an artificial environment, e.g., in a test tube or reaction vessel (e.g., a bioreactor), in cell culture, etc., rather than within a multi-cellular organism.

In vitro transcription: As used herein, the term “in vitro transcription” or “IVT” refers to the process whereby transcription occurs in vitro in a non-cellular system to produce a synthetic RNA product for use in various applications, including, e.g., production of protein or polypeptides. Such synthetic RNA products can be translated in vitro or introduced directly into cells, where they can be translated. Such synthetic RNA products include, e.g., but not limited to mRNAs, antisense RNA molecules, shRNA molecules, long non-coding RNA molecules, ribozymes, aptamers, guide RNAs (e.g., for CRISPR), ribosomal RNAs, small nuclear RNAs, small nucleolar RNAs, and the like. An IVT reaction typically utilizes a DNA template (e.g., a linear DNA template) as described and/or utilized herein, ribonucleotides (e.g., non-modified ribonucleotide triphosphates or modified ribonucleotide triphosphates), and an appropriate RNA polymerase.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for parenteral administration, for example, by subcutaneous, intramuscular, intravenous or epidural injection as, for example, a sterile solution or suspension, or sustained-release formulation.

Polypeptide: As used herein refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide.

Prevent or prevention: as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Risk: as will be understood from context, “risk” of a disease, disorder, and/or condition refers to a likelihood that a particular individual will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0, 1, 2, 3, 4, 5, 6, 7, 8, 9, 10, or more. In some embodiments, risk may reflect one or more genetic attributes, e.g., which may predispose an individual toward development (or not) of a particular disease, disorder and/or condition. In some embodiments, risk may reflect one or more epigenetic events or attributes and/or one or more lifestyle or environmental events or attributes.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Vaccination: As used herein, the term “vaccination” refers to the administration of a composition intended to generate an immune response, for example to a disease-associated (e.g., disease-causing) agent. In some embodiments, vaccination can be administered before, during, and/or after exposure to a disease-associated agent, and in certain embodiments, before, during, and/or shortly after exposure to the agent. In some embodiments, vaccination includes multiple administrations, appropriately spaced in time, of a vaccine composition. In some embodiments, vaccination generates an immune response to an infectious agent. In some embodiments, vaccination generates an immune response to a tumor; in some such embodiments, vaccination is “personalized” in that it is partly or wholly directed to epitope(s) (e.g., which may be or include one or more neoepitopes) determined to be present in a particular individual's tumors.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature.

### DETAILED DESCRIPTION OF CERTAIN EMBODIMENTS

The present disclosure provides, among other things, an RNA polynucleotide comprising (i) a 5′ cap; (ii) a 5′ UTR sequence comprising a cap proximal sequence, e.g., as disclosed herein; and (iii) a sequence encoding a payload. Also provided herein are compositions and medical preparations comprising the same, as well as methods of making and using the same. In some embodiments, translation efficiency of an RNA encoding a payload, and/or expression of a payload encoded by an RNA, can be improved with an RNA polynucleotide comprising a 5′ cap having a structure disclosed herein; a 5′ UTR comprising a cap proximal sequence disclosed herein, and a sequence encoding a payload. In some embodiments, absence of a self-hybridizing sequence in an RNA polynucleotide encoding a payload can further improve translation efficiency of an RNA encoding a payload, and/or expression of a payload encoded by an RNA payload.

### RNA Polynucleotides

The term “polynucleotide” or “nucleic acid”, as used herein, refers to DNA and RNA such as genomic DNA, cDNA, mRNA, recombinantly produced and chemically synthesized molecules. A nucleic acid may be single-stranded or double-stranded. RNA includes synthetic RNA. In some embodiments, synthetic RNA is or comprises in vitro transcribed RNA (IVT RNA). According to the invention, a polynucleotide is preferably isolated.

In some embodiments, nucleic acids may be comprised in a vector. The term “vector” as used herein includes any vectors known to the skilled person including plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). In some embodiments, a vector may be an expression vector; alternatively or additionally, in some embodiments, a vector may be a cloning vector. Those skilled in the art will appreciate that, in some embodiments, an expression vector may be, for example, a plasmid; alternatively or additionally, in some embodiments, an expression vector may be a viral vector. Typically, an expression vector will contain a desired coding sequence and appropriate other sequences necessary for the expression of the operably linked coding sequence in a particular host organism (e.g., bacteria, yeast, plant, insect, or mammal) or in in vitro expression systems. Cloning vectors are generally used to engineer and amplify a certain desired fragment (typically a DNA fragment), and may lack functional sequences needed for expression of the desired fragment(s).

In some embodiments, a nucleic acid as described and/or utilized herein may be or comprise recombinant and/or isolated molecules.

Those skilled in the art, reading the present disclosure, will understand that the term “RNA” typically refers to a nucleic acid molecule which includes ribonucleotide residues. In some embodiments, an RNA contains all or a majority of ribonucleotide residues. As used herein, “ribonucleotide” refers to a nucleotide with a hydroxyl group at the 2-position of a 3-D-ribofuranosyl group. In some embodiments, an RNA may be partly or fully double stranded RNA; in some embodiments, an RNA may comprise two or more distinct nucleic acid strands (e.g., separate molecules) that are partly or fully hybridized with one another. In many embodiments, an RNA is a single strand, which may in some embodiments, self-hybridize or otherwise fold into secondary and/or tertiary structures. In some embodiments, an RNA as described and/or utilized herein does not self-hybridize, at least with respect to certain sequences as described herein. In some embodiments, an RNA may be an isolated RNA such as partially purified RNA, essentially pure RNA, synthetic RNA, recombinantly produced RNA, and/or a modified RNA (where the term “modified” is understood to indicate that one or more residues or other structural elements of the RNA differs from naturally occurring RNA; for example, in some embodiments, a modified RNA differs by the addition, deletion, substitution and/or alteration of one or more nucleotides and/or by one or more moieties or characteristics of a nucleotide—e.g., of a nucleoside or of a backbone structure or linkage). In some embodiments, a modification may be or comprise addition of non-nucleotide material to internal RNA nucleotides or to the end(s) of RNA. It is also contemplated herein that nucleotides in RNA (e.g., in a modified RNA) may be non-standard nucleotides, such as chemically synthesized nucleotides or deoxynucleotides. For the present disclosure, these altered RNAs are considered analogs of naturally-occurring RNA.

As appreciated by a person skilled in the art, the RNA polynucleotides disclosed herein can comprise or consist of naturally occurring ribonucleotides and/or modified ribonucleotides. Therefore, a person skilled in the art will understand references to A, U, G, or C throughout the specification described herein can refer to a naturally occurring ribonucleotide and/or a modified ribonucleotide described herein. For example, in some embodiments, a U is uridine. In some embodiments, a U is modified uridine (e.g., pseudouridine, 1-methyl pseudouridine).

In some embodiments of the present disclosure, an RNA is or comprises messenger RNA (mRNA) that relates to an RNA transcript which encodes a polypeptide.

In some embodiments, an RNA disclosed herein comprises: a 5′ cap disclosed herein; a 5′ untranslated region comprising a cap proximal sequence (5′-UTR), a sequence encoding a payload (e.g., a polypeptide); a 3′ untranslated region (3′-UTR); and/or a polyadenylate (PolyA) sequence.

In some embodiments, an RNA disclosed herein comprises the following components in 5′ to 3′ orientation: a 5′ cap disclosed herein; a 5′ untranslated region comprising a cap proximal sequence (5′-UTR), a sequence encoding a payload (e.g., a polypeptide); a 3′ untranslated region (3′-UTR); and a PolyA sequence.

In some embodiments, an RNA is produced by in vitro transcription or chemical synthesis. In some embodiments, an mRNA is produced by in vitro transcription using a DNA template where DNA refers to a nucleic acid that contains deoxyribonucleotides.

In some embodiments, an RNA disclosed herein is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In some embodiments, an RNA is “replicon RNA” or simply a “replicon”, in particular “self-replicating RNA” or “self-amplifying RNA”. In some embodiments, a replicon or self-replicating RNA is derived from or comprises elements derived from a ssRNA virus, in particular a positive-stranded ssRNA virus such as an alphavirus. Alphaviruses are typical representatives of positive-stranded RNA viruses. Alphaviruses replicate in the cytoplasm of infected cells (for review of the alphaviral life cycle see Jose et al., Future Microbiol., 2009, vol. 4, pp. 837-856). The total genome length of many alphaviruses typically ranges between 11,000 and 12,000 nucleotides, and the genomic RNA typically has a 5′-cap and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA polynucleotide that resembles eukaryotic messenger RNA (mRNA; Gould et al., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234). Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

In some embodiments, an RNA described herein may have modified nucleosides. In some embodiments, an RNA comprises a modified nucleoside in place of at least one (e.g., every) uridine.

The term “uracil,” as used herein, describes one of the nucleobases that can occur in the nucleic acid of RNA. The structure of uracil is:

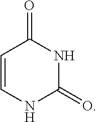

The term “uridine,” as used herein, describes one of the nucleosides that can occur in RNA. The structure of uridine is:

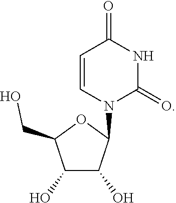 [SMILES: O=c1ccn([C@@H]2O[C@H](CO)[C@@H](O)[C@H]2O)c(=O)[nH]1]

UTP (uridine 5′-triphosphate) has the following structure:

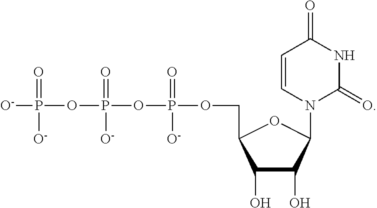

Pseudo-UTP (pseudouridine-5′-triphosphate) has the following structure:

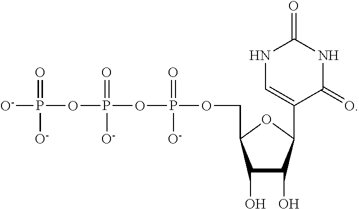

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methylpseudouridine (m1Ψ), which has the structure:

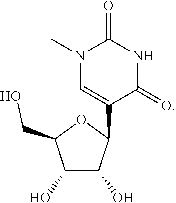 [SMILES: Cn1cc([C@@H]2O[C@H](CO)[C@@H](O)[C@H]2O)c(=O)[nH]c1=O]

N1-methylpseudouridine-5′-triphosphate (m1ΨTP) has the following structure:

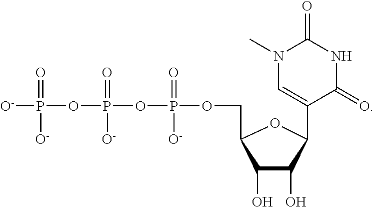

Another exemplary modified nucleoside is 5-methyluridine (m5U), which has the structure:

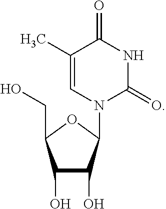

In some embodiments, one or more uridines in an RNA described herein is replaced by a modified nucleoside. In some embodiments, a modified nucleoside is a modified uridine. In some embodiments, an RNA comprises a modified nucleoside in place of at least one uridine. In some embodiments, an RNA comprises a modified nucleoside in place of each uridine.

In some embodiments, a modified nucleoside is independently selected from pseudouridine (Ψ), N1-methylpseudouridine (m1Ψ), and 5-methyluridine (m5U). In some embodiments, a modified nucleoside comprises pseudouridine (Ψ). In some embodiments, a modified nucleoside comprises N1-methyl-pseudouridine (m1Ψ). In some embodiments, a modified nucleoside comprises 5-methyluridine (m5U). In some embodiments, an RNA may comprise more than one type of modified nucleoside, and the modified nucleosides are independently selected from pseudouridine (Ψ), N1-methylpseudouridine (m1Ψ), and 5-methyluridine (m5U). In some embodiments, the modified nucleosides comprise pseudouridine (Ψ) and N1-methylpseudouridine (m1Ψ). In some embodiments, the modified nucleosides comprise pseudouridine (Ψ) and 5-methyluridine (m5U). In some embodiments, the modified nucleosides comprise N1-methylpseudouridine (m1Ψ) and 5-methyluridine (m5U). In some embodiments, the modified nucleosides comprise pseudouridine (Ψ), N1-methylpseudouridine (m1Ψ), and 5-methyluridine (m5U).

In some embodiments, a modified nucleoside replacing one or more, e.g., all, uridines in the RNA may be any one or more of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (rm⁵U), 1-taurinomethyl-pseudouridine, 5-taurinomethyl-2-thio-uridine(τm5s²U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N₁-methyl-pseudouridine, 3-(3-amino-3-carboxypropyl)uridine (acp³U), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In some embodiments, an RNA comprises other modified nucleosides or comprises further modified nucleosides, e.g., modified cytidine. For example, in some embodiments of an RNA, 5-methylcytidine is substituted partially or completely, preferably completely, for cytidine. In some embodiments, an RNA comprises 5-methylcytidine and one or more nucleosides selected from pseudouridine (ψ), N1-methyl-pseudouridine (m1ψ), and 5-methyl-uridine (m5U). In some embodiments, an RNA comprises 5-methylcytidine and N1-methyl-pseudouridine (m1ψ). In some embodiments, the RNA comprises 5-methylcytidine in place of each cytidine and N1-methyl-pseudouridine (m1ψ) in place of each uridine.

In some embodiments, an RNA encoding a payload, e.g., a vaccine antigen, is expressed in cells of a subject treated to provide a payload, e.g., vaccine antigen. In some embodiments, the RNA is transiently expressed in cells of the subject. In some embodiments, the RNA is in vitro transcribed RNA. In some embodiments, expression of a payload, e.g., a vaccine antigen is at the cell surface. In some embodiments, a payload, e.g., a vaccine antigen is expressed and presented in the context of MHC. In some embodiments, expression of a payload, e.g., a vaccine antigen is into the extracellular space, i.e., the vaccine antigen is secreted.

In the context of the present disclosure, the term “transcription” relates to a process, wherein the genetic code in a DNA sequence is transcribed into RNA. Subsequently, the RNA may be translated into peptide or protein.

According to the present invention, the term “transcription” comprises “in vitro transcription”, wherein the term “in vitro transcription” relates to a process wherein RNA, in particular mRNA, is in vitro synthesized in a cell-free system, preferably using appropriate cell extracts. Preferably, cloning vectors are applied for the generation of transcripts. These cloning vectors are generally designated as transcription vectors and are according to the present invention encompassed by the term “vector”. According to the present invention, the RNA used in the present invention preferably is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription according to the invention is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

With respect to RNA, the term “expression” or “translation” relates to the process in the ribosomes of a cell by which a strand of mRNA directs the assembly of a sequence of amino acids to make a peptide or protein.

In some embodiments, after administration of an RNA described herein, e.g., formulated as RNA lipid particles, at least a portion of the RNA is delivered to a target cell. In some embodiments, at least a portion of the RNA is delivered to the cytosol of the target cell. In some embodiments, the RNA is translated by the target cell to produce the peptide or protein it encodes. In some embodiments, the target cell is a spleen cell. In some embodiments, the target cell is an antigen presenting cell such as a professional antigen presenting cell in the spleen. In some embodiments, the target cell is a dendritic cell or macrophage. RNA particles such as RNA lipid particles described herein may be used for delivering RNA to such target cell. Accordingly, the present disclosure also relates to a method for delivering RNA to a target cell in a subject comprising the administration of the RNA particles described herein to the subject. In some embodiments, the RNA is delivered to the cytosol of the target cell. In some embodiments, the RNA is translated by the target cell to produce the peptide or protein encoded by the RNA. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

In some embodiments, nucleic acid compositions described herein, e.g., compositions comprising a lipid nanoparticle encapsulated mRNA are characterized by (e.g., when administered to a subject) sustained expression of an encoded polypeptide. For example, in some embodiments, such compositions are characterized in that, when administered to a human, they achieve detectable polypeptide expression in a biological sample (e.g., serum) from such human and, in some embodiments, such expression persists for a period of time that is at least 36 hours or longer, including, e.g., at least 48 hours, at least 60 hours, at least 72 hours, at least 96 hours, at least 120 hours, at least 148 hours, or longer.

In some embodiments, an RNA encoding a payload to be administered according to the present disclosure is non-immunogenic. RNA encoding immunostimulant may be administered according to the invention to provide an adjuvant effect. The RNA encoding immunostimulant may be standard RNA or non-immunogenic RNA.

The term “non-immunogenic RNA” as used herein refers to RNA that does not induce a response by the immune system upon administration, e.g., to a mammal, or induces a weaker response than would have been induced by the same RNA that differs only in that it has not been subjected to the modifications and treatments that render the immunogenic RNA non-immunogenic, i.e., than would have been induced by standard RNA (stdRNA). In one preferred embodiment, non-immunogenic RNA, which is also termed modified RNA (modRNA) herein, is rendered non-immunogenic by incorporating modified nucleosides suppressing RNA-mediated activation of innate immune receptors into the RNA and removing double-stranded RNA (dsRNA).

For rendering the immunogenic RNA non-immunogenic by the incorporation of modified nucleosides, any modified nucleoside may be used as long as it lowers or suppresses immunogenicity of the RNA. Particularly preferred are modified nucleosides that suppress RNA-mediated activation of innate immune receptors. In some embodiments, the modified nucleosides comprise a replacement of one or more uridines with a nucleoside comprising a modified nucleobase. In some embodiments, the modified nucleobase is a modified uracil. In some embodiments, the nucleoside comprising a modified nucleobase is selected from the group consisting of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(τm5s2U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N₁-methyl-pseudouridine, 3-(3-amino-3-carboxypropyl)uridine (acp³U), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (Wm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl)uridine, and 5-[3-(1-E-propenylamino)uridine. In one particularly preferred embodiment, the nucleoside comprising a modified nucleobase is pseudouridine (ψ), N₁-methyl-pseudouridine (m1ψ) or 5-methyl-uridine (m5U), in particular N₁-methyl-pseudouridine.

In some embodiments, the replacement of one or more uridines with a nucleoside comprising a modified nucleobase comprises a replacement of at least 1%, at least 2%, at least 3%, at least 4%, at least 5%, at least 10%, at least 25%, at least 50%, at least 75%, at least 90%, at least 95%, at least 96%, at least 97%, at least 98%, at least 99% or 100% of the uridines. During synthesis of mRNA by in vitro transcription (IVT) using T7 RNA polymerase significant amounts of aberrant products, including double-stranded RNA (dsRNA) are produced due to unconventional activity of the enzyme. dsRNA induces inflammatory cytokines and activates effector enzymes leading to protein synthesis inhibition. dsRNA can be removed from RNA such as IVT RNA, for example, by ion-pair reversed phase HPLC using a non-porous or porous C-18 polystyrene-divinylbenzene (PS-DVB) matrix. Alternatively, an enzymatic based method using E. coli RNaseIII that specifically hydrolyzes dsRNA but not ssRNA, thereby eliminating dsRNA contaminants from IVT RNA preparations can be used. Furthermore, dsRNA can be separated from ssRNA by using a cellulose material. In some embodiments, an RNA preparation is contacted with a cellulose material and the ssRNA is separated from the cellulose material under conditions which allow binding of dsRNA to the cellulose material and do not allow binding of ssRNA to the cellulose material.

As the term is used herein, “remove” or “removal” refers to the characteristic of a population of first substances, such as non-immunogenic RNA, being separated from the proximity of a population of second substances, such as dsRNA, wherein the population of first substances is not necessarily devoid of the second substance, and the population of second substances is not necessarily devoid of the first substance. However, a population of first substances characterized by the removal of a population of second substances has a measurably lower content of second substances as compared to the non-separated mixture of first and second substances.

In some embodiments, the removal of dsRNA from non-immunogenic RNA comprises a removal of dsRNA such that less than 10%, less than 5%, less than 4%, less than 3%, less than 2%, less than 1%, less than 0.5%, less than 0.3%, or less than 0.1% of the RNA in the non-immunogenic RNA composition is dsRNA. In some embodiments, the non-immunogenic RNA is free or essentially free of dsRNA. In some embodiments, the non-immunogenic RNA composition comprises a purified preparation of single-stranded nucleoside modified RNA. For example, in some embodiments, the purified preparation of single-stranded nucleoside modified RNA is substantially free of double stranded RNA (dsRNA). In some embodiments, the purified preparation is at least 90%, at least 91%, at least 92%, at least 93%, at least 94%, at least 95%, at least 96%, at least 97%, at least 98%, at least 99%, at least 99.5%, or at least 99.9% single stranded nucleoside modified RNA, relative to all other nucleic acid molecules (DNA, dsRNA, etc.).

In some embodiments, the non-immunogenic RNA is translated in a cell more efficiently than standard RNA with the same sequence. In some embodiments, translation is enhanced by a factor of 2-fold relative to its unmodified counterpart. In some embodiments, translation is enhanced by a 3-fold factor. In some embodiments, translation is enhanced by a 4-fold factor. In some embodiments, translation is enhanced by a 5-fold factor. In some embodiments, translation is enhanced by a 6-fold factor. In some embodiments, translation is enhanced by a 7-fold factor. In some embodiments, translation is enhanced by an 8-fold factor. In some embodiments, translation is enhanced by a 9-fold factor. In some embodiments, translation is enhanced by a 10-fold factor. In some embodiments, translation is enhanced by a 15-fold factor. In some embodiments, translation is enhanced by a 20-fold factor. In some embodiments, translation is enhanced by a 50-fold factor. In some embodiments, translation is enhanced by a 100-fold factor. In some embodiments, translation is enhanced by a 200-fold factor. In some embodiments, translation is enhanced by a 500-fold factor. In some embodiments, translation is enhanced by a 1000-fold factor. In some embodiments, translation is enhanced by a 2000-fold factor. In some embodiments, the factor is 10-1000-fold. In some embodiments, the factor is 10-100-fold. In some embodiments, the factor is 10-200-fold. In some embodiments, the factor is 10-300-fold. In some embodiments, the factor is 10-500-fold. In some embodiments, the factor is 20-1000-fold. In some embodiments, the factor is 30-1000-fold. In some embodiments, the factor is 50-1000-fold. In some embodiments, the factor is 100-1000-fold. In some embodiments, the factor is 200-1000-fold. In some embodiments, translation is enhanced by any other significant amount or range of amounts.

In some embodiments, the non-immunogenic RNA exhibits significantly less innate immunogenicity than standard RNA with the same sequence. In some embodiments, the non-immunogenic RNA exhibits an innate immune response that is 2-fold less than its unmodified counterpart. In some embodiments, innate immunogenicity is reduced by a 3-fold factor. In some embodiments, innate immunogenicity is reduced by a 4-fold factor. In some embodiments, innate immunogenicity is reduced by a 5-fold factor. In some embodiments, innate immunogenicity is reduced by a 6-fold factor. In some embodiments, innate immunogenicity is reduced by a 7-fold factor. In some embodiments, innate immunogenicity is reduced by a 8-fold factor. In some embodiments, innate immunogenicity is reduced by a 9-fold factor. In some embodiments, innate immunogenicity is reduced by a 10-fold factor. In some embodiments, innate immunogenicity is reduced by a 15-fold factor. In some embodiments, innate immunogenicity is reduced by a 20-fold factor. In some embodiments, innate immunogenicity is reduced by a 50-fold factor. In some embodiments, innate immunogenicity is reduced by a 100-fold factor. In some embodiments, innate immunogenicity is reduced by a 200-fold factor. In some embodiments, innate immunogenicity is reduced by a 500-fold factor. In some embodiments, innate immunogenicity is reduced by a 1000-fold factor. In some embodiments, innate immunogenicity is reduced by a 2000-fold factor.

The term “exhibits significantly less innate immunogenicity” refers to a detectable decrease in innate immunogenicity. In some embodiments, the term refers to a decrease such that an effective amount of the non-immunogenic RNA can be administered without triggering a detectable innate immune response. In some embodiments, the term refers to a decrease such that the non-immunogenic RNA can be repeatedly administered without eliciting an innate immune response sufficient to detectably reduce production of the protein encoded by the non-immunogenic RNA. In some embodiments, the decrease is such that the non-immunogenic RNA can be repeatedly administered without eliciting an innate immune response sufficient to eliminate detectable production of the protein encoded by the non-immunogenic RNA. “Immunogenicity” is the ability of a foreign substance, such as RNA, to provoke an immune response in the body of a human or other animal. The innate immune system is the component of the immune system that is relatively unspecific and immediate. It is one of two main components of the vertebrate immune system, along with the adaptive immune system.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence.

As used herein, the terms “linked,” “fused”, or “fusion” are used interchangeably. These terms refer to the joining together of two or more elements or components or domains.

In some embodiments, the present disclosure provides an RNA polynucleotide comprising:
a 5′ cap; a cap proximal sequence comprising positions +1, +2, +3, +4, and +5 of the RNA polynucleotide; and a sequence encoding a payload, wherein:
(i) the 5′ cap is a trinucleotide cap structure comprises N₁pN₂, wherein N₁ is position +1 of the RNA polynucleotide and N₂ is position +2 of the RNA polynucleotide, and
   wherein
      N₁ is A or an analog thereof; and
      N₂ is U or an analog thereof; and
   (ii) the cap proximal sequence comprises:
      N₁ and N₂ of the trinucleotide cap structure and a sequence comprising N₃N₄N₅ at positions +3, +4, and +5 respectively of the RNA polynucleotide, wherein N₃, N₄, and N₅ are each independently selected from: A, C, G, and U.

### Codon Optimization

In some embodiments, a payload (e.g., a polypeptide) described herein is encoded by a coding sequence which is codon-optimized and/or the G/C content of which is increased compared to wild type coding sequence. In some embodiments, one or more sequence regions of the coding sequence are codon-optimized and/or increased in the G/C content compared to the corresponding sequence regions of the wild type coding sequence. In some embodiments, codon-optimization and/or increased the G/C content does not change the sequence of the encoded amino acid sequence.

The term “codon-optimized” is understood by those in the art to refer to alteration of codons in the coding region of a nucleic acid molecule to reflect the typical codon usage of a host organism without preferably altering the amino acid sequence encoded by the nucleic acid molecule. Within the context of the present disclosure, coding regions are preferably codon-optimized for optimal expression in a subject to be treated using an RNA polynucleotide described herein. Codon-optimization is based on the finding that the translation efficiency is also determined by a different frequency in the occurrence of tRNAs in cells. Thus, the sequence of RNA may be modified such that codons for which frequently occurring tRNAs are available are inserted in place of “rare codons”.

In some embodiments, guanosine/cytidine (G/C) content of a coding region (e.g., of a payload sequence) of an RNA is increased compared to the G/C content of the corresponding coding sequence of a wild type RNA encoding the payload, wherein the amino acid sequence encoded by the RNA is preferably not modified compared to the amino acid sequence encoded by the wild type RNA. This modification of the RNA sequence is based on the fact that the sequence of any RNA region to be translated is important for efficient translation of that mRNA. Sequences having an increased G (guanosine)/C (cytidine) content are more stable than sequences having an increased A (adenosine)/U (uridine) content. In respect to the fact that several codons code for one and the same amino acid (so-called degeneration of the genetic code), the most favourable codons for the stability can be determined (so-called alternative codon usage). Depending on the amino acid to be encoded by the RNA, there are various possibilities for modification of the RNA sequence, compared to its wild type sequence. In particular, codons which contain A and/or U nucleosides can be modified by substituting these codons by other codons, which code for the same amino acids but contain no A and/or U or contain a lower content of A and/or U nucleosides.

In some embodiments, G/C content of a coding region of an RNA described herein is increased by at least 10%, at least 20%, at least 30%, at least 40%, at least 50%, at least 55%, or even more compared to the G/C content of a coding region of a wild type RNA.

### 5′ Cap

A structural feature of mRNAs is a cap structure at the five-prime (5′) terminus. Natural eukaryotic mRNA comprises a 7-methylguanosine cap linked to the mRNA via a 5′ to 5′-triphosphate bridge resulting in a cap0 structure (m7GpppN). In most eukaryotic mRNA and some viral mRNA, further modifications can occur at the 2′-hydroxy-group (2′-OH) (e.g., the 2′-hydroxyl group may be methylated to form 2′-O-Me) of the first and subsequent nucleotides producing “cap1” and “cap2” five-prime ends, respectively. Diamond, et al., (2014) Cytokine & growth Factor Reviews, 25:543-550 reported that cap0-mRNA cannot be translated as efficiently as cap1-mRNA in which the role of 2′-O-Me in the penultimate position at the mRNA 5′ end is determinant. Lack of the 2′-O-Me has been shown to trigger innate immunity and activate IFN response. Daffis, et al. (2010) Nature, 468:452-456; and Zust et al. (2011) Nature Immunology, 12:137-143.

RNA capping is well researched and is described, e.g., in Decroly E et al. (2012) Nature Reviews 10: 51-65; and in Ramanathan A. et al., (2016) Nucleic Acids Res; 44(16): 7511-7526, the entire contents of each of which is hereby incorporated by reference. In some embodiments, to imitate the 5′ cap structure of natural mRNA, in vitro-transcribed mRNA (IVT mRNA) can be capped either post-transcriptionally using recombinant Vaccinia virus-derived enzymes (see., e.g., Kyrieleis, et al. (1993) Structure 22:452-465; and Corbett, et al. (2020) The New England Journal of Medicine 383:1544-1555) or co-transcriptionally by adding cap analogs immediately into the in vitro transcription reaction (see, e.g., Jemielity, et al. (2003) RNA 9:1108-1122; and Kocmik, et al. (2018) Cell Cycle 17:1624-1636). In some embodiments, enzymatic capping can yield cap1-mRNA, but can be time-consuming since it requires an extra purification step and demands a heating step to improve the accessibility of structured 5′ends, thereby further increasing the risk of RNA degradation. Among other things, cotranscriptional capping can be highly reproducible and less expensive than enzymatic capping. mRNA generated in the presence of cap analogs can be resistant to the human decapping enzymes (see, e.g., Kowalska et al. (2008) RNA 14:1119-1131) and/or interferon-induced proteins with tetratricopeptide repeats (IFITs) which inhibits cap0-dependent translation (see, e.g., Diamond et al. (2014) Cytokine & Growth Factor Reviews 25:543-550; and Miedziak, et al. (2019) RNA 26:58-68). However, in cotranscriptional capping, GTP is typically competing with cap analogs during transcription, which can lead to poor capping efficiency and result inweak translational capacity. Certain cap1 structures can be incorporated into IVT mRNA in the right orientation for producing cap1-mRNA with high capping efficiency in a rapid co-transcriptional reaction. See, e.g., Henderson et al., (2021) Current Protocols 1:e39. For example, a trinucleotide cap1 structure comprising an AG initiator can reduce the slippage of RNA polymerases on a DNA template strand (e.g., as compared to a DNA template containing a G triplet as a transcriptional start site). See, e.g., Imburgio, et al. (2000) Biochemistry 39:10419-10430.

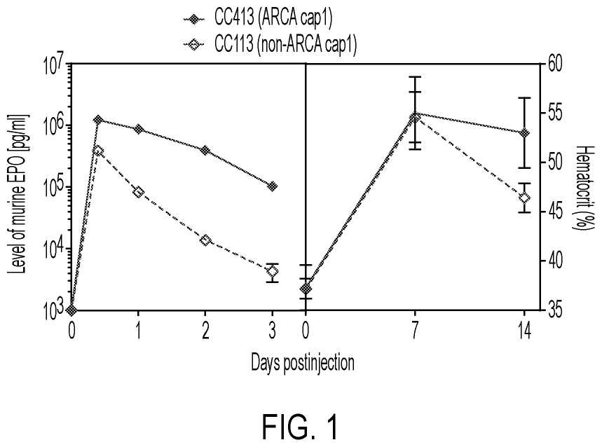

In some embodiments, a 5′ cap includes a Cap-0 structure (also referred herein as “Cap0”), a Cap-1 structure (also referred herein as “Cap1”), or a Cap-2 structure (also referred herein as “Cap2”). See, e.g., FIG. 1 of Ramanathan A et al., and FIG. 1 of Decroly E et al.

The term “5′-cap” as used herein refers to a structure found on the 5′-end of an RNA, e.g., mRNA, and generally includes a guanosine nucleotide connected to an RNA, e.g., mRNA, via a 5′- to 5′-triphosphate linkage (also referred to as Gppp or G(5′)ppp(5′)). In some embodiments, a guanosine nucleoside included in a 5′ cap may be modified, for example, by methylation at one or more positions (e.g., at the 7-position) on a base (guanine), and/or by methylation at one or more positions of a ribose. In some embodiments, a guanosine nucleoside included in a 5′ cap comprises a 3′O methylation at a ribose (denoted as “(m^{3′-O})G” or “3′OMeG”). In some embodiments, a guanosine nucleoside included in a 5′ cap comprises methylation at the 7-position of guanine (denoted as “(m⁷)G” or “m7G”). In some embodiments, a guanosine nucleoside included in a 5′ cap comprises methylation at the 7-position of guanine and a 3′ O methylation at a ribose (denoted as “(m₂^{7,3′-O})G” or “m7(3′OMeG)”). In some embodiments, a guanosine nucleoside included in a 5′ cap comprises a 2′ O methylation at a ribose (denoted as “(m^{2′-O})G” or “2′OMeG”). In some embodiments, a guanosine nucleoside included in a 5′ cap comprises methylation at the 7-position of guanine and a 2′ O methylation at a ribose (denoted as “(m₂^{7,2′-O})G” or “m7(2′OMeG)”). It will be understood that the notation used in the above paragraph, e.g., “(m₂^{7,3′-O})G” or “m7(3′OMeG)”, applies to other structures described herein.

In some embodiments, providing an RNA with a 5′-cap disclosed herein or a 5′-cap analog may be achieved by in vitro transcription, in which a 5′-cap is co-transcriptionally incorporated into an RNA strand. In some embodiments, a 5′ cap may be attached to an RNA post-transcriptionally using capping enzymes. In some embodiments, co-transcriptional capping with a cap disclosed herein, e.g., a cap0, cap1, or cap2 structure, improves the capping efficiency of an RNA compared to co-transcriptional capping with an appropriate reference comparator. In some embodiments, improving capping efficiency can increase a translation efficiency and/or translation rate of an RNA, and/or increase expression of an encoded polypeptide.

In some embodiments, an RNA described herein comprises a 5′-cap or a 5′ cap analog, e.g., a 5′-cap comprising a Cap0, a Cap1 or a Cap2 structure. In some embodiments, a provided RNA does not have uncapped 5′-triphosphates. In some embodiments, an RNA may be capped with a 5′-cap analog. In some embodiments, an RNA described herein comprises a Cap0 structure. In some embodiments, an RNA described herein comprises a Cap1 structure, e.g., as described herein. In some embodiments, an RNA described herein comprises a Cap2 structure.

In some embodiments, a Cap0 structure comprises a guanosine nucleoside methylated at the 7-position of guanine (m7G). In some embodiments, a Cap0 structure is connected to an RNA via a 5′- to 5′-triphosphate linkage and is also referred to herein as m7Gppp or m7G(5′)ppp(5′).

In some embodiments, a Cap1 structure comprises a guanosine nucleoside methylated at the 7-position of guanine (m7G) and a 2′O methylated first nucleotide in an RNA (2′OMeN₁). In some embodiments, a Cap1 structure is connected to an RNA via a 5′- to 5′-triphosphate linkage and is also referred to herein as m7Gppp(2′OMeN₁) or m7G(5′)ppp(5′)(2′OMeN₁), wherein N₁ is as defined and described herein. In some embodiments, a m7G(5′)ppp(5′)(2′OMeN₁) Cap1 structure comprises a second nucleotide, N₂ which is a cap proximal nucleotide at position 2 (m7G(5′)ppp(5′)(2′OMeN₁)N₂) wherein each of N₁ and N₂ is as defined and described herein.

In some embodiments, the 5′ cap is a trinucleotide cap structure. In some embodiments, the 5′ cap is a trinucleotide cap structure comprising N₁pN₂, wherein N₁ and N₂ are as defined and described herein. In some embodiments, the 5′ cap is a trinucleotide cap G*N₁pN₂, wherein N₁ and N₂ are as defined above and herein, and: G* comprises a structure of formula (I):

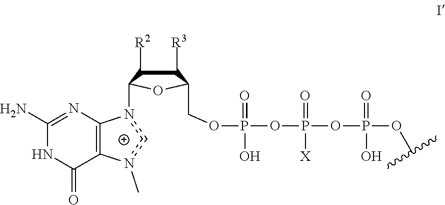

or a salt thereof,
wherein
each R² and R³ is —OH or —OCH₃; and
X is OH or SH.

It will be understood that each nucleotide, e.g., N₁ and N₂ are linked via a phosphate group “p” (e.g., —P(═O)(OH)—, or a salt thereof such as —P(═O)(O⁻)—.

In some embodiments, R² is —OH. In some embodiments, R² is —OCH₃. In some embodiments, R³ is —OH. In some embodiments, R³ is —OCH₃. In some embodiments, R² is —OH and R³ is —OH. In some embodiments, R² is —OH and R³ is —CH₃. In some embodiments, R² is —CH₃ and R³ is —OH. In some embodiments, R² is —CH₃ and R³ is —CH₃. In some embodiments, R² is —OH and R³ is —OCH₃. In some embodiments, R² is —OCH₃ and R³ is —OH. In some embodiments, R² is —OCH₃ and R³ is —OCH₃.

It will be understood, that X being OH or SH includes salts thereof, e.g., O⁻ or S⁻. In some embodiments, X is OH. In some embodiments, X is SH. In some embodiments, X is O⁻. In some embodiments, X is S⁻.

In some embodiments, the 5′ cap is a trinucleotide Cap0 structure (e.g. (m⁷)GpppN₁pN₂, (m₂^{7,2′-O})GpppN₁pN₂, or (m₂^{7,3′-O})GpppN₁pN₂, wherein N₁ and N₂ are as defined and described herein). In some embodiments, the 5′ cap is a trinucleotide Cap1 structure (e.g., (m⁷)Gppp(m^{2′-O})N₁pN₂, (m₂^{7,2′-O})Gppp(m^{2′-O})N₁pN₂, (m₂^{7,3′-O})Gppp(m^{2′-O})N₁pN₂, wherein N₁ and N₂ are as defined and described herein. In some embodiments, the 5′ cap is a trinucleotide Cap2 structure (e.g., (m⁷)Gppp(m^{2′-O})N₁p(m^{2′-O})N₂, (m₂^{7,2′-O})Gppp(m^{2′-O})N₁p(m^{2′-O})N₂, (m₂^{7,3′-O})Gppp(m^{2′-O})N₁p(m^{2′-O})N₂, wherein N₁ and N₂ are as defined and described herein.

In some embodiments, N₁ is A or an analog thereof. In some embodiments, N₁ is adenosine. In some embodiments, N₁ is 6-methyladenosine. In some embodiments, N₁ is:

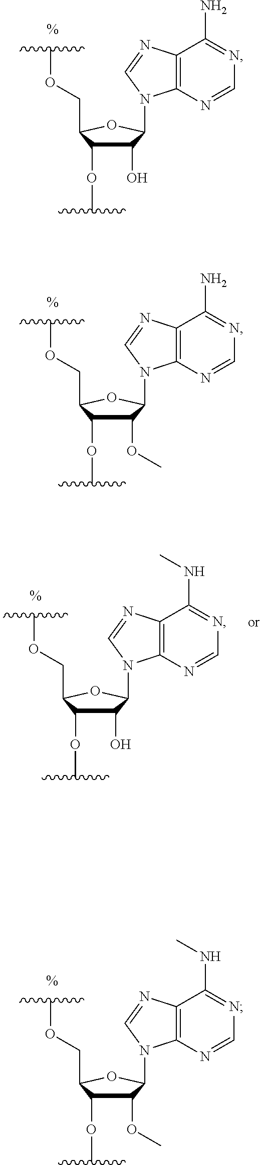

wherein % represents the point of attachment to G*.

In some embodiments, N₂ is U or an analog thereof. In some embodiments, N₂ is a modified U. In some embodiments, N₂ is 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (rm⁵U), 1-taurinomethyl-pseudouridine, 5-taurinomethyl-2-thio-uridine(τm5s2U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N₁-methyl-pseudouridine, 3-(3-amino-3-carboxypropyl)uridine (acp³U), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art. In some embodiments, N₂ is 5-methyluridine (m⁵U). In some embodiments, N₂ is 1-methyl-pseudouridine (m¹ψ). In some embodiments, N₂ is pseudouridine (ψ). In some embodiments, N₂ is 1-(2,2,2-trifluoroethyl)pseudouridine (tfet¹ψ). In some embodiments, N₂ is 1-propargylpseudouridine (ppg)¹ψ). In some embodiments, N₂ is 1-benzylpseudouridine (bn¹ψ). In some embodiments, N₂ is 1-(cyclopropylmethyl)pseudouridine (cpm¹ψ). In some embodiments, N₂ is 1-(pyridin-4-ylmethyl)pseudouridine ((4-pm)¹ψ).

In some embodiments, N₂ is of formula II:

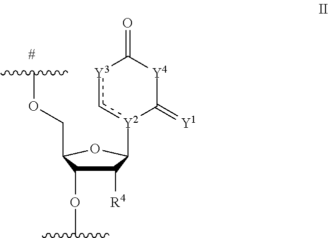

or a salt thereof, wherein:
each is independently a single or double bond, as allowed by valency;
Y¹ is O or S;
Y² is N, C, or CH;
Y³ is N, NR^{a1}, CR^{a1}, or CHR^{a1};
Y⁴ is NR^{a2} or CHR^{a2};
each of R^{a1} or R^{a2} is independently hydrogen or C₁₋₆ aliphatic;
R⁴ is —OH or —OMe; and
#represents the point of attachment to p of N₁p.

In some embodiments, Y¹ is O. In some embodiments, Y¹ is S.

In some embodiments, Y² is N. In some embodiments, Y² is C or CH. In some embodiments, Y² is C. In some embodiments, Y² is CH.

In some embodiments, Y³ is N or CR^{a1}. In some embodiments, Y³ is N. In some embodiments, Y³ is CR^{a1}. In some embodiments, Y³ is CH or C(CH₃). In some embodiments, Y³ is CH. In some embodiments, Y³ is C(CH₃). In some embodiments, Y³ is NR^{a1} or CHR^{a1}. In some embodiments, Y³ is NH or N(CH₃). In some embodiments, Y³ is NH. In some embodiments, Y³ is N(CH₃). In some embodiments, Y³ is CH₂ or CH(CH₃). In some embodiments, Y³ is CH₂. In some embodiments, Y³ is CH(CH₃).

In some embodiments, Y⁴ is NR^{a2}. In some embodiments, Y⁴ is NH or NCH₃. In some embodiments, Y⁴ is NH. In some embodiments, Y⁴ is NCH₃. In some embodiments, Y⁴ is CHR^{a2}. In some embodiments, Y⁴ is CH₂ or CH(CH₃). In some embodiments, Y⁴ is CH₂. In some embodiments, Y⁴ is CH(CH₃).

In some embodiments, R^{a1} is hydrogen. In some embodiments, R^{a1} is C₁₋₆ aliphatic. In some embodiments, R^{a1} is methyl, ethyl, n-propyl, or isopropyl. In some embodiments, R^{a1} is methyl.

In some embodiments, R^{a2} is hydrogen. In some embodiments, R^{a2} is C₁₋₆ aliphatic. In some embodiments, R^{a2} is methyl, ethyl, n-propyl, or isopropyl. In some embodiments, R^{a2} is methyl.

In some embodiments, R⁴ is —OH. In some embodiments, R⁴ is —OMe.

In some embodiments, N₂ is of formula IIa:

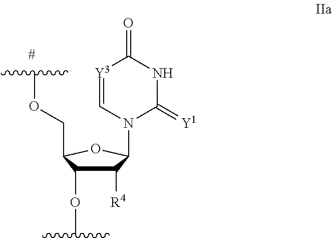

or a salt thereof, wherein each of Y¹, Y³, R⁴, and # is as defined above and described herein.

In some embodiments of formula IIa, Y¹ is O. In some embodiments of formula IIa, Y³ is CR^{a1}. In some such embodiments, R^{a1} is hydrogen, C₁₋₆ aliphatic or —O(C₁₋₄ alkyl). In some embodiments of formula IIa, R^{a1} is hydrogen, C₁₋₃ aliphatic or —O(C₁₋₂ alkyl). In some embodiments of formula IIa, R^{a1} is —CH₃ or —OCH₃.

In some embodiments, N₂ is of formula IIb:

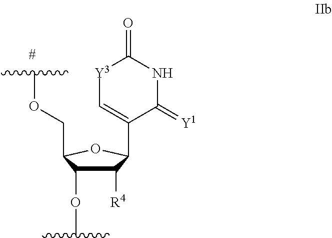

or a salt thereof, wherein each of Y¹, Y³, R⁴, and # is as defined above and described herein.

In some embodiments of formula IIb, Y³ is CR^{a1}. In some embodiments of formula IIb, R^{a1} is hydrogen. In some embodiments, R^{a1} is C₁₋₆ aliphatic. In some embodiments of formula IIb, R^{a1} is C₁₋₃ aliphatic. In some embodiments of formula IIb, R^{a1} is —CH₃. In some embodiments of formula IIb, R^{a1} is —CH₂R. In some such embodiments, R is C₁₋₄ aliphatic substituted with halogen. In some embodiments of formula IIb, R^{a1} is —CH₂R, wherein R is C₁₋₂ aliphatic substituted with halogen. In some embodiments of formula IIb, R^{a1} is —CH₂R, wherein R is —CF₃. In some embodiments of formula IIb, R^{a1} is —CH₂R, wherein R is phenyl. In some embodiments of formula IIb, R^{a1} is —CH₂R, wherein R is a 3- to 6-membered saturated carbocyclic ring. In some embodiments of formula IIb, R^{a1} is —CH₂R, wherein R is a 3- to 4-membered saturated carbocyclic ring. In some embodiments of formula IIb, R^{a1} is —CH₂R, wherein R is a 3-membered saturated carbocyclic ring. In some embodiments of formula IIb, R^{a1} is —CH₂R, wherein R is a 5- to 6-membered heteroaryl ring having 1-3 heteroatoms independently selected from nitrogen, oxygen, and sulfur. In some embodiments of formula IIb, R^{a1} is —CH₂R, wherein R is a 6-membered heteroaryl ring having 1-3 nitrogen atoms. In some embodiments of formula IIb, R^{a1} is —CH₂R, wherein R is a 6-membered heteroaryl ring having 1 nitrogen atom.

In some embodiments, N₂ is of formula II″:

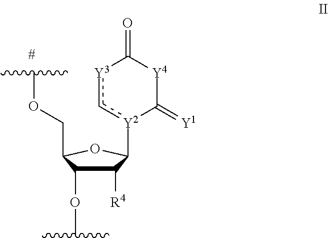

or a salt thereof, wherein:
each [symbol] is independently a single or double bond, as allowed by valency;
Y¹ is O or S;
Y² is N, C, or CH;
Y³ is N, NR^{a1}, CR^{a1}, or CHR^{a1};
Y⁴ is NR^{a2} or CHR^{a2};
each of R¹ or R^{a2} is independently hydrogen, C₁₋₆ aliphatic, —CH₂R, or —O(C₁₋₄ alkyl);
R is C₁₋₄ aliphatic substituted with halogen, phenyl, a 3- to 6-membered saturated carbocyclic ring, or a 5- to 6-membered heteroaryl ring having 1-3 heteroatoms independently selected from nitrogen, oxygen, and sulfur;
R⁴ is —OH or —OMe; and
#represents the point of attachment to p of N₁p.

In some embodiments of formula II″, Y¹ is O. In some embodiments of formula II″, Y¹ is S.

In some embodiments of formula II″, Y² is N. In some embodiments of formula II″, Y² is C or CH. In some embodiments of formula I″, Y² is C. In some embodiments of formula II″, Y² is CH.

In some embodiments of formula II″, Y³ is N or CR^{a1}. In some embodiments of formula II″, Y³ is N. In some embodiments of formula II″, Y³ is CR^{a1}. In some embodiments of formula II″, Y³ is NR^{a1} or CHR^{a1}. In some embodiments of formula II″, Y³ is NR^{a1}. In some embodiments of formula II″, Y³ is CHR^{a1}.

In some embodiments of formula II″, Y⁴ is NR^{a2}. In some embodiments of formula II″, Y⁴ is CHR^{a2}.

In some embodiments of formula II″, R^{a1} is hydrogen. In some embodiments of formula II″, R^{a1} is C₁₋₆ aliphatic. In some embodiments of formula II″, R^{a1} is C₁₋₃ aliphatic. In some embodiments of formula II″, R^{a1} is methyl, ethyl, n-propyl, or isopropyl. In some embodiments of formula II″, R^{a1} is methyl. In some embodiments of formula II″, R^{a1} is ethyl. In some embodiments of formula II″, R^{a1} is —CH═CH₂. In some embodiments of formula II″, R^{a1} is n-propyl. In some embodiments of formula II″, R^{a1} is isopropyl. In some embodiments of formula II″, R^{a1} is —CH₂C≡CH. In some embodiments of formula II″, R^{a1} is —CH₂CH═CH₂. In some embodiments of formula II″, R^{a1} is —CH₂R. In some embodiments of formula II″, R^{a1} is —O(C₁₋₄ alkyl). In some embodiments of formula II″, R^{a1} is —OMe.

In some embodiments of formula II″, R^{a2} is hydrogen. In some embodiments of formula II″, R^{a2} is C₁₋₆ aliphatic. In some embodiments of formula II″, R^{a2} is C₁₋₃ aliphatic. In some embodiments of formula II″, R^{a2} is methyl, ethyl, n-propyl, or isopropyl. In some embodiments of formula II″, R^{a2} is methyl. In some embodiments of formula II″, R^{a2} is ethyl. In some embodiments of formula II″, R^{a2} is —CH═CH₂. In some embodiments of formula II″, R^{a2} is n-propyl. In some embodiments of formula II″, R^{a2} is isopropyl. In some embodiments of formula II″, R^{a2} is —CH₂C≡CH. In some embodiments of formula II″, R^{a2} is —CH₂CH═CH₂. In some embodiments of formula II″, R^{a2} is —CH₂R. In some embodiments of formula II″, R^{a2} is —O(C₁₋₄ alkyl). In some embodiments of formula II″, R^{a2} is —OMe.

In some embodiments of formula II″, R is C₁₋₄ aliphatic substituted with halogen. In some embodiments of formula II″, R is C₁₋₂ aliphatic substituted with halogen. In some embodiments of formula II″, R is —CF₃. Accordingly, in some embodiments of formula II″, R^{a1} or R^{a2} is —CH₂CF₃.

In some embodiments of formula II″, R is phenyl. Accordingly, in some embodiments of formula II″, R^{a1} or R^{a2} is benzyl

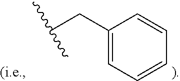 [SMILES: *Cc1ccccc1]

In some embodiments of formula II″, R is a 3- to 6-membered saturated carbocyclic ring. In some embodiments of formula II″, R is a 3- to 4-membered saturated carbocyclic ring. In some embodiments of formula II″, R is a 3-membered saturated carbocyclic ring. Accordingly, in some embodiments of formula II″, R^{a1} or R^{a2} is

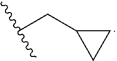 [SMILES: *CC1CC1]

In some embodiments of formula II″, R is a 5- to 6-membered heteroaryl ring having 1-3 heteroatoms independently selected from nitrogen, oxygen, and sulfur. In some embodiments of formula II″, R is a 6-membered heteroaryl ring having 1-3 nitrogen atoms. In some embodiments of formula II″, R is a 6-membered heteroaryl ring having 1-2 nitrogen atoms. In some embodiments of formula II″, R is a 6-membered heteroaryl ring having 1 nitrogen atom. In some embodiments of formula II″, R is 4-pyridyl. Accordingly, in some embodiments of formula II″, R^{a1} or R^{a2} is

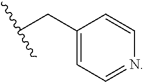 [SMILES: *Cc1ccncc1]

In some embodiments of formula II″, R⁴ is —OH. In some embodiments of formula II″, R⁴ is —OMe.

In some embodiments, N₂ is of formula IIa″

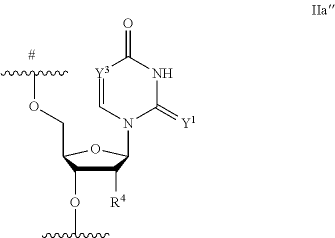

or a salt thereof, wherein each of Y¹, Y³, R⁴, and # is as defined above and described herein for formula II″.

In some embodiments, N₂ is of formula IIb″:

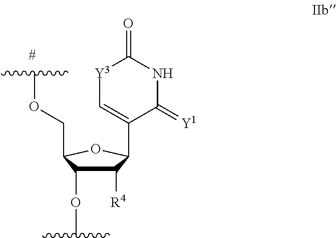

or a salt thereof, wherein each of Y¹, Y³, R⁴, and # is as defined above and described herein for formula II″.

In some embodiments, N₂ is of formula II′″:

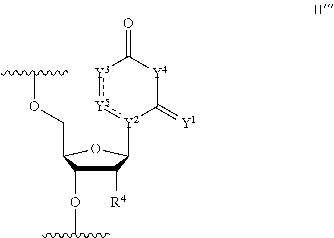

or a salt thereof, wherein:
each [symbol] is independently a single or double bond, as allowed by valency;
Y¹ is 0 or S;
Y² is N, C, or CH;
Y³ is N, NR^{a1}, CR^{a1}, or CHR^{a1};
Y⁴ is NR^{a2} or CHR^{a2};
Y⁵ is CR^{a3};
each of R^{a1}, R^{a2} or R^{a3} is independently hydrogen, C₁₋₆ aliphatic, —CH₂R, or —O(C₁₋₄ alkyl);
R is C₁₋₄ aliphatic substituted with halogen, phenyl, a 3- to 6-membered saturated carbocyclic ring, or a 5- to 6-membered heteroaryl ring having 1-3 heteroatoms independently selected from nitrogen, oxygen, and sulfur;
R⁴ is —OH or —OMe; and
#represents the point of attachment to p of N₁p.

In some embodiments of formula II′″, Y¹ is O. In some embodiments of formula II′″, Y¹ is S.

In some embodiments of formula II′″, Y² is N. In some embodiments of formula II′″, Y² is C or CH. In some embodiments of formula II′″, Y² is C. In some embodiments of formula II′″, Y² is CH.

In some embodiments of formula II′″, Y³ is N or CR^{a1}. In some embodiments of formula II′″, Y³ is N. In some embodiments of formula II′″, Y³ is CR^{a1}. In some embodiments of formula II′″, Y³ is NR^{a1} or CHR^{a1}. In some embodiments of formula II′″, Y³ is NR^{a1}. In some embodiments of formula II′″, Y³ is CHR^{a1} In some embodiments of formula II′″, Y⁴ is NR^{a2}. In some embodiments of formula II′″, Y⁴ is CHR².

In some embodiments of formula II′″, R^{a1} is hydrogen. In some embodiments of formula II′″, R^{a1} is C₁₋₆ aliphatic, —CH₂R, or —O(C₁₋₄ alkyl). In some embodiments of formula II′″, R^{a1} is C₁₋₆ aliphatic. In some embodiments of formula II′″, R^{a1} is C₁₋₃ aliphatic. In some embodiments of formula II′″, R^{a1} is methyl, ethyl, n-propyl, or isopropyl. In some embodiments of formula II′″, R^{a1} is methyl. In some embodiments of formula II′″, R^{a1} is ethyl. In some embodiments of formula II′″, R^{a1} is —CH═CH₂. In some embodiments of formula II′″, R^{a1} is n-propyl. In some embodiments of formula II′″, R^{a1} is isopropyl. In some embodiments of formula II′″, R^{a1} is —CH₂C≡CH. In some embodiments of formula II′″, R^{a1} is —CH₂CH═CH₂. In some embodiments of formula II′″, R^{a1} is —CH₂R. In some embodiments of formula II′″, R^{a1} is —O(C₁₋₄ alkyl). In some embodiments of formula II′″, R^{a1} is —OMe.

In some embodiments of formula II′″, R^{a2} is hydrogen. In some embodiments of formula II′″, R^{a2} is C₁₋₆ aliphatic, —CH₂R, or —O(C₁₋₄ alkyl). In some embodiments of formula II′″, R^{a2} is C₁₋₆ aliphatic. In some embodiments of formula II′″, R^{a2} is C₁₋₃ aliphatic. In some embodiments of formula II′″, R^{a2} is methyl, ethyl, n-propyl, or isopropyl. In some embodiments of formula II′″, R^{a2} is methyl. In some embodiments of formula II′″, R^{a2} is ethyl. In some embodiments of formula II′″, R^{a2} is —CH═CH₂. In some embodiments of formula II′″, R^{a2} is n-propyl. In some embodiments of formula II′″, R^{a2} is isopropyl. In some embodiments of formula II′″, R^{a2} is —CH₂C≡CH. In some embodiments of formula II′″, R^{a2} is —CH₂CH═CH₂. In some embodiments of formula II′″, R^{a2} is —CH₂R. In some embodiments of formula II′″, R^{a2} is —O(C₁₋₄ alkyl). In some embodiments of formula II′″, R^{a2} is —OMe.

In some embodiments of formula II′″, R^{a3} is hydrogen. In some embodiments of formula II′″, R^{a3} is C₁₋₆ aliphatic, —CH₂R, or —O(C₁₋₄ alkyl). In some embodiments of formula II′″, R^{a3} is C₁₋₆ aliphatic. In some embodiments of formula II′″, R^{a3} is C₁₋₃ aliphatic. In some embodiments of formula II′″, R^{a3} is methyl, ethyl, n-propyl, or isopropyl. In some embodiments of formula II′″, R^{a3} is methyl. In some embodiments of formula II′″, R^{a3} is ethyl. In some embodiments of formula II′″, R^{a3} is n-propyl. In some embodiments of formula II′″, R^{a3} is isopropyl.

In some embodiments of formula II′″, R is C₁₋₄ aliphatic substituted with halogen. In some embodiments of formula II′″, R is C₁₋₂ aliphatic substituted with halogen. In some embodiments of formula II′″, R is —CF₃. Accordingly, in some embodiments of formula II′″, R^{a1}, R^{a2}, or R^{a3} is —CH₂CF₃.

In some embodiments of formula II′″, R is phenyl. Accordingly, in some embodiments of formula II′″, R^{a1}, R^{a2}, or R^{a3} is benzyl

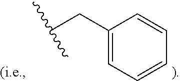 [SMILES: *Cc1ccccc1]

In some embodiments of formula II′″, R is a 3- to 6-membered saturated carbocyclic ring. In some embodiments of formula II′″, R is a 3- to 4-membered saturated carbocyclic ring. In some embodiments of formula II′″, R is a 3-membered saturated carbocyclic ring. Accordingly, in some embodiments of formula II′″, R^{a1}, R^{a2}, or R^{a3} is

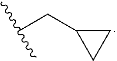 [SMILES: *CC1CC1]

In some embodiments of formula II′″, R is a 5- to 6-membered heteroaryl ring having 1-3 heteroatoms independently selected from nitrogen, oxygen, and sulfur. In some embodiments of formula II′″, R is a 6-membered heteroaryl ring having 1-3 nitrogen atoms. In some embodiments of formula II′″, R is a 6-membered heteroaryl ring having 1-2 nitrogen atoms. In some embodiments of formula II′″, R is a 6-membered heteroaryl ring having 1 nitrogen atom. In some embodiments of formula II′″, R is 4-pyridyl. Accordingly, in some embodiments of formula II′″, R^{a1}, R^{a2}, or R^{a3} is

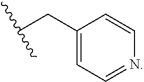 [SMILES: *Cc1ccncc1]

In some embodiments of formula II′″, R⁴ is —OH. In some embodiments of formula II′″, R⁴ is —OMe.

In some embodiments of formula II″ or formula II′″, Y³ is NR^{a1} and Y⁴ is NH, wherein R^{a1} is C₁₋₆ aliphatic or —CH₂R. In some embodiments of formula II″ or formula II′″, Y³ is NH and Y⁴ is NR^{a2}, wherein R^{a2} is C₁₋₆ aliphatic or —CH₂R. In some embodiments of formula II″ or formula II′″, Y³ is NR^{a1} and Y⁴ is NR^{a2}, wherein each of R^{a1} and R^{a2} is independently C₁₋₆ aliphatic or —CH₂R.

In some embodiments, N₂ is uridine, 1-methylpseudouridine, 2-thio-uridine, or 5-methyluridine.

In some embodiments N₂ is:

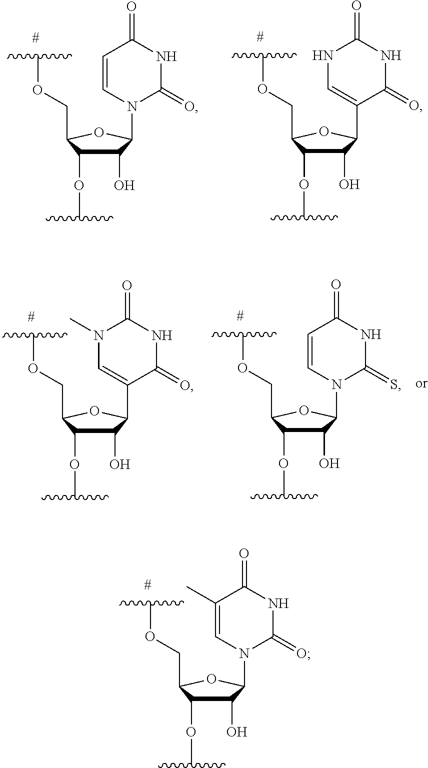

or a salt thereof, wherein #represents the point of attachment to p of N₁p.

Tn some embodiments N₂ is

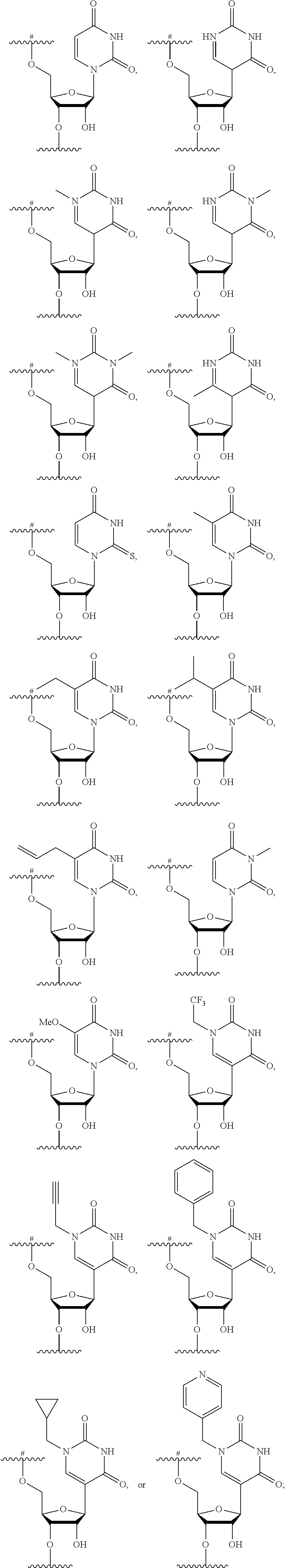

or a salt thereof, wherein #represents the point of attachment to p of N₁p.

In some embodiments N₂ is:

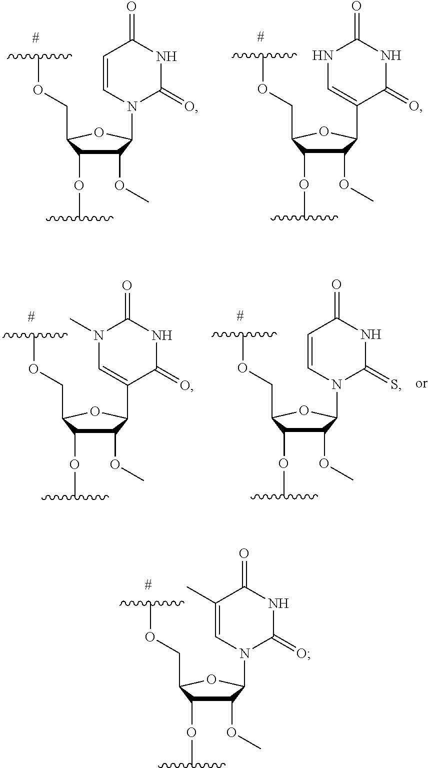

or a salt thereof, wherein #represents the point of attachment to p of N₁p.

In some embodiments N₂ is:

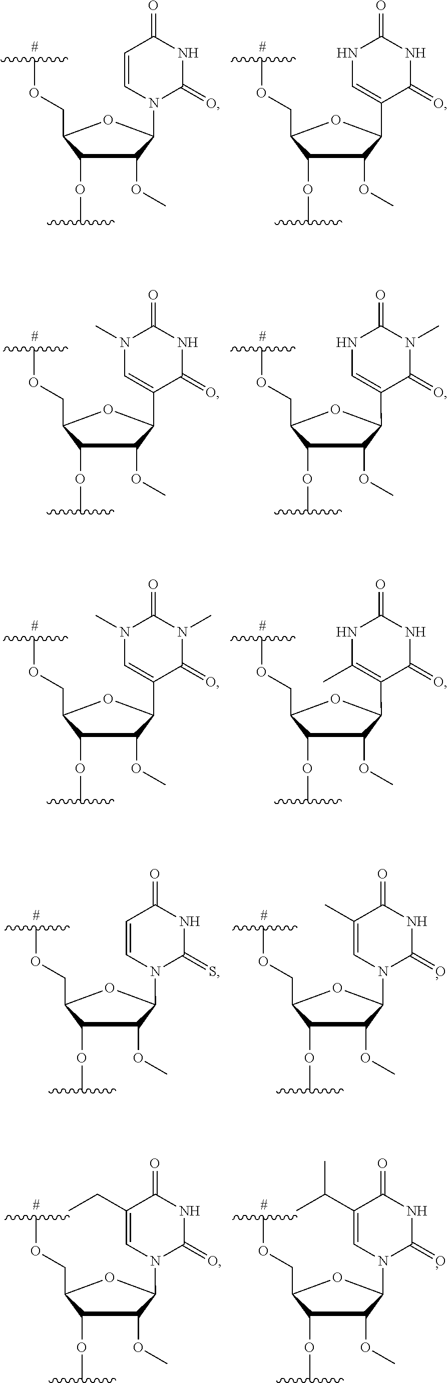

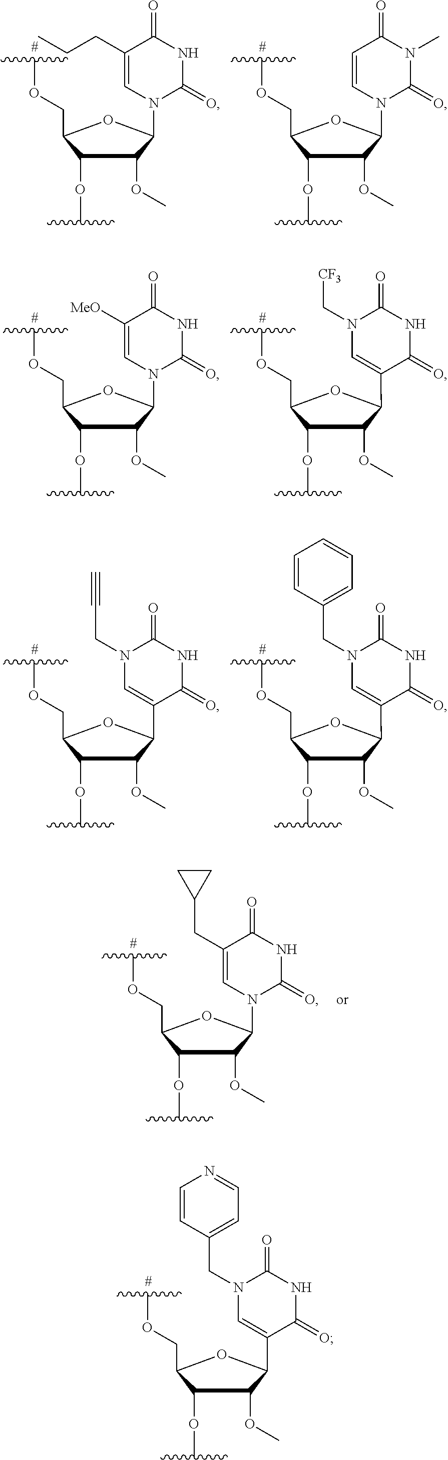

or a salt thereof, wherein #represents the point of attachment to p of N₁p.

In some embodiments, p is —P(═O)(OH)—, or a salt thereof.

In some embodiments, the 5′ cap is (m^{7,2′-O})Gppp(m^{2′-O})A₁pU₂, (m^{7,3′-O})Gppp(m^{2′-O})A₁pU₂, (m^{7,2′-O})Gppp(m^{2′-O})A₁pΨ₂, (m^{7,3′-O})Gppp(m^{2′-O})A₁pΨ₂, (m^{7,2′-O})Gppp(m^{2′-O})A₁p(m¹)Ψ₂, (m^{7,3′-O})Gppp(m^{2′-O})A₁p(m¹)Ψ₂, (m^{7,2′-O})Gppp(m^{2′-O})A₁pS²U₂, (m^{7,3′-O})Gppp(m^{2′-O})A₁pS²U₂, (m^{7,2′-O})Gppp(m^{2′-O})A₁p(m⁵)U₂, or (m^{7,3′-O})Gppp(m^{2′-O})A₁p(m⁵)U₂.

In some embodiments, the 5′ cap is (m^{7,2′-O})Gppp(m^{6,2′-O})A₁pU₂, (m^{7,3′-O})Gppp(m^{6,2′-O})A₁pU₂, (m^{7,2′-O})Gppp(m^{6,2′-O})A₁pΨ₂, (m^{7,3′-O})Gppp(m^{6,2′-O})A₁pΨ₂, (m^{7,2′-O})Gppp(m^{6,2′-O})A₁p(m¹)Ψ₂, (m^{7,3′-O})Gppp(m^{6,2′-O})A₁p(m¹)Ψ₂, (m^{7,2′-O})Gppp(m^{6,2′-O})A₁pS²U₂, (m^{7,3′-O})Gppp(m^{6,2′-O})A₁pS²U₂, (m^{7,2′-O})Gppp(m^{6,2′-O})A₁p(m⁵)U₂, or (m^{7,3′-O})Gppp(m^{6,2′-O})A₁p(m⁵)U₂.

In some embodiments, the 5′ cap is (m^{7,2′-O})GpppA₁(m^{2′-O})pU₂, (m^{7,3′-O})GpppA₁(m^{2′-O})pU₂, (m^{7,2′-O})GpppA₁(m^{2′-O})pΨ₂ (m^{7,3′-O})GpppA₁(m^{2′-O})pΨ₂ (m^{7,2′-O})GpppA₁(m^{2′-O})p(m¹)Ψ₂, (m^{7,3′-O})GpppA₁(m^{2′-O})p(m¹)Ψ₂, (m^{7,2′-O})Gppp(m^{2′-O})A₁(m^{2′-O})pS²U₂, (m^{7,3′-O})GpppA₁(m^{2′-O})pS²U₂, (m^{7,2′-O})GpppA₁(m^{2′-O})p(m⁵)U₂, or (m^{7,3′-O})GpppA₁(m^{2′-O})p(m⁵)U₂.

In some embodiments, the 5′ cap is (m^{7,2′-O})Gppp(m^{6,2′-O})A₁pU₂, (m^{7,3′-O})Gppp(m^{6,2′-O})A₁pU₂, (m^{7,2′-O})Gppp(m^{6,2′-O})A₁pΨ₂, (m^{7,3′-O})Gppp(m^{6,2′-O})A₁pΨ₂, (m^{7,2′-O})Gppp(m^{6,2′-O})A₁p(m¹)Ψ₂, (m^{7,3′-O})Gppp(m^{6,2′-O})A₁p(m¹)Ψ₂, (m^{7,2′-O})Gppp(m^{6,2′-O})A₁pS²U₂, (m^{7,3′-O})Gppp(m^{6,2′-O})A₁pS²U₂, (m^{7,2′-O})Gppp(m^{6,2′-O})A₁p(m⁵)U₂, or (m^{7,3′-O})Gppp(m^{6,2′-O})A₁p(m⁵)U₂.

In some embodiments, the 5′ cap is m⁷G(^{3′-OMe})pppA₁(^{2′-OMe})pm³U₂, m⁷G(^{3′-OMe})pppA₁(^{2′-OMe})pmO⁵U₂, m⁷GpppA₁(^{2′-OMe})pm¹Ψ₂, m⁷G(^{3′-OMe})pppA₁(^{2′-OMe})pM³U₂, m⁷G(^{3′-OMe})pppA₁(^{2′-OMe})ptfet¹Ψ₂, m⁷G(^{3′-OMe})pppA₁(^{2′-OMe})P(Ppg)¹Ψ₂, m⁷G(^{3′-OMe})pppA₁(^{2′-OMe})pbn¹Ψ₂, m⁷G(^{3′-OMe})pppA₁(^{2′-OMe})pcpm¹Ψ₂, or m⁷G(^{3′-OMe})pppA₁(^{2′-OMe})p(4-pm)¹Ψ₂.

In some embodiments, a 5′ cap provided herein is selected from those in Table 1:

| Compound | |
|---|---|
| No. | Structure |
| I′-1 | 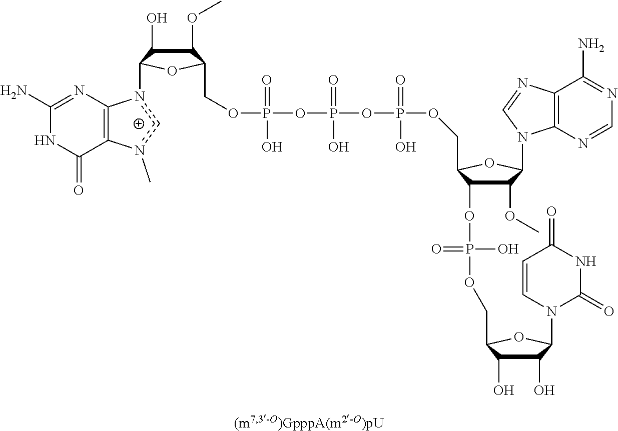 |
| I′-2 | 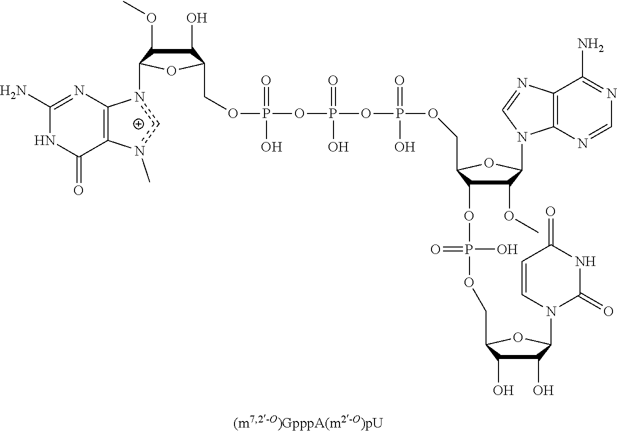 |
| I′-3 | 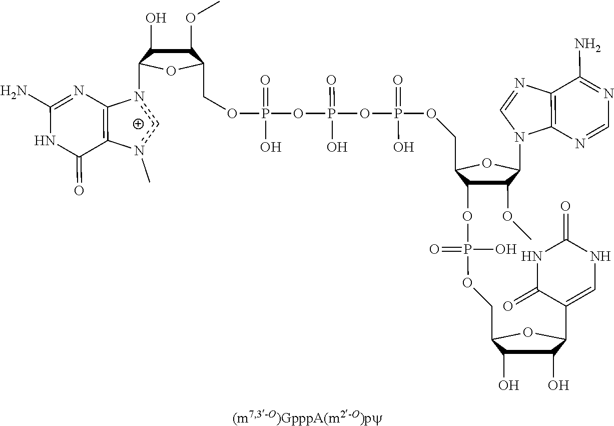 |
| I′-4 | 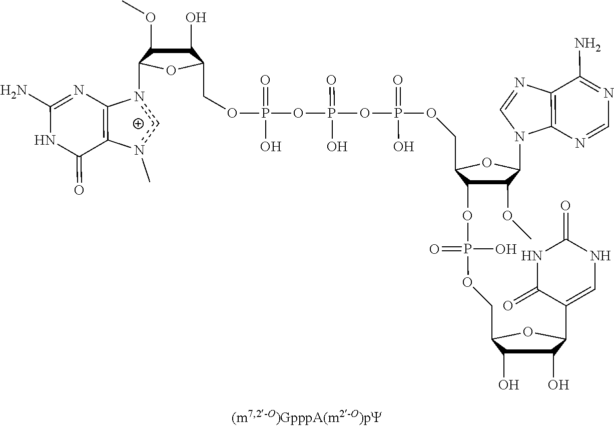 |
| I′-5 | 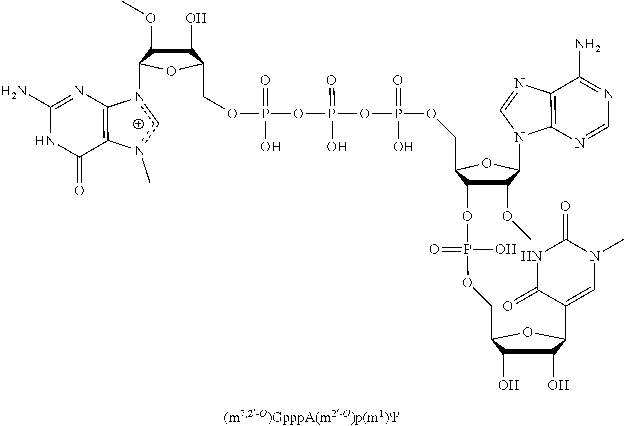 |
| I′-6 | 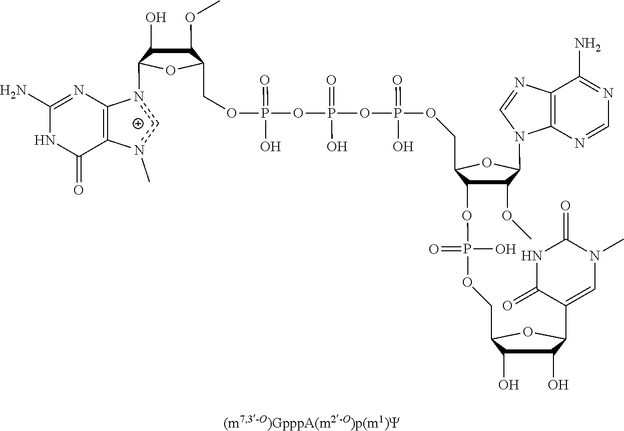 |
| I′-7 | 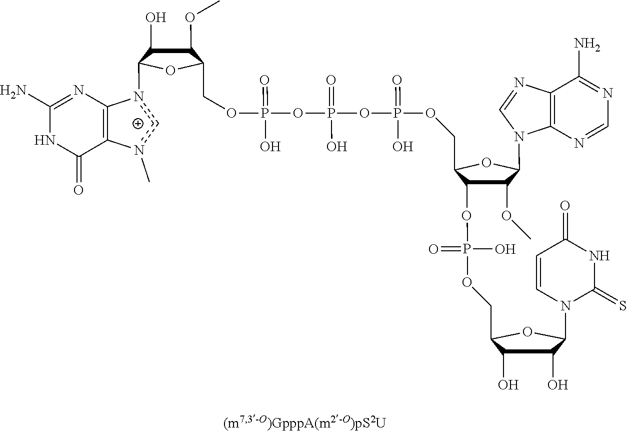 |
| I′-8 | 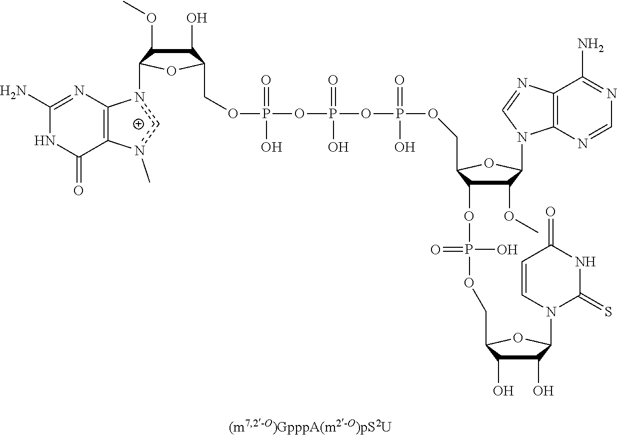 |
| I′-9 | 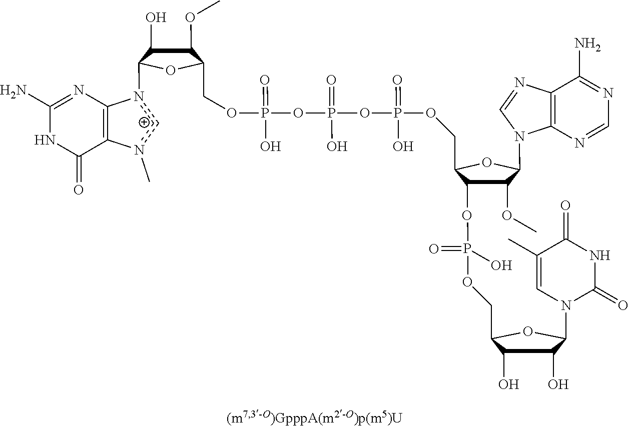 |
| I′-10 | 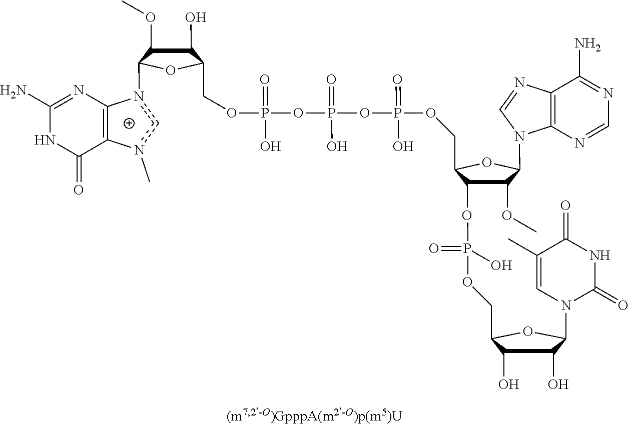 |
| I′-11 | 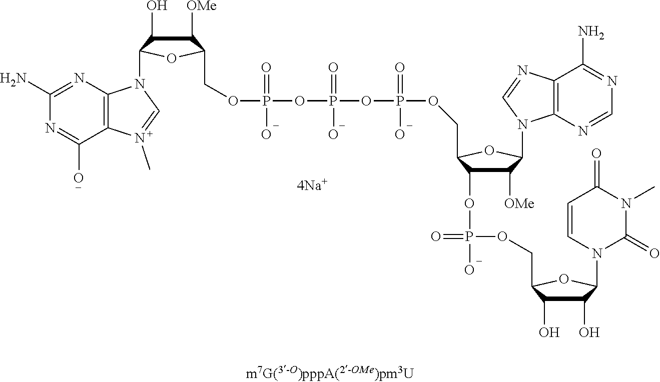 |
| I′-12 | 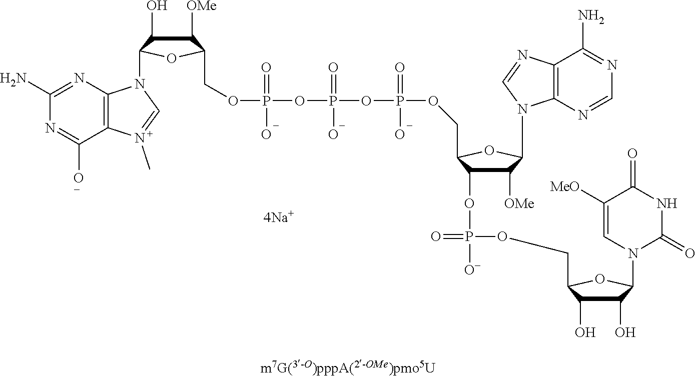 |
| I′-13 | 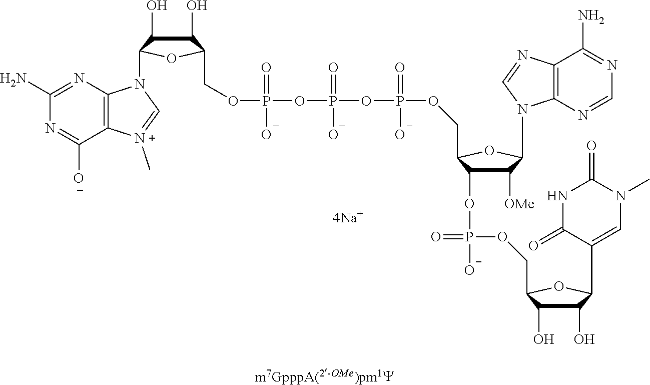 |
| I′-14 | 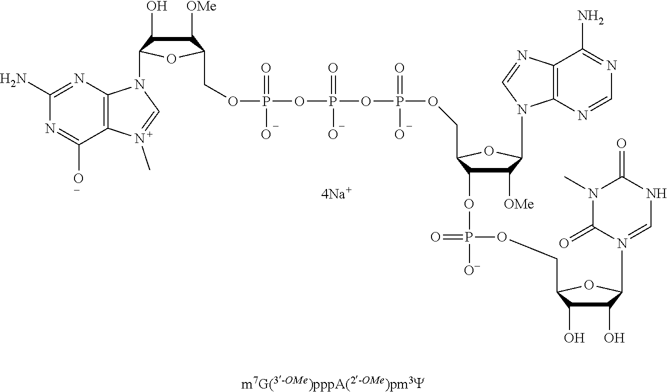 |
| I′-15 | 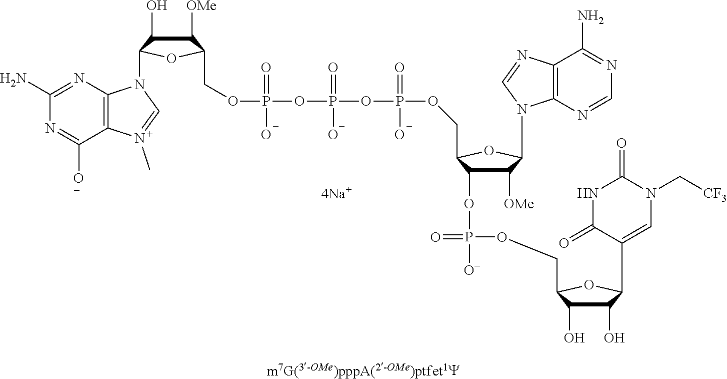 |
| I′-16 | 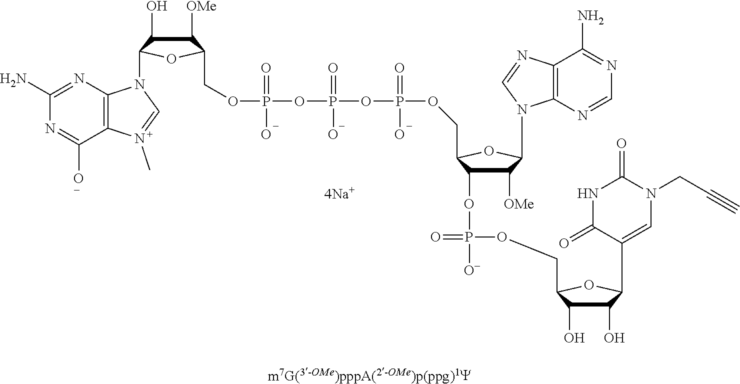 |
| I′-17 | 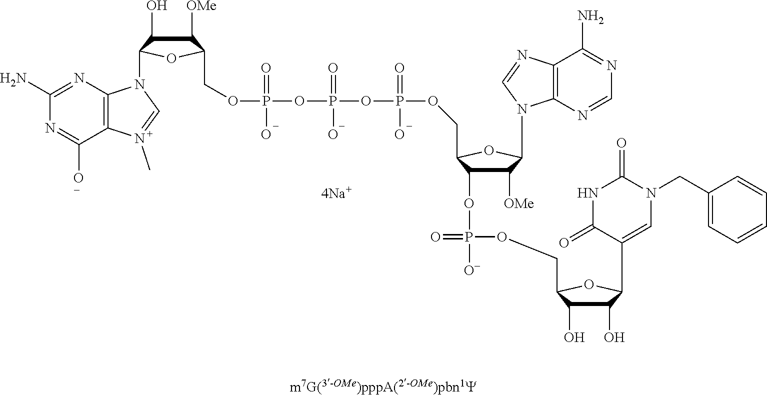 |
| I′-18 | 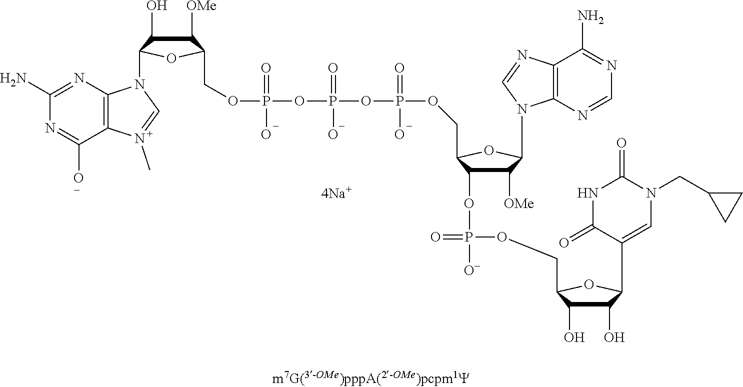 |
| I′-19 | 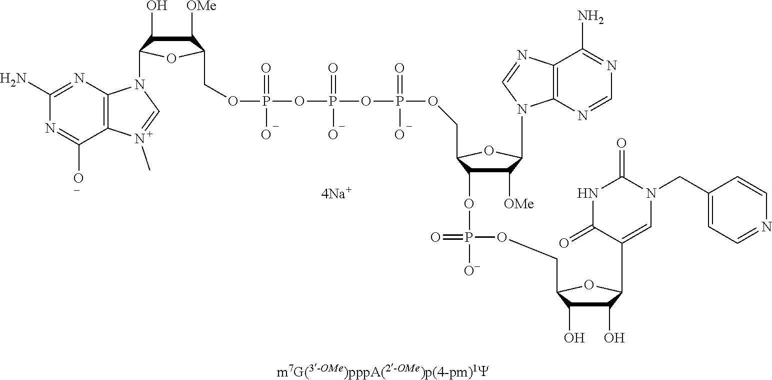 |

or a salt thereof.

In some embodiments, the 5′ cap is (m^{7,2′-O})Gppp(m^{2′-0})A₁pU₂, having a structure:

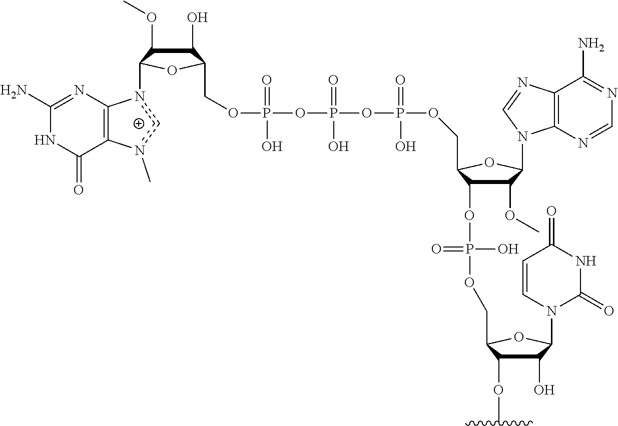

or a salt thereof.

In some embodiments, the 5′ cap is (m^{7,3′-O})Gppp(m^{2′-O})A₁pU₂,

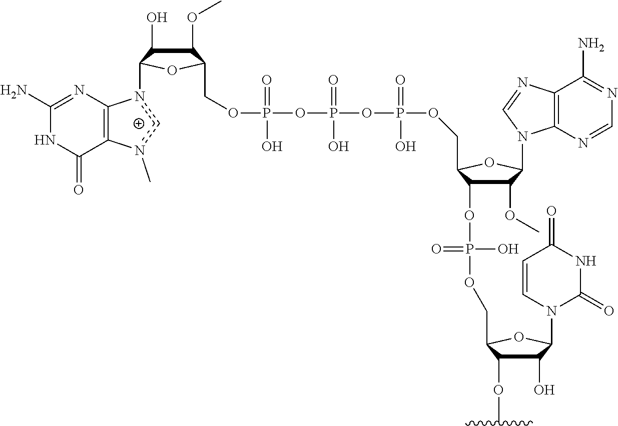

or a salt thereof.

In some embodiments, the 5′ cap is (m^{7,3′-O})Gppp(m^{2′-O})A₁pΨ₂,

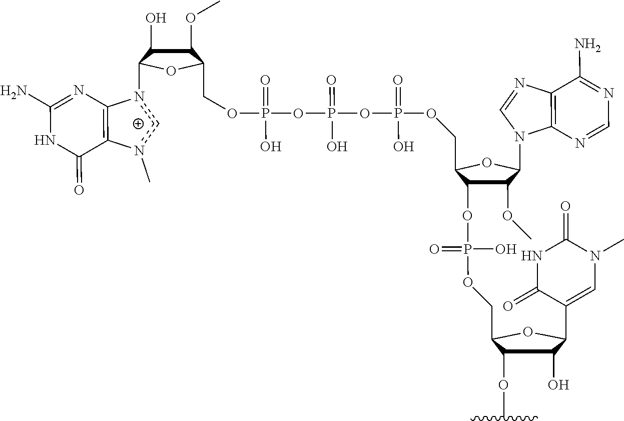

or a salt thereof.

In some embodiments, the 5′ cap is (m^{7,2′-O})Gppp(m^{2′-O})A₁pΨ₂,

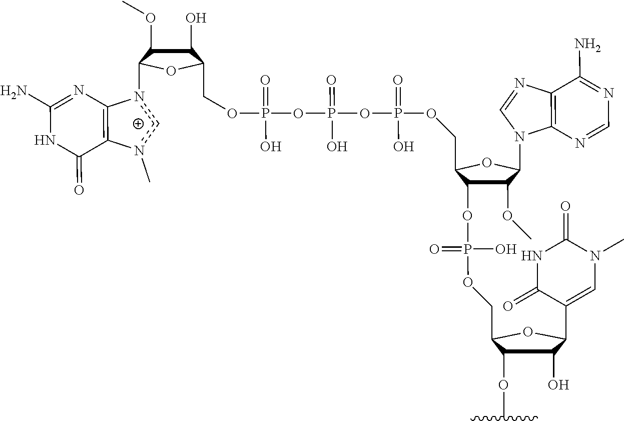

or a salt thereof.

In some embodiments, the 5′ cap is (m^{7,2′-O})Gppp(m^{2′-O})A₁p(m¹)Ψ₂,

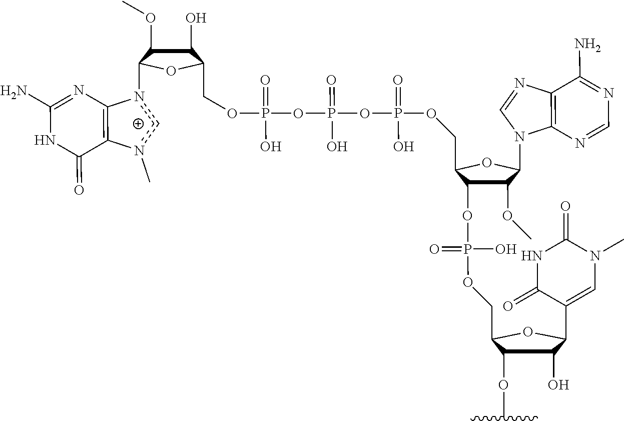

or a salt thereof.

In some embodiments, the 5′ cap is (m^{7,3′-O})Gppp(m^{2′-O})A₁p(m¹)Ψ₂,

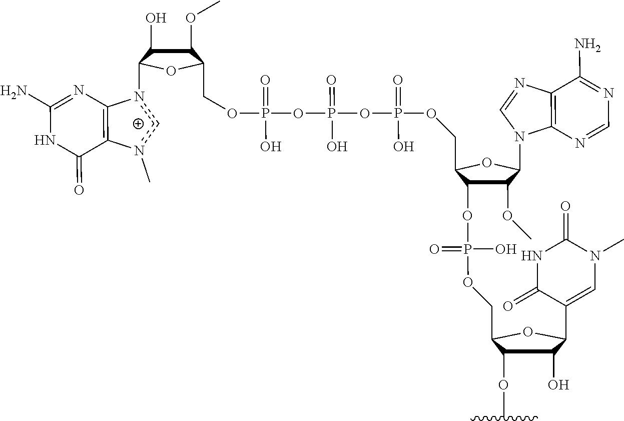

or a salt thereof.

In some embodiments, the 5′ cap is (m^{7,3′-O})Gppp(m^{2′-O})A₁pS²U₂,

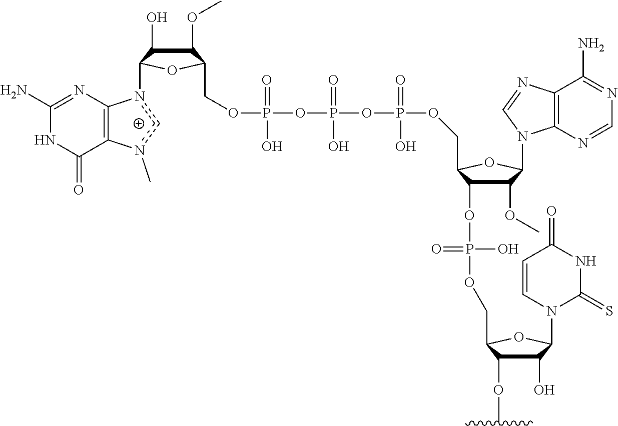

or a salt thereof.

In some embodiments, the 5′ cap is (m^{7,2′-O})Gppp(m^{2′-O})A₁pS²U₂,

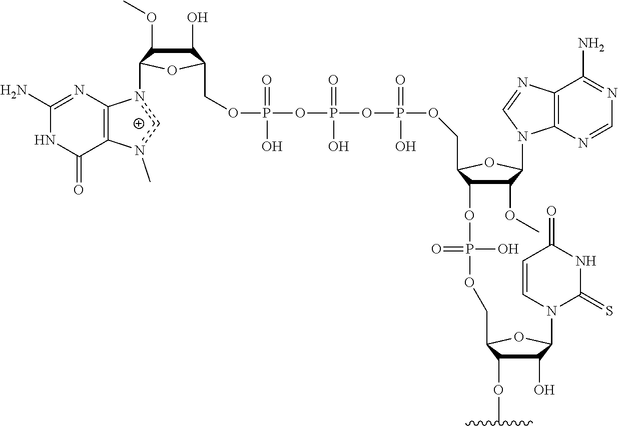

or a salt thereof.

In some embodiments, the 5′ cap is (m^{7,3′-O})Gppp(m^{2′-O})A₁p(m⁵)U₂,

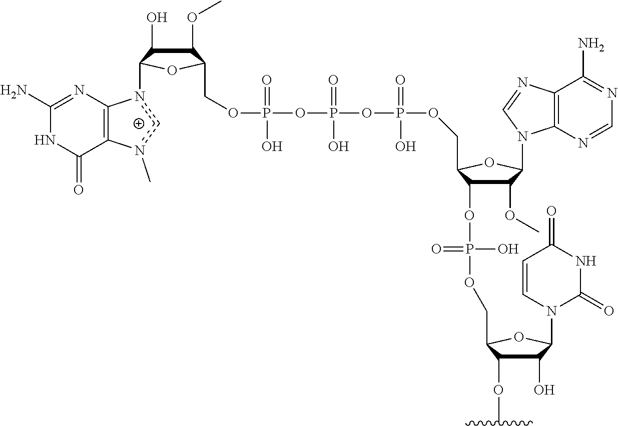

or a salt thereof.

In some embodiments, the 5′ cap is (m^{7,2′-O})Gppp(m^{2′-O})A₁p(m⁵)U₂,

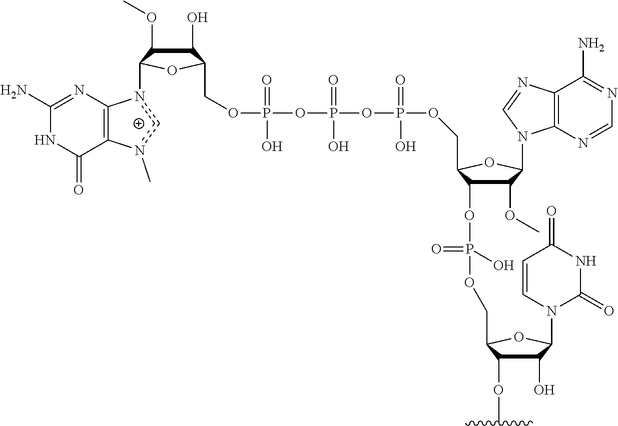

or a salt thereof.

In some embodiments, it will be appreciated that the disclosure of 5′ caps above and herein encompasses 5′ caps themselves or as part of a larger molecule (e.g., an RNA). For example, the structures drawn above encompass a 3′ ether linkage to the next nucleotide or as a free —OH.

In some embodiments, the present disclosure provides a compound of formula G*N₁pN₂, wherein:
G* is of formula I′:

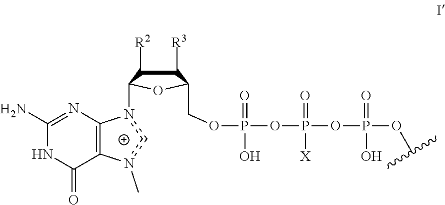

or a salt thereof, wherein each R², R³, X, N₁, p, and N₂ is as defined above and described herein.

In some embodiments, N₂ is of formula II′:

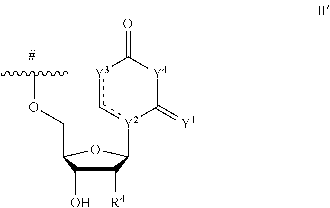

or a salt thereof, wherein each [symbol] , Y¹, Y², Y³, Y⁴, R^{a1}, R^{a2}, R⁴, and # is as defined above and described herein.

In some embodiments, N₂ is of formula IIa′:

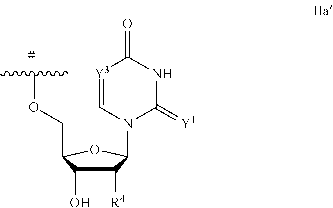

or a salt thereof, wherein each Y¹, Y³, R⁴ and # is as defined above and described herein.

In some embodiments, N₂ is of formula IIb′:

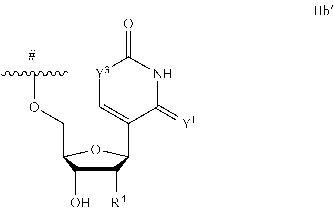

or a salt thereof, wherein each of Y¹, Y³, R⁴ and # is as defined above and described herein.

In some embodiments, N₂ is of formula IIa′ or IIb′, wherein each of Y¹, Y³, and R⁴ is as defined above for formula II″.

In some embodiments, N₂ is:

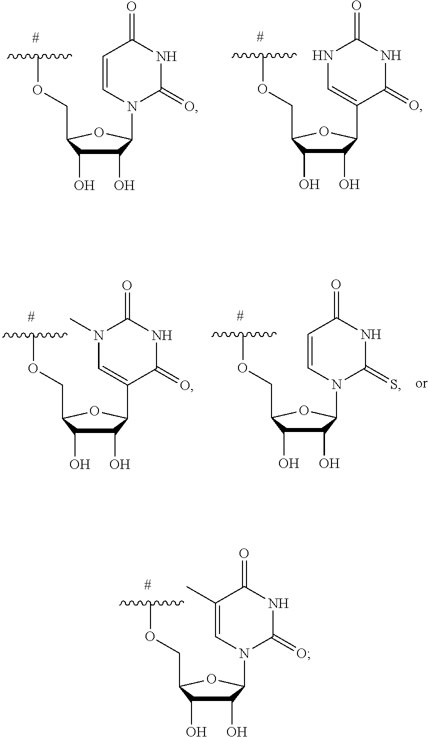

or a salt thereof;
wherein #represents the point of attachment to p of N₁p.

In some embodiments, N₂ is:

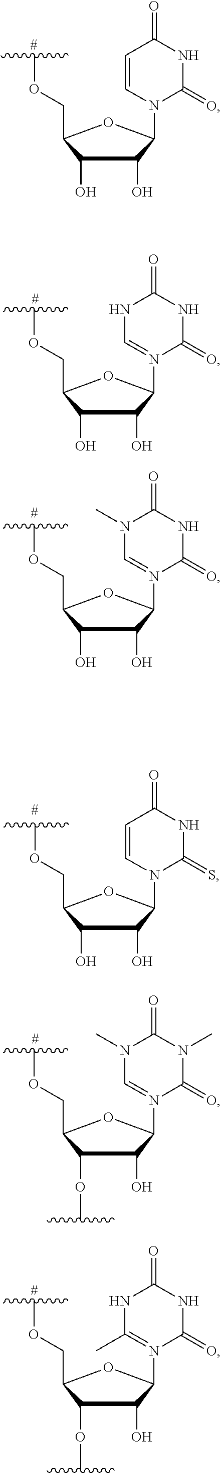

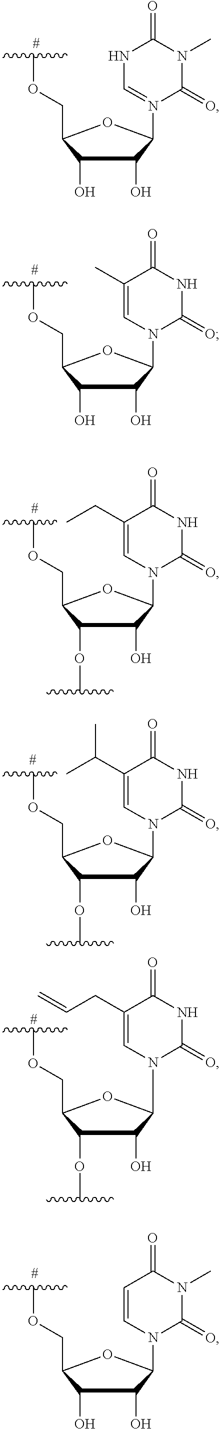

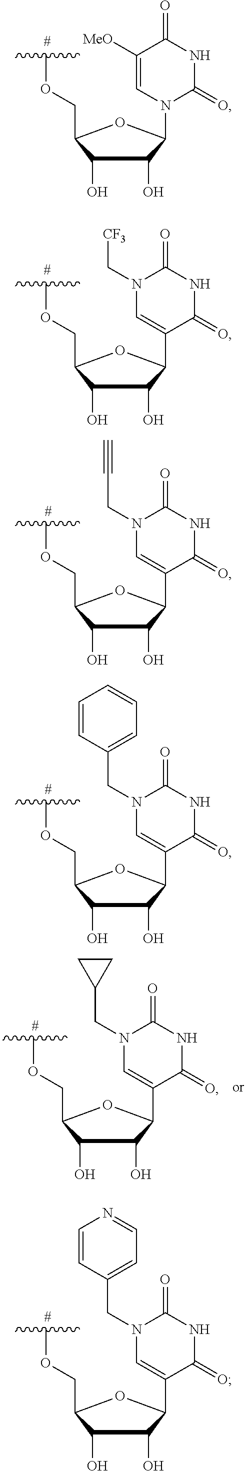

or a salt thereof;
wherein #represents the point of attachment to p of N₁p.

In some embodiments, N₂ is:

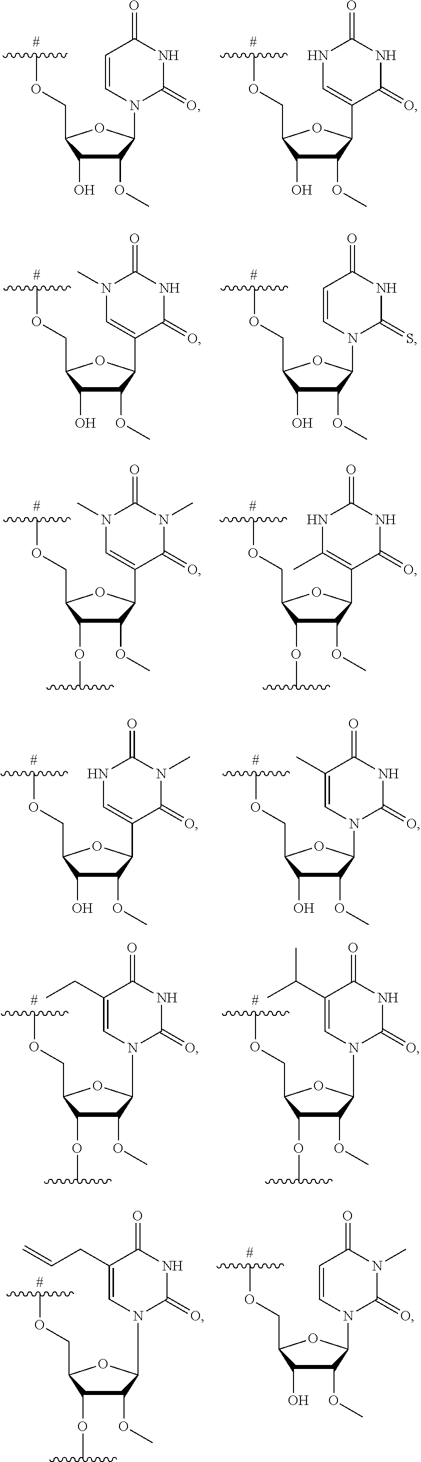

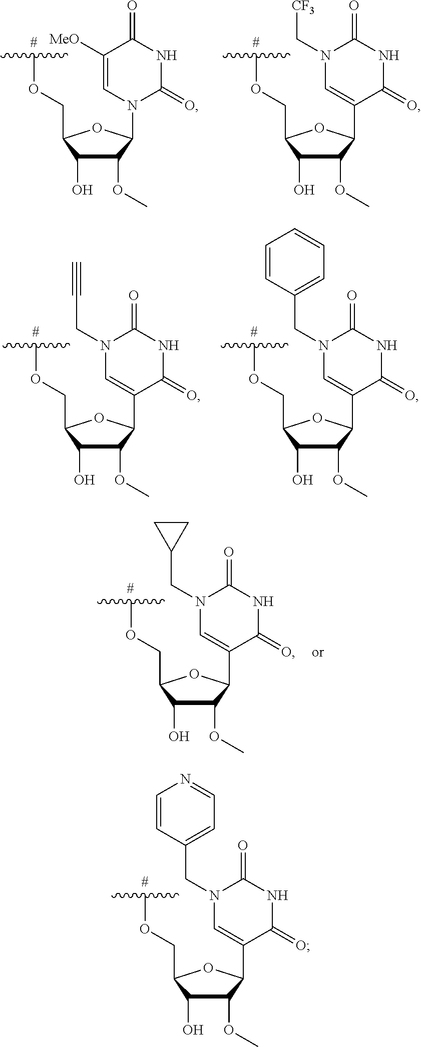

or a salt thereof;
wherein #represents the point of attachment to p of N₁p.

In some embodiments, N₁ is:

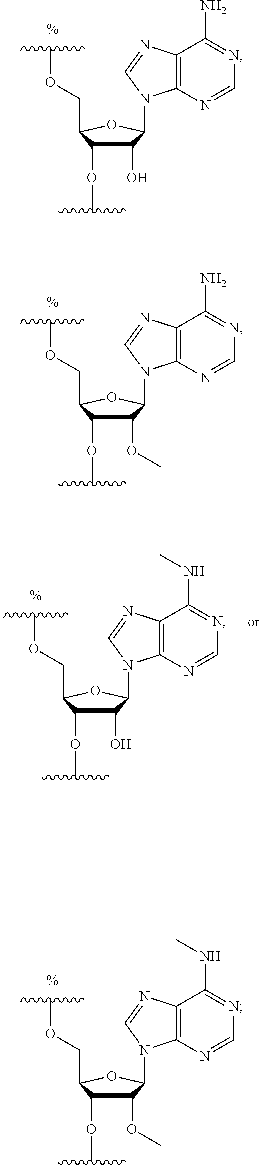

or a salt thereof,

In some embodiments, p is —P(═O)(OH)—, or a salt thereof such as —P(═O)(O⁻)—.

In some embodiments, the present disclosure provides a compound (m^{7,2′-O})Gppp(m^{2′-O})A₁pU₂, having a structure:

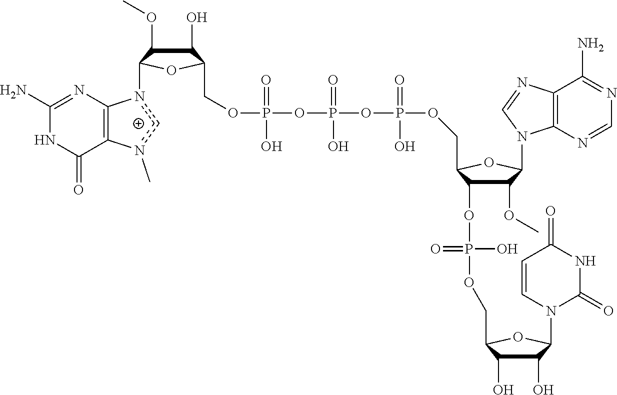

or a salt thereof.

In some embodiments the present disclosure provides a compound (m^{7,3′-O})Gppp(m^{2′-O})A₁pU₂,

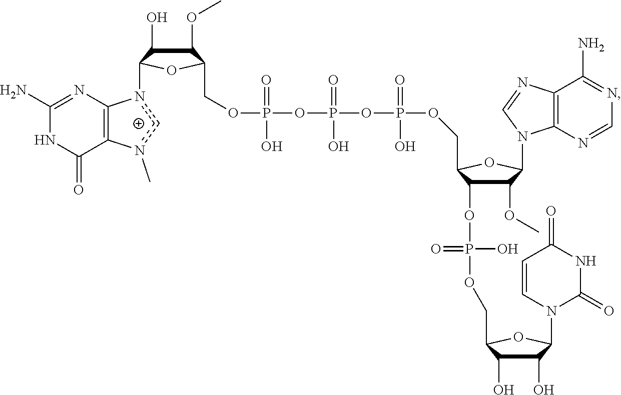

or a salt thereof.

In some embodiments, the present disclosure provides a compound (m^{7,3′-O})Gppp(m^{2-O})A₁pΨ₂,

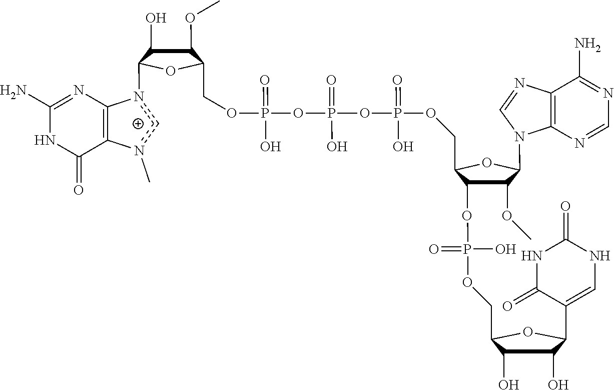

or a salt thereof.

In some embodiments, the present disclosure provides a compound (m^{7,2′-O})Gppp(m^{2′-O})A₁pΨ₂,

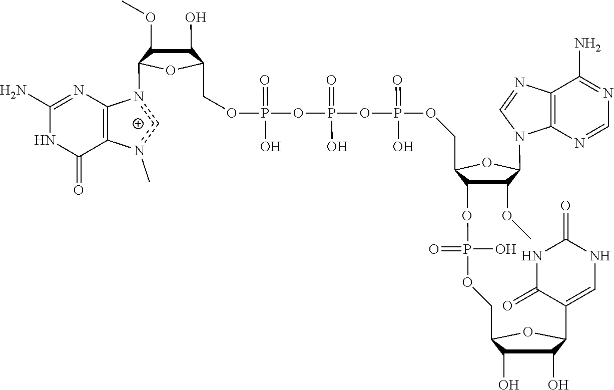

or a salt thereof.

In some embodiments, the present disclosure provides a compound (m^{7,2′-O})Gppp(m^{2′-O})A₁p(m¹)Ψ₂,

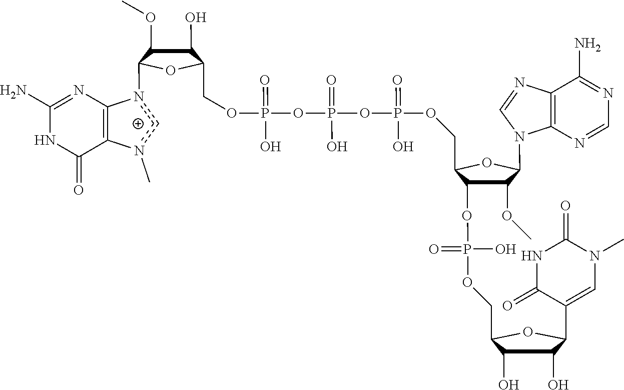

or a salt thereof.

In some embodiments, the present disclosure provides a compound (m^{7,3′-O})Gppp(m^{2′-O})A₁p(m¹)Ψ₂,

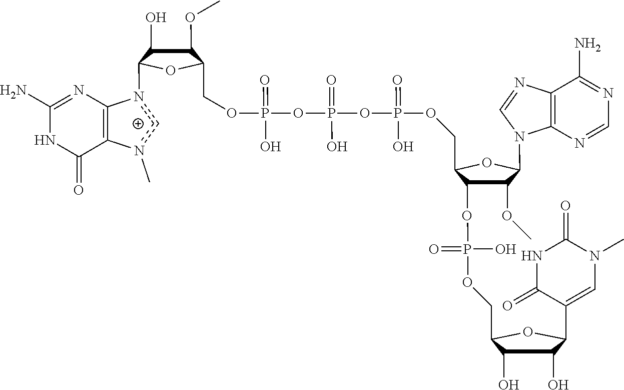

or a salt thereof.

In some embodiments, the present disclosure provides a compound (m^{7,3′-O})Gppp(m^{2′-O})A₁pS²U₂,

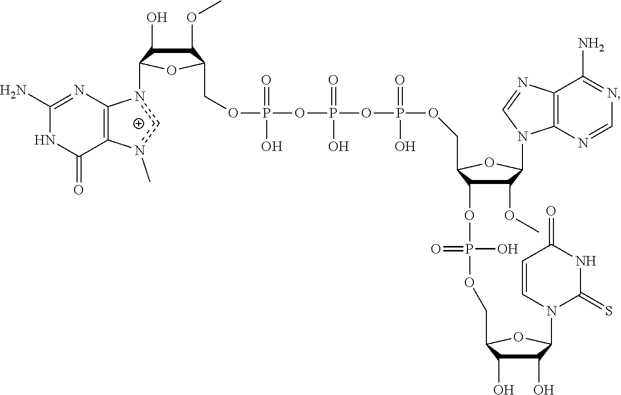

or a salt thereof.

In some embodiments, the present disclosure provides a compound (m^{7,2′-O})Gppp(m^{2′-O})A₁pS²U₂,

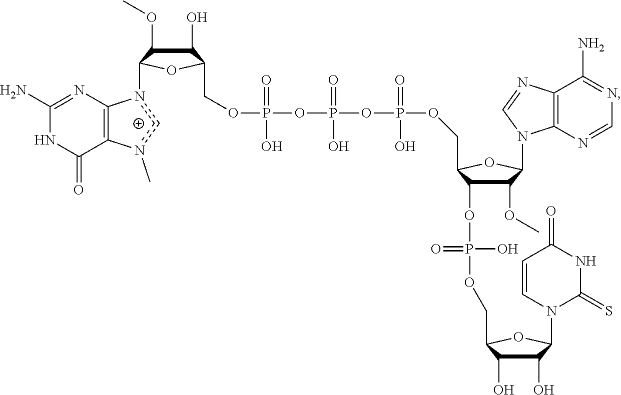

or a salt thereof.

In some embodiments, the present disclosure provides a compound (m^{7,3′-O})Gppp(m^{2′-O})A₁p(m⁵)U₂,

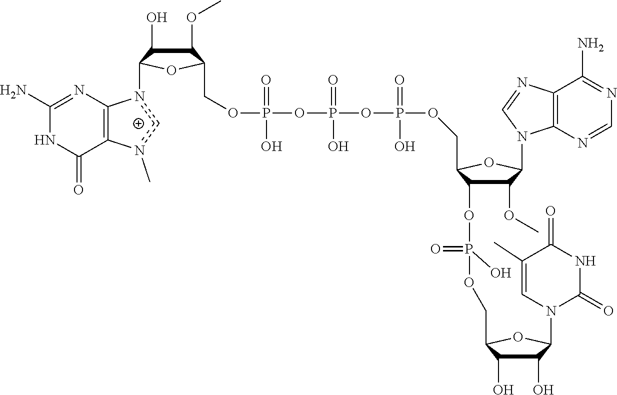

or a salt thereof.

In some embodiments, the present disclosure provides a compound (m^{7,2′-O})Gppp(m^{2′-O})A₁p(m⁵)U₂,

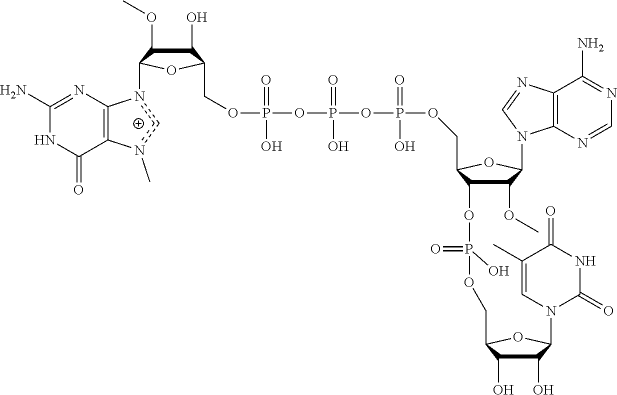

or a salt thereof.

In some embodiments, a provided compound is a salt. In some embodiments, a provided compound is a pharmaceutically acceptable salt.

### 5′ UTR and Cap Proximal Sequences

In some embodiments, an RNA disclosed herein comprises a 5′-UTR. The term “untranslated region” or “UTR” relates to a region in a DNA molecule which is transcribed but is not translated into an amino acid sequence, or to the corresponding region in an RNA polynucleotide, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′ end of an RNA, upstream of the start codon of a protein-encoding region. A 5′-UTR can be downstream of the 5′-cap (if present), e.g. directly adjacent to the 5′-cap.

In some embodiments, a 5′ UTR disclosed herein comprises a cap proximal sequence, e.g., as disclosed herein. In some embodiments, a cap proximal sequence comprises a sequence adjacent to a 5′ cap (e.g., 1, 2, 3, 4, 5, 6, 7, 8, 9, or 10 nucleotides immediately adjacent to a 5′ cap). In some embodiments, a cap proximal sequence comprises nucleotides in positions +1, +2, +3, +4, and/or +5 of an RNA polynucleotide.

In some embodiments a 5′ UTR comprises a Kozak sequence (e.g., GCCACC). In some embodiments a Kozak sequence is immediately adjacent to a payload sequence (e.g., immediately upstream of a start codon).

In some embodiments, a Cap structure comprises one or more polynucleotides of a cap proximal sequence. In some embodiments, a Cap structure comprises an m7 Guanosine cap and nucleotides +1 and +2 (N₁ and N₂) of an RNA polynucleotide.

Those skilled in the art, reading the present disclosure, will appreciate that, in some embodiments, one or more residues of a cap proximal sequence (e.g., one or more of residues +1, +2, +3, +4, and/or +5) may be included in an RNA by virtue of having been included in a cap entity (e.g., a Cap1 or Cap2 structure, etc); alternatively, in some embodiments, at least some of the residues in a cap proximal sequence may be enzymatically added (e.g., by a polymerase such as a T7 polymerase). For example, in certain exemplified embodiments where a m₂^{7,3′-O}Gppp(m₁^{2′-O})ApU cap is utilized, +1 (i.e., N₁) and +2 (i.e. N₂) are the (m₁^{2′-O})A and U residues of the cap, and +3, +4, and +5 are added by polymerase (e.g., T7 polymerase).

In some embodiments, the 5′ cap is a trinucleotide cap structure (e.g., the trinucleotide cap structures described above and herein), wherein the cap proximal sequence comprises N₁ and N₂ of the 5′ cap, wherein N₁ is as defined above and described herein, and N₂ is as defined above and described herein.

In some embodiments, e.g., where the 5′ cap is a trinucleotide cap structure, a cap proximal sequence comprises N₁ and N₂ of a the 5′ cap, and N₃, N₄ and N₅, wherein N₁ to N₅ correspond to positions +1, +2, +3, +4, and/or +5 of an RNA polynucleotide. In some embodiments, N₃ is A. In some embodiments, N₃ is C. In some embodiments, N₃ is G. In some embodiments, N₃ is U. In some embodiments, N₄ is A. In some embodiments, N₄ is C. In some embodiments, N₄ is G. In some embodiments, N₄ is U. In some embodiments, N₅ is A. In some embodiments, N₅ is C. In some embodiments, N₅ is G. In some embodiments, N₅ is U.

In some embodiments, N₃ is A, N₄ is A, and N₅ is A. In some embodiments, N₃ is A, N₄ is A, and N₅ is C. In some embodiments, N₃ is A, N₄ is A, and N₅ is G. In some embodiments, N₃ is A, N₄ is A, and N₅ is U. In some embodiments, N₃ is A, N₄ is C, and N₅ is C. In some embodiments, N₃ is A, N₄ is C, and N₅ is G. In some embodiments, N₃ is A, N₄ is C, and N₅ is U. In some embodiments, N₃ is A, N₄ is G, and N₅ is C. In some embodiments, N₃ is A, N₄ is G, and N₅ is G. In some embodiments, N₃ is A, N₄ is G, and N₅ is U. In some embodiments, N₃ is A, N₄ is U, and N₅ is C. In some embodiments, N₃ is A, N₄ is U, and N₅ is G. In some embodiments, N₃ is A, N₄ is U, and N₅ is U.

In some embodiments, N₃ is C, N₄ is A, and N₅ is A. In some embodiments, N₃ is C, N₄ is A, and N₅ is C. In some embodiments, N₃ is C, N₄ is A, and N₅ is G. In some embodiments, N₃ is C, N₄ is A, and N₅ is U. In some embodiments, N₃ is C, N₄ is C, and N₅ is C. In some embodiments, N₃ is C, N₄ is C, and N₅ is G. In some embodiments, N₃ is C, N₄ is C, and N₅ is U. In some embodiments, N₃ is C, N₄ is G, and N₅ is C. In some embodiments, N₃ is C, N₄ is G, and N₅ is G. In some embodiments, N₃ is C, N₄ is G, and N₅ is U. In some embodiments, N₃ is C, N₄ is U, and N₅ is C. In some embodiments, N₃ is C, N₄ is U, and N₅ is G. In some embodiments, N₃ is C, N₄ is U, and N₅ is U.

In some embodiments, N₃ is G, N₄ is A, and N₅ is A. In some embodiments, N₃ is G, N₄ is A, and N₅ is C. In some embodiments, N₃ is G, N₄ is A, and N₅ is G. In some embodiments, N₃ is G, N₄ is A, and N₅ is U. In some embodiments, N₃ is G, N₄ is C, and N₅ is C. In some embodiments, N₃ is G, N₄ is C, and N₅ is G. In some embodiments, N₃ is G, N₄ is C, and N₅ is U. In some embodiments, N₃ is G, N₄ is G, and N₅ is C. In some embodiments, N₃ is G, N₄ is G, and N₅ is G. In some embodiments, N₃ is G, N₄ is G, and N₅ is U. In some embodiments, N₃ is G, N₄ is U, and N₅ is C. In some embodiments, N₃ is G, N₄ is U, and N₅ is G. In some embodiments, N₃ is G, N₄ is U, and N₅ is U.

In some embodiments, N₃ is U, N₄ is A, and N₅ is A. In some embodiments, N₃ is U, N₄ is A, and N₅ is C. In some embodiments, N₃ is U, N₄ is A, and N₅ is G. In some embodiments, N₃ is U, N₄ is A, and N₅ is U. In some embodiments, N₃ is U, N₄ is C, and N₅ is C. In some embodiments, N₃ is U, N₄ is C, and N₅ is G. In some embodiments, N₃ is U, N₄ is C, and N₅ is U. In some embodiments, N₃ is U, N₄ is G, and N₅ is C. In some embodiments, N₃ is U, N₄ is G, and N₅ is G. In some embodiments, N₃ is U, N₄ is G, and N₅ is U. In some embodiments, N₃ is U, N₄ is U, and N₅ is C. In some embodiments, N₃ is U, N₄ is U, and N₅ is G. In some embodiments, N₃ is U, N₄ is U, and N₅ is U.

Exemplary 5′ UTRs include a human alpha globin (hAg) 5′UTR or a fragment thereof, a TEV 5′ UTR or a fragment thereof, a HSP70 5′ UTR or a fragment thereof, or a c-Jun 5′ UTR or a fragment thereof.

In some embodiments, an RNA disclosed herein comprises a hAg 5′ UTR sequence or a fragment thereof. In some embodiments, an RNA disclosed herein comprises comprises a 5′ UTR comprising an AUAGU cap proximal sequence and an hAg 5′ UTR sequence (e.g., a 5′ UTR having at least 99%, 98%, 97%, 96%, 95%, 90%, 85%, or 80% identity to a human alpha globin 5′ UTR provided in SEQ ID NO: 11). In some embodiments, an RNA disclosed herein comprises a 5′ UTR as provided in SEQ ID NO: 11). In some embodiments, an RNA disclosed herein comprises a hAg 5′ UTR having at least 99%, 98%, 97%, 96%, 95%, 90%, 85%, or 80% identity to a human alpha globin 5′ UTR provided in SEQ ID NO: 12. In some embodiments, an RNA disclosed herein comprises a hAg 5′ UTR provided in SEQ ID NO: 12.

### 3′ UTR

In some embodiments, an RNA disclosed herein comprises a 3′-UTR. A 3′-UTR, if present, is located at the 3′ end of an RNA, downstream of the termination codon of a protein-encoding region, but the term “3′-UTR” preferably does not include a poly(A) sequence. Thus, the 3′-UTR is upstream of a poly(A) sequence (if present), e.g. directly adjacent to upstream of a poly(A) sequence.

In some embodiments, an RNA disclosed herein comprises a 3′ UTR comprising a first sequence from the amino terminal enhancer of split (AES) messenger RNA (an “F element”) and/or a second sequence from the mitochondrial encoded 12S ribosomal RNA (“an I element”). In some embodiments, a 3′ UTR or a proximal sequence thereto comprises a restriction site. In some embodiments, a restriction site is a BamHI site. In some embodiments, a restriction site is a XhoI site.

In some embodiments, an RNA disclosed herein comprises a 3′ UTR having at least 99%, 98%, 97%, 96%, 95%, 90%, 85%, or 80% identity to a 3′ UTR provided in SEQ ID NO: 13. In some embodiments, an RNA disclosed herein comprises a 3′ UTR provided in SEQ ID NO: 13.

### PolyA

In some embodiments, an RNA disclosed herein comprises a polyadenylate (PolyA) sequence, e.g., as described herein. In some embodiments, a PolyA sequence is situated downstream of a 3′-UTR, e.g., adjacent to a 3′-UTR.

As used herein, the terms “poly(A) sequence” or “PolyA sequence” or “poly-A tail” refers to an uninterrupted or interrupted sequence of adenylate residues which is typically located at the 3′-end of an RNA polynucleotide. Poly(A) sequences are known to those of skill in the art and may follow the 3′-UTR in RNAs described herein. An uninterrupted poly(A) sequence is characterized by consecutive adenylate residues. In nature, an uninterrupted poly(A) sequence is typical. RNAs disclosed herein can have a poly(A) sequence attached to the free 3′-end of the RNA by a template-independent RNA polymerase after transcription or a poly(A) sequence encoded by DNA and transcribed by a template-dependent RNA polymerase.

It has been demonstrated that a poly(A) sequence of about 120 A nucleotides has a beneficial influence on the levels of RNA in transfected eukaryotic cells, as well as on the levels of protein that is translated from an open reading frame that is present upstream (5′) of the poly(A) sequence (Holtkamp et al., 2006, Blood, vol. 108, pp. 4009-4017).

A poly(A) sequence may be of any length. In some embodiments, a poly(A) sequence comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly(A) sequence, typically at least 75%, at least 80%, at least 85%, at least 90%, at least 95%, at least 96%, at least 97%, at least 98%, or at least 99% by number of nucleotides in the poly(A) sequence are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly(A) sequence, i.e., 100% by number of nucleotides in the poly(A) sequence, are A nucleotides. The term “A nucleotide” or “A” refers to adenylate.

In some embodiments, a poly(A) sequence is attached during RNA transcription, e.g., during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette.

In some embodiments, the poly(A) cassette present in the coding strand of a DNA template essentially consists of dA nucleotides, but is interrupted by a random sequence of the four nucleotides (dA, dC, dG, and dT). Such random sequence may be 5 to 50, 10 to 30, or 10 to nucleotides in length. Such a cassette is disclosed in WO 2016/005324 A1, hereby incorporated by reference. Any poly(A) cassette disclosed in WO 2016/005324 A1 may be used in the present invention. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. In some embodiments, the poly(A) sequence contained in an RNA polynucleotide described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly(A) sequence at its 3′-end, i.e., the poly(A) sequence is not masked or followed at its 3′-end by a nucleotide other than A.

In some embodiments, the poly(A) sequence may comprise at least 20, at least 30, at least 40, at least 80, or at least 100 and up to 500, up to 400, up to 300, up to 200, or up to 150 nucleotides. In some embodiments, the poly(A) sequence may essentially consist of at least 20, at least 30, at least 40, at least 80, or at least 100 and up to 500, up to 400, up to 300, up to 200, or up to 150 nucleotides. In some embodiments, the poly(A) sequence may consist of at least 20, at least 30, at least 40, at least 80, or at least 100 and up to 500, up to 400, up to 300, up to 200, or up to 150 nucleotides. In some embodiments, the poly(A) sequence comprises at least 100 nucleotides. In some embodiments, the poly(A) sequence comprises about 150 nucleotides. In some embodiments, the poly(A) sequence comprises about 120 nucleotides.

In some embodiments, an RNA disclosed herein comprises a poly(A) sequence comprising the nucleotide sequence of SEQ ID NO: 14, or a nucleotide sequence having at least 99%, 98%, 97%, 96%, 95%, 90%, 85%, or 80% identity to the nucleotide sequence of SEQ ID NO: 14. In some embodiments, an RNA disclosed herein comprises a poly(A) sequence of SEQ ID NO: 14.

### Payloads

In some embodiments, an RNA polynucleotide disclosed herein comprises a sequence encoding a payload, e.g., as described herein. In some embodiments, a sequence encoding a payload comprises a promoter sequence. In some embodiments, a sequence encoding a payload comprises a sequence encoding a secretory signal peptide.

In some embodiments, a payload is chosen from: a protein replacement polypeptide; an antibody agent; a cytokine; an antigenic polypeptide; a gene editing component; a regenerative medicine component or combinations thereof.

In some embodiments, a payload is or comprises a protein replacement polypeptide. In some embodiments, a protein replacement polypeptide comprises a polypeptide with aberrant expression in a disease or disorder. In some embodiments, a protein replacement polypeptide comprises an intracellular protein, an extracellular protein, or a transmembrane protein. In some embodiments, a protein replacement polypeptide comprises an enzyme.

In some embodiments, a disease or disorder with aberrant expression of a polypeptide includes but is not limited to: a rare disease, a metabolic disorder, a muscular dystrophy, a cardiovascular disease, or a monogenic disease.

In some embodiments, a payload is or comprises an antibody agent. In some embodiments, an antibody agent binds to a polypeptide expressed on a cell. In some embodiments, an antibody agent comprises a CD3 antibody, a Claudin 6 antibody, or a combination thereof.

In some embodiments, a payload is or comprises a cytokine or a fragment or a variant thereof. In some embodiments, a cytokine comprises: IL-12 or a fragment or variant or a fusion thereof, IL-15 or a fragment or a variant or a fusion thereof, GM-CSF or a fragment or a variant thereof; or IFN-alpha or a fragment or a variant thereof.

In some embodiments, a payload is or comprises an antigenic polypeptide or an immunogenic variant or an immunogenic fragment thereof. In some embodiments, an antigenic polypeptide comprises one epitope from an antigen. In some embodiments, an antigenic polypeptide comprises a plurality of distinct epitopes from an antigen. In some embodiments, an antigenic polypeptide comprising a plurality of distinct epitopes from an antigen is polyepitopic.

In some embodiments, an antigenic polypeptide comprises: an antigenic polypeptide from an allergen, a viral antigenic polypeptide, a bacterial antigenic polypeptide, a fungal antigenic polypeptide, a parasitic antigenic polypeptide, an antigenic polypeptide from an infectious agent, an antigenic polypeptide from a pathogen, a tumor antigenic polypeptide, or a self-antigenic polypeptide.

In some embodiments, a viral antigenic polypeptide comprises an HIV antigenic polypeptide, an influenza antigenic polypeptide, a respiratory syncytial virus antigenic polypeptide, a Coronavirus antigenic polypeptide, a Rabies antigenic polypeptide, or a Zika virus antigenic polypeptide. In some embodiments, a viral antigenic polypeptide comprises an antigenic polypeptide of a virus that is associated with a respiratory infectious disease.

In some embodiments, a viral antigenic polypeptide is or comprises a Coronavirus antigenic polypeptide. In some embodiments, a Coronavirus antigen is or comprises a SARS-CoV-2 protein. In some embodiments, a SARS-CoV-2 protein comprises a SARS-CoV-2 Spike (S) protein, or an immunogenic variant or an immunogenic fragment thereof. In some embodiments, a SARS-CoV-2 protein, or immunogenic variant or immunogenic fragment thereof, comprises proline residues at positions 986 and 987.

In some embodiments, a SARS-CoV-2 S polypeptide has at least 99%, 98%, 97%, 96%, 95%, 90%, 85%, or 80% identity to a SARS-CoV-2 S polypeptide disclosed herein. In some embodiments, a SARS-CoV-2 S polypeptide has at least 99%, 98%, 97%, 96%, 95%, 90%, 85%, or 80% identity to SEQ ID NO: 9.

In some embodiments, a SARS-CoV-2 S polypeptide is encoded by an RNA having at least 99%, 98%, 97%, 96%, 95%, 90%, 85%, or 80% identity to a SARS-CoV-2 S polynucleotide disclosed herein. In some embodiments, a SARS-CoV-2 S polypeptide is encoded by an RNA having at least 99%, 98%, 97%, 96%, 95%, 90%, 85%, or 80% identity to SEQ ID NO: 10.

In some embodiments, a payload is or comprises a tumor antigenic polypeptide or an immunogenic variant or an immunogenic fragment thereof. In some embodiments, a tumor antigenic polypeptide comprises a tumor specific antigen, a tumor associated antigen, a tumor neoantigen, or a combination thereof. In some embodiments, a tumor antigenic polypeptide comprises p53, ART-4, BAGE, ss-catenin/m, Bcr-abL CAMEL, CAP-1, CASP-8, CDC27/m, CDK4/m, CEA, CLAUDIN-12, c-MYC, CT, Cyp-B, DAM, ELF2M, ETV6-AML1, G250, GAGE, GnT-V, Gap100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1R, Myosin/m, MUC1, MUM-1, MUM-2, MUM-3, NA88-A, NF1, NY-ESO-1, NY-BR-1, p190 minor BCR-abL, Plac-1, Pm1/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE, WT, WT-1, or a combination thereof.

In some embodiments, a tumor antigenic polypeptide comprises a tumor antigen from a carcinoma, a sarcoma, a melanoma, a lymphoma, a leukemia, or a combination thereof. In some embodiments, a tumor antigenic polypeptide comprises a melanoma tumor antigen. In some embodiments, a tumor antigenic polypeptide comprises a prostate cancer antigen. In some embodiments, a tumor antigenic polypeptide comprises a HPV16 positive head and neck cancer antigen. In some embodiments, a tumor antigenic polypeptide comprises a breast cancer antigen. In some embodiments, a tumor antigenic polypeptide comprises an ovarian cancer antigen. In some embodiments, a tumor antigenic polypeptide comprises a lung cancer antigen. In some embodiments, a tumor antigenic polypeptide comprises an NSCLC antigen.

In some embodiments, a payload is or comprises a self-antigenic polypeptide or an immunogenic variant or an immunogenic fragment thereof. In some embodiments, a self-antigenic polypeptide comprises an antigen that is typically expressed on cells and is recognized as a self-antigen by an immune system. In some embodiments, a self-antigenic polypeptide comprises: a multiple sclerosis antigenic polypeptide, a Rheumatoid arthritis antigenic polypeptide, a lupus antigenic polypeptide, a celiac disease antigenic polypeptide, a Sjogren's syndrome antigenic polypeptide, or an ankylosing spondylitis antigenic polypeptide, or a combination thereof.

### In Vitro Synthesis of RNA Polynucleotides

Commonly, in vitro transcription reactions include a double stranded DNA template comprised of a template strand (also known as a non-coding strand) and a coding strand. Those skilled in the art appreciate that a “Transcription Start Site” sequence, when presented as single stranded (SS) sequence, typically relates to the coding strand sequence and reflects the canonical position at which the relevant RNA polymerase begins transcription. Those skilled in the art, reading the present disclosure will appreciate that, in some embodiments, a cap (e.g., a co-transcriptional cap) may include one or more residues corresponding to a position of such a “transcriptional start site sequence”, such that the first residue added by the RNA polymerase may in fact represent the second (or later) residue of the canonical Transcription Start Site.

In some embodiments, a DNA template is a linear DNA molecule. In some embodiments, a DNA template is a circular DNA molecule. DNA can be obtained or generated using methods known in the art, including, e.g., gene synthesis, recombinant DNA technology, or a combination thereof. In some embodiments, a DNA template comprises a nucleotide sequence coding for a transcribed region of interest (e.g., coding for a RNA described herein) and a promoter sequence that is recognized by an RNA polymerase selected for use in in vitro transcription. Various RNA polymerases are known in the art, including, e.g., DNA dependent RNA polymerases (e.g., a T7 RNA polymerase, a T3 RNA polymerase, a SP6 RNA polymerase, a N₄ virion RNA polymerase, or a variant or functional domain thereof). A person skilled in the art will readily understand that an RNA polymerase utilized herein may be a recombinant RNA polymerase, and/or a purified RNA polymerase, i.e., not as part of a cell extract, which contains other components in addition to the RNA polymerases. One skilled in the art will recognize an appropriate promoter sequence for the selected RNA polymerase. In some embodiments, a DNA template can comprise a promoter sequence for a T7 RNA polymerase.

In some embodiments, the present disclosure provides an insight that a double stranded DNA template containing an A and U at the +1 and +2 positions, respectively, of a Transcription Start Site downstream from a RNA polymerase promoter (e.g., T7 promoter) can be useful for improving capping efficiency (e.g., percentage of capped transcripts in an in vitro transcription reaction), quality of an RNA preparation (e.g., of an in vitro transcribed RNA, e.g., the amount of short polynucleotide byproducts produced), translation efficiency of an RNA encoding a payload, and/or expression of a polypeptide payload encoded by an RNA.

In some embodiments, such improvements can be observed independent of the identity of a 5′ UTR, capping method (e.g., enzymatic capping vs. co-transcriptional capping), cap structures (e.g., Cap0, Cap1, or Cap2), coding sequences, types of ribonucleotides (e.g., modified nucleotides vs. non-modified nucleotides), formulation (e.g., lipoplex vs. lipid nanoparticles) or combinations thereof. In some particular embodiments, a double stranded DNA template comprises an A and U at the +1 and +2 positions, respectively, of a Transcription Start Site. In some embodiments, a pyrimidine base (e.g., C or U) or a purine base (e.g., G or A) can be independently present at +3, +4, or +5 positions of a Transcription Start Site of a double stranded DNA template. In some particular embodiments, such a double stranded DNA template comprises a A at +3 position of the Transcription Start Site.

As appreciated by a person skilled in the art, the 3′ end of a cap structure can be extended by an RNA polymerase using naturally occurring ribonucleotides and/or modified ribonucleotides. Therefore, a person skilled in the art will understand references to A, U, G, or C throughout the specification described herein can mean a naturally occurring ribonucleotide and/or a modified ribonucleotide described herein. For example, in some embodiments, a U is uridine. In some embodiments, a U is modified uridine (e.g., pseudouridine, 1-methyl pseudouridine).

In some embodiments, provided RNA polynucleotides are produced by in vitro transcription reaction described herein, e.g., using different combinations of cap structures (e.g., as described herein) and transcription start sites.

### AUA Transcription Start Site

In some embodiments, a Transcription Start Site that may be useful in accordance with the present disclosure is AUA. In some embodiments, an in vitro transcription reaction comprises: (i) a template DNA strand comprising a polynucleotide sequence complementary to an RNA polynucleotide sequence described herein, wherein the template DNA strand comprises a sequence that is complementary to an AUA transcription start site; (ii) a polymerase (e.g., an RNA polymerase such as, e.g., T7 polymerase); (iii) ribonucleotides; and (iv) a trinucleotide cap comprising N₁pN₂; wherein N₁ is A or an analog thereof (e.g., as described above and herein) and N₂ is U or an analog thereof (e.g., as described above and herein); and wherein the sequence in the template DNA strand that is complementary to AUA is the start site of an RNA polymerase promoter. A skilled person in the art reading the present disclosure will appreciate that when an AUA Transcription Start Site is referenced with respect to a double-stranded DNA template, a coding strand of the double-stranded DNA template comprises an AUA start sequence, while a template DNA strand of the double stranded DNA template comprises a TAT which is the start site of an RNA polymerase promoter.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide, wherein N₁ is A or an analog thereof (e.g., as described above and herein), and N₂ is U or an analog thereof (e.g., as described above and herein); and (iii) the cap proximal sequence comprises: N₁ and N₂ of the cap structure and a sequence comprising N₃N₄N₅ at positions +3, +4, and +5 respectively of the RNA polynucleotide, wherein each of N₃, N₄, and N₅ is independently selected from: A, C, G, and U (e.g., as described above and herein). By way of example only, in some embodiments, an RNA polynucleotide resulting from such an in vitro transcription reaction comprises a 5′ cap and a cap proximal sequence comprising A₁U₂A₃N₄N₅. In some embodiments, an RNA polynucleotide resulting from such an in vitro transcription reaction can be an RNA polynucleotide described herein.

### AUC Transcription Start Site

In some embodiments, a Transcription Start Site that may be useful in accordance with the present disclosure is AUA. In some embodiments, an in vitro transcription reaction comprises: (i) a template DNA strand comprising a polynucleotide sequence complementary to an RNA polynucleotide sequence described herein, wherein the template DNA strand comprises a sequence that is complementary to an AUC transcription start site; (ii) a polymerase (e.g., an RNA polymerase such as, e.g., T7 polymerase); (iii) ribonucleotides; and (iv) a trinucleotide cap comprising N₁pN₂; wherein N₁ is A or an analog thereof (e.g., as described above and herein) and N₂ is U or an analog thereof (e.g., as described above and herein); and wherein the sequence in the template DNA strand that is complementary to AUC is the start site of an RNA polymerase promoter. A skilled person in the art reading the present disclosure will appreciate that when an AUC Transcription Start Site is referenced with respect to a double-stranded DNA template, a coding strand of the double-stranded DNA template comprises an AUC start sequence, while a template DNA strand of the double stranded DNA template comprises a TAG which is the start site of an RNA polymerase promoter.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide, wherein N₁ is A or an analog thereof (e.g., as described above and herein), and N₂ is U or an analog thereof (e.g., as described above and herein); and (iii) the cap proximal sequence comprises: N₁ and N₂ of the cap structure and a sequence comprising N₃N₄N₅ at positions +3, +4, and +5 respectively of the RNA polynucleotide, wherein each of N₃, N₄, and N₅ is independently selected from: A, C, G, and U (e.g., as described above and herein). By way of example only, in some embodiments, an RNA polynucleotide resulting from such an in vitro transcription reaction comprises a 5′ cap and a cap proximal sequence comprising A₁U₂C₃N₄N₅. In some embodiments, an RNA polynucleotide resulting from such an in vitro transcription reaction can be an RNA polynucleotide described herein.

### AUG Transcription Start Site

In some embodiments, a Transcription Start Site that may be useful in accordance with the present disclosure is AUA. In some embodiments, an in vitro transcription reaction comprises: (i) a template DNA strand comprising a polynucleotide sequence complementary to an RNA polynucleotide sequence described herein, wherein the template DNA strand comprises a sequence that is complementary to an AUG transcription start site; (ii) a polymerase (e.g., an RNA polymerase such as, e.g., T7 polymerase); (iii) ribonucleotides; and (iv) a trinucleotide cap comprising N₁pN₂; wherein N₁ is A or an analog thereof (e.g., as described above and herein) and N₂ is U or an analog thereof (e.g., as described above and herein); and wherein the sequence in the template DNA strand that is complementary to AUG is the start site of an RNA polymerase promoter. A skilled person in the art reading the present disclosure will appreciate that when an AUG Transcription Start Site is referenced with respect to a double-stranded DNA template, a coding strand of the double-stranded DNA template comprises an AUG start sequence, while a template DNA strand of the double stranded DNA template comprises a TAC which is the start site of an RNA polymerase promoter.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide, wherein N₁ is A or an analog thereof (e.g., as described above and herein), and N₂ is U or an analog thereof (e.g., as described above and herein); and (iii) the cap proximal sequence comprises: N₁ and N₂ of the cap structure and a sequence comprising N₃N₄N₅ at positions +3, +4, and +5 respectively of the RNA polynucleotide, wherein each of N₃, N₄, and N₅ is independently selected from: A, C, G, and U (e.g., as described above and herein). By way of example only, in some embodiments, an RNA polynucleotide resulting from such an in vitro transcription reaction comprises a 5′ cap and a cap proximal sequence comprising A₁U₂G3N₄N₅. In some embodiments, an RNA polynucleotide resulting from such an in vitro transcription reaction can be an RNA polynucleotide described herein.

### AUU Transcription Start Site

In some embodiments, a Transcription Start Site that may be useful in accordance with the present disclosure is AUA. In some embodiments, an in vitro transcription reaction comprises: (i) a template DNA strand comprising a polynucleotide sequence complementary to an RNA polynucleotide sequence described herein, wherein the template DNA strand comprises a sequence that is complementary to an AL:UU transcription start site; (ii) a polymerase (e.g., an RNA polymerase such as, e.g., T7 polymerase); (iii) ribonucleotides; and (iv) a trinucleotide cap comprising N₁pN₂; wherein N₁ is A or an analog thereof (e.g., as described above and herein) and N₂ is U or an analog thereof (e.g., as described above and herein); and wherein the sequence in the template DNA strand that is complementary to AUG is the start site of an RNA polymerase promoter. A skilled person in the art reading the present disclosure will appreciate that when an AUU Transcription Start Site is referenced with respect to a double-stranded DNA template, a coding strand of the double-stranded DNA template comprises an AUU start sequence, while a template DNA strand of the double stranded DNA template comprises a TAA which is the start site of an RNA polymerase promoter.

In some embodiments, such in vitro transcription reactions can produce an RNA polynucleotide comprising a 5′ cap, a cap proximal sequence comprising positions +1, +2, +3, +4, and +5 of the RNA polynucleotide; and a sequence encoding a payload, wherein: (i) N₁ is position +1 of the RNA polynucleotide, (ii) N₂ is position +2 of the RNA polynucleotide, wherein N₁ is A or an analog thereof (e.g., as described above and herein), and N₂ is U or an analog thereof (e.g., as described above and herein); and (iii) the cap proximal sequence comprises: N₁ and N₂ of the cap structure and a sequence comprising N₃N₄N₅ at positions +3, +4, and +5 respectively of the RNA polynucleotide, wherein each of N₃, N₄, and N₅ is independently selected from: A, C, G, and U (e.g., as described above and herein). By way of example only, in some embodiments, an RNA polynucleotide resulting from such an in vitro transcription reaction comprises a 5′ cap and a cap proximal sequence comprising A₁U₂U₃N₄N₅. In some embodiments, an RNA polynucleotide resulting from such an in vitro transcription reaction can be an RNA polynucleotide described herein.

### Complexes

In certain aspects, provided herein are complexes formed during in vitro transcription reactions described herein, e.g., using different combinations of caps (e.g., as described herein) and transcription start sites (e.g., as described herein).

In some embodiments, the present disclosure provides a complex comprising a DNA template strand and a 5′ cap analog, wherein the DNA template strand comprises an RNA polymerase promoter sequence and a sequence that is complementary to a transcription start site; wherein the 5′ cap analog comprises a structure of N₁pN₂, and wherein N₁ is A or an analog thereof (e.g., as described above and herein) and N₂ is U or an analog thereof (e.g., as described above and herein); wherein N₁ interacts with the +1 position of the DNA template strand (corresponding to the first nucleotide of the transcription start site) and N₂ interacts with the +2 position of the DNA template strand (corresponding to the second nucleotide of the transcription start site); and wherein the sequence in the template strand that is complementary to the transcription start site is the start site of an RNA polymerase promoter. In some embodiments, N₁ is A and N₂ is U, and position +1 and position +2 of the DNA template strand are T and A, respectively.

In various aspects described herein, one or more nucleotides of a cap (e.g., ones described herein) interact with one or more nucleotides in the RNA polymerase start site the template DNA strand via canonical Watson-Crick base pairing. In some embodiments, a provided complex comprises a DNA template strand comprises an RNA polymerase promoter sequence, which in some embodiments may be or comprise a T7 RNA polymerase promoter sequence. In some embodiments, the complexes disclosed herein further comprise an RNA polymerase (e.g., a T7 RNA polymerase).

### Exemplary Polynucleotides

In some embodiments, an RNA polynucleotide described herein or a composition or medical preparation comprising the same comprises a nucleotide sequence disclosed herein. In some embodiments, an RNA polynucleotide comprises a sequence having at least 80% identity to a nucleotide sequence disclosed herein. In some embodiments, an RNA polynucleotide comprises a sequence encoding a polypeptide having at least 80% identity to a polypeptide sequence disclosed herein. Exemplary nucleotide and polypeptide sequences are provided e.g., in Table 2 or in this section titled “Exemplary polynucleotides” or in Example 1 or 2.

In some embodiments, an RNA polynucleotide described herein or a composition or medical preparation comprising the same is transcribed by a DNA template. In some embodiments, a DNA template used to transcribe an RNA polynucleotide described herein comprises a sequence complementary to an RNA polynucleotide.

In some embodiments, a payload described herein is encoded by an RNA polynucleotide described herein comprising a nucleotide sequence disclosed herein, e.g., in Table 2 or in this section titled “Exemplary polynucleotides” or in Example 1 or 2. In some embodiments, an RNA polynucleotide encodes a polypeptide payload having at least 80% identity to a polypeptide payload sequence disclosed herein. In some embodiments, a payload described herein is encoded by an RNA polynucleotide transcribed by a DNA template comprising a sequence complementary to an RNA polynucleotide.

TABLE 2
Exemplary sequences of RNA constructs disclosed herein

| Sequence | |
|---|---|
| information | Exemplary RNA Sequences |
| Cap proximal | AUAN₄N₅, wherein N₄ and N₅ can be each |
| consensus | independently any nucleotide (e.g., A, U, G, |
| sequence | C) |
| (nucleotides 1-5 | |
| of a 5′ UTR) | |
| comprising AUA | |
| start sequence | |
| Cap proximal | AUCN₄N₅, wherein N₄ and N₅ can be each |
| consensus | independently any nucleotide (e.g., A, U, G, |
| sequence | C) |
| (nucleotides 1-5 | |
| of a 5′ UTR) | |
| comprising AUC | |
| start sequence | |
| Cap proximal | AUGN₄N₅, wherein N₄ and N₅ can be each |
| consensus | independently any nucleotide (e.g., A, U, G, |
| sequence | C) |
| (nucleotides 1-5 | |
| of a 5′ UTR) | |
| comprising AUG | |
| start sequence | |
| Cap proximal | AUUNAN₅, wherein N₄ and N₅ can be each |
| consensus | independently any nucleotide (e.g., A, U, G, |
| sequence | C) |
| (nucleotides 1-5 | |
| of a 5′ UTR) | |
| comprising AUU | |
| start sequence | |
| Ligation 3 | GAGUCGCUAGCCGCGUCGCU |
| sequence | |
| (SEQ ID NO: 42) | |
| SARS-CoV-2 S | MFVFLVLLPLVSSQCVNLTTRTQLPPAYTNSFTRGVYYPDKVER |
| protein PP (amino | SSVLHSTQDLFLPFFSNVTWFHAIHVSGTNGTKRFDNPVLPEND |
| acid) (V08/V09) | GVYFASTEKSNIIRGWIFGTTLDSKTQSLLIVNNATNVVIKVCE |
| (SEQ ID NO: 9) | FQFCNDPFLGVYYHKNNKSWMESEFRVYSSANNCTFEYVSQPFL |
| | MDLEGKQGNEKNLREFVFKNIDGYFKIYSKHTPINLVRDLPQGE |
| | SALEPLVDLPIGINITRFQTLLALHRSYLTPGDSSSGWTAGAAA |
| | YYVGYLQPRTELLKYNENGTITDAVDCALDPLSETKCTLKSFTV |
| | EKGIYQTSNERVQPTESIVRFPNITNLCPFGEVENATRFASVYA |
| | WNRKRISNCVADYSVLYNSASFSTFKCYGVSPTKLNDLCFTNVY |
| | ADSFVIRGDEVRQIAPGQTGKIADYNYKLPDDFTGCVIAWNSNN |
| | LDSKVGGNYNYLYRLFRKSNLKPFERDISTEIYQAGSTPCNGVE |
| | GENCYFPLQSYGFQPTNGVGYQPYRVVVLSFELLHAPATVCGPK |
| | KSTNLVKNKCVNFNFNGLTGTGVLTESNKKELPFQQFGRDIADT |
| | TDAVRDPQTLEILDITPCSFGGVSVITPGTNTSNQVAVLYQDVN |
| | CTEVPVAIHADQLTPTWRVYSTGSNVFQTRAGCLIGAEHVNNSY |
| | ECDIPIGAGICASYQTQTNSPRRARSVASQSIIAYTMSLGAENS |
| | VAYSNNSIAIPTNFTISVTTEILPVSMTKTSVDCTMYICGDSTE |
| | CSNLLLQYGSFCTQLNRALTGIAVEQDKNTQEVFAQVKQIYKTP |
| | PIKDFGGFNFSQILPDPSKPSKRSFIEDLLENKVTLADAGFIKQ |
| | YGDCLGDIAARDLICAQKENGLTVLPPLLTDEMIAQYTSALLAG |
| | TITSGWTFGAGAALQIPFAMQMAYRENGIGVTQNVLYENQKLIA |
| | NQFNSAIGKIQDSLSSTASALGKLQDVVNQNAQALNTLVKQLSS |
| | NFGAISSVLNDILSRLDPPEAEVQIDRLITGRLQSLQTYVTQQL |
| | IRAAEIRASANLAATKMSECVLGQSKRVDFCGKGYHLMSFPQSA |
| | PHGVVFLHVTYVPAQEKNFTTAPAICHDGKAHFPREGVFVSNGT |
| | HWFVTQRNFYEPQIITTDNTFVSGNCDVVIGIVNNTVYDPLQPE |
| | LDSFKEELDKYFKNHTSPDVDLGDISGINASVVNIQKEIDRLNE |
| | VAKNLNESLIDLQELGKYEQYIKWPWYIWLGFIAGLIAIVMVTI |
| | MLCCMTSCCSCLKGCCSCGSCCKFDEDDSEPVLKGVKLHYT |
| RBP020.2 | agaauaaacu aguauucuuc ugguccccac agacucagag |
| (SEQ ID NO: 10) | agaacccgcc accauguucg |
| | uguuccuggu gcugcugccu cuggugucca gccagugugu |
| | gaaccugacc accagaacac agcugccucc agccuacacc |
| | aacagcuuua ccagaggcgu guacuacccc gacaaggugu |
| | ucagauccag cgugcugcac ucuacccagg accuguuccu |
| | gccuuucuuc agcaacguga ccugguucca cgccauccac |
| | guguccggca ccaauggcac caagagauuc gacaaccccg |
| | ugcugcccuu caacgacggg guguacuuug ccagcaccga |
| | gaaguccaac aucaucagag gcuggaucuu cggcaccaca |
| | cuggacagca agacccagag ccugcugauc gugaacaacg |
| | ccaccaacgu ggucaucaaa gugugcgagu uccaguucug |
| | caacgacccc uuccugggcg ucuacuacca caagaacaac |
| | aagagcugga uggaaagcga guuccgggug uacagcagcg |
| | ccaacaacug caccuucgag uacguguccc agccuuuccu |
| | gauggaccug gaaggcaagc agggcaacuu caagaaccug |
| | cgcgaguucg uguuuaagaa caucgacggc uacuucaaga |
| | ucuacagcaa gcacaccccu aucaaccucg ygcgggaucu |
| | gccucagggc uucucugcuc |
| | uggaaccccu gguggaucug cccaucggca ucaacaucac |
| | ccgguuucag acacugcugg cccugcacag aagcuaccug |
| | acaccuggcg auagcagcag cggauggaca gcuggugccg |
| | ccgcuuacua ugugggcuac cugcagccua gaaccuuccu |
| | gcugaaguac aacgagaacg gcaccaucac cgacgccgug |
| | gauugugcuc uggauccucu gagcgagaca aagugcaccc |
| | ugaaguccuu caccguggaa aagggcaucu accagaccag |
| | caacuuccgg gugcagccca |
| | ccgaauccau cgugcgguuc cccaauauca ccaaucugug |
| | ccccuucggc gagguguuca augccaccag auucgccucu |
| | guguacgccu ggaaccggaa gcggaucagc aauugcgugg |
| | ccgacuacuc cgugcuguac aacuccgcca gcuucagcac |
| | cuucaagugc uacggcgugu ccccuaccaa gcugaacgac |
| | cugugcuuca caaacgugua cgccgacagc uucgugaucc |
| | ggggagauga agugcggcag auugccccug gacagacagg |
| | caagaucgcc gacuacaacu |
| | acaagcugcc cgacgacuuc accggcugug ugauugccug |
| | gaacagcaac aaccuggacu ccaaagucgg cggcaacuac |
| | aauuaccugu accggcuguu ccggaagucc aaucugaagc |
| | ccuucgagcg ggacaucucc accgagaucu aucaggccgg |
| | cagcaccccu uguaacggcg uggaaggcuu caacugcuac |
| | uucccacugc aguccuacgg cuuucagccc acaaauggcg |
| | ugggcuauca gcccuacaga gugguggugc ugagcuucga |
| | acugcugcau gccccugcca |
| | cagugugcgg cccuaagaaa agcaccaauc ucgugaagaa |
| | caaaugcgug aacuucaacu |
| | ucaacggccu gaccggcacc ggcgugcuga cagagagcaa |
| | caagaaguuc cugccauucc agcaguuugg ccgggauauc |
| | gccgauacca cagacgccgu uagagauccc cagacacugg |
| | aaauccugga caucaccccu ugcagcuucg gcggaguguc |
| | ugugaucacc ccuggcacca acaccagcaa ucagguggca |
| | gugcuguacc aggacgugaa cuguaccgaa gugcccgugg |
| | ccauucacgc cgaucagcug acaccuacau ggcgggugua |
| | cuccaccggc agcaaugugu |
| | uucagaccag agccggcugu cugaucggag ccgagcacgu |
| | gaacaauagc uacgagugcg acauccccau cggcgcugga |
| | aucugcgcca gcuaccagac acagacaaac agcccucgga |
| | gagccagaag cguggccagc cagagcauca uugccuacac |
| | aaugucucug ggcgccgaga acagcguggc cuacuccaac |
| | aacucuaucg cuauccccac caacuucacc aucagcguga |
| | ccacagagau ccugccugug uccaugacca agaccagcgu |
| | ggacugcacc auguacaucu gcggcgauuc caccgagugc |
| | uccaaccugc ugcugcagua cggcagcuuc ugcacccagc |
| | ugaauagagc ccugacaggg aucgccgugg aacaggacaa |
| | gaacacccaa gagguguucg cccaagugaa gcagaucuac |
| | aagaccccuc cuaucaagga cuucggcggc uucaauuuca |
| | gccagauucu gcccgauccu agcaagccca gcaagcggag |
| | cuucaucgag gaccugcugu |
| | ucaacaaagu gacacuggcc gacgccggcu ucaucaagca |
| | guauggcgau ugucugggcg acauugccgc cagggaucug |
| | auuugcgccc agaaguuuaa cggacugaca gugcugccuc |
| | cucugcugac cgaugagaug aucgcccagu acacaucugc |
| | ccugcuggcc ggcacaauca caagcggcug gacauuugga |
| | gcaggcgccg cucugcagau ccccuuugcu augcagaugg |
| | ccuaccgguu caacggcauc ggagugaccc agaaugugcu |
| | guacgagaac cagaagcuga |
| | ucgccaacca guucaacagc gccaucggca agauccagga |
| | cagccugagc agcacagcaa |
| | gcgcccuggg aaagcugcag gacgugguca accagaaugc |
| | ccaggcacug aacacccugg ucaagcagcu guccuccaac |
| | uucggcgcca ucagcucugu gcugaacgau auccugagca |
| | gacuggaccc uccugaggcc gaggugcaga ucgacagacu |
| | gaucacaggc agacugcaga gccuccagac auacgugacc |
| | cagcagcuga ucagagccgc cgagauuaga gccucugcca |
| | aucuggccgc caccaagaug ucugagugug ugcugggcca |
| | gagcaagaga guggacuuuu gcggcaaggg cuaccaccug |
| | augagcuucc cucagucugc cccucacggc gugguguuuc |
| | ugcacgugac auaugugccc gcucaagaga agaauuucac |
| | caccgcucca gccaucugcc |
| | acgacggcaa agcccacuuu ccuagagaag gcguguucgu |
| | guccaacggc acccauuggu ucgugacaca gcggaacuuc |
| | uacgagcccc agaucaucac caccgacaac accuucgugu |
| | cuggcaacug cgacgucgug aucggcauug ugaacaauac |
| | cguguacgac ccucugcage ccgagcugga cagcuucaaa |
| | gaggaacugg acaaguacuu uaagaaccac acaagccccg |
| | acguggaccu gggcgauauc agcggaauca augccagcgu |
| | cgugaacauc cagaaagaga ucgaccggcu gaacgaggug |
| | gccaagaauc ugaacgagag ccugaucgac cugcaagaac |
| | uggggaagua cgagcaguac aucaaguggc ccugguacau |
| | cuggcugggc uuuaucgccg gacugauugc caucgugaug |
| | gucacaauca ugcuguguug caugaccagc ugcuguagcu |
| | gccugaaggg cuguuguagc uguggcagcu gcugcaaguu |
| | cgacgaggac gauucugagc ccgugcugaa gggcgugaaa |
| | cugcacuaca caugaugacu cgagcuggua cugcaugcac |
| | gcaaugcuag cugccccuuu cccguccugg guaccccgag |
| | ucucccccga ccucgggucc cagguaugcu cccaccucca |
| | ccugccccac ucaccaccuc ugcuaguucc agacaccucc |
| | caagcacgca gcaaugcagc ucaaaacgcu uagccuagcc |
| | acacccccac gggaaacage agugauuaac cuuuagcaau |
| | aaacgaaagu uuaacuaagc uauacuaacc ccaggguugg |
| | ucaauuucgu gccagccaca cccuggagcu agcaaaaaaa |
| | aaaaaaaaaa aaaaaaaaaa aaagcauaug acuaaaaaaa |
| | aaaaaaaaaa aaaaaaaaaa aaaaaaaaaa aaaaaaaaaa |
| | aaaaaaaaaa aaaaaaaaaa aaa |
| 5′ UTR | AUAGUAAACUAGUAUUCUUCUGGUCCCCACAGACUCAGAGAGAA |
| comprising an | CCC |
| AUAGU cap | |
| proximal | |
| sequence and a | |
| human alpha | |
| globin 5′ UTR | |
| sequence | |
| (SEQ ID NO: 11) | |
| Human alpha | AAACUAGUAUUCUUCUGGUCCCCACAGACUCAGAGAGAACCC |
| globin 5′ UTR | |
| (without first 5 | |
| nucleotides) | |
| (SEQ ID NO: 12) | |
| 3′ UTR (FI | CUGGUACUGCAUGCACGCAAUGCUAGCUGCCCCUUUCCCGUCCU |
| Element) | GGGUACCCCGAGUCUCCCCCGACCUCGGGUCCCAGGUAUGCUCC |
| (SEQ ID NO: 13) | CACCUCCACCUGCCCCACUCACCACCUCUGCUAGUUCCAGACAC |
| | CUCCCAAGCACGCAGCAAUGCAGCUCAAAACGCUUAGCCUAGCC |
| | ACACCCCCACGGGAAACAGCAGUGAUUAACCUUUAGCAAUAAAC |
| | GAAAGUUUAACUAAGCUAUACUAACCCCAGGGUUGGUCAAUUUC |
| | GUGCCAGCCACACC |
| A30L70 PolyA | AAAAAAAAAAAAAAAAAAAAAAAAAAAAAAGCAUAUGACUAAAA |
| (SEQ ID NO: 14) | AAAAAAAAAAAAAAAAAAAAAAAAAAAAAAAAAAAAAAAAAAAA |
| | AAAAAAAAAAAAAAAAAAAAAA |
| Exemplary RNA | AUAGUAAACUAGUAUUCUUCUGGUCCCCACAGACUCAGAGAGAA |
| polynucleotide | CCCGCCACCCUCGAGCUGGUACUGCAUGCACGCAAUGCUAGCUG |
| (vA 3.0.1) | CCCCUUUCCCGUCCUGGGUACCCCGAGUCUCCCCCGACCUCGGG |
| without coding | UCCCAGGUAUGCUCCCACCUCCACCUGCCCCACUCACCACCUCU |
| sequence of a | GCUAGUUCCAGACACCUCCCAAGCACGCAGCAAUGCAGCUCAAA |
| payload | ACGCUUAGCCUAGCCACACCCCCACGGGAAACAGCAGUGAUUAA |
| Bold = 5′ UTR | CCUUUAGCAAUAAACGAAAGUUUAACUAAGCUAUACUAACCCCA |
| (Bold and | GGGUUGGUCAAUUUCGUGCCAGCCACACCCUGGAGCUAGCAAAA |
| underlined = cap | AAAAAAAAAAAAAAAAAAAAAAAAAAGCAUAUGACUAAAAAAAA |
| proximal | AAAAAAAAAAAAAAAAAAAAAAAAAAAAAAAAAAAAAAAAAAAA |
| sequence within | AAAAAAAAAAAAAAAAAA |
| the 5′UTR, Bold | |
| and italicized = | |
| Kozak sequence | |
| within the | |
| 5′UTR)Italicized | |
| and underlined = | |
| 3′ UTR | |
| Italicized = PolyA | |
| (SEQ ID NO: 43) | |
| Exemplary RNA | AGAAUAAACUAGUAUUCUUCUGGUCCCCACAGACUCAGAGAGAA |
| polynucleotide | CCCGCCACCAUGUUCGUGUUCCUGGUGCUGCUGCCUCUGGUGUC |
| (vA 3.0.1) with a | CAGCCAGUGUGUGAACCUGACCACCAGAACACAGCUGCCUCCAG |
| payload sequence | CCUACACCAACAGCUUUACCAGAGGCGUGUACUACCCCGACAAG |
| Underline = | GUGUUCAGAUCCAGCGUGCUGCACUCUACCCAGGACCUGUUCCU |
| exemplary | GCCUUUCUUCAGCAACGUGACCUGGUUCCACGCCAUCCACGUGU |
| payload sequence | CCGGCACCAAUGGCACCAAGAGAUUCGACAACCCCGUGCUGCCC |
| (BNT162b2) | UUCAACGACGGGGUGUACUUUGCCAGCACCGAGAAGUCCAACAU |
| (SEQ ID NO: 10) | CAUCAGAGGCUGGAUCUUCGGCACCACACUGGACAGCAAGACCC |
| | AGAGCCUGCUGAUCGUGAACAACGCCACCAACGUGGUCAUCAAA |
| | GUGUGCGAGUUCCAGUUCUGCAACGACCCCUUCCUGGGCGUCUA |
| | CUACCACAAGAACAACAAGAGCUGGAUGGAAAGCGAGUUCCGGG |
| | UGUACAGCAGCGCCAACAACUGCACCUUCGAGUACGUGUCCCAG |
| | CCUUUCCUGAUGGACCUGGAAGGCAAGCAGGGCAACUUCAAGAA |
| | CCUGCGCGAGUUCGUGUUUAAGAACAUCGACGGCUACUUCAAGA |
| | UCUACAGCAAGCACACCCCUAUCAACCUCGUGCGGGAUCUGCCU |
| | CAGGGCUUCUCUGCUCUGGAACCCCUGGUGGAUCUGCCCAUCGG |
| | CAUCAACAUCACCCGGUUUCAGACACUGCUGGCCCUGCACAGAA |
| | GCUACCUGACACCUGGCGAUAGCAGCAGCGGAUGGACAGCUGGU |
| | GCCGCCGCUUACUAUGUGGGCUACCUGCAGCCUAGAACCUUCCU |
| | GCUGAAGUACAACGAGAACGGCACCAUCACCGACGCCGUGGAUU |
| | GUGCUCUGGAUCCUCUGAGCGAGACAAAGUGCACCCUGAAGUCC |
| | UUCACCGUGGAAAAGGGCAUCUACCAGACCAGCAACUUCCGGGU |
| | GCAGCCCACCGAAUCCAUCGUGCGGUUCCCCAAUAUCACCAAUC |
| | UGUGCCCCUUCGGCGAGGUGUUCAAUGCCACCAGAUUCGCCUCU |
| | GUGUACGCCUGGAACCGGAAGCGGAUCAGCAAUUGCGUGGCCGA |
| | CUACUCCGUGCUGUACAACUCCGCCAGCUUCAGCACCUUCAAGU |
| | GCUACGGCGUGUCCCCUACCAAGCUGAACGACCUGUGCUUCACA |
| | AACGUGUACGCCGACAGCUUCGUGAUCCGGGGAGAUGAAGUGCG |
| | GCAGAUUGCCCCUGGACAGACAGGCAAGAUCGCCGACUACAACU |
| | ACAAGCUGCCCGACGACUUCACCGGCUGUGUGAUUGCCUGGAAC |
| | AGCAACAACCUGGACUCCAAAGUCGGCGGCAACUACAAUUACCU |
| | GUACCGGCUGUUCCGGAAGUCCAAUCUGAAGCCCUUCGAGCGGG |
| | ACAUCUCCACCGAGAUCUAUCAGGCCGGCAGCACCCCUUGUAAC |
| | GGCGUGGAAGGCUUCAACUGCUACUUCCCACUGCAGUCCUACGG |
| | CUUUCAGCCCACAAAUGGCGUGGGCUAUCAGCCCUACAGAGUGG |
| | UGGUGCUGAGCUUCGAACUGCUGCAUGCCCCUGCCACAGUGUGC |
| | GGCCCUAAGAAAAGCACCAAUCUCGUGAAGAACAAAUGCGUGAA |
| | CUUCAACUUCAACGGCCUGACCGGCACCGGCGUGCUGACAGAGA |
| | GCAACAAGAAGUUCCUGCCAUUCCAGCAGUUUGGCCGGGAUAUC |
| | GCCGAUACCACAGACGCCGUUAGAGAUCCCCAGACACUGGAAAU |
| | CCUGGACAUCACCCCUUGCAGCUUCGGCGGAGUGUCUGUGAUCA |
| | CCCCUGGCACCAACACCAGCAAUCAGGUGGCAGUGCUGUACCAG |
| | GACGUGAACUGUACCGAAGUGCCCGUGGCCAUUCACGCCGAUCA |
| | GCUGACACCUACAUGGCGGGUGUACUCCACCGGCAGCAAUGUGU |
| | UUCAGACCAGAGCCGGCUGUCUGAUCGGAGCCGAGCACGUGAAC |
| | AAUAGCUACGAGUGCGACAUCCCCAUCGGCGCUGGAAUCUGCGC |
| | CAGCUACCAGACACAGACAAACAGCCCUCGGAGAGCCAGAAGCG |
| | UGGCCAGCCAGAGCAUCAUUGCCUACACAAUGUCUCUGGGCGCC |
| | GAGAACAGCGUGGCCUACUCCAACAACUCUAUCGCUAUCCCCAC |
| | CAACUUCACCAUCAGCGUGACCACAGAGAUCCUGCCUGUGUCCA |
| | UGACCAAGACCAGCGUGGACUGCACCAUGUACAUCUGCGGCGAU |
| | UCCACCGAGUGCUCCAACCUGCUGCUGCAGUACGGCAGCUUCUG |
| | CACCCAGCUGAAUAGAGCCCUGACAGGGAUCGCCGUGGAACAGG |
| | ACAAGAACACCCAAGAGGUGUUCGCCCAAGUGAAGCAGAUCUAC |
| | AAGACCCCUCCUAUCAAGGACUUCGGCGGCUUCAAUUUCAGCCA |
| | GAUUCUGCCCGAUCCUAGCAAGCCCAGCAAGCGGAGCUUCAUCG |
| | AGGACCUGCUGUUCAACAAAGUGACACUGGCCGACGCCGGCUUC |
| | AUCAAGCAGUAUGGCGAUUGUCUGGGCGACAUUGCCGCCAGGGA |
| | UCUGAUUUGCGCCCAGAAGUUUAACGGACUGACAGUGCUGCCUC |
| | CUCUGCUGACCGAUGAGAUGAUCGCCCAGUACACAUCUGCCCUG |
| | CUGGCCGGCACAAUCACAAGCGGCUGGACAUUUGGAGCAGGCGC |
| | CGCUCUGCAGAUCCCCUUUGCUAUGCAGAUGGCCUACCGGUUCA |
| | ACGGCAUCGGAGUGACCCAGAAUGUGCUGUACGAGAACCAGAAG |
| | CUGAUCGCCAACCAGUUCAACAGCGCCAUCGGCAAGAUCCAGGA |
| | CAGCCUGAGCAGCACAGCAAGCGCCCUGGGAAAGCUGCAGGACG |
| | UGGUCAACCAGAAUGCCCAGGCACUGAACACCCUGGUCAAGCAG |
| | CUGUCCUCCAACUUCGGCGCCAUCAGCUCUGUGCUGAACGAUAU |
| | CCUGAGCAGACUGGACCCUCCUGAGGCCGAGGUGCAGAUCGACA |
| | GACUGAUCACAGGCAGACUGCAGAGCCUCCAGACAUACGUGACC |
| | CAGCAGCUGAUCAGAGCCGCCGAGAUUAGAGCCUCUGCCAAUCU |
| | GGCCGCCACCAAGAUGUCUGAGUGUGUGCUGGGCCAGAGCAAGA |
| | GAGUGGACUUUUGCGGCAAGGGCUACCACCUGAUGAGCUUCCCU |
| | CAGUCUGCCCCUCACGGCGUGGUGUUUCUGCACGUGACAUAUGU |
| | GCCCGCUCAAGAGAAGAAUUUCACCACCGCUCCAGCCAUCUGCC |
| | ACGACGGCAAAGCCCACUUUCCUAGAGAAGGCGUGUUCGUGUCC |
| | AACGGCACCCAUUGGUUCGUGACACAGCGGAACUUCUACGAGCC |
| | CCAGAUCAUCACCACCGACAACACCUUCGUGUCUGGCAACUGCG |
| | ACGUCGUGAUCGGCAUUGUGAACAAUACCGUGUACGACCCUCUG |
| | CAGCCCGAGCUGGACAGCUUCAAAGAGGAACUGGACAAGUACUU |
| | UAAGAACCACACAAGCCCCGACGUGGACCUGGGCGAUAUCAGCG |
| | GAAUCAAUGCCAGCGUCGUGAACAUCCAGAAAGAGAUCGACCGG |
| | CUGAACGAGGUGGCCAAGAAUCUGAACGAGAGCCUGAUCGACCU |
| | GCAAGAACUGGGGAAGUACGAGCAGUACAUCAAGUGGCCCUGGU |
| | ACAUCUGGCUGGGCUUUAUCGCCGGACUGAUUGCCAUCGUGAUG |
| | GUCACAAUCAUGCUGUGUUGCAUGACCAGCUGCUGUAGCUGCCU |
| | GAAGGGCUGUUGUAGCUGUGGCAGCUGCUGCAAGUUCGACGAGG |
| | ACGAUUCUGAGCCCGUGCUGAAGGGCGUGAAACUGCACUACACA |
| | UGAUGACUCGAGCUGGUACUGCAUGCACGCAAUGCUAGCUGCCC |
| | CUUUCCCGUCCUGGGUACCCCGAGUCUCCCCCGACCUCGGGUCC |
| | CAGGUAUGCUCCCACCUCCACCUGCCCCACUCACCACCUCUGCU |
| | AGUUCCAGACACCUCCCAAGCACGCAGCAAUGCAGCUCAAAACG |
| | CUUAGCCUAGCCACACCCCCACGGGAAACAGCAGUGAUUAACCU |
| | UUAGCAAUAAACGAAAGUUUAACUAAGCUAUACUAACCCCAGGG |
| | UUGGUCAAUUUCGUGCCAGCCACACCCUGGAGCUAGCAAAAAAA |
| | AAAAAAAAAAAAAAAAAAAAAAAGCAUAUGACUAAAAAAAAAAA |
| | AAAAAAAAAAAAAAAAAAAAAAAAAAAAAAAAAAAAAAAAAAAA |
| | AAAAAAAAAAAAAAA |

RBL063.1 (SEQ ID NO: 28 nucleotide; SEQ ID NO: 9 amino acid)
Structure beta-S-ARCA(D1)-hAg-Kozak-S1S2-PP-FI-A30L70
Encoded antigen Viral spike protein (S1S2 protein) of the SARS-CoV-2 (S1S2 full-length protein, sequence variant)

SEQ ID NO: 28
gggcgaacua guauucuucu gguccccaca gacucagaga gaacccgcca ccauguuugu 60
guuucuugug cugcugccuc uugugucuuc ucagugugug aauuugacaa caagaacaca 120
gcugccacca gcuuauacaa auucuuuuac cagaggagug uauuauccug auaaaguguu 180
uagaucuucu gugcugcaca gcacacagga ccuguuucug ccauuuuuua gcaaugugac 240
augguuucau gcaauucaug ugucuggaac aaauggaaca aaaagauuug auaauccugu 300
gcugccuuuu aaugauggag uguauuuugc uucaacagaa aagucaaaua uuauuagagg 360
auggauuuuu ggaacaacac uggauucuaa aacacagucu cugcugauug ugaauaaugc 420
aacaaaugug gugauuaaag ugugugaauu ucaguuuugu aaugauccuu uucugggagu 480
guauuaucac aaaaauaaua aaucuuggau ggaaucugaa uuuagagugu auuccucugc 540
aaauaauugu acauuugaau augugucuca gccuuuucug auggaucugg aaggaaaaca 600
gggcaauuuu aaaaaucuga gagaauuugu guuuaaaaau auugauggau auuuuaaaau 660
uuauucuaaa cacacaccaa uuaauuuagu gagagaucug ccucagggau uuucugcucu 720
ggaaccucug guggaucugc caauuggcau uaauauuaca agauuucaga cacugcuggc 780
ucugcacaga ucuuaucuga caccuggaga uucuucuucu ggauggacag ccggagcugc 840
agcuuauuau gugggcuauc ugcagccaag aacauuucug cugaaauaua augaaaaugg 900
aacaauuaca gaugcugugg auugugcucu ggauccucug ucugaaacaa aauguacauu 960
aaaaucuuuu acaguggaaa aaggcauuua ucagacaucu aauuuuagag ugcagccaac 1020
agaaucuauu gugagauuuc caaauauuac aaaucugugu ccauuuggag aaguguuuaa 1080
ugcaacaaga uuugcaucug uguaugcaug gaauagaaaa agaauuucua auuguguggc 1140
ugauuauucu gugcuguaua auagugcuuc uuuuuccaca uuuaaauguu auggaguguc 1200
uccaacaaaa uuaaaugauu uauguuuuac aaauguguau gcugauucuu uugugaucag 1260
aggugaugaa gugagacaga uugcccccgg acagacagga aaaauugcug auuacaauua 1320
caaacugccu gaugauuuua caggaugugu gauugcuugg aauucuaaua auuuagauuc 1380
uaaaguggga ggaaauuaca auuaucugua cagacuguuu agaaaaucaa aucugaaacc 1440
uuuugaaaga gauauuucaa cagaaauuua ucaggcugga ucaacaccuu guaauggagu 1500
ggaaggauuu aauuguuauu uuccauuaca gagcuaugga uuucagccaa ccaauggugu 1560
gggauaucag ccauauagag ugguggugcu gucuuuugaa cugcugcaug caccugcaac 1620
agugugugga ccuaaaaaau cuacaaauuu agugaaaaau aaauguguga auuuuaauuu 1680
uaauggauua acaggaacag gagugcugac agaaucuaau aaaaaauuuc ugccuuuuca 1740
gcaguuuggc agagauauug cagauaccac agaugcagug agagauccuc agacauuaga 1800
aauucuggau auuacaccuu guucuuuugg gggugugucu gugauuacac cuggaacaaa 1860
uacaucuaau cagguggcug ugcuguauca ggaugugaau uguacagaag ugccaguggc 1920
aauucaugca gaucagcuga caccaacaug gagaguguau ucuacaggau cuaauguguu 1980
ucagacaaga gcaggauguc ugauuggagc agaacaugug aauaauucuu augaauguga 2040
uauuccaauu ggagcaggca uuugugcauc uuaucagaca cagacaaauu ccccaaggag 2100
agcaagaucu guggcaucuc agucuauuau ugcauacacc augucucugg gagcagaaaa 2160
uucuguggca uauucuaaua auucuauugc uauuccaaca aauuuuacca uuucugugac 2220
aacagaaauu uuaccugugu cuaugacaaa aacaucugug gauuguacca uguacauuug 2280
uggagauucu acagaauguu cuaaucugcu gcugcaguau ggaucuuuuu guacacagcu 2340
gaauagagcu uuaacaggaa uugcugugga acaggauaaa aauacacagg aaguguuugc 2400
ucaggugaaa cagauuuaca aaacaccacc aauuaaagau uuuggaggau uuaauuuuag 2460
ccagauucug ccugauccuu cuaaaccuuc uaaaagaucu uuuauugaag aucugcuguu 2520
uaauaaagug acacuggcag augcaggauu uauuaaacag uauggagauu gccuggguga 2580
uauugcugca agagaucuga uuugugcuca gaaauuuaau ggacugacag ugcugccucc 2640
ucugcugaca gaugaaauga uugcucagua cacaucugcu uuacuggcug gaacaauuac 2700
aagcggaugg acauuuggag cuggagcugc ucugcagauu ccuuuugcaa ugcagauggc 2760
uuacagauuu aauggaauug gagugacaca gaauguguua uaugaaaauc agaaacugau 2820
ugcaaaucag uuuaauucug caauuggcaa aauucaggau ucucugucuu cuacagcuuc 2880
ugcucuggga aaacugcagg auguggugaa ucagaaugca caggcacuga auacucuggu 2940
gaaacagcug ucuagcaauu uuggggcaau uucuucugug cugaaugaua uucugucuag 3000
acuggauccu ccugaagcug aagugcagau ugauagacug aucacaggaa gacugcaguc 3060
ucugcagacu uaugugacac agcagcugau uagagcugcu gaaauuagag cuucugcuaa 3120
ucuggcugcu acaaaaaugu cugaaugugu gcugggacag ucaaaaagag uggauuuuug 3180
uggaaaagga uaucaucuga ugucuuuucc acagucugcu ccacauggag ugguguuuuu 3240
acaugugaca uaugugccag cacaggaaaa gaauuuuacc acagcaccag caauuuguca 3300
ugauggaaaa gcacauuuuc caagagaagg aguguuugug ucuaauggaa cacauugguu 3360
ugugacacag agaaauuuuu augaaccuca gauuauuaca acagauaaua cauuuguguc 3420
aggaaauugu gaugugguga uuggaauugu gaauaauaca guguaugauc cacugcagcc 3480
agaacuggau ucuuuuaaag aagaacugga uaaauauuuu aaaaaucaca caucuccuga 3540
uguggauuua ggagauauuu cuggaaucaa ugcaucugug gugaauauuc agaaagaaau 3600
ugauagacug aaugaagugg ccaaaaaucu gaaugaaucu cugauugauc ugcaggaacu 3660
uggaaaauau gaacaguaca uuaaauggcc uugguacauu uggcuuggau uuauugcagg 3720
auuaauugca auugugaugg ugacaauuau guuauguugu augacaucau guuguucuug 3780
uuuaaaagga uguuguucuu guggaagcug uuguaaauuu gaugaagaug auucugaacc 3840
uguguuaaaa ggagugaaau ugcauuacac augaugacuc gagcugguac ugcaugcacg 3900
caaugcuagc ugccccuuuc ccguccuggg uaccccgagu cucccccgac cucggguccc 3960
agguaugcuc ccaccuccac cugccccacu caccaccucu gcuaguucca gacaccuccc 4020
aagcacgcag caaugcagcu caaaacgcuu agccuagcca cacccccacg ggaaacagca 4080
gugauuaacc uuuagcaaua aacgaaaguu uaacuaagcu auacuaaccc caggguuggu 4140
caauuucgug ccagccacac ccuggagcua gcaaaaaaaa aaaaaaaaaa aaaaaaaaaa 4200
aagcauauga cuaaaaaaaa aaaaaaaaaa aaaaaaaaaa aaaaaaaaaa aaaaaaaaaa 4260
aaaaaaaaaa aaaaaaaaaa aa 4282

RBL063.2 (SEQ ID NO: 29 nucleotide; SEQ ID NO: 9 amino acid)
Structure beta-S-ARCA(D1)-hAg-Kozak-S1S2-PP-FI-A30L70 Encoded antigen Viral spike protein (S1S2 protein) of the SARS-CoV-2 (S1S2 full-length protein, sequence variant)

SEQ ID NO: 29
gggcgaacua guauucuucu gguccccaca gacucagaga gaacccgcca ccauguucgu 60
guuccuggug cugcugccuc ugguguccag ccagugugug aaccugacca ccagaacaca 120
gcugccucca gccuacacca acagcuuuac cagaggcgug uacuaccccg acaagguguu 180
cagauccagc gugcugcacu cuacccagga ccuguuccug ccuuucuuca gcaacgugac 240
cugguuccac gccauccacg uguccggcac caauggcacc aagagauucg acaaccccgu 300
gcugcccuuc aacgacgggg uguacuuugc cagcaccgag aaguccaaca ucaucagagg 360
cuggaucuuc ggcaccacac uggacagcaa gacccagagc cugcugaucg ugaacaacgc 420
caccaacgug gucaucaaag ugugcgaguu ccaguucugc aacgaccccu uccugggcgu 480
cuacuaccac aagaacaaca agagcuggau ggaaagcgag uuccgggugu acagcagcgc 540
caacaacugc accuucgagu acguguccca gccuuuccug auggaccugg aaggcaagca 600
gggcaacuuc aagaaccugc gcgaguucgu guuuaagaac aucgacggcu acuucaagau 660
cuacagcaag cacaccccua ucaaccucgu gcgggaucug ccucagggcu ucucugcucu 720
ggaaccccug guggaucugc ccaucggcau caacaucacc cgguuucaga cacugcuggc 780
ccugcacaga agcuaccuga caccuggcga uagcagcagc ggauggacag cuggugccgc 840
cgcuuacuau gugggcuacc ugcagccuag aaccuuccug cugaaguaca acgagaacgg 900
caccaucacc gacgccgugg auugugcucu ggauccucug agcgagacaa agugcacccu 960
gaaguccuuc accguggaaa agggcaucua ccagaccagc aacuuccggg ugcagcccac 1020
cgaauccauc gugcgguucc ccaauaucac caaucugugc cccuucggcg agguguucaa 1080
ugccaccaga uucgccucug uguacgccug gaaccggaag cggaucagca auugcguggc 1140
cgacuacucc gugcuguaca acuccgccag cuucagcacc uucaagugcu acggcguguc 1200
cccuaccaag cugaacgacc ugugcuucac aaacguguac gccgacagcu ucgugauccg 1260
gggagaugaa gugcggcaga uugccccugg acagacaggc aagaucgccg acuacaacua 1320
caagcugccc gacgacuuca ccggcugugu gauugccugg aacagcaaca accuggacuc 1380
caaagucggc ggcaacuaca auuaccugua ccggcuguuc cggaagucca aucugaagcc 1440
cuucgagcgg gacaucucca ccgagaucua ucaggccggc agcaccccuu guaacggcgu 1500
ggaaggcuuc aacugcuacu ucccacugca guccuacggc uuucagccca caaauggcgu 1560
gggcuaucag cccuacagag ugguggugcu gagcuucgaa cugcugcaug ccccugccac 1620
agugugcggc ccuaagaaaa gcaccaaucu cgugaagaac aaaugcguga acuucaacuu 1680
caacggccug accggcaccg gcgugcugac agagagcaac aagaaguucc ugccauucca 1740
gcaguuuggc cgggauaucg ccgauaccac agacgccguu agagaucccc agacacugga 1800
aauccuggac aucaccccuu gcagcuucgg cggagugucu gugaucaccc cuggcaccaa 1860
caccagcaau cagguggcag ugcuguacca ggacgugaac uguaccgaag ugcccguggc 1920
cauucacgcc gaucagcuga caccuacaug gcggguguac uccaccggca gcaauguguu 1980
ucagaccaga gccggcuguc ugaucggagc cgagcacgug aacaauagcu acgagugcga 2040
cauccccauc ggcgcuggaa ucugcgccag cuaccagaca cagacaaaca gcccucggag 2100
agccagaagc guggccagcc agagcaucau ugccuacaca augucucugg gcgccgagaa 2160
cagcguggcc uacuccaaca acucuaucgc uauccccacc aacuucacca ucagcgugac 2220
cacagagauc cugccugugu ccaugaccaa gaccagcgug gacugcacca uguacaucug 2280
cggcgauucc accgagugcu ccaaccugcu gcugcaguac ggcagcuucu gcacccagcu 2340
gaauagagcc cugacaggga ucgccgugga acaggacaag aacacccaag agguguucgc 2400
ccaagugaag cagaucuaca agaccccucc uaucaaggac uucggcggcu ucaauuucag 2460
ccagauucug cccgauccua gcaagcccag caagcggagc uucaucgagg accugcuguu 2520
caacaaagug acacuggccg acgccggcuu caucaagcag uauggcgauu gucugggcga 2580
cauugccgcc agggaucuga uuugcgccca gaaguuuaac ggacugacag ugcugccucc 2640
ucugcugacc gaugagauga ucgcccagua cacaucugcc cugcuggccg gcacaaucac 2700
aagcggcugg acauuuggag caggcgccgc ucugcagauc cccuuugcua ugcagauggc 2760
cuaccgguuc aacggcaucg gagugaccca gaaugugcug uacgagaacc agaagcugau 2820
cgccaaccag uucaacagcg ccaucggcaa gauccaggac agccugagca gcacagcaag 2880
cgcccuggga aagcugcagg acguggucaa ccagaaugcc caggcacuga acacccuggu 2940
caagcagcug uccuccaacu ucggcgccau cagcucugug cugaacgaua uccugagcag 3000
acuggacccu ccugaggccg aggugcagau cgacagacug aucacaggca gacugcagag 3060
ccuccagaca uacgugaccc agcagcugau cagagccgcc gagauuagag ccucugccaa 3120
ucuggccgcc accaagaugu cugagugugu gcugggccag agcaagagag uggacuuuug 3180
cggcaagggc uaccaccuga ugagcuuccc ucagucugcc ccucacggcg ugguguuucu 3240
gcacgugaca uaugugcccg cucaagagaa gaauuucacc accgcuccag ccaucugcca 3300
cgacggcaaa gcccacuuuc cuagagaagg cguguucgug uccaacggca cccauugguu 3360
cgugacacag cggaacuucu acgagcccca gaucaucacc accgacaaca ccuucguguc 3420
uggcaacugc gacgucguga ucggcauugu gaacaauacc guguacgacc cucugcagcc 3480
cgagcuggac agcuucaaag aggaacugga caaguacuuu aagaaccaca caagccccga 3540
cguggaccug ggcgauauca gcggaaucaa ugccagcguc gugaacaucc agaaagagau 3600
cgaccggcug aacgaggugg ccaagaaucu gaacgagagc cugaucgacc ugcaagaacu 3660
ggggaaguac gagcaguaca ucaaguggcc cugguacauc uggcugggcu uuaucgccgg 3720
acugauugcc aucgugaugg ucacaaucau gcuguguugc augaccagcu gcuguagcug 3780
ccugaagggc uguuguagcu guggcagcug cugcaaguuc gacgaggacg auucugagcc 3840
cgugcugaag ggcgugaaac ugcacuacac augaugacuc gagcugguac ugcaugcacg 3900
caaugcuagc ugccccuuuc ccguccuggg uaccccgagu cucccccgac cucggguccc 3960
agguaugcuc ccaccuccac cugccccacu caccaccucu gcuaguucca gacaccuccc 4020
aagcacgcag caaugcagcu caaaacgcuu agccuagcca cacccccacg ggaaacagca 4080
gugauuaacc uuuagcaaua aacgaaaguu uaacuaagcu auacuaaccc caggguuggu 4140
caauuucgug ccagccacac ccuggagcua gcaaaaaaaa aaaaaaaaaa aaaaaaaaaa 4200
aagcauauga cuaaaaaaaa aaaaaaaaaa aaaaaaaaaa aaaaaaaaaa aaaaaaaaaa 4260
aaaaaaaaaa aaaaaaaaaa aa 4282

BNT162a1; RBL063.3 (SEQ ID NO: 30 nucleotide; SEQ ID NO: 21 amino acid)
Structure beta-S-ARCA(D1)-hAg-Kozak-RBD-GS-Fibritin-FI-A30L70
Encoded antigen Viral spike protein (S protein) of the SARS-CoV-2 (partial sequence, Receptor Binding Domain (RBD) of S1S2 protein)

SEQ ID NO: 30
gggcgaacua guauucuucu gguccccaca gacucagaga gaacccgcca ccauguuugu 60
guuucuugug cugcugccuc uugugucuuc ucagugugug gugagauuuc caaauauuac 120
aaaucugugu ccauuuggag aaguguuuaa ugcaacaaga uuugcaucug uguaugcaug 180
gaauagaaaa agaauuucua auuguguggc ugauuauucu gugcuguaua auagugcuuc 240
uuuuuccaca uuuaaauguu auggaguguc uccaacaaaa uuaaaugauu uauguuuuac 300
aaauguguau gcugauucuu uugugaucag aggugaugaa gugagacaga uugcccccgg 360
acagacagga aaaauugcug auuacaauua caaacugccu gaugauuuua caggaugugu 420
gauugcuugg aauucuaaua auuuagauuc uaaaguggga ggaaauuaca auuaucugua 480
cagacuguuu agaaaaucaa aucugaaacc uuuugaaaga gauauuucaa cagaaauuua 540
ucaggcugga ucaacaccuu guaauggagu ggaaggauuu aauuguuauu uuccauuaca 600
gagcuaugga uuucagccaa ccaauggugu gggauaucag ccauauagag ugguggugcu 660
gucuuuugaa cugcugcaug caccugcaac agugugugga ccuaaaggcu cccccggcuc 720
cggcuccgga ucugguuaua uuccugaagc uccaagagau gggcaagcuu acguucguaa 780
agauggcgaa uggguauuac uuucuaccuu uuuaggccgg ucccuggagg ugcuguucca 840
gggccccggc ugaugacucg agcugguacu gcaugcacgc aaugcuagcu gccccuuucc 900
cguccugggu accccgaguc ucccccgacc ucggguccca gguaugcucc caccuccacc 960
ugccccacuc accaccucug cuaguuccag acaccuccca agcacgcagc aaugcagcuc 1020
aaaacgcuua gccuagccac acccccacgg gaaacagcag ugauuaaccu uuagcaauaa 1080
acgaaaguuu aacuaagcua uacuaacccc aggguugguc aauuucgugc cagccacacc 1140
cuggagcuag caaaaaaaaa aaaaaaaaaa aaaaaaaaaa agcauaugac uaaaaaaaaa 1200
aaaaaaaaaa aaaaaaaaaa aaaaaaaaaa aaaaaaaaaa aaaaaaaaaa aaaaaaaaaa 1260
a 1261

BNT162b2; RBP020.1 (SEQ ID NO: 31 nucleotide; SEQ ID NO: 9 amino acid)
Structure m₂^{7,3′-O}Gppp(m₁^{2′-O})ApG)-hAg-Kozak-S1S2-PP-FI-A30L70
Encoded antigen Viral spike protein (S1S2 protein) of the SARS-CoV-2 (S1S2 full-length protein, sequence variant)

SEQ ID NO: 31
agaauaaacu aguauucuuc ugguccccac agacucagag agaacccgcc accauguuug 60
uguuucuugu gcugcugccu cuugugucuu cucagugugu gaauuugaca acaagaacac 120
agcugccacc agcuuauaca aauucuuuua ccagaggagu guauuauccu gauaaagugu 180
uuagaucuuc ugugcugcac agcacacagg accuguuucu gccauuuuuu agcaauguga 240
caugguuuca ugcaauucau gugucuggaa caaauggaac aaaaagauuu gauaauccug 300
ugcugccuuu uaaugaugga guguauuuug cuucaacaga aaagucaaau auuauuagag 360
gauggauuuu uggaacaaca cuggauucua aaacacaguc ucugcugauu gugaauaaug 420
caacaaaugu ggugauuaaa gugugugaau uucaguuuug uaaugauccu uuucugggag 480
uguauuauca caaaaauaau aaaucuugga uggaaucuga auuuagagug uauuccucug 540
caaauaauug uacauuugaa uaugugucuc agccuuuucu gauggaucug gaaggaaaac 600
agggcaauuu uaaaaaucug agagaauuug uguuuaaaaa uauugaugga uauuuuaaaa 660
uuuauucuaa acacacacca auuaauuuag ugagagaucu gccucaggga uuuucugcuc 720
uggaaccucu gguggaucug ccaauuggca uuaauauuac aagauuucag acacugcugg 780
cucugcacag aucuuaucug acaccuggag auucuucuuc uggauggaca gccggagcug 840
cagcuuauua ugugggcuau cugcagccaa gaacauuucu gcugaaauau aaugaaaaug 900
gaacaauuac agaugcugug gauugugcuc uggauccucu gucugaaaca aaauguacau 960
uaaaaucuuu uacaguggaa aaaggcauuu aucagacauc uaauuuuaga gugcagccaa 1020
cagaaucuau ugugagauuu ccaaauauua caaaucugug uccauuugga gaaguguuua 1080
augcaacaag auuugcaucu guguaugcau ggaauagaaa aagaauuucu aauugugugg 1140
cugauuauuc ugugcuguau aauagugcuu cuuuuuccac auuuaaaugu uauggagugu 1200
cuccaacaaa auuaaaugau uuauguuuua caaaugugua ugcugauucu uuugugauca 1260
gaggugauga agugagacag auugcccccg gacagacagg aaaaauugcu gauuacaauu 1320
acaaacugcc ugaugauuuu acaggaugug ugauugcuug gaauucuaau aauuuagauu 1380
cuaaaguggg aggaaauuac aauuaucugu acagacuguu uagaaaauca aaucugaaac 1440
cuuuugaaag agauauuuca acagaaauuu aucaggcugg aucaacaccu uguaauggag 1500
uggaaggauu uaauuguuau uuuccauuac agagcuaugg auuucagcca accaauggug 1560
ugggauauca gccauauaga gugguggugc ugucuuuuga acugcugcau gcaccugcaa 1620
cagugugugg accuaaaaaa ucuacaaauu uagugaaaaa uaaaugugug aauuuuaauu 1680
uuaauggauu aacaggaaca ggagugcuga cagaaucuaa uaaaaaauuu cugccuuuuc 1740
agcaguuugg cagagauauu gcagauacca cagaugcagu gagagauccu cagacauuag 1800
aaauucugga uauuacaccu uguucuuuug gggguguguc ugugauuaca ccuggaacaa 1860
auacaucuaa ucagguggcu gugcuguauc aggaugugaa uuguacagaa gugccagugg 1920
caauucaugc agaucagcug acaccaacau ggagagugua uucuacagga ucuaaugugu 1980
uucagacaag agcaggaugu cugauuggag cagaacaugu gaauaauucu uaugaaugug 2040
auauuccaau uggagcaggc auuugugcau cuuaucagac acagacaaau uccccaagga 2100
gagcaagauc uguggcaucu cagucuauua uugcauacac caugucucug ggagcagaaa 2160
auucuguggc auauucuaau aauucuauug cuauuccaac aaauuuuacc auuucuguga 2220
caacagaaau uuuaccugug ucuaugacaa aaacaucugu ggauuguacc auguacauuu 2280
guggagauuc uacagaaugu ucuaaucugc ugcugcagua uggaucuuuu uguacacagc 2340
ugaauagagc uuuaacagga auugcugugg aacaggauaa aaauacacag gaaguguuug 2400
cucaggugaa acagauuuac aaaacaccac caauuaaaga uuuuggagga uuuaauuuua 2460
gccagauucu gccugauccu ucuaaaccuu cuaaaagauc uuuuauugaa gaucugcugu 2520
uuaauaaagu gacacuggca gaugcaggau uuauuaaaca guauggagau ugccugggug 2580
auauugcugc aagagaucug auuugugcuc agaaauuuaa uggacugaca gugcugccuc 2640
cucugcugac agaugaaaug auugcucagu acacaucugc uuuacuggcu ggaacaauua 2700
caagcggaug gacauuugga gcuggagcug cucugcagau uccuuuugca augcagaugg 2760
cuuacagauu uaauggaauu ggagugacac agaauguguu auaugaaaau cagaaacuga 2820
uugcaaauca guuuaauucu gcaauuggca aaauucagga uucucugucu ucuacagcuu 2880
cugcucuggg aaaacugcag gaugugguga aucagaaugc acaggcacug aauacucugg 2940
ugaaacagcu gucuagcaau uuuggggcaa uuucuucugu gcugaaugau auucugucua 3000
gacuggaucc uccugaagcu gaagugcaga uugauagacu gaucacagga agacugcagu 3060
cucugcagac uuaugugaca cagcagcuga uuagagcugc ugaaauuaga gcuucugcua 3120
aucuggcugc uacaaaaaug ucugaaugug ugcugggaca gucaaaaaga guggauuuuu 3180
guggaaaagg auaucaucug augucuuuuc cacagucugc uccacaugga gugguguuuu 3240
uacaugugac auaugugcca gcacaggaaa agaauuuuac cacagcacca gcaauuuguc 3300
augauggaaa agcacauuuu ccaagagaag gaguguuugu gucuaaugga acacauuggu 3360
uugugacaca gagaaauuuu uaugaaccuc agauuauuac aacagauaau acauuugugu 3420
caggaaauug ugauguggug auuggaauug ugaauaauac aguguaugau ccacugcagc 3480
cagaacugga uucuuuuaaa gaagaacugg auaaauauuu uaaaaaucac acaucuccug 3540
auguggauuu aggagauauu ucuggaauca augcaucugu ggugaauauu cagaaagaaa 3600
uugauagacu gaaugaagug gccaaaaauc ugaaugaauc ucugauugau cugcaggaac 3660
uuggaaaaua ugaacaguac auuaaauggc cuugguacau uuggcuugga uuuauugcag 3720
gauuaauugc aauugugaug gugacaauua uguuauguug uaugacauca uguuguucuu 3780
guuuaaaagg auguuguucu uguggaagcu guuguaaauu ugaugaagau gauucugaac 3840
cuguguuaaa aggagugaaa uugcauuaca caugaugacu cgagcuggua cugcaugcac 3900
gcaaugcuag cugccccuuu cccguccugg guaccccgag ucucccccga ccucgggucc 3960
cagguaugcu cccaccucca ccugccccac ucaccaccuc ugcuaguucc agacaccucc 4020
caagcacgca gcaaugcagc ucaaaacgcu uagccuagcc acacccccac gggaaacagc 4080
agugauuaac cuuuagcaau aaacgaaagu uuaacuaagc uauacuaacc ccaggguugg 4140
ucaauuucgu gccagccaca cccuggagcu agcaaaaaaa aaaaaaaaaa aaaaaaaaaa 4200
aaagcauaug acuaaaaaaa aaaaaaaaaa aaaaaaaaaa aaaaaaaaaa aaaaaaaaaa 4260
aaaaaaaaaa aaaaaaaaaa aaa 4283

RBP020.2 (SEQ ID NO: 10 nucleotide; SEQ ID NO: 9 amino acid) (see Table 2)
Structure m₂^{7,3′-O}Gppp(m₁^{2′-O})ApG)-hAg-Kozak-S1S2-PP-FI-A30L70
Encoded antigen Viral spike protein (S1S2 protein) of the SARS-CoV-2 (S1S2 full-length protein, sequence variant)
   BNT162b1; RBP020.3 (SEQ ID NO: 32; SEQ ID NO: 21 amino acid)
Structure m₂^{7,3′-O}Gppp(m₁^{2′-O})ApG)-hAg-Kozak-RBD-GS-Fibritin-FI-A30L70
Encoded antigen Viral spike protein (S1S2 protein) of the SARS-CoV-2 (partial sequence, Receptor Binding Domain (RBD) of S1S2 protein fused to fibritin)

SEQ ID NO: 32
agaauaaacu aguauucuuc ugguccccac agacucagag agaacccgcc accauguuug 60
agaauaaacu aguauucuuc ugguccccac agacucagag agaacccgcc accauguuug 120
caaaucugug uccauuugga gaaguguuua augcaacaag auuugcaucu guguaugcau 180
ggaauagaaa aagaauuucu aauugugugg cugauuauuc ugugcuguau aauagugcuu 240
cuuuuuccac auuuaaaugu uauggagugu cuccaacaaa auuaaaugau uuauguuuua 300
caaaugugua ugcugauucu uuugugauca gaggugauga agugagacag auugcccccg 360
gacagacagg aaaaauugcu gauuacaauu acaaacugcc ugaugauuuu acaggaugug 420
ugauugcuug gaauucuaau aauuuagauu cuaaaguggg aggaaauuac aauuaucugu 480
acagacuguu uagaaaauca aaucugaaac cuuuugaaag agauauuuca acagaaauuu 540
aucaggcugg aucaacaccu uguaauggag uggaaggauu uaauuguuau uuuccauuac 600
agagcuaugg auuucagcca accaauggug ugggauauca gccauauaga gugguggugc 660
ugucuuuuga acugcugcau gcaccugcaa cagugugugg accuaaaggc ucccccggcu 720
ccggcuccgg aucugguuau auuccugaag cuccaagaga ugggcaagcu uacguucgua 780
aagauggcga auggguauua cuuucuaccu uuuuaggccg gucccuggag gugcuguucc 840
agggccccgg cugaugacuc gagcugguac ugcaugcacg caaugcuagc ugccccuuuc 900
ccguccuggg uaccccgagu cucccccgac cucggguccc agguaugcuc ccaccuccac 960
cugccccacu caccaccucu gcuaguucca gacaccuccc aagcacgcag caaugcagcu 1020
caaaacgcuu agccuagcca cacccccacg ggaaacagca gugauuaacc uuuagcaaua 1080
aacgaaaguu uaacuaagcu auacuaaccc caggguuggu caauuucgug ccagccacac 1140
aaaaaaaaaa aaaaaaaaaa aaaaaaaaaa aaaaaaaaaa aaaaaaaaaa aaaaaaaaaa 1260
aa 1262

RBS004.1 (SEQ ID NO: 33; SEQ ID NO: 9 amino acid)
Structure beta-S-ARCA(D1)-replicase-S1S2-PP-FI-A30L70
Encoded antigen Viral spike protein (S protein) of the SARS-CoV-2 (S1S2 full-length protein, sequence variant)

SEQ ID NO: 33
gaugggcggc gcaugagaga agcccagacc aauuaccuac ccaaaaugga gaaaguucac 60
guugacaucg aggaagacag cccauuccuc agagcuuugc agcggagcuu cccgcaguuu 120
gagguagaag ccaagcaggu cacugauaau gaccaugcua augccagagc guuuucgcau 180
cuggcuucaa aacugaucga aacggaggug gacccauccg acacgauccu ugacauugga 240
agugcgcccg cccgcagaau guauucuaag cacaaguauc auuguaucug uccgaugaga 300
ugugcggaag auccggacag auuguauaag uaugcaacua agcugaagaa aaacuguaag 360
gaaauaacug auaaggaauu ggacaagaaa augaaggagc ucgccgccgu caugagcgac 420
ccugaccugg aaacugagac uaugugccuc cacgacgacg agucgugucg cuacgaaggg 480
caagucgcug uuuaccagga uguauacgcg guugacggac cgacaagucu cuaucaccaa 540
gccaauaagg gaguuagagu cgccuacugg auaggcuuug acaccacccc uuuuauguuu 600
aagaacuugg cuggagcaua uccaucauac ucuaccaacu gggccgacga aaccguguua 660
acggcucgua acauaggccu augcagcucu gacguuaugg agcggucacg uagagggaug 720
uccauucuua gaaagaagua uuugaaacca uccaacaaug uucuauucuc uguuggcucg 780
accaucuacc acgaaaagag ggacuuacug aggagcuggc accugccguc uguauuucac 840
uuacguggca agcaaaauua cacaugucgg ugugagacua uaguuaguug cgacggguac 900
gucguuaaaa gaauagcuau caguccaggc cuguauggga agccuucagg cuaugcugcu 960
acgaugcacc gcgagggauu cuugugcugc aaagugacag acacauugaa cggggagagg 1020
gucucuuuuc ccgugugcac guaugugcca gcuacauugu gugaccaaau gacuggcaua 1080
cuggcaacag augucagugc ggacgacgcg caaaaacugc ugguugggcu caaccagcgu 1140
auagucguca acggucgcac ccagagaaac accaauacca ugaaaaauua ccuuuugccc 1200
guaguggccc aggcauuugc uaggugggca aaggaauaua aggaagauca agaagaugaa 1260
aggccacuag gacuacgaga uagacaguua gucauggggu guuguugggc uuuuagaagg 1320
cacaagauaa caucuauuua uaagcgcccg gauacccaaa ccaucaucaa agugaacagc 1380
gauuuccacu cauucgugcu gcccaggaua ggcaguaaca cauuggagau cgggcugaga 1440
acaagaauca ggaaaauguu agaggagcac aaggagccgu caccucucau uaccgccgag 1500
gacguacaag aagcuaagug cgcagccgau gaggcuaagg aggugcguga agccgaggag 1560
uugcgcgcag cucuaccacc uuuggcagcu gauguugagg agcccacucu ggaagccgau 1620
gucgacuuga uguuacaaga ggcuggggcc ggcucagugg agacaccucg uggcuugaua 1680
aagguuacca gcuacgcugg cgaggacaag aucggcucuu acgcugugcu uucuccgcag 1740
gcuguacuca agagugaaaa auuaucuugc auccacccuc ucgcugaaca agucauagug 1800
auaacacacu cuggccgaaa agggcguuau gccguggaac cauaccaugg uaaaguagug 1860
gugccagagg gacaugcaau acccguccag gacuuucaag cucugaguga aagugccacc 1920
auuguguaca acgaacguga guucguaaac agguaccugc accauauugc cacacaugga 1980
ggagcgcuga acacugauga agaauauuac aaaacuguca agcccagcga gcacgacggc 2040
gaauaccugu acgacaucga caggaaacag ugcgucaaga aagagcuagu cacugggcua 2100
gggcucacag gcgagcuggu cgauccuccc uuccaugaau ucgccuacga gagucugaga 2160
acacgaccag ccgcuccuua ccaaguacca accauagggg uguauggcgu gccaggauca 2220
ggcaagucug gcaucauuaa aagcgcaguc accaaaaaag aucuaguggu gagcgccaag 2280
aaagaaaacu gugcagaaau uauaagggac gucaagaaaa ugaaagggcu ggacgucaau 2340
gccagaacug uggacucagu gcucuugaau ggaugcaaac accccguaga gacccuguau 2400
auugacgagg cuuuugcuug ucaugcaggu acucucagag cgcucauagc cauuauaaga 2460
ccuaaaaagg cagugcucug cggagauccc aaacagugcg guuuuuuuaa caugaugugc 2520
cugaaagugc auuuuaacca cgagauuugc acacaagucu uccacaaaag caucucucgc 2580
cguugcacua aaucugugac uucggucguc ucaaccuugu uuuacgacaa aaaaaugaga 2640
acgacgaauc cgaaagagac uaagauugug auugacacua ccggcaguac caaaccuaag 2700
caggacgauc ucauucucac uuguuucaga ggguggguga agcaguugca aauagauuac 2760
aaaggcaacg aaauaaugac ggcagcugcc ucucaagggc ugacccguaa agguguguau 2820
gccguucggu acaaggugaa ugaaaauccu cuguacgcac ccaccucaga acaugugaac 2880
guccuacuga cccgcacgga ggaccgcauc guguggaaaa cacuagccgg cgacccaugg 2940
auaaaaacac ugacugccaa guacccuggg aauuucacug ccacgauaga ggaguggcaa 3000
gcagagcaug augccaucau gaggcacauc uuggagagac cggacccuac cgacgucuuc 3060
cagaauaagg caaacgugug uugggccaag gcuuuagugc cggugcugaa gaccgcuggc 3120
auagacauga ccacugaaca auggaacacu guggauuauu uugaaacgga caaagcucac 3180
ucagcagaga uaguauugaa ccaacuaugc gugagguucu uuggacucga ucuggacucc 3240
ggucuauuuu cugcacccac uguuccguua uccauuagga auaaucacug ggauaacucc 3300
ccgucgccua acauguacgg gcugaauaaa gaaguggucc gucagcucuc ucgcagguac 3360
ccacaacugc cucgggcagu ugccacuggu agagucuaug acaugaacac ugguacacug 3420
cgcaauuaug auccgcgcau aaaccuagua ccuguaaaca gaagacugcc ucaugcuuua 3480
guccuccacc auaaugaaca cccacagagu gacuuuucuu cauucgucag caaauugaag 3540
ggcagaacug uccugguggu cggggaaaag uuguccgucc caggcaaaau gguugacugg 3600
uugucagacc ggccugaggc uaccuucaga gcucggcugg auuuaggcau cccaggugau 3660
gugcccaaau augacauaau auuuguuaau gugaggaccc cauauaaaua ccaucacuau 3720
cagcagugug aagaccaugc cauuaagcua agcauguuga ccaagaaagc augucugcau 3780
cugaaucccg gcggaaccug ugucagcaua gguuaugguu acgcugacag ggccagcgaa 3840
agcaucauug gugcuauagc gcggcaguuc aaguuuuccc gaguaugcaa accgaaaucc 3900
ucacuugagg agacggaagu ucuguuugua uucauugggu acgaucgcaa ggcccguacg 3960
cacaauccuu acaagcuauc aucaaccuug accaacauuu auacagguuc cagacuccac 4020
gaagccggau gugcacccuc auaucaugug gugcgagggg auauugccac ggccaccgaa 4080
ggagugauua uaaaugcugc uaacagcaaa ggacaaccug gcggaggggu gugcggagcg 4140
cuguauaaga aauucccgga aaguuucgau uuacagccga ucgaaguagg aaaagcgcga 4200
cuggucaaag gugcagcuaa acauaucauu caugccguag gaccaaacuu caacaaaguu 4260
ucggagguug aaggugacaa acaguuggca gaggcuuaug aguccaucgc uaagauuguc 4320
aacgauaaca auuacaaguc aguagcgauu ccacuguugu ccaccggcau cuuuuccggg 4380
aacaaagauc gacuaaccca aucauugaac cauuugcuga cagcuuuaga caccacugau 4440
gcagauguag ccauauacug cagggacaag aaaugggaaa ugacucucaa ggaagcagug 4500
gcuaggagag aagcagugga ggagauaugc auauccgacg auucuucagu gacagaaccu 4560
gaugcagagc uggugagggu gcaucccaag aguucuuugg cuggaaggaa gggcuacagc 4620
acaagcgaug gcaaaacuuu cucauauuug gaagggacca aguuucacca ggcggccaag 4680
gauauagcag aaauuaaugc cauguggccc guugcaacgg aggccaauga gcagguaugc 4740
auguauaucc ucggagaaag caugagcagu auuaggucga aaugccccgu cgaggagucg 4800
gaagccucca caccaccuag cacgcugccu ugcuugugca uccaugccau gacuccagaa 4860
agaguacagc gccuaaaagc cucacgucca gaacaaauua cugugugcuc auccuuucca 4920
uugccgaagu auagaaucac uggugugcag aagauccaau gcucccagcc uauauuguuc 4980
ucaccgaaag ugccugcgua uauucaucca aggaaguauc ucguggaaac accaccggua 5040
gacgagacuc cggagccauc ggcagagaac caauccacag aggggacacc ugaacaacca 5100
ccacuuauaa ccgaggauga gaccaggacu agaacgccug agccgaucau caucgaagaa 5160
gaagaagaag auagcauaag uuugcuguca gauggcccga cccaccaggu gcugcaaguc 5220
gaggcagaca uucacgggcc gcccucugua ucuagcucau ccugguccau uccucaugca 5280
uccgacuuug auguggacag uuuauccaua cuugacaccc uggagggagc uagcgugacc 5340
agcggggcaa cgucagccga gacuaacucu uacuucgcaa agaguaugga guuucuggcg 5400
cgaccggugc cugcgccucg aacaguauuc aggaacccuc cacaucccgc uccgcgcaca 5460
agaacaccgu cacuugcacc cagcagggcc ugcuccagaa ccagccuagu uuccaccccg 5520
ccaggcguga auagggugau cacuagagag gagcucgaag cgcuuacccc gucacgcacu 5580
ccuagcaggu cggucuccag aaccagccug gucuccaacc cgccaggcgu aaauagggug 5640
auuacaagag aggaguuuga ggcguucgua gcacaacaac aaugacgguu ugaugcgggu 5700
gcauacaucu uuuccuccga caccggucaa gggcauuuac aacaaaaauc aguaaggcaa 5760
acggugcuau ccgaaguggu guuggagagg accgaauugg agauuucgua ugccccgcgc 5820
cucgaccaag aaaaagaaga auuacuacgc aagaaauuac aguuaaaucc cacaccugcu 5880
aacagaagca gauaccaguc caggaaggug gagaacauga aagccauaac agcuagacgu 5940
auucugcaag gccuagggca uuauuugaag gcagaaggaa aaguggagug cuaccgaacc 6000
cugcauccug uuccuuugua uucaucuagu gugaaccgug ccuuuucaag ccccaagguc 6060
gcaguggaag ccuguaacgc cauguugaaa gagaacuuuc cgacuguggc uucuuacugu 6120
auuauuccag aguacgaugc cuauuuggac augguugacg gagcuucaug cugcuuagac 6180
acugccaguu uuugcccugc aaagcugcgc agcuuuccaa agaaacacuc cuauuuggaa 6240
cccacaauac gaucggcagu gccuucagcg auccagaaca cgcuccagaa cguccuggca 6300
gcugccacaa aaagaaauug caaugucacg caaaugagag aauugcccgu auuggauucg 6360
gcggccuuua auguggaaug cuucaagaaa uaugcgugua auaaugaaua uugggaaacg 6420
uuuaaagaaa accccaucag gcuuacugaa gaaaacgugg uaaauuacau uaccaaauua 6480
aaaggaccaa aagcugcugc ucuuuuugcg aagacacaua auuugaauau guugcaggac 6540
auaccaaugg acagguuugu aauggacuua aagagagacg ugaaagugac uccaggaaca 6600
aaacauacug aagaacggcc caagguacag gugauccagg cugccgaucc gcuagcaaca 6660
gcguaucugu gcggaaucca ccgagagcug guuaggagau uaaaugcggu ccugcuuccg 6720
aacauucaua cacuguuuga uaugucggcu gaagacuuug acgcuauuau agccgagcac 6780
uuccagccug gggauugugu ucuggaaacu gacaucgcgu cguuugauaa aagugaggac 6840
gacgccaugg cucugaccgc guuaaugauu cuggaagacu uaggugugga cgcagagcug 6900
uugacgcuga uugaggcggc uuucggcgaa auuucaucaa uacauuugcc cacuaaaacu 6960
aaauuuaaau ucggagccau gaugaaaucu ggaauguucc ucacacuguu ugugaacaca 7020
gucauuaaca uuguaaucgc aagcagagug uugagagaac ggcuaaccgg aucaccaugu 7080
gcagcauuca uuggagauga caauaucgug aaaggaguca aaucggacaa auuaauggca 7140
gacaggugcg ccaccugguu gaauauggaa gucaagauua uagaugcugu ggugggcgag 7200
aaagcgccuu auuucugugg aggguuuauu uugugugacu ccgugaccgg cacagcgugc 7260
cguguggcag acccccuaaa aaggcuguuu aagcuaggca aaccucuggc agcagacgau 7320
gaacaugaug augacaggag aagggcauug caugaggagu caacacgcug gaaccgagug 7380
gguauucuuu cagagcugug caaggcagua gaaucaaggu augaaaccgu aggaacuucc 7440
aucauaguua uggccaugac uacucuagcu agcaguguua aaucauucag cuaccugaga 7500
ggggccccua uaacucucua cggcuaaccu gaauggacua cgacauaguc uaguccgcca 7560
agacuaguau guuuguguuu cuugugcugc ugccucuugu gucuucucag ugugugaauu 7620
ugacaacaag aacacagcug ccaccagcuu auacaaauuc uuuuaccaga ggaguguauu 7680
auccugauaa aguguuuaga ucuucugugc ugcacagcac acaggaccug uuucugccau 7740
uuuuuagcaa ugugacaugg uuucaugcaa uucauguguc uggaacaaau ggaacaaaaa 7800
gauuugauaa uccugugcug ccuuuuaaug auggagugua uuuugcuuca acagaaaagu 7860
caaauauuau uagaggaugg auuuuuggaa caacacugga uucuaaaaca cagucucugc 7920
ugauugugaa uaaugcaaca aaugugguga uuaaagugug ugaauuucag uuuuguaaug 7980
auccuuuucu gggaguguau uaucacaaaa auaauaaauc uuggauggaa ucugaauuua 8040
gaguguauuc cucugcaaau aauuguacau uugaauaugu gucucagccu uuucugaugg 8100
aucuggaagg aaaacagggc aauuuuaaaa aucugagaga auuuguguuu aaaaauauug 8160
auggauauuu uaaaauuuau ucuaaacaca caccaauuaa uuuagugaga gaucugccuc 8220
agggauuuuc ugcucuggaa ccucuggugg aucugccaau uggcauuaau auuacaagau 8280
uucagacacu gcuggcucug cacagaucuu aucugacacc uggagauucu ucuucuggau 8340
ggacagccgg agcugcagcu uauuaugugg gcuaucugca gccaagaaca uuucugcuga 8400
aauauaauga aaauggaaca auuacagaug cuguggauug ugcucuggau ccucugucug 8460
aaacaaaaug uacauuaaaa ucuuuuacag uggaaaaagg cauuuaucag acaucuaauu 8520
uuagagugca gccaacagaa ucuauuguga gauuuccaaa uauuacaaau cuguguccau 8580
uuggagaagu guuuaaugca acaagauuug caucugugua ugcauggaau agaaaaagaa 8640
uuucuaauug uguggcugau uauucugugc uguauaauag ugcuucuuuu uccacauuua 8700
aauguuaugg agugucucca acaaaauuaa augauuuaug uuuuacaaau guguaugcug 8760
auucuuuugu gaucagaggu gaugaaguga gacagauugc ccccggacag acaggaaaaa 8820
uugcugauua caauuacaaa cugccugaug auuuuacagg augugugauu gcuuggaauu 8880
cuaauaauuu agauucuaaa gugggaggaa auuacaauua ucuguacaga cuguuuagaa 8940
aaucaaaucu gaaaccuuuu gaaagagaua uuucaacaga aauuuaucag gcuggaucaa 9000
caccuuguaa uggaguggaa ggauuuaauu guuauuuucc auuacagagc uauggauuuc 9060
agccaaccaa ugguguggga uaucagccau auagaguggu ggugcugucu uuugaacugc 9120
ugcaugcacc ugcaacagug uguggaccua aaaaaucuac aaauuuagug aaaaauaaau 9180
gugugaauuu uaauuuuaau ggauuaacag gaacaggagu gcugacagaa ucuaauaaaa 9240
aauuucugcc uuuucagcag uuuggcagag auauugcaga uaccacagau gcagugagag 9300
auccucagac auuagaaauu cuggauauua caccuuguuc uuuugggggu gugucuguga 9360
uuacaccugg aacaaauaca ucuaaucagg uggcugugcu guaucaggau gugaauugua 9420
cagaagugcc aguggcaauu caugcagauc agcugacacc aacauggaga guguauucua 9480
caggaucuaa uguguuucag acaagagcag gaugucugau uggagcagaa caugugaaua 9540
auucuuauga augugauauu ccaauuggag caggcauuug ugcaucuuau cagacacaga 9600
caaauucccc aaggagagca agaucugugg caucucaguc uauuauugca uacaccaugu 9660
cucugggagc agaaaauucu guggcauauu cuaauaauuc uauugcuauu ccaacaaauu 9720
uuaccauuuc ugugacaaca gaaauuuuac cugugucuau gacaaaaaca ucuguggauu 9780
guaccaugua cauuugugga gauucuacag aauguucuaa ucugcugcug caguauggau 9840
cuuuuuguac acagcugaau agagcuuuaa caggaauugc uguggaacag gauaaaaaua 9900
cacaggaagu guuugcucag gugaaacaga uuuacaaaac accaccaauu aaagauuuug 9960
gaggauuuaa uuuuagccag auucugccug auccuucuaa accuucuaaa agaucuuuua 10020
uugaagaucu gcuguuuaau aaagugacac uggcagaugc aggauuuauu aaacaguaug 10080
gagauugccu gggugauauu gcugcaagag aucugauuug ugcucagaaa uuuaauggac 10140
ugacagugcu gccuccucug cugacagaug aaaugauugc ucaguacaca ucugcuuuac 10200
uggcuggaac aauuacaagc ggauggacau uuggagcugg agcugcucug cagauuccuu 10260
uugcaaugca gauggcuuac agauuuaaug gaauuggagu gacacagaau guguuauaug 10320
aaaaucagaa acugauugca aaucaguuua auucugcaau uggcaaaauu caggauucuc 10380
ugucuucuac agcuucugcu cugggaaaac ugcaggaugu ggugaaucag aaugcacagg 10440
cacugaauac ucuggugaaa cagcugucua gcaauuuugg ggcaauuucu ucugugcuga 10500
augauauucu gucuagacug gauccuccug aagcugaagu gcagauugau agacugauca 10560
caggaagacu gcagucucug cagacuuaug ugacacagca gcugauuaga gcugcugaaa 10620
uuagagcuuc ugcuaaucug gcugcuacaa aaaugucuga augugugcug ggacagucaa 10680
aaagagugga uuuuugugga aaaggauauc aucugauguc uuuuccacag ucugcuccac 10740
auggaguggu guuuuuacau gugacauaug ugccagcaca ggaaaagaau uuuaccacag 10800
caccagcaau uugucaugau ggaaaagcac auuuuccaag agaaggagug uuugugucua 10860
auggaacaca uugguuugug acacagagaa auuuuuauga accucagauu auuacaacag 10920
auaauacauu ugugucagga aauugugaug uggugauugg aauugugaau aauacagugu 10980
augauccacu gcagccagaa cuggauucuu uuaaagaaga acuggauaaa uauuuuaaaa 11040
aucacacauc uccugaugug gauuuaggag auauuucugg aaucaaugca ucugugguga 11100
auauucagaa agaaauugau agacugaaug aaguggccaa aaaucugaau gaaucucuga 11160
uugaucugca ggaacuugga aaauaugaac aguacauuaa auggccuugg uacauuuggc 11220
uuggauuuau ugcaggauua auugcaauug ugauggugac aauuauguua uguuguauga 11280
caucauguug uucuuguuua aaaggauguu guucuugugg aagcuguugu aaauuugaug 11340
aagaugauuc ugaaccugug uuaaaaggag ugaaauugca uuacacauga ugacucgagc 11400
ugguacugca ugcacgcaau gcuagcugcc ccuuucccgu ccuggguacc ccgagucucc 11460
cccgaccucg ggucccaggu augcucccac cuccaccugc cccacucacc accucugcua 11520
guuccagaca ccucccaagc acgcagcaau gcagcucaaa acgcuuagcc uagccacacc 11580
cccacgggaa acagcaguga uuaaccuuua gcaauaaacg aaaguuuaac uaagcuauac 11640
uaaccccagg guuggucaau uucgugccag ccacaccgcg gccgcaugaa uacagcagca 11700
auuggcaagc ugcuuacaua gaacucgcgg cgauuggcau gccgccuuaa aauuuuuauu 11760
uuauuuuuuc uuuucuuuuc cgaaucggau uuuguuuuua auauuucaaa aaaaaaaaaa 11820
aaaaaaaaaa aaaaaaagca uaugacuaaa aaaaaaaaaa aaaaaaaaaa aaaaaaaaaa 11880
aaaaaaaaaa aaaaaaaaaa aaaaaaaaaa aaaaaaa 11917

RBS004.2(SEQ ID NO: 34; SEQ ID NO: 9 amino acid)
Structure beta-S-ARCA(D1)-replicase-S1S2-PP-FI-A30L70
Encoded antigen Viral spike protein (S protein) of the SARS-CoV-2 (S1S2 full-length protein, sequence variant)

SEQ ID NO: 34
gaugggcggc gcaugagaga agcccagacc aauuaccuac ccaaaaugga gaaaguucac 60
guugacaucg aggaagacag cccauuccuc agagcuuugc agcggagcuu cccgcaguuu 120
gagguagaag ccaagcaggu cacugauaau gaccaugcua augccagagc guuuucgcau 180
cuggcuucaa aacugaucga aacggaggug gacccauccg acacgauccu ugacauugga 240
agugcgcccg cccgcagaau guauucuaag cacaaguauc auuguaucug uccgaugaga 300
ugugcggaag auccggacag auuguauaag uaugcaacua agcugaagaa aaacuguaag 360
gaaauaacug auaaggaauu ggacaagaaa augaaggagc ucgccgccgu caugagcgac 420
ccugaccugg aaacugagac uaugugccuc cacgacgacg agucgugucg cuacgaaggg 480
caagucgcug uuuaccagga uguauacgcg guugacggac cgacaagucu cuaucaccaa 540
gccaauaagg gaguuagagu cgccuacugg auaggcuuug acaccacccc uuuuauguuu 600
aagaacuugg cuggagcaua uccaucauac ucuaccaacu gggccgacga aaccguguua 660
acggcucgua acauaggccu augcagcucu gacguuaugg agcggucacg uagagggaug 720
uccauucuua gaaagaagua uuugaaacca uccaacaaug uucuauucuc uguuggcucg 780
accaucuacc acgaaaagag ggacuuacug aggagcuggc accugccguc uguauuucac 840
uuacguggca agcaaaauua cacaugucgg ugugagacua uaguuaguug cgacggguac 900
gucguuaaaa gaauagcuau caguccaggc cuguauggga agccuucagg cuaugcugcu 960
acgaugcacc gcgagggauu cuugugcugc aaagugacag acacauugaa cggggagagg 1020
gucucuuuuc ccgugugcac guaugugcca gcuacauugu gugaccaaau gacuggcaua 1080
cuggcaacag augucagugc ggacgacgcg caaaaacugc ugguugggcu caaccagcgu 1140
auagucguca acggucgcac ccagagaaac accaauacca ugaaaaauua ccuuuugccc 1200
guaguggccc aggcauuugc uaggugggca aaggaauaua aggaagauca agaagaugaa 1260
aggccacuag gacuacgaga uagacaguua gucauggggu guuguugggc uuuuagaagg 1320
cacaagauaa caucuauuua uaagcgcccg gauacccaaa ccaucaucaa agugaacagc 1380
gauuuccacu cauucgugcu gcccaggaua ggcaguaaca cauuggagau cgggcugaga 1440
acaagaauca ggaaaauguu agaggagcac aaggagccgu caccucucau uaccgccgag 1500
gacguacaag aagcuaagug cgcagccgau gaggcuaagg aggugcguga agccgaggag 1560
uugcgcgcag cucuaccacc uuuggcagcu gauguugagg agcccacucu ggaagccgau 1620
gucgacuuga uguuacaaga ggcuggggcc ggcucagugg agacaccucg uggcuugaua 1680
aagguuacca gcuacgcugg cgaggacaag aucggcucuu acgcugugcu uucuccgcag 1740
gcuguacuca agagugaaaa auuaucuugc auccacccuc ucgcugaaca agucauagug 1800
auaacacacu cuggccgaaa agggcguuau gccguggaac cauaccaugg uaaaguagug 1860
gugccagagg gacaugcaau acccguccag gacuuucaag cucugaguga aagugccacc 1920
auuguguaca acgaacguga guucguaaac agguaccugc accauauugc cacacaugga 1980
ggagcgcuga acacugauga agaauauuac aaaacuguca agcccagcga gcacgacggc 2040
gaauaccugu acgacaucga caggaaacag ugcgucaaga aagagcuagu cacugggcua 2100
gggcucacag gcgagcuggu cgauccuccc uuccaugaau ucgccuacga gagucugaga 2160
acacgaccag ccgcuccuua ccaaguacca accauagggg uguauggcgu gccaggauca 2220
ggcaagucug gcaucauuaa aagcgcaguc accaaaaaag aucuaguggu gagcgccaag 2280
aaagaaaacu gugcagaaau uauaagggac gucaagaaaa ugaaagggcu ggacgucaau 2340
gccagaacug uggacucagu gcucuugaau ggaugcaaac accccguaga gacccuguau 2400
auugacgagg cuuuugcuug ucaugcaggu acucucagag cgcucauagc cauuauaaga 2460
ccuaaaaagg cagugcucug cggagauccc aaacagugcg guuuuuuuaa caugaugugc 2520
cugaaagugc auuuuaacca cgagauuugc acacaagucu uccacaaaag caucucucgc 2580
cguugcacua aaucugugac uucggucguc ucaaccuugu uuuacgacaa aaaaaugaga 2640
acgacgaauc cgaaagagac uaagauugug auugacacua ccggcaguac caaaccuaag 2700
caggacgauc ucauucucac uuguuucaga ggguggguga agcaguugca aauagauuac 2760
aaaggcaacg aaauaaugac ggcagcugcc ucucaagggc ugacccguaa agguguguau 2820
gccguucggu acaaggugaa ugaaaauccu cuguacgcac ccaccucaga acaugugaac 2880
guccuacuga cccgcacgga ggaccgcauc guguggaaaa cacuagccgg cgacccaugg 2940
auaaaaacac ugacugccaa guacccuggg aauuucacug ccacgauaga ggaguggcaa 3000
gcagagcaug augccaucau gaggcacauc uuggagagac cggacccuac cgacgucuuc 3060
cagaauaagg caaacgugug uugggccaag gcuuuagugc cggugcugaa gaccgcuggc 3120
auagacauga ccacugaaca auggaacacu guggauuauu uugaaacgga caaagcucac 3180
ucagcagaga uaguauugaa ccaacuaugc gugagguucu uuggacucga ucuggacucc 3240
ggucuauuuu cugcacccac uguuccguua uccauuagga auaaucacug ggauaacucc 3300
ccgucgccua acauguacgg gcugaauaaa gaaguggucc gucagcucuc ucgcagguac 3360
ccacaacugc cucgggcagu ugccacuggu agagucuaug acaugaacac ugguacacug 3420
cgcaauuaug auccgcgcau aaaccuagua ccuguaaaca gaagacugcc ucaugcuuua 3480
guccuccacc auaaugaaca cccacagagu gacuuuucuu cauucgucag caaauugaag 3540
ggcagaacug uccugguggu cggggaaaag uuguccgucc caggcaaaau gguugacugg 3600
uugucagacc ggccugaggc uaccuucaga gcucggcugg auuuaggcau cccaggugau 3660
gugcccaaau augacauaau auuuguuaau gugaggaccc cauauaaaua ccaucacuau 3720
cagcagugug aagaccaugc cauuaagcua agcauguuga ccaagaaagc augucugcau 3780
cugaaucccg gcggaaccug ugucagcaua gguuaugguu acgcugacag ggccagcgaa 3840
agcaucauug gugcuauagc gcggcaguuc aaguuuuccc gaguaugcaa accgaaaucc 3900
ucacuugagg agacggaagu ucuguuugua uucauugggu acgaucgcaa ggcccguacg 3960
cacaauccuu acaagcuauc aucaaccuug accaacauuu auacagguuc cagacuccac 4020
gaagccggau gugcacccuc auaucaugug gugcgagggg auauugccac ggccaccgaa 4080
ggagugauua uaaaugcugc uaacagcaaa ggacaaccug gcggaggggu gugcggagcg 4140
cuguauaaga aauucccgga aaguuucgau uuacagccga ucgaaguagg aaaagcgcga 4200
cuggucaaag gugcagcuaa acauaucauu caugccguag gaccaaacuu caacaaaguu 4260
ucggagguug aaggugacaa acaguuggca gaggcuuaug aguccaucgc uaagauuguc 4320
aacgauaaca auuacaaguc aguagcgauu ccacuguugu ccaccggcau cuuuuccggg 4380
aacaaagauc gacuaaccca aucauugaac cauuugcuga cagcuuuaga caccacugau 4440
gcagauguag ccauauacug cagggacaag aaaugggaaa ugacucucaa ggaagcagug 4500
gcuaggagag aagcagugga ggagauaugc auauccgacg auucuucagu gacagaaccu 4560
gaugcagagc uggugagggu gcaucccaag aguucuuugg cuggaaggaa gggcuacagc 4620
acaagcgaug gcaaaacuuu cucauauuug gaagggacca aguuucacca ggcggccaag 4680
gauauagcag aaauuaaugc cauguggccc guugcaacgg aggccaauga gcagguaugc 4740
auguauaucc ucggagaaag caugagcagu auuaggucga aaugccccgu cgaggagucg 4800
gaagccucca caccaccuag cacgcugccu ugcuugugca uccaugccau gacuccagaa 4860
agaguacagc gccuaaaagc cucacgucca gaacaaauua cugugugcuc auccuuucca 4920
uugccgaagu auagaaucac uggugugcag aagauccaau gcucccagcc uauauuguuc 4980
ucaccgaaag ugccugcgua uauucaucca aggaaguauc ucguggaaac accaccggua 5040
gacgagacuc cggagccauc ggcagagaac caauccacag aggggacacc ugaacaacca 5100
ccacuuauaa ccgaggauga gaccaggacu agaacgccug agccgaucau caucgaagaa 5160
gaagaagaag auagcauaag uuugcuguca gauggcccga cccaccaggu gcugcaaguc 5220
gaggcagaca uucacgggcc gcccucugua ucuagcucau ccugguccau uccucaugca 5280
uccgacuuug auguggacag uuuauccaua cuugacaccc uggagggagc uagcgugacc 5340
agcggggcaa cgucagccga gacuaacucu uacuucgcaa agaguaugga guuucuggcg 5400
cgaccggugc cugcgccucg aacaguauuc aggaacccuc cacaucccgc uccgcgcaca 5460
agaacaccgu cacuugcacc cagcagggcc ugcuccagaa ccagccuagu uuccaccccg 5520
ccaggcguga auagggugau cacuagagag gagcucgaag cgcuuacccc gucacgcacu 5580
ccuagcaggu cggucuccag aaccagccug gucuccaacc cgccaggcgu aaauagggug 5640
auuacaagag aggaguuuga ggcguucgua gcacaacaac aaugacgguu ugaugcgggu 5700
gcauacaucu uuuccuccga caccggucaa gggcauuuac aacaaaaauc aguaaggcaa 5760
acggugcuau ccgaaguggu guuggagagg accgaauugg agauuucgua ugccccgcgc 5820
cucgaccaag aaaaagaaga auuacuacgc aagaaauuac aguuaaaucc cacaccugcu 5880
aacagaagca gauaccaguc caggaaggug gagaacauga aagccauaac agcuagacgu 5940
auucugcaag gccuagggca uuauuugaag gcagaaggaa aaguggagug cuaccgaacc 6000
cugcauccug uuccuuugua uucaucuagu gugaaccgug ccuuuucaag ccccaagguc 6060
gcaguggaag ccuguaacgc cauguugaaa gagaacuuuc cgacuguggc uucuuacugu 6120
auuauuccag aguacgaugc cuauuuggac augguugacg gagcuucaug cugcuuagac 6180
acugccaguu uuugcccugc aaagcugcgc agcuuuccaa agaaacacuc cuauuuggaa 6240
cccacaauac gaucggcagu gccuucagcg auccagaaca cgcuccagaa cguccuggca 6300
gcugccacaa aaagaaauug caaugucacg caaaugagag aauugcccgu auuggauucg 6360
gcggccuuua auguggaaug cuucaagaaa uaugcgugua auaaugaaua uugggaaacg 6420
uuuaaagaaa accccaucag gcuuacugaa gaaaacgugg uaaauuacau uaccaaauua 6480
aaaggaccaa aagcugcugc ucuuuuugcg aagacacaua auuugaauau guugcaggac 6540
auaccaaugg acagguuugu aauggacuua aagagagacg ugaaagugac uccaggaaca 6600
aaacauacug aagaacggcc caagguacag gugauccagg cugccgaucc gcuagcaaca 6660
gcguaucugu gcggaaucca ccgagagcug guuaggagau uaaaugcggu ccugcuuccg 6720
aacauucaua cacuguuuga uaugucggcu gaagacuuug acgcuauuau agccgagcac 6780
uuccagccug gggauugugu ucuggaaacu gacaucgcgu cguuugauaa aagugaggac 6840
gacgccaugg cucugaccgc guuaaugauu cuggaagacu uaggugugga cgcagagcug 6900
uugacgcuga uugaggcggc uuucggcgaa auuucaucaa uacauuugcc cacuaaaacu 6960
aaauuuaaau ucggagccau gaugaaaucu ggaauguucc ucacacuguu ugugaacaca 7020
gucauuaaca uuguaaucgc aagcagagug uugagagaac ggcuaaccgg aucaccaugu 7080
gcagcauuca uuggagauga caauaucgug aaaggaguca aaucggacaa auuaauggca 7140
gacaggugcg ccaccugguu gaauauggaa gucaagauua uagaugcugu ggugggcgag 7200
aaagcgccuu auuucugugg aggguuuauu uugugugacu ccgugaccgg cacagcgugc 7260
cguguggcag acccccuaaa aaggcuguuu aagcuaggca aaccucuggc agcagacgau 7320
gaacaugaug augacaggag aagggcauug caugaggagu caacacgcug gaaccgagug 7380
gguauucuuu cagagcugug caaggcagua gaaucaaggu augaaaccgu aggaacuucc 7440
aucauaguua uggccaugac uacucuagcu agcaguguua aaucauucag cuaccugaga 7500
ggggccccua uaacucucua cggcuaaccu gaauggacua cgacauaguc uaguccgcca 7560
agacuaguau guucguguuc cuggugcugc ugccucuggu guccagccag ugugugaacc 7620
ugaccaccag aacacagcug ccuccagccu acaccaacag cuuuaccaga ggcguguacu 7680
accccgacaa gguguucaga uccagcgugc ugcacucuac ccaggaccug uuccugccuu 7740
ucuucagcaa cgugaccugg uuccacgcca uccacguguc cggcaccaau ggcaccaaga 7800
gauucgacaa ccccgugcug cccuucaacg acggggugua cuuugccagc accgagaagu 7860
ccaacaucau cagaggcugg aucuucggca ccacacugga cagcaagacc cagagccugc 7920
ugaucgugaa caacgccacc aacgugguca ucaaagugug cgaguuccag uucugcaacg 7980
accccuuccu gggcgucuac uaccacaaga acaacaagag cuggauggaa agcgaguucc 8040
ggguguacag cagcgccaac aacugcaccu ucgaguacgu gucccagccu uuccugaugg 8100
accuggaagg caagcagggc aacuucaaga accugcgcga guucguguuu aagaacaucg 8160
acggcuacuu caagaucuac agcaagcaca ccccuaucaa ccucgugcgg gaucugccuc 8220
agggcuucuc ugcucuggaa ccccuggugg aucugcccau cggcaucaac aucacccggu 8280
uucagacacu gcuggcccug cacagaagcu accugacacc uggcgauagc agcagcggau 8340
ggacagcugg ugccgccgcu uacuaugugg gcuaccugca gccuagaacc uuccugcuga 8400
aguacaacga gaacggcacc aucaccgacg ccguggauug ugcucuggau ccucugagcg 8460
agacaaagug cacccugaag uccuucaccg uggaaaaggg caucuaccag accagcaacu 8520
uccgggugca gcccaccgaa uccaucgugc gguuccccaa uaucaccaau cugugccccu 8580
ucggcgaggu guucaaugcc accagauucg ccucugugua cgccuggaac cggaagcgga 8640
ucagcaauug cguggccgac uacuccgugc uguacaacuc cgccagcuuc agcaccuuca 8700
agugcuacgg cguguccccu accaagcuga acgaccugug cuucacaaac guguacgccg 8760
acagcuucgu gauccgggga gaugaagugc ggcagauugc cccuggacag acaggcaaga 8820
ucgccgacua caacuacaag cugcccgacg acuucaccgg cugugugauu gccuggaaca 8880
gcaacaaccu ggacuccaaa gucggcggca acuacaauua ccuguaccgg cuguuccgga 8940
aguccaaucu gaagcccuuc gagcgggaca ucuccaccga gaucuaucag gccggcagca 9000
ccccuuguaa cggcguggaa ggcuucaacu gcuacuuccc acugcagucc uacggcuuuc 9060
agcccacaaa uggcgugggc uaucagcccu acagaguggu ggugcugagc uucgaacugc 9120
ugcaugcccc ugccacagug ugcggcccua agaaaagcac caaucucgug aagaacaaau 9180
gcgugaacuu caacuucaac ggccugaccg gcaccggcgu gcugacagag agcaacaaga 9240
aguuccugcc auuccagcag uuuggccggg auaucgccga uaccacagac gccguuagag 9300
auccccagac acuggaaauc cuggacauca ccccuugcag cuucggcgga gugucuguga 9360
ucaccccugg caccaacacc agcaaucagg uggcagugcu guaccaggac gugaacugua 9420
ccgaagugcc cguggccauu cacgccgauc agcugacacc uacauggcgg guguacucca 9480
ccggcagcaa uguguuucag accagagccg gcugucugau cggagccgag cacgugaaca 9540
auagcuacga gugcgacauc cccaucggcg cuggaaucug cgccagcuac cagacacaga 9600
caaacagccc ucggagagcc agaagcgugg ccagccagag caucauugcc uacacaaugu 9660
cucugggcgc cgagaacagc guggccuacu ccaacaacuc uaucgcuauc cccaccaacu 9720
ucaccaucag cgugaccaca gagauccugc cuguguccau gaccaagacc agcguggacu 9780
gcaccaugua caucugcggc gauuccaccg agugcuccaa ccugcugcug caguacggca 9840
gcuucugcac ccagcugaau agagcccuga cagggaucgc cguggaacag gacaagaaca 9900
cccaagaggu guucgcccaa gugaagcaga ucuacaagac cccuccuauc aaggacuucg 9960
gcggcuucaa uuucagccag auucugcccg auccuagcaa gcccagcaag cggagcuuca 10020
ucgaggaccu gcuguucaac aaagugacac uggccgacgc cggcuucauc aagcaguaug 10080
gcgauugucu gggcgacauu gccgccaggg aucugauuug cgcccagaag uuuaacggac 10140
ugacagugcu gccuccucug cugaccgaug agaugaucgc ccaguacaca ucugcccugc 10200
uggccggcac aaucacaagc ggcuggacau uuggagcagg cgccgcucug cagauccccu 10260
uugcuaugca gauggccuac cgguucaacg gcaucggagu gacccagaau gugcuguacg 10320
agaaccagaa gcugaucgcc aaccaguuca acagcgccau cggcaagauc caggacagcc 10380
ugagcagcac agcaagcgcc cugggaaagc ugcaggacgu ggucaaccag aaugcccagg 10440
cacugaacac ccuggucaag cagcuguccu ccaacuucgg cgccaucagc ucugugcuga 10500
acgauauccu gagcagacug gacccuccug aggccgaggu gcagaucgac agacugauca 10560
caggcagacu gcagagccuc cagacauacg ugacccagca gcugaucaga gccgccgaga 10620
uuagagccuc ugccaaucug gccgccacca agaugucuga gugugugcug ggccagagca 10680
agagagugga cuuuugcggc aagggcuacc accugaugag cuucccucag ucugccccuc 10740
acggcguggu guuucugcac gugacauaug ugcccgcuca agagaagaau uucaccaccg 10800
cuccagccau cugccacgac ggcaaagccc acuuuccuag agaaggcgug uucgugucca 10860
acggcaccca uugguucgug acacagcgga acuucuacga gccccagauc aucaccaccg 10920
acaacaccuu cgugucuggc aacugcgacg ucgugaucgg cauugugaac aauaccgugu 10980
acgacccucu gcagcccgag cuggacagcu ucaaagagga acuggacaag uacuuuaaga 11040
accacacaag ccccgacgug gaccugggcg auaucagcgg aaucaaugcc agcgucguga 11100
acauccagaa agagaucgac cggcugaacg agguggccaa gaaucugaac gagagccuga 11160
ucgaccugca agaacugggg aaguacgagc aguacaucaa guggcccugg uacaucuggc 11220
ugggcuuuau cgccggacug auugccaucg ugauggucac aaucaugcug uguugcauga 11280
ccagcugcug uagcugccug aagggcuguu guagcugugg cagcugcugc aaguucgacg 11340
aggacgauuc ugagcccgug cugaagggcg ugaaacugca cuacacauga ugacucgagc 11400
ugguacugca ugcacgcaau gcuagcugcc ccuuucccgu ccuggguacc ccgagucucc 11460
cccgaccucg ggucccaggu augcucccac cuccaccugc cccacucacc accucugcua 11520
guuccagaca ccucccaagc acgcagcaau gcagcucaaa acgcuuagcc uagccacacc 11580
cccacgggaa acagcaguga uuaaccuuua gcaauaaacg aaaguuuaac uaagcuauac 11640
uaaccccagg guuggucaau uucgugccag ccacaccgcg gccgcaugaa uacagcagca 11700
auuggcaagc ugcuuacaua gaacucgcgg cgauuggcau gccgccuuaa aauuuuuauu 11760
uuauuuuuuc uuuucuuuuc cgaaucggau uuuguuuuua auauuucaaa aaaaaaaaaa 11820
aaaaaaaaaa aaaaaaagca uaugacuaaa aaaaaaaaaa aaaaaaaaaa aaaaaaaaaa 11880
aaaaaaaaaa aaaaaaaaaa aaaaaaaaaa aaaaaaa 11917

BNT162c1; RBS004.3 (SEQ ID NO: 35; SEQ ID NO: 21 amino acid)
Structure beta-S-ARCA(D1)-replicase-RBD-GS-Fibritin-FI-A30L70
Encoded antigen Viral spike protein (S protein) of the SARS-CoV-2 (partial sequence, Receptor Binding Domain (RBD) of S1S2 protein)

SEQ ID NO: 35
gaugggcggc gcaugagaga agcccagacc aauuaccuac ccaaaaugga gaaaguucac 60
guugacaucg aggaagacag cccauuccuc agagcuuugc agcggagcuu cccgcaguuu 120
gagguagaag ccaagcaggu cacugauaau gaccaugcua augccagagc guuuucgcau 180
cuggcuucaa aacugaucga aacggaggug gacccauccg acacgauccu ugacauugga 240
agugcgcccg cccgcagaau guauucuaag cacaaguauc auuguaucug uccgaugaga 300
ugugcggaag auccggacag auuguauaag uaugcaacua agcugaagaa aaacuguaag 360
gaaauaacug auaaggaauu ggacaagaaa augaaggagc ucgccgccgu caugagcgac 420
ccugaccugg aaacugagac uaugugccuc cacgacgacg agucgugucg cuacgaaggg 480
caagucgcug uuuaccagga uguauacgcg guugacggac cgacaagucu cuaucaccaa 540
gccaauaagg gaguuagagu cgccuacugg auaggcuuug acaccacccc uuuuauguuu 600
aagaacuugg cuggagcaua uccaucauac ucuaccaacu gggccgacga aaccguguua 660
acggcucgua acauaggccu augcagcucu gacguuaugg agcggucacg uagagggaug 720
uccauucuua gaaagaagua uuugaaacca uccaacaaug uucuauucuc uguuggcucg 780
accaucuacc acgaaaagag ggacuuacug aggagcuggc accugccguc uguauuucac 840
uuacguggca agcaaaauua cacaugucgg ugugagacua uaguuaguug cgacggguac 900
gucguuaaaa gaauagcuau caguccaggc cuguauggga agccuucagg cuaugcugcu 960
acgaugcacc gcgagggauu cuugugcugc aaagugacag acacauugaa cggggagagg 1020
gucucuuuuc ccgugugcac guaugugcca gcuacauugu gugaccaaau gacuggcaua 1080
cuggcaacag augucagugc ggacgacgcg caaaaacugc ugguugggcu caaccagcgu 1140
auagucguca acggucgcac ccagagaaac accaauacca ugaaaaauua ccuuuugccc 1200
guaguggccc aggcauuugc uaggugggca aaggaauaua aggaagauca agaagaugaa 1260
aggccacuag gacuacgaga uagacaguua gucauggggu guuguugggc uuuuagaagg 1320
cacaagauaa caucuauuua uaagcgcccg gauacccaaa ccaucaucaa agugaacagc 1380
gauuuccacu cauucgugcu gcccaggaua ggcaguaaca cauuggagau cgggcugaga 1440
acaagaauca ggaaaauguu agaggagcac aaggagccgu caccucucau uaccgccgag 1500
gacguacaag aagcuaagug cgcagccgau gaggcuaagg aggugcguga agccgaggag 1560
uugcgcgcag cucuaccacc uuuggcagcu gauguugagg agcccacucu ggaagccgau 1620
gucgacuuga uguuacaaga ggcuggggcc ggcucagugg agacaccucg uggcuugaua 1680
aagguuacca gcuacgcugg cgaggacaag aucggcucuu acgcugugcu uucuccgcag 1740
gcuguacuca agagugaaaa auuaucuugc auccacccuc ucgcugaaca agucauagug 1800
auaacacacu cuggccgaaa agggcguuau gccguggaac cauaccaugg uaaaguagug 1860
gugccagagg gacaugcaau acccguccag gacuuucaag cucugaguga aagugccacc 1920
auuguguaca acgaacguga guucguaaac agguaccugc accauauugc cacacaugga 1980
ggagcgcuga acacugauga agaauauuac aaaacuguca agcccagcga gcacgacggc 2040
gaauaccugu acgacaucga caggaaacag ugcgucaaga aagagcuagu cacugggcua 2100
gggcucacag gcgagcuggu cgauccuccc uuccaugaau ucgccuacga gagucugaga 2160
acacgaccag ccgcuccuua ccaaguacca accauagggg uguauggcgu gccaggauca 2220
ggcaagucug gcaucauuaa aagcgcaguc accaaaaaag aucuaguggu gagcgccaag 2280
aaagaaaacu gugcagaaau uauaagggac gucaagaaaa ugaaagggcu ggacgucaau 2340
gccagaacug uggacucagu gcucuugaau ggaugcaaac accccguaga gacccuguau 2400
auugacgagg cuuuugcuug ucaugcaggu acucucagag cgcucauagc cauuauaaga 2460
ccuaaaaagg cagugcucug cggagauccc aaacagugcg guuuuuuuaa caugaugugc 2520
cugaaagugc auuuuaacca cgagauuugc acacaagucu uccacaaaag caucucucgc 2580
cguugcacua aaucugugac uucggucguc ucaaccuugu uuuacgacaa aaaaaugaga 2640
acgacgaauc cgaaagagac uaagauugug auugacacua ccggcaguac caaaccuaag 2700
caggacgauc ucauucucac uuguuucaga ggguggguga agcaguugca aauagauuac 2760
aaaggcaacg aaauaaugac ggcagcugcc ucucaagggc ugacccguaa agguguguau 2820
gccguucggu acaaggugaa ugaaaauccu cuguacgcac ccaccucaga acaugugaac 2880
guccuacuga cccgcacgga ggaccgcauc guguggaaaa cacuagccgg cgacccaugg 2940
auaaaaacac ugacugccaa guacccuggg aauuucacug ccacgauaga ggaguggcaa 3000
gcagagcaug augccaucau gaggcacauc uuggagagac cggacccuac cgacgucuuc 3060
cagaauaagg caaacgugug uugggccaag gcuuuagugc cggugcugaa gaccgcuggc 3120
auagacauga ccacugaaca auggaacacu guggauuauu uugaaacgga caaagcucac 3180
ucagcagaga uaguauugaa ccaacuaugc gugagguucu uuggacucga ucuggacucc 3240
ggucuauuuu cugcacccac uguuccguua uccauuagga auaaucacug ggauaacucc 3300
ccgucgccua acauguacgg gcugaauaaa gaaguggucc gucagcucuc ucgcagguac 3360
ccacaacugc cucgggcagu ugccacuggu agagucuaug acaugaacac ugguacacug 3420
cgcaauuaug auccgcgcau aaaccuagua ccuguaaaca gaagacugcc ucaugcuuua 3480
guccuccacc auaaugaaca cccacagagu gacuuuucuu cauucgucag caaauugaag 3540
ggcagaacug uccugguggu cggggaaaag uuguccgucc caggcaaaau gguugacugg 3600
uugucagacc ggccugaggc uaccuucaga gcucggcugg auuuaggcau cccaggugau 3660
gugcccaaau augacauaau auuuguuaau gugaggaccc cauauaaaua ccaucacuau 3720
cagcagugug aagaccaugc cauuaagcua agcauguuga ccaagaaagc augucugcau 3780
cugaaucccg gcggaaccug ugucagcaua gguuaugguu acgcugacag ggccagcgaa 3840
agcaucauug gugcuauagc gcggcaguuc aaguuuuccc gaguaugcaa accgaaaucc 3900
ucacuugagg agacggaagu ucuguuugua uucauugggu acgaucgcaa ggcccguacg 3960
cacaauccuu acaagcuauc aucaaccuug accaacauuu auacagguuc cagacuccac 4020
gaagccggau gugcacccuc auaucaugug gugcgagggg auauugccac ggccaccgaa 4080
ggagugauua uaaaugcugc uaacagcaaa ggacaaccug gcggaggggu gugcggagcg 4140
cuguauaaga aauucccgga aaguuucgau uuacagccga ucgaaguagg aaaagcgcga 4200
cuggucaaag gugcagcuaa acauaucauu caugccguag gaccaaacuu caacaaaguu 4260
ucggagguug aaggugacaa acaguuggca gaggcuuaug aguccaucgc uaagauuguc 4320
aacgauaaca auuacaaguc aguagcgauu ccacuguugu ccaccggcau cuuuuccggg 4380
aacaaagauc gacuaaccca aucauugaac cauuugcuga cagcuuuaga caccacugau 4440
gcagauguag ccauauacug cagggacaag aaaugggaaa ugacucucaa ggaagcagug 4500
gcuaggagag aagcagugga ggagauaugc auauccgacg auucuucagu gacagaaccu 4560
gaugcagagc uggugagggu gcaucccaag aguucuuugg cuggaaggaa gggcuacagc 4620
acaagcgaug gcaaaacuuu cucauauuug gaagggacca aguuucacca ggcggccaag 4680
gauauagcag aaauuaaugc cauguggccc guugcaacgg aggccaauga gcagguaugc 4740
auguauaucc ucggagaaag caugagcagu auuaggucga aaugccccgu cgaggagucg 4800
gaagccucca caccaccuag cacgcugccu ugcuugugca uccaugccau gacuccagaa 4860
agaguacagc gccuaaaagc cucacgucca gaacaaauua cugugugcuc auccuuucca 4920
uugccgaagu auagaaucac uggugugcag aagauccaau gcucccagcc uauauuguuc 4980
ucaccgaaag ugccugcgua uauucaucca aggaaguauc ucguggaaac accaccggua 5040
gacgagacuc cggagccauc ggcagagaac caauccacag aggggacacc ugaacaacca 5100
ccacuuauaa ccgaggauga gaccaggacu agaacgccug agccgaucau caucgaagaa 5160
gaagaagaag auagcauaag uuugcuguca gauggcccga cccaccaggu gcugcaaguc 5220
gaggcagaca uucacgggcc gcccucugua ucuagcucau ccugguccau uccucaugca 5280
uccgacuuug auguggacag uuuauccaua cuugacaccc uggagggagc uagcgugacc 5340
agcggggcaa cgucagccga gacuaacucu uacuucgcaa agaguaugga guuucuggcg 5400
cgaccggugc cugcgccucg aacaguauuc aggaacccuc cacaucccgc uccgcgcaca 5460
agaacaccgu cacuugcacc cagcagggcc ugcuccagaa ccagccuagu uuccaccccg 5520
ccaggcguga auagggugau cacuagagag gagcucgaag cgcuuacccc gucacgcacu 5580
ccuagcaggu cggucuccag aaccagccug gucuccaacc cgccaggcgu aaauagggug 5640
auuacaagag aggaguuuga ggcguucgua gcacaacaac aaugacgguu ugaugcgggu 5700
gcauacaucu uuuccuccga caccggucaa gggcauuuac aacaaaaauc aguaaggcaa 5760
acggugcuau ccgaaguggu guuggagagg accgaauugg agauuucgua ugccccgcgc 5820
cucgaccaag aaaaagaaga auuacuacgc aagaaauuac aguuaaaucc cacaccugcu 5880
aacagaagca gauaccaguc caggaaggug gagaacauga aagccauaac agcuagacgu 5940
auucugcaag gccuagggca uuauuugaag gcagaaggaa aaguggagug cuaccgaacc 6000
cugcauccug uuccuuugua uucaucuagu gugaaccgug ccuuuucaag ccccaagguc 6060
gcaguggaag ccuguaacgc cauguugaaa gagaacuuuc cgacuguggc uucuuacugu 6120
auuauuccag aguacgaugc cuauuuggac augguugacg gagcuucaug cugcuuagac 6180
acugccaguu uuugcccugc aaagcugcgc agcuuuccaa agaaacacuc cuauuuggaa 6240
cccacaauac gaucggcagu gccuucagcg auccagaaca cgcuccagaa cguccuggca 6300
gcugccacaa aaagaaauug caaugucacg caaaugagag aauugcccgu auuggauucg 6360
gcggccuuua auguggaaug cuucaagaaa uaugcgugua auaaugaaua uugggaaacg 6420
uuuaaagaaa accccaucag gcuuacugaa gaaaacgugg uaaauuacau uaccaaauua 6480
aaaggaccaa aagcugcugc ucuuuuugcg aagacacaua auuugaauau guugcaggac 6540
auaccaaugg acagguuugu aauggacuua aagagagacg ugaaagugac uccaggaaca 6600
aaacauacug aagaacggcc caagguacag gugauccagg cugccgaucc gcuagcaaca 6660
gcguaucugu gcggaaucca ccgagagcug guuaggagau uaaaugcggu ccugcuuccg 6720
aacauucaua cacuguuuga uaugucggcu gaagacuuug acgcuauuau agccgagcac 6780
uuccagccug gggauugugu ucuggaaacu gacaucgcgu cguuugauaa aagugaggac 6840
gacgccaugg cucugaccgc guuaaugauu cuggaagacu uaggugugga cgcagagcug 6900
uugacgcuga uugaggcggc uuucggcgaa auuucaucaa uacauuugcc cacuaaaacu 6960
aaauuuaaau ucggagccau gaugaaaucu ggaauguucc ucacacuguu ugugaacaca 7020
gucauuaaca uuguaaucgc aagcagagug uugagagaac ggcuaaccgg aucaccaugu 7080
gcagcauuca uuggagauga caauaucgug aaaggaguca aaucggacaa auuaauggca 7140
gacaggugcg ccaccugguu gaauauggaa gucaagauua uagaugcugu ggugggcgag 7200
aaagcgccuu auuucugugg aggguuuauu uugugugacu ccgugaccgg cacagcgugc 7260
cguguggcag acccccuaaa aaggcuguuu aagcuaggca aaccucuggc agcagacgau 7320
gaacaugaug augacaggag aagggcauug caugaggagu caacacgcug gaaccgagug 7380
gguauucuuu cagagcugug caaggcagua gaaucaaggu augaaaccgu aggaacuucc 7440
aucauaguua uggccaugac uacucuagcu agcaguguua aaucauucag cuaccugaga 7500
ggggccccua uaacucucua cggcuaaccu gaauggacua cgacauaguc uaguccgcca 7560
agacuaguau guuuguguuu cuugugcugc ugccucuugu gucuucucag ugugugguga 7620
gauuuccaaa uauuacaaau cuguguccau uuggagaagu guuuaaugca acaagauuug 7680
caucugugua ugcauggaau agaaaaagaa uuucuaauug uguggcugau uauucugugc 7740
uguauaauag ugcuucuuuu uccacauuua aauguuaugg agugucucca acaaaauuaa 7800
augauuuaug uuuuacaaau guguaugcug auucuuuugu gaucagaggu gaugaaguga 7860
gacagauugc ccccggacag acaggaaaaa uugcugauua caauuacaaa cugccugaug 7920
auuuuacagg augugugauu gcuuggaauu cuaauaauuu agauucuaaa gugggaggaa 7980
auuacaauua ucuguacaga cuguuuagaa aaucaaaucu gaaaccuuuu gaaagagaua 8040
uuucaacaga aauuuaucag gcuggaucaa caccuuguaa uggaguggaa ggauuuaauu 8100
guuauuuucc auuacagagc uauggauuuc agccaaccaa ugguguggga uaucagccau 8160
auagaguggu ggugcugucu uuugaacugc ugcaugcacc ugcaacagug uguggaccua 8220
aaggcucccc cggcuccggc uccggaucug guuauauucc ugaagcucca agagaugggc 8280
aagcuuacgu ucguaaagau ggcgaauggg uauuacuuuc uaccuuuuua ggccgguccc 8340
uggaggugcu guuccagggc cccggcugau gacucgagcu gguacugcau gcacgcaaug 8400
cuagcugccc cuuucccguc cuggguaccc cgagucuccc ccgaccucgg gucccaggua 8460
ugcucccacc uccaccugcc ccacucacca ccucugcuag uuccagacac cucccaagca 8520
cgcagcaaug cagcucaaaa cgcuuagccu agccacaccc ccacgggaaa cagcagugau 8580
uaaccuuuag caauaaacga aaguuuaacu aagcuauacu aaccccaggg uuggucaauu 8640
ucgugccagc cacaccgcgg ccgcaugaau acagcagcaa uuggcaagcu gcuuacauag 8700
aacucgcggc gauuggcaug ccgccuuaaa auuuuuauuu uauuuuuucu uuucuuuucc 8760
gaaucggauu uuguuuuuaa uauuucaaaa aaaaaaaaaa aaaaaaaaaa aaaaaagcau 8820
augacuaaaa aaaaaaaaaa aaaaaaaaaa aaaaaaaaaa aaaaaaaaaa aaaaaaaaaa 8880
aaaaaaaaaa aaaaaa 8896

RBS004.4 (SEQ ID NO: 36; SEQ ID NO: 37)
Structure beta-S-ARCA(D1)-replicase-RBD-GS-Fibritin-TM-FI-A30L70
Encoded antigen Viral spike protein (S protein) of the SARS-CoV-2 (partial sequence, Receptor Binding Domain (RBD) of S1S2 protein)

| SEQ ID NO: 36 | |
|---|---|
| gaugggcggc gcaugagaga agcccagacc aauuaccuac ccaaaaugga gaaaguucac | 60 |
| guugacaucg aggaagacag cccauuccuc agagcuuugc agcggagcuu cccgcaguuu | 120 |
| gagguagaag ccaagcaggu cacugauaau gaccaugcua augccagagc guuuucgcau | 180 |
| cuggcuucaa aacugaucga aacggaggug gacccauccg acacgauccu ugacauugga | 240 |
| agugcgcccg cccgcagaau guauucuaag cacaaguauc auuguaucug uccgaugaga | 300 |
| ugugcggaag auccggacag auuguauaag uaugcaacua agcugaagaa aaacuguaag | 360 |
| gaaauaacug auaaggaauu ggacaagaaa augaaggagc ucgccgccgu caugagcgac | 420 |
| ccugaccugg aaacugagac uaugugccuc cacgacgacg agucgugucg cuacgaaggg | 480 |
| caagucgcug uuuaccagga uguauacgcg guugacggac cgacaagucu cuaucaccaa | 540 |
| gccaauaagg gaguuagagu cgccuacugg auaggcuuug acaccacccc uuuuauguuu | 600 |
| aagaacuugg cuggagcaua uccaucauac ucuaccaacu gggccgacga aaccguguua | 660 |
| acggcucgua acauaggccu augcagcucu gacguuaugg agcggucacg uagagggaug | 720 |
| uccauucuua gaaagaagua uuugaaacca uccaacaaug uucuauucuc uguuggcucg | 780 |
| accaucuacc acgaaaagag ggacuuacug aggagcuggc accugccguc uguauuucac | 840 |
| uuacguggca agcaaaauua cacaugucgg ugugagacua uaguuaguug cgacggguac | 900 |
| gucguuaaaa gaauagcuau caguccaggc cuguauggga agccuucagg cuaugcugcu | 960 |
| acgaugcacc gcgagggauu cuugugcugc aaagugacag acacauugaa cggggagagg | 1020 |
| gucucuuuuc ccgugugcac guaugugcca gcuacauugu gugaccaaau gacuggcaua | 1080 |
| cuggcaacag augucagugc ggacgacgcg caaaaacugc ugguugggcu caaccagcgu | 1140 |
| auagucguca acggucgcac ccagagaaac accaauacca ugaaaaauua ccuuuugccc | 1200 |
| guaguggccc aggcauuugc uaggugggca aaggaauaua aggaagauca agaagaugaa | 1260 |
| aggccacuag gacuacgaga uagacaguua gucauggggu guuguugggc uuuuagaagg | 1320 |
| cacaagauaa caucuauuua uaagcgcccg gauacccaaa ccaucaucaa agugaacagc | 1380 |
| gauuuccacu cauucgugcu gcccaggaua ggcaguaaca cauuggagau cgggcugaga | 1440 |
| acaagaauca ggaaaauguu agaggagcac aaggagccgu caccucucau uaccgccgag | 1500 |
| gacguacaag aagcuaagug cgcagccgau gaggcuaagg aggugcguga agccgaggag | 1560 |
| uugcgcgcag cucuaccacc uuuggcagcu gauguugagg agcccacucu ggaagccgau | 1620 |
| gucgacuuga uguuacaaga ggcuggggcc ggcucagugg agacaccucg uggcuugaua | 1680 |
| aagguuacca gcuacgcugg cgaggacaag aucggcucuu acgcugugcu uucuccgcag | 1740 |
| gcuguacuca agagugaaaa auuaucuugc auccacccuc ucgcugaaca agucauagug | 1800 |
| auaacacacu cuggccgaaa agggcguuau gccguggaac cauaccaugg uaaaguagug | 1860 |
| gugccagagg gacaugcaau acccguccag gacuuucaag cucugaguga aagugccacc | 1920 |
| auuguguaca acgaacguga guucguaaac agguaccugc accauauugc cacacaugga | 1980 |
| ggagcgcuga acacugauga agaauauuac aaaacuguca agcccagcga gcacgacggc | 2040 |
| gaauaccugu acgacaucga caggaaacag ugcgucaaga aagagcuagu cacugggcua | 2100 |
| gggcucacag gcgagcuggu cgauccuccc uuccaugaau ucgccuacga gagucugaga | 2160 |
| acacgaccag ccgcuccuua ccaaguacca accauagggg uguauggcgu gccaggauca | 2220 |
| ggcaagucug gcaucauuaa aagcgcaguc accaaaaaag aucuaguggu gagcgccaag | 2280 |
| aaagaaaacu gugcagaaau uauaagggac gucaagaaaa ugaaagggcu ggacgucaau | 2340 |
| gccagaacug uggacucagu gcucuugaau ggaugcaaac accccguaga gacccuguau | 2400 |
| auugacgagg cuuuugcuug ucaugcaggu acucucagag cgcucauagc cauuauaaga | 2460 |
| ccuaaaaagg cagugcucug cggagauccc aaacagugcg guuuuuuuaa caugaugugc | 2520 |
| cugaaagugc auuuuaacca cgagauuugc acacaagucu uccacaaaag caucucucgc | 2580 |
| cguugcacua aaucugugac uucggucguc ucaaccuugu uuuacgacaa aaaaaugaga | 2640 |
| acgacgaauc cgaaagagac uaagauugug auugacacua ccggcaguac caaaccuaag | 2700 |
| caggacgauc ucauucucac uuguuucaga ggguggguga agcaguugca aauagauuac | 2760 |
| aaaggcaacg aaauaaugac ggcagcugcc ucucaagggc ugacccguaa agguguguau | 2820 |
| gccguucggu acaaggugaa ugaaaauccu cuguacgcac ccaccucaga acaugugaac | 2880 |
| guccuacuga cccgcacgga ggaccgcauc guguggaaaa cacuagccgg cgacccaugg | 2940 |
| auaaaaacac ugacugccaa guacccuggg aauuucacug ccacgauaga ggaguggcaa | 3000 |
| gcagagcaug augccaucau gaggcacauc uuggagagac cggacccuac cgacgucuuc | 3060 |
| cagaauaagg caaacgugug uugggccaag gcuuuagugc cggugcugaa gaccgcuggc | 3120 |
| auagacauga ccacugaaca auggaacacu guggauuauu uugaaacgga caaagcucac | 3180 |
| ucagcagaga uaguauugaa ccaacuaugc gugagguucu uuggacucga ucuggacucc | 3240 |
| ggucuauuuu cugcacccac uguuccguua uccauuagga auaaucacug ggauaacucc | 3300 |
| ccgucgccua acauguacgg gcugaauaaa gaaguggucc gucagcucuc ucgcagguac | 3360 |
| ccacaacugc cucgggcagu ugccacuggu agagucuaug acaugaacac ugguacacug | 3420 |
| cgcaauuaug auccgcgcau aaaccuagua ccuguaaaca gaagacugcc ucaugcuuua | 3480 |
| guccuccacc auaaugaaca cccacagagu gacuuuucuu cauucgucag caaauugaag | 3540 |
| ggcagaacug uccugguggu cggggaaaag uuguccgucc caggcaaaau gguugacugg | 3600 |
| uugucagacc ggccugaggc uaccuucaga gcucggcugg auuuaggcau cccaggugau | 3660 |
| gugcccaaau augacauaau auuuguuaau gugaggaccc cauauaaaua ccaucacuau | 3720 |
| cagcagugug aagaccaugc cauuaagcua agcauguuga ccaagaaagc augucugcau | 3780 |
| cugaaucccg gcggaaccug ugucagcaua gguuaugguu acgcugacag ggccagcgaa | 3840 |
| agcaucauug gugcuauagc gcggcaguuc aaguuuuccc gaguaugcaa accgaaaucc | 3900 |
| ucacuugagg agacggaagu ucuguuugua uucauugggu acgaucgcaa ggcccguacg | 3960 |
| cacaauccuu acaagcuauc aucaaccuug accaacauuu auacagguuc cagacuccac | 4020 |
| gaagccggau gugcacccuc auaucaugug gugcgagggg auauugccac ggccaccgaa | 4080 |
| ggagugauua uaaaugcugc uaacagcaaa ggacaaccug gcggaggggu gugcggagcg | 4140 |
| cuguauaaga aauucccgga aaguuucgau uuacagccga ucgaaguagg aaaagcgcga | 4200 |
| cuggucaaag gugcagcuaa acauaucauu caugccguag gaccaaacuu caacaaaguu | 4260 |
| ucggagguug aaggugacaa acaguuggca gaggcuuaug aguccaucgc uaagauuguc | 4320 |
| aacgauaaca auuacaaguc aguagcgauu ccacuguugu ccaccggcau cuuuuccggg | 4380 |
| aacaaagauc gacuaaccca aucauugaac cauuugcuga cagcuuuaga caccacugau | 4440 |
| gcagauguag ccauauacug cagggacaag aaaugggaaa ugacucucaa ggaagcagug | 4500 |
| gcuaggagag aagcagugga ggagauaugc auauccgacg auucuucagu gacagaaccu | 4560 |
| gaugcagagc uggugagggu gcaucccaag aguucuuugg cuggaaggaa gggcuacagc | 4620 |
| acaagcgaug gcaaaacuuu cucauauuug gaagggacca aguuucacca ggcggccaag | 4680 |
| gauauagcag aaauuaaugc cauguggccc guugcaacgg aggccaauga gcagguaugc | 4740 |
| auguauaucc ucggagaaag caugagcagu auuaggucga aaugccccgu cgaggagucg | 4800 |
| gaagccucca caccaccuag cacgcugccu ugcuugugca uccaugccau gacuccagaa | 4860 |
| agaguacagc gccuaaaagc cucacgucca gaacaaauua cugugugcuc auccuuucca | 4920 |
| uugccgaagu auagaaucac uggugugcag aagauccaau gcucccagcc uauauuguuc | 4980 |
| ucaccgaaag ugccugcgua uauucaucca aggaaguauc ucguggaaac accaccggua | 5040 |
| gacgagacuc cggagccauc ggcagagaac caauccacag aggggacacc ugaacaacca | 5100 |
| ccacuuauaa ccgaggauga gaccaggacu agaacgccug agccgaucau caucgaagaa | 5160 |
| gaagaagaag auagcauaag uuugcuguca gauggcccga cccaccaggu gcugcaaguc | 5220 |
| gaggcagaca uucacgggcc gcccucugua ucuagcucau ccugguccau uccucaugca | 5280 |
| uccgacuuug auguggacag uuuauccaua cuugacaccc uggagggagc uagcgugacc | 5340 |
| agcggggcaa cgucagccga gacuaacucu uacuucgcaa agaguaugga guuucuggcg | 5400 |
| cgaccggugc cugcgccucg aacaguauuc aggaacccuc cacaucccgc uccgcgcaca | 5460 |
| agaacaccgu cacuugcacc cagcagggcc ugcuccagaa ccagccuagu uuccaccccg | 5520 |
| ccaggcguga auagggugau cacuagagag gagcucgaag cgcuuacccc gucacgcacu | 5580 |
| ccuagcaggu cggucuccag aaccagccug gucuccaacc cgccaggcgu aaauagggug | 5640 |
| auuacaagag aggaguuuga ggcguucgua gcacaacaac aaugacgguu ugaugcgggu | 5700 |
| gcauacaucu uuuccuccga caccggucaa gggcauuuac aacaaaaauc aguaaggcaa | 5760 |
| acggugcuau ccgaaguggu guuggagagg accgaauugg agauuucgua ugccccgcgc | 5820 |
| cucgaccaag aaaaagaaga auuacuacgc aagaaauuac aguuaaaucc cacaccugcu | 5880 |
| aacagaagca gauaccaguc caggaaggug gagaacauga aagccauaac agcuagacgu | 5940 |
| auucugcaag gccuagggca uuauuugaag gcagaaggaa aaguggagug cuaccgaacc | 6000 |
| cugcauccug uuccuuugua uucaucuagu gugaaccgug ccuuuucaag ccccaagguc | 6060 |
| gcaguggaag ccuguaacgc cauguugaaa gagaacuuuc cgacuguggc uucuuacugu | 6120 |
| auuauuccag aguacgaugc cuauuuggac augguugacg gagcuucaug cugcuuagac | 6180 |
| acugccaguu uuugcccugc aaagcugcgc agcuuuccaa agaaacacuc cuauuuggaa | 6240 |
| cccacaauac gaucggcagu gccuucagcg auccagaaca cgcuccagaa cguccuggca | 6300 |
| gcugccacaa aaagaaauug caaugucacg caaaugagag aauugcccgu auuggauucg | 6360 |
| gcggccuuua auguggaaug cuucaagaaa uaugcgugua auaaugaaua uugggaaacg | 6420 |
| uuuaaagaaa accccaucag gcuuacugaa gaaaacgugg uaaauuacau uaccaaauua | 6480 |
| aaaggaccaa aagcugcugc ucuuuuugcg aagacacaua auuugaauau guugcaggac | 6540 |
| auaccaaugg acagguuugu aauggacuua aagagagacg ugaaagugac uccaggaaca | 6600 |
| aaacauacug aagaacggcc caagguacag gugauccagg cugccgaucc gcuagcaaca | 6660 |
| gcguaucugu gcggaaucca ccgagagcug guuaggagau uaaaugcggu ccugcuuccg | 6720 |
| aacauucaua cacuguuuga uaugucggcu gaagacuuug acgcuauuau agccgagcac | 6780 |
| uuccagccug gggauugugu ucuggaaacu gacaucgcgu cguuugauaa aagugaggac | 6840 |
| gacgccaugg cucugaccgc guuaaugauu cuggaagacu uaggugugga cgcagagcug | 6900 |
| uugacgcuga uugaggcggc uuucggcgaa auuucaucaa uacauuugcc cacuaaaacu | 6960 |
| aaauuuaaau ucggagccau gaugaaaucu ggaauguucc ucacacuguu ugugaacaca | 7020 |
| gucauuaaca uuguaaucgc aagcagagug uugagagaac ggcuaaccgg aucaccaugu | 7080 |
| gcagcauuca uuggagauga caauaucgug aaaggaguca aaucggacaa auuaauggca | 7140 |
| gacaggugcg ccaccugguu gaauauggaa gucaagauua uagaugcugu ggugggcgag | 7200 |
| aaagcgccuu auuucugugg aggguuuauu uugugugacu ccgugaccgg cacagcgugc | 7260 |
| cguguggcag acccccuaaa aaggcuguuu aagcuaggca aaccucuggc agcagacgau | 7320 |
| gaacaugaug augacaggag aagggcauug caugaggagu caacacgcug gaaccgagug | 7380 |
| gguauucuuu cagagcugug caaggcagua gaaucaaggu augaaaccgu aggaacuucc | 7440 |
| aucauaguua uggccaugac uacucuagcu agcaguguua aaucauucag cuaccugaga | 7500 |
| ggggccccua uaacucucua cggcuaaccu gaauggacua cgacauaguc uaguccgcca | 7560 |
| agacuaguau guuuguguuu cuugugcugc ugccucuugu gucuucucag ugugugguga | 7620 |
| gauuuccaaa uauuacaaau cuguguccau uuggagaagu guuuaaugca acaagauuug | 7680 |
| caucugugua ugcauggaau agaaaaagaa uuucuaauug uguggcugau uauucugugc | 7740 |
| uguauaauag ugcuucuuuu uccacauuua aauguuaugg agugucucca acaaaauuaa | 7800 |
| augauuuaug uuuuacaaau guguaugcug auucuuuugu gaucagaggu gaugaaguga | 7860 |
| gacagauugc ccccggacag acaggaaaaa uugcugauua caauuacaaa cugccugaug | 7920 |
| auuuuacagg augugugauu gcuuggaauu cuaauaauuu agauucuaaa gugggaggaa | 7980 |
| auuacaauua ucuguacaga cuguuuagaa aaucaaaucu gaaaccuuuu gaaagagaua | 8040 |
| uuucaacaga aauuuaucag gcuggaucaa caccuuguaa uggaguggaa ggauuuaauu | 8100 |
| guuauuuucc auuacagagc uauggauuuc agccaaccaa ugguguggga uaucagccau | 8160 |
| auagaguggu ggugcugucu uuugaacugc ugcaugcacc ugcaacagug uguggaccua | 8220 |
| aaggcucccc cggcuccggc uccggaucug guuauauucc ugaagcucca agagaugggc | 8280 |
| aagcuuacgu ucguaaagau ggcgaauggg uauuacuuuc uaccuuuuua ggaagcggca | 8340 |
| gcggaucuga acaguacauu aaauggccuu gguacauuug gcuuggauuu auugcaggau | 8400 |
| uaauugcaau ugugauggug acaauuaugu uauguuguau gacaucaugu uguucuuguu | 8460 |
| uaaaaggaug uuguucuugu ggaagcuguu guaaauuuga ugaagaugau ucugaaccug | 8520 |
| uguuaaaagg agugaaauug cauuacacau gaugacucga gcugguacug caugcacgca | 8580 |
| augcuagcug ccccuuuccc guccugggua ccccgagucu cccccgaccu cgggucccag | 8640 |
| guaugcuccc accuccaccu gccccacuca ccaccucugc uaguuccaga caccucccaa | 8700 |
| gcacgcagca augcagcuca aaacgcuuag ccuagccaca cccccacggg aaacagcagu | 8760 |
| gauuaaccuu uagcaauaaa cgaaaguuua acuaagcuau acuaacccca ggguugguca | 8820 |
| auuucgugcc agccacaccg cggccgcaug aauacagcag caauuggcaa gcugcuuaca | 8880 |
| uagaacucgc ggcgauuggc augccgccuu aaaauuuuua uuuuauuuuu ucuuuucuuu | 8940 |
| uccgaaucgg auuuuguuuu uaauauuuca aaaaaaaaaa aaaaaaaaaa aaaaaaaaaa | 9000 |
| cauaugacua aaaaaaaaaa aaaaaaaaaa aaaaaaaaaa aaaaaaaaaa aaaaaaaaaa | 9060 |
| aaaaaaaaaa aaaaaaaaa | 9079 |

```
SEQ ID NO: 37
Met Phe Val Phe Leu Val Leu Leu Pro Leu Val Ser Ser Gln Cys Val
1 5 10 15
Val Arg Phe Pro Asn Ile Thr Asn Leu Cys Pro Phe Gly Glu Val Phe
                        20 25 30
Asn Ala Thr Arg Phe Ala Ser Val Tyr Ala Trp Asn Arg Lys Arg Ile
                35 40 45
Ser Asn Cys Val Ala Asp Tyr Ser Val Leu Tyr Asn Ser Ala Ser Phe
        50 55 60
Ser Thr Phe Lys Cys Tyr Gly Val Ser Pro Thr Lys Leu Asn Asp Leu
65 70 75 80
Cys Phe Thr Asn Val Tyr Ala Asp Ser Phe Val Ile Arg Gly Asp Glu
                                85 90 95
Val Arg Gln Ile Ala Pro Gly Gln Thr Gly Lys Ile Ala Asp Tyr Asn
                        100 105 110
Tyr Lys Leu Pro Asp Asp Phe Thr Gly Cys Val Ile Ala Trp Asn Ser
                115 120 125
Asn Asn Leu Asp Ser Lys Val Gly Gly Asn Tyr Asn Tyr Leu Tyr Arg
        130 135 140
Leu Phe Arg Lys Ser Asn Leu Lys Pro Phe Glu Arg Asp Ile Ser Thr
145 150 155 160
Glu Ile Tyr Gln Ala Gly Ser Thr Pro Cys Asn Gly Val Glu Gly Phe
                                165 170 175
Asn Cys Tyr Phe Pro Leu Gln Ser Tyr Gly Phe Gln Pro Thr Asn Gly
                        180 185 190
Val Gly Tyr Gln Pro Tyr Arg Val Val Val Leu Ser Phe Glu Leu Leu
                195 200 205
His Ala Pro Ala Thr Val Cys Gly Pro Lys Gly Ser Pro Gly Ser Gly
        210 215 220
Ser Gly Ser Gly Tyr Ile Pro Glu Ala Pro Arg Asp Gly Gln Ala Tyr
225 230 235 240
Val Arg Lys Asp Gly Glu Trp Val Leu Leu Ser Thr Phe Leu Gly Ser
                                245 250 255
Gly Ser Gly Ser Glu Gln Tyr Ile Lys Trp Pro Trp Tyr Ile Trp Leu
                        260 265 270
Gly Phe Ile Ala Gly Leu Ile Ala Ile Val Met Val Thr Ile Met Leu
                275 280 285
Cys Cys Met Thr Ser Cys Cys Ser Cys Leu Lys Gly Cys Cys Ser Cys
        290 295 300
Gly Ser Cys Cys Lys Phe Asp Glu Asp Asp Ser Glu Pro Val Leu Lys
305 310 315 320
Gly Val Lys Leu His Tyr Thr
                                325
```

BNT162b3c (SEQ ID NO: 38; SEQ ID NO: 39)
Structure m₂^{7,3′-O}Gppp(m₁^{2′-O})ApG-hAg-Kozak-RBD-GS-Fibritin-GS-TM-FI-A30L70
Encoded antigen Viral spike protein (S1S2 protein) of the SARS-CoV-2 (partial sequence, Receptor Binding Domain (RBD) of S1S2 protein fused to Fibritin fused to Transmembrane Domain (TM) of S1S2 protein); intrinsic S1S2 protein secretory signal peptide (aa 1-19) at the N-terminus of the antigen sequence

| SEQ ID NO: 38 | |
|---|---|
| Met Phe Val Phe Leu Val Leu Leu Pro Leu Val Ser Ser Gln Cys Val | |
| 1 5 10 15 | |
| Asn Leu Thr Val Arg Phe Pro Asn Ile Thr Asn Leu Cys Pro Phe Gly | |
| 20 25 30 | |
| Glu Val Phe Asn Ala Thr Arg Phe Ala Ser Val Tyr Ala Trp Asn Arg | |
| 35 40 45 | |
| Lys Arg Ile Ser Asn Cys Val Ala Asp Tyr Ser Val Leu Tyr Asn Ser | |
| 50 55 60 | |
| Ala Ser Phe Ser Thr Phe Lys Cys Tyr Gly Val Ser Pro Thr Lys Leu | |
| 65 70 75 80 | |
| Asn Asp Leu Cys Phe Thr Asn Val Tyr Ala Asp Ser Phe Val Ile Arg | |
| 85 90 95 | |
| Gly Asp Glu Val Arg Gln Ile Ala Pro Gly Gln Thr Gly Lys Ile Ala | |
| 100 105 110 | |
| Asp Tyr Asn Tyr Lys Leu Pro Asp Asp Phe Thr Gly Cys Val Ile Ala | |
| 115 120 125 | |
| Trp Asn Ser Asn Asn Leu Asp Ser Lys Val Gly Gly Asn Tyr Asn Tyr | |
| 130 135 140 | |
| Leu Tyr Arg Leu Phe Arg Lys Ser Asn Leu Lys Pro Phe Glu Arg Asp | |
| 145 150 155 160 | |
| Ile Ser Thr Glu Ile Tyr Gln Ala Gly Ser Thr Pro Cys Asn Gly Val | |
| 165 170 175 | |
| Glu Gly Phe Asn Cys Tyr Phe Pro Leu Gln Ser Tyr Gly Phe Gln Pro | |
| 180 185 190 | |
| Thr Asn Gly Val Gly Tyr Gln Pro Tyr Arg Val Val Val Leu Ser Phe | |
| 195 200 205 | |
| Glu Leu Leu His Ala Pro Ala Thr Val Cys Gly Pro Lys Gly Ser Pro | |
| 210 215 220 | |
| Gly Ser Gly Ser Gly Ser Gly Tyr Ile Pro Glu Ala Pro Arg Asp Gly | |
| 225 230 235 240 | |
| Gln Ala Tyr Val Arg Lys Asp Gly Glu Trp Val Leu Leu Ser Thr Phe | |
| 245 250 255 | |
| Leu Gly Ser Gly Ser Gly Ser Glu Gln Tyr Ile Lys Trp Pro Trp Tyr | |
| 260 265 270 | |
| Ile Trp Leu Gly Phe Ile Ala Gly Leu Ile Ala Ile Val Met Val Thr | |
| 275 280 285 | |
| Ile Met Leu Cys Cys Met Thr Ser Cys Cys Ser Cys Leu Lys Gly Cys | |
| 290 295 300 | |
| Cys Ser Cys Gly Ser Cys Cys | |
| 305 310 | |
| SEQ ID NO: 39 | |
| agaauaaacu aguauucuuc ugguccccac agacucagag agaacccgcc accauguuug | 60 |
| uguuucuugu gcugcugccu cuugugucuu cucagugugu gaauuugaca gugagauuuc | 120 |
| caaauauuac aaaucugugu ccauuuggag aaguguuuaa ugcaacaaga uuugcaucug | 180 |
| uguaugcaug gaauagaaaa agaauuucua auuguguggc ugauuauucu gugcuguaua | 240 |
| auagugcuuc uuuuuccaca uuuaaauguu auggaguguc uccaacaaaa uuaaaugauu | 300 |
| uauguuuuac aaauguguau gcugauucuu uugugaucag aggugaugaa gugagacaga | 360 |
| uugcccccgg acagacagga aaaauugcug auuacaauua caaacugccu gaugauuuua | 420 |
| caggaugugu gauugcuugg aauucuaaua auuuagauuc uaaaguggga ggaaauuaca | 480 |
| auuaucugua cagacuguuu agaaaaucaa aucugaaacc uuuugaaaga gauauuucaa | 540 |
| cagaaauuua ucaggcugga ucaacaccuu guaauggagu ggaaggauuu aauuguuauu | 600 |
| uuccauuaca gagcuaugga uuucagccaa ccaauggugu gggauaucag ccauauagag | 660 |
| ugguggugcu gucuuuugaa cugcugcaug caccugcaac agugugugga ccuaaaggcu | 720 |
| cccccggcuc cggcuccgga ucugguuaua uuccugaagc uccaagagau gggcaagcuu | 780 |
| acguucguaa agauggcgaa uggguauuac uuucuaccuu uuuaggaagc ggcagcggau | 840 |
| cugaacagua cauuaaaugg ccuugguaca uuuggcuugg auuuauugca ggauuaauug | 900 |
| caauugugau ggugacaauu auguuauguu guaugacauc auguuguucu uguuuaaaag | 960 |
| gauguuguuc uuguggaagc uguuguugau gacucgagcu gguacugcau gcacgcaaug | 1020 |
| cuagcugccc cuuucccguc cuggguaccc cgagucuccc ccgaccucgg gucccaggua | 1080 |
| ugcucccacc uccaccugcc ccacucacca ccucugcuag uuccagacac cucccaagca | 1140 |
| cgcagcaaug cagcucaaaa cgcuuagccu agccacaccc ccacgggaaa cagcagugau | 1200 |
| uaaccuuuag caauaaacga aaguuuaacu aagcuauacu aaccccaggg uuggucaauu | 1260 |
| ucgugccagc cacacccugg agcuagcaaa aaaaaaaaaa aaaaaaaaaa aaaaaaagca | 1320 |
| uaugacuaaa aaaaaaaaaa aaaaaaaaaa aaaaaaaaaa aaaaaaaaaa aaaaaaaaaa | 1380 |
| aaaaaaaaaa aaaaaaa | 1397 |

BNT162b3d (SEQ ID NO: 40; SEQ ID NO: 41)
Structure m₂^{7,3′-O}Gppp(m₁^{2′-O})ApG-hAg-Kozak-RBD-GS-Fibritin-GS-TM-FI-A30L70
Encoded antigen Viral spike protein (S1S2 protein) of the SARS-CoV-2 (partial sequence, Receptor Binding Domain (RBD) of S1S2 protein fused to Fibritin fused to Transmembrane Domain (TM) of S1S2 protein); immunoglobulin secretory signal peptide (aa 1-22) at the N-terminus of the antigen sequence

| SEQ ID NO: 40 | |
|---|---|
| Met Asp Trp Ile Trp Arg Ile Leu Phe Leu Val Gly Ala Ala Thr Gly | |
| 1 5 10 15 | |
| Ala His Ser Gln Met Gln Val Arg Phe Pro Asn Ile Thr Asn Leu Cys | |
| 20 25 30 | |
| Pro Phe Gly Glu Val Phe Asn Ala Thr Arg Phe Ala Ser Val Tyr Ala | |
| 35 40 45 | |
| Trp Asn Arg Lys Arg Ile Ser Asn Cys Val Ala Asp Tyr Ser Val Leu | |
| 50 55 60 | |
| Tyr Asn Ser Ala Ser Phe Ser Thr Phe Lys Cys Tyr Gly Val Ser Pro | |
| 65 70 75 80 | |
| Thr Lys Leu Asn Asp Leu Cys Phe Thr Asn Val Tyr Ala Asp Ser Phe | |
| 85 90 95 | |
| Val Ile Arg Gly Asp Glu Val Arg Gln Ile Ala Pro Gly Gln Thr Gly | |
| 100 105 110 | |
| Lys Ile Ala Asp Tyr Asn Tyr Lys Leu Pro Asp Asp Phe Thr Gly Cys | |
| 115 120 125 | |
| Val Ile Ala Trp Asn Ser Asn Asn Leu Asp Ser Lys Val Gly Gly Asn | |
| 130 135 140 | |
| Tyr Asn Tyr Leu Tyr Arg Leu Phe Arg Lys Ser Asn Leu Lys Pro Phe | |
| 145 150 155 160 | |
| Glu Arg Asp Ile Ser Thr Glu Ile Tyr Gln Ala Gly Ser Thr Pro Cys | |
| 165 170 175 | |
| Asn Gly Val Glu Gly Phe Asn Cys Tyr Phe Pro Leu Gln Ser Tyr Gly | |
| 180 185 190 | |
| Phe Gln Pro Thr Asn Gly Val Gly Tyr Gln Pro Tyr Arg Val Val Val | |
| 195 200 205 | |
| Leu Ser Phe Glu Leu Leu His Ala Pro Ala Thr Val Cys Gly Pro Lys | |
| 210 215 220 | |
| Gly Ser Pro Gly Ser Gly Ser Gly Ser Gly Tyr Ile Pro Glu Ala Pro | |
| 225 230 235 240 | |
| Arg Asp Gly Gln Ala Tyr Val Arg Lys Asp Gly Glu Trp Val Leu Leu | |
| 245 250 255 | |
| Ser Thr Phe Leu Gly Ser Gly Ser Gly Ser Glu Gln Tyr Ile Lys Trp | |
| 260 265 270 | |
| Pro Trp Tyr Ile Trp Leu Gly Phe Ile Ala Gly Leu Ile Ala Ile Val | |
| 275 280 285 | |
| Met Val Thr Ile Met Leu Cys Cys Met Thr Ser Cys Cys Ser Cys Leu | |
| 290 295 300 | |
| Lys Gly Cys Cys Ser Cys Gly Ser Cys Cys | |
| 305 310 | |
| SEQ ID NO: 41 | |
| agaauaaacu aguauucuuc ugguccccac agacucagag agaacccgcc accauggauu | 60 |
| ggauuuggag aauccuguuc cucgugggag ccgcuacagg agcccacucc cagaugcagg | 120 |
| ugagauuucc aaauauuaca aaucuguguc cauuuggaga aguguuuaau gcaacaagau | 180 |
| uugcaucugu guaugcaugg aauagaaaaa gaauuucuaa uuguguggcu gauuauucug | 240 |
| ugcuguauaa uagugcuucu uuuuccacau uuaaauguua uggagugucu ccaacaaaau | 300 |
| uaaaugauuu auguuuuaca aauguguaug cugauucuuu ugugaucaga ggugaugaag | 360 |
| ugagacagau ugcccccgga cagacaggaa aaauugcuga uuacaauuac aaacugccug | 420 |
| augauuuuac aggaugugug auugcuugga auucuaauaa uuuagauucu aaagugggag | 480 |
| gaaauuacaa uuaucuguac agacuguuua gaaaaucaaa ucugaaaccu uuugaaagag | 540 |
| auauuucaac agaaauuuau caggcuggau caacaccuug uaauggagug gaaggauuua | 600 |
| auuguuauuu uccauuacag agcuauggau uucagccaac caauggugug ggauaucagc | 660 |
| cauauagagu gguggugcug ucuuuugaac ugcugcaugc accugcaaca guguguggac | 720 |
| cuaaaggcuc ccccggcucc ggcuccggau cugguuauau uccugaagcu ccaagagaug | 780 |
| ggcaagcuua cguucguaaa gauggcgaau ggguauuacu uucuaccuuu uuaggaagcg | 840 |
| gcagcggauc ugaacaguac auuaaauggc cuugguacau uuggcuugga uuuauugcag | 900 |
| gauuaauugc aauugugaug gugacaauua uguuauguug uaugacauca uguuguucuu | 960 |
| guuuaaaagg auguuguucu uguggaagcu guuguugaug acucgagcug guacugcaug | 1020 |
| cacgcaaugc uagcugcccc uuucccgucc uggguacccc gagucucccc cgaccucggg | 1080 |
| ucccagguau gcucccaccu ccaccugccc cacucaccac cucugcuagu uccagacacc | 1140 |
| ucccaagcac gcagcaaugc agcucaaaac gcuuagccua gccacacccc cacgggaaac | 1200 |
| agcagugauu aaccuuuagc aauaaacgaa aguuuaacua agcuauacua accccagggu | 1260 |
| uggucaauuu cgugccagcc acacccugga gcuagcaaaa aaaaaaaaaa aaaaaaaaaa | 1320 |
| aaaaaagcau augacuaaaa aaaaaaaaaa aaaaaaaaaa aaaaaaaaaa aaaaaaaaaa | 1380 |
| aaaaaaaaaa aaaaaaaaaa aaaaaa | 1406 |

### Nucleic Acid Containing Particles

Nucleic acids described herein such as RNA encoding a payload may be administered formulated as particles. In the context of the present disclosure, the term “particle” relates to a structured entity formed by molecules or molecule complexes. In some embodiments, the term “particle” relates to a micro- or nano-sized structure, such as a micro- or nano-sized compact structure dispersed in a medium. In some embodiments, a particle is a nucleic acid containing particle such as a particle comprising DNA, RNA or a mixture thereof.

Electrostatic interactions between positively charged molecules such as polymers and lipids and negatively charged nucleic acid are involved in particle formation. This results in complexation and spontaneous formation of nucleic acid particles. In some embodiments, a nucleic acid particle is a nanoparticle.

As used in the present disclosure, “nanoparticle” refers to a particle having an average diameter suitable for parenteral administration.

A “nucleic acid particle” can be used to deliver nucleic acid to a target site of interest (e.g., cell, tissue, organ, and the like). A nucleic acid particle may be formed from at least one cationic or cationically ionizable lipid or lipid-like material, at least one cationic polymer such as protamine, or a mixture thereof and nucleic acid. Nucleic acid particles include lipid nanoparticle (LNP)-based and lipoplex (LPX)-based formulations.

Without intending to be bound by any theory, it is believed that the cationic or cationically ionizable lipid or lipid-like material and/or the cationic polymer combine together with the nucleic acid to form aggregates, and this aggregation results in colloidally stable particles.

In some embodiments, particles described herein further comprise at least one lipid or lipid-like material other than a cationic or cationically ionizable lipid or lipid-like material, at least one polymer other than a cationic polymer, or a mixture thereof.

In some embodiments, nucleic acid particles comprise more than one type of nucleic acid molecules, where the molecular parameters of the nucleic acid molecules may be similar or different from each other, like with respect to molar mass or fundamental structural elements such as molecular architecture, capping, coding regions or other features. Nucleic acid particles described herein may have an average diameter that in some embodiments ranges from about 30 nm to about 1000 nm, from about 50 nm to about 800 nm, from about 70 nm to about 600 nm, from about 90 nm to about 400 nm, or from about 100 nm to about 300 nm.

Nucleic acid particles described herein may exhibit a polydispersity index less than about 0.5, less than about 0.4, less than about 0.3, or about 0.2 or less. By way of example, the nucleic acid particles can exhibit a polydispersity index in a range of about 0.1 to about 0.3 or about 0.2 to about 0.3.

With respect to RNA lipid particles, the N/P ratio gives the ratio of the nitrogen groups in the lipid to the number of phosphate groups in the RNA. It is correlated to the charge ratio, as the nitrogen atoms (depending on the pH) are usually positively charged and the phosphate groups are negatively charged. The N/P ratio, where a charge equilibrium exists, depends on the pH. Lipid formulations are frequently formed at N/P ratios larger than four up to twelve, because positively charged nanoparticles are considered favorable for transfection. In that case, RNA is considered to be completely bound to nanoparticles.

Nucleic acid particles described herein can be prepared using a wide range of methods that may involve obtaining a colloid from at least one cationic or cationically ionizable lipid or lipid-like material and/or at least one cationic polymer and mixing the colloid with nucleic acid to obtain nucleic acid particles.

The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid or lipid-like material and/or at least one cationic polymer methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media).

In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the RNA lipoplex particles described herein are obtainable by adding RNA to a colloidal liposome dispersion. Using the ethanol injection technique, such colloidal liposome dispersion is, in some embodiments, formed as follows: an ethanol solution comprising lipids, such as cationic lipids and additional lipids, is injected into an aqueous solution under stirring. In some embodiments, the RNA lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

LNPs typically comprise four components: ionizable cationic lipids, neutral lipids such as phospholipids, a steroid such as cholesterol, and a polymer conjugated lipid such as polyethylene glycol (PEG)-lipids. Each component is responsible for payload protection, and enables effective intracellular delivery. LNPs may be prepared by mixing lipids dissolved in ethanol rapidly with nucleic acid in an aqueous buffer.

The term “average diameter” refers to the mean hydrodynamic diameter of particles as measured by dynamic laser light scattering (DLS) with data analysis using the so-called cumulant algorithm, which provides as results the so-called Zaverage with the dimension of a length, and the polydispersity index (PI), which is dimensionless (Koppel, D., J. Chem. Phys. 57, 1972, pp 4814-4820, ISO 13321). Here “average diameter”, “diameter” or “size” for particles is used synonymously with this value of the Zaverage.

The “polydispersity index” is preferably calculated based on dynamic light scattering measurements by the so-called cumulant analysis as mentioned in the definition of the “average diameter”. Under certain prerequisites, it can be taken as a measure of the size distribution of an ensemble of nanoparticles.

Different types of nucleic acid containing particles have been described previously to be suitable for delivery of nucleic acid in particulate form (e.g. Kaczmarek, J. C. et al., 2017, Genome Medicine 9, 60). For non-viral nucleic acid delivery vehicles, nanoparticle encapsulation of nucleic acid physically protects nucleic acid from degradation and, depending on the specific chemistry, can aid in cellular uptake and endosomal escape.

The present disclosure describes particles comprising nucleic acid, at least one cationic or cationically ionizable lipid or lipid-like material, and/or at least one cationic polymer which associate with nucleic acid to form nucleic acid particles and compositions comprising such particles. The nucleic acid particles may comprise nucleic acid which is complexed in different forms by non-covalent interactions to the particle. The particles described herein are not viral particles, in particular infectious viral particles, i.e., they are not able to virally infect cells.

Suitable cationic or cationically ionizable lipids or lipid-like materials and cationic polymers are those that form nucleic acid particles and are included by the term “particle forming components” or “particle forming agents”. The term “particle forming components” or “particle forming agents” relates to any components which associate with nucleic acid to form nucleic acid particles. Such components include any component which can be part of nucleic acid particles.

Some embodiments described herein relate to compositions, methods and uses involving more than one, e.g., 2, 3, 4, 5, 6 or even more nucleic acid species such as RNA species, e.g., a) a nucleic acid comprising a first nucleotide sequence encoding an amino acid sequence comprising at least a fragment of a parental virus protein, wherein amino acid positions in the at least a fragment of a parental virus protein are modified to comprise amino acids found in the corresponding amino acid positions of one or more virus protein variants; and b) a nucleic acid comprising a second nucleotide sequence encoding an amino acid sequence comprising at least a fragment of a parental virus protein, wherein amino acid positions in the at least a fragment of a parental virus protein are modified to comprise amino acids found in the corresponding amino acid positions of one or more virus protein variants.

In a particulate formulation, it is possible that each nucleic acid species is separately formulated as an individual particulate formulation. In that case, each individual particulate formulation will comprise one nucleic acid species. The individual particulate formulations may be present as separate entities, e.g. in separate containers. Such formulations are obtainable by providing each nucleic acid species separately (typically each in the form of a nucleic acid-containing solution) together with a particle-forming agent, thereby allowing the formation of particles. Respective particles will contain exclusively the specific nucleic acid species that is being provided when the particles are formed (individual particulate formulations).

In some embodiments, a composition such as a pharmaceutical composition comprises more than one individual particle formulation. Respective pharmaceutical compositions are referred to as mixed particulate formulations. Mixed particulate formulations according to the invention are obtainable by forming, separately, individual particulate formulations, as described above, followed by a step of mixing of the individual particulate formulations. By the step of mixing, a formulation comprising a mixed population of nucleic acid-containing particles is obtainable. Individual particulate populations may be together in one container, comprising a mixed population of individual particulate formulations.

Alternatively, it is possible that different nucleic acid species are formulated together as a combined particulate formulation. Such formulations are obtainable by providing a combined formulation (typically combined solution) of different RNA species together with a particle-forming agent, thereby allowing the formation of particles. As opposed to a mixed particulate formulation, a combined particulate formulation will typically comprise particles which comprise more than one RNA species. In a combined particulate composition different RNA species are typically present together in a single particle.

### Cationic Polymeric Materials (e.g., Polymers)

Given their high degree of chemical flexibility, polymeric materials are commonly used for nanoparticle-based delivery. Typically, cationic materials are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic materials useful in some embodiments herein. In addition, some investigators have synthesized polymeric materials specifically for nucleic acid delivery. Poly(β-amino esters), in particular, have gained widespread use in nucleic acid delivery owing to their ease of synthesis and biodegradability. In some embodiments, such synthetic materials may be suitable for use as cationic materials herein.

A “polymeric material”, as used herein, is given its ordinary meaning, i.e., a molecular structure comprising one or more repeat units (monomers), connected by covalent bonds. In some embodiments, such repeat units can all be identical; alternatively, in some cases, there can be more than one type of repeat unit present within the polymeric material. In some cases, a polymeric material is biologically derived, e.g., a biopolymer such as a protein. In some cases, additional moieties can also be present in the polymeric material, for example targeting moieties such as those described herein.

Those skilled in the art are aware that, when more than one type of repeat unit is present within a polymer (or polymeric moiety), then the polymer (or polymeric moiety) is said to be a “copolymer.” In some embodiments, a polymer (or polymeric moiety) utilized in accordance with the present disclosure may be a copolymer. Repeat units forming the copolymer can be arranged in any fashion. For example, in some embodiments, repeat units can be arranged in a random order; alternatively or additionally, in some embodiments, repeat units may be arranged in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In certain embodiments, a polymeric material for use in accordance with the present disclosure is biocompatible. Biocompatible materials are those that typically do not result in significant cell death at moderate concentrations. In certain embodiments, a biocompatible material is biodegradable, i.e., is able to degrade, chemically and/or biologically, within a physiological environment, such as within the body.

In certain embodiments, a polymeric material may be or comprise protamine or polyalkyleneimine, in particular protamine.

As those skilled in the art are aware term “protamine” is often used to refer to any of various strongly basic proteins of relatively low molecular weight that are rich in arginine and are found associated especially with DNA in place of somatic histones in the sperm cells of various animals (as fish). In particular, the term “protamine” is often used to refer to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

In some embodiments, the term “protamine” as used herein is refers to a protamine amino acid sequence obtained or derived from natural or biological sources, including fragments thereof and/or multimeric forms of said amino acid sequence or fragment thereof, as well as (synthesized) polypeptides which are artificial and specifically designed for specific purposes and cannot be isolated from native or biological sources.

In some embodiments, a polyalkyleneimine comprises polyethylenimine and/or polypropylenimine, preferably polyethyleneimine. In some embodiments, a preferred polyalkyleneimine is polyethyleneimine (PEI). In some embodiments, the average molecular weight of PEI is preferably 0.75-102 to 107 Da, preferably 1000 to 105 Da, more preferably 10000 to 40000 Da, more preferably 15000 to 30000 Da, even more preferably 20000 to 25000 Da.

Preferred according to certain embodiments of the disclosure is linear polyalkyleneimine such as linear polyethyleneimine (PEI).

Cationic materials (e.g., polymeric materials, including polycationic polymers) contemplated for use herein include those which are able to electrostatically bind nucleic acid. In some embodiments, cationic polymeric materials contemplated for use herein include any cationic polymeric materials with which nucleic acid can be associated, e.g. by forming complexes with the nucleic acid or forming vesicles in which the nucleic acid is enclosed or encapsulated.

In some embodiments, particles described herein may comprise polymers other than cationic polymers, e.g., non-cationic polymeric materials and/or anionic polymeric materials. Collectively, anionic and neutral polymeric materials are referred to herein as non-cationic polymeric materials.

### Lipid and Lipid-Like Material

The terms “lipid” and “lipid-like material” are used herein to refer to molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). In some embodiments, hydrophilic groups may comprise polar and/or charged groups and include carbohydrates, phosphate, carboxylic, sulfate, amino, sulfhydryl, nitro, hydroxyl, and other like groups.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids. As used herein, the term “lipid” is to be construed to cover both lipids and lipid-like materials unless otherwise indicated herein or clearly contradicted by context.

Specific examples of amphiphilic compounds that may be included in an amphiphilic layer include, but are not limited to, phospholipids, aminolipids and sphingolipids. In certain embodiments, the amphiphilic compound is a lipid. The term “lipid” refers to a group of organic compounds that are characterized by being insoluble in water, but soluble in many organic solvents. Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides. Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes. According to the disclosure, lipids and lipid-like materials may be cationic, anionic or neutral. Neutral lipids or lipid-like materials exist in an uncharged or neutral zwitterionic form at a selected pH.

### Cationic or Cationically Ionizable Lipids or Lipid-Like Materials

In some embodiments, nucleic acid particles described and/or utilized in accordance with the present disclosure may comprise at least one cationic or cationically ionizable lipid or lipid-like material as particle forming agent. Cationic or cationically ionizable lipids or lipid-like materials contemplated for use herein include any cationic or cationically ionizable lipids or lipid-like materials which are able to electrostatically bind nucleic acid. In some embodiments, cationic or cationically ionizable lipids or lipid-like materials contemplated for use herein can be associated with nucleic acid, e.g. by forming complexes with the nucleic acid or forming vesicles in which the nucleic acid is enclosed or encapsulated.

As used herein, a “cationic lipid” or “cationic lipid-like material” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In certain embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

For purposes of the present disclosure, such “cationically ionizable” lipids or lipid-like materials are comprised by the term “cationic lipid or lipid-like material” unless contradicted by the circumstances.

In some embodiments, a cationic or cationically ionizable lipid or lipid-like material comprises a head group which includes at least one nitrogen atom (N) which is positive charged or capable of being protonated.

Examples of cationic lipids include, but are not limited to: ((4-hydroxybutyl)azanediyl)bis(hexane-6,1-diyl)bis(2-hexyldecanoate); 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)—N-(3-aminopropyl)-N,N-dimethyl-2,3-bis(cis-9-tetradecenyloxy)-1-propanaminium bromide (GAP-DMORIE), (±)—N-(3-aminopropyl)-N,N-dimethyl-2,3-bis(dodecyloxy)-1-propanaminium bromide (GAP-DLRIE), (±)—N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (βAE-DMRIE), N-(4-carboxybenzyl)-N,N-dimethyl-2,3-bis(oleoyloxy)propan-1-aminium (DOBAQ), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200); or heptadecan-9-yl 8-((2-hydroxyethyl) (6-oxo-6-(undecyloxy) hexyl) amino) octanoate (SM-102).

In some embodiments, a cationic lipid is or comprises heptadecan-9-yl 8-((2-hydroxyethyl) (6-oxo-6-(undecyloxy) hexyl) amino) octanoate (SM-102). In some embodiments, a cationic lipid is or comprises a cationic lipid shown in the structure below.

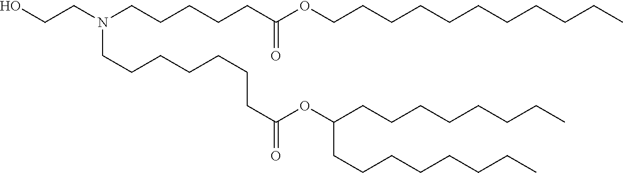 [SMILES: CCCCCCCCCCCOC(=O)CCCCCN(CCO)CCCCCCCC(=O)OC(CCCCCCCC)CCCCCCCC]

In some embodiments, a cationic lipid is or comprises ((4-hydroxybutyl)azanediyl)bis(hexane-6,1-diyl)bis(2-hexyldecanoate) which is also referred to as ALC-0315 herein.

In some embodiments, a cationic lipid may comprise from about 10 mol % to about 100 mol %. about 20 mol % to about 100 mol %. about 30 mol % to about 100 mol %. about 40 mol % to about 100 mol %, or about 50 mol % to about 100 mol % of the total lipid present in the particle.

In some particular embodiments, a particle for use in accordance with the present disclosure includes ALC-0315, for example in a weight percent within a range of about 40-55 mol percent of total lipids.

### Additional Lipids or Lipid-Like Materials

In some embodiments, particles described herein comprise (e.g., in addition to a cationic lipid such as ALC315), one or more lipids or lipid-like materials other than cationic or cationically ionizable lipids or lipid-like materials, e.g., non-cationic lipids or lipid-like materials (including non-cationically ionizable lipids or lipid-like materials). Collectively, anionic and neutral lipids or lipid-like materials are referred to herein as non-cationic lipids or lipid-like materials. Optimizing the formulation of nucleic acid particles by addition of other hydrophobic moieties, such as cholesterol and lipids, in addition to an ionizable/cationic lipid or lipid-like material may enhance particle stability and efficacy of nucleic acid delivery.

An additional lipid or lipid-like material may be incorporated which may or may not affect the overall charge of the nucleic acid particles. In certain embodiments, the additional lipid or lipid-like material is a non-cationic lipid or lipid-like material. The non-cationic lipid may comprise, e.g., one or more anionic lipids and/or neutral lipids. As used herein, an “anionic lipid” refers to any lipid that is negatively charged at a selected pH. As used herein, a “neutral lipid” refers to any of a number of lipid species that exist either in an uncharged or neutral zwitterionic form at a selected pH. In preferred embodiments, the additional lipid comprises one of the following neutral lipid components: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

In certain preferred embodiments, the additional lipid is DSPC or DSPC and cholesterol. In certain embodiments, the nucleic acid particles include both a cationic lipid and an additional lipid.

In some embodiments, particles described herein include a polymer conjugated lipid such as a pegylated lipid. The term “pegylated lipid” refers to a molecule comprising both a lipid portion and a polyethylene glycol portion. Pegylated lipids are known in the art. In some embodiments, a pegylated lipid is ALC-0159, also referred to herein as (2-[(polyethylene glycol)-2000]—N,N-ditetradecylacetamide).

Without wishing to be bound by theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some embodiments, the molar ratio of the at least one cationic lipid to the at least one additional lipid is from about 10:0 to about 1:9, about 4:1 to about 1:2, or about 3:1 to about 1:1.

In some embodiments, the non-cationic lipid, in particular neutral lipid, (e.g., one or more phospholipids and/or cholesterol) may comprise from about 0 mol % to about 90 mol %, from about 0 mol % to about 80 mol %, from about 0 mol % to about 70 mol %, from about 0 mol % to about 60 mol %, or from about 0 mol % to about 50 mol %. of the total lipid present in the particle.

In some embodiments, particles for use in accordance with the present disclosure may include, for example, ALC-0315, DSPC, CHOL, and ALC-0159, for example, wherein ALC-0315 is at about 40 to 55 mol percent; DSPC is at about 5 to 15 mol percent; CHOL is at about 30 to 50 mol percent; and ALC-0159 is at about 1 to 10 mol percent.

### Lipoplex Particles

In certain embodiments of the present disclosure, an RNA may be present in RNA lipoplex particles.

In the context of the present disclosure, the term “RNA lipoplex particle” relates to a particle that contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE. In some embodiments, a RNA lipoplex particle is a nanoparticle.

In certain embodiments, the RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

RNA lipoplex particles described herein have an average diameter that in some embodiments ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

In some embodiments, RNA lipoplex particles and/or compositions comprising RNA lipoplex particles described herein are useful for delivery of RNA to a target tissue after parenteral administration, in particular after intravenous administration. In some embodiments, RNA lipoplex particles may be prepared using liposomes that may be obtained by injecting a solution of the lipids in ethanol into water or a suitable aqueous phase. In some embodiments, the aqueous phase has an acidic pH. In some embodiments, the aqueous phase comprises acetic acid, e.g., in an amount of about 5 mM. Liposomes may be used for preparing RNA lipoplex particles by mixing the liposomes with RNA. In some embodiments, the liposomes and RNA lipoplex particles comprise at least one cationic lipid and at least one additional lipid. In some embodiments, the at least one cationic lipid comprises 1,2-di-O-octadecenyl-3-trimethylammonium propane (DOTMA) and/or 1,2-dioleoyl-3-trimethylammonium-propane (DOTAP). In some embodiments, the at least one additional lipid comprises 1,2-di-(9Z-octadecenoyl)-sn-glycero-3-phosphoethanolamine (DOPE), cholesterol (Chol) and/or 1,2-dioleoyl-sn-glycero-3-phosphocholine (DOPC). In some embodiments, the at least one cationic lipid comprises 1,2-di-O-octadecenyl-3-trimethylammonium propane (DOTMA) and the at least one additional lipid comprises 1,2-di-(9Z-octadecenoyl)-sn-glycero-3-phosphoethanolamine (DOPE). In some embodiments, the liposomes and RNA lipoplex particles comprise 1,2-di-O-octadecenyl-3-trimethylammonium propane (DOTMA) and 1,2-di-(9Z-octadecenoyl)-sn-glycero-3-phosphoethanolamine (DOPE).

Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In some embodiments, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In some embodiments, the antigen presenting cells are dendritic cells and/or macrophages.

### Lipid Nanoparticles (LNPs)

In some embodiments, nucleic acid such as RNA described herein is administered in the form of lipid nanoparticles (LNPs). The LNP may comprise any lipid capable of forming a particle to which the one or more nucleic acid molecules are attached, or in which the one or more nucleic acid molecules are encapsulated.

In some embodiments, the LNP comprises one or more cationic lipids, and one or more stabilizing lipids. Stabilizing lipids include neutral lipids and pegylated lipids.

In some embodiments, the LNP comprises a cationic lipid, a neutral lipid, a steroid, a polymer conjugated lipid; and the RNA, encapsulated within or associated with the lipid nanoparticle.

In some embodiments, an LNP comprises from 40 to 55 mol percent, from 40 to 50 mol percent, from 41 to 49 mol percent, from 41 to 48 mol percent, from 42 to 48 mol percent, from 43 to 48 mol percent, from 44 to 48 mol percent, from 45 to 48 mol percent, from 46 to 48 mol percent, from 47 to 48 mol percent, or from 47.2 to 47.8 mol percent of the cationic lipid. In some embodiments, the LNP comprises about 47.0, 47.1, 47.2, 47.3, 47.4, 47.5, 47.6, 47.7, 47.8, 47.9 or 48.0 mol percent of the cationic lipid.

In some embodiments, the neutral lipid is present in a concentration ranging from 5 to 15 mol percent, from 7 to 13 mol percent, or from 9 to 11 mol percent. In some embodiments, the neutral lipid is present in a concentration of about 9.5, 10 or 10.5 mol percent.

In some embodiments, the steroid is present in a concentration ranging from 30 to 50 mol percent, from 35 to 45 mol percent or from 38 to 43 mol percent. In some embodiments, the steroid is present in a concentration of about 40, 41, 42, 43, 44, 45 or 46 mol percent.

In some embodiments, the LNP comprises from 1 to 10 mol percent, from 1 to 5 mol percent, or from 1 to 2.5 mol percent of the polymer conjugated lipid.

In some embodiments, the LNP comprises from 40 to 50 mol percent a cationic lipid; from 5 to 15 mol percent of a neutral lipid; from 35 to 45 mol percent of a steroid; from 1 to 10 mol percent of a polymer conjugated lipid; and the RNA, encapsulated within or associated with the lipid nanoparticle.

In some embodiments, the mol percent is determined based on total mol of lipid present in the lipid nanoparticle.

In some embodiments, the neutral lipid is selected from the group consisting of DSPC, DPPC, DMPC, DOPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DSPC.

In some embodiments, the steroid is cholesterol.

In some embodiments, the polymer conjugated lipid is a pegylated lipid. In some embodiments, the pegylated lipid has the following structure:

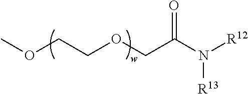

or a pharmaceutically acceptable salt, tautomer or stereoisomer thereof, wherein: R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In some embodiments, R¹² and R¹³ are each independently straight, saturated alkyl chains containing from 12 to 16 carbon atoms. In some embodiments, w has a mean value ranging from 40 to 55. In some embodiments, the average w is about 45. In some embodiments, R¹² and R¹³ are each independently a straight, saturated alkyl chain containing about 14 carbon atoms, and w has a mean value of about 45.

In some embodiments, the pegylated lipid is DMG-PEG 2000, e.g., having the following structure:

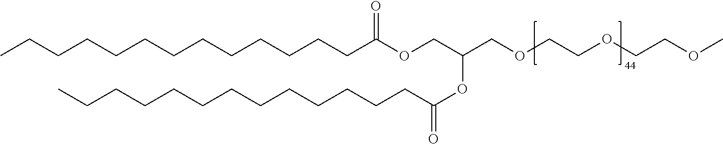

In some embodiments, the cationic lipid component of the LNPs has the structure of Formula (III):

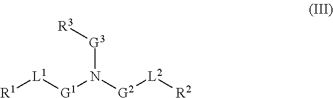

or a pharmaceutically acceptable salt, tautomer, prodrug or stereoisomer thereof, wherein: one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)N^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C3-C₈ cycloalkenylene;
R^{a1} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

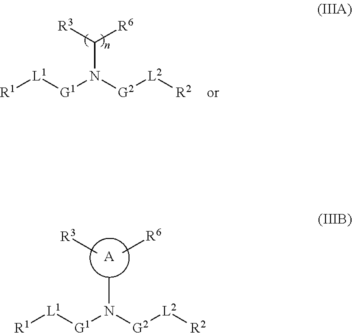

wherein:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

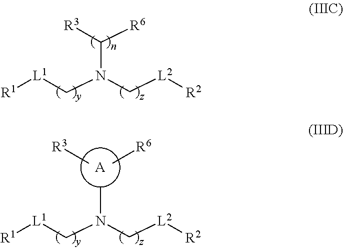

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (III), the lipid has one of the following structures (IIIE) or (IIIF):

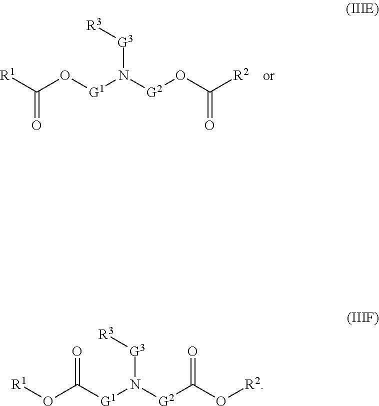

In some of the foregoing embodiments of Formula (III), the lipid has one of the following structures (IIIG), (IIIH), (IIII), or (IIIJ):

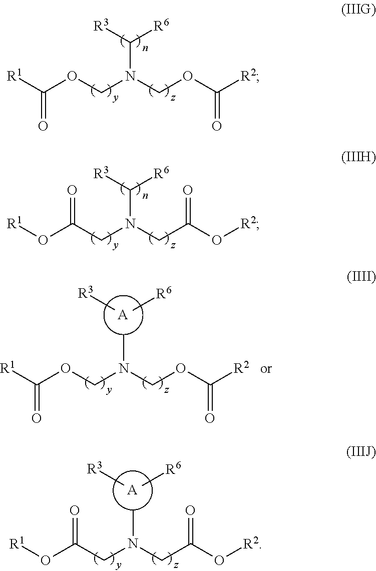

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G³ is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

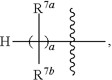

wherein:
R^{7a} and R^{7b} are, at each occurrence, independently H or C₁-C₁₂ alkyl; and
a is an integer from 2 to 12,
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

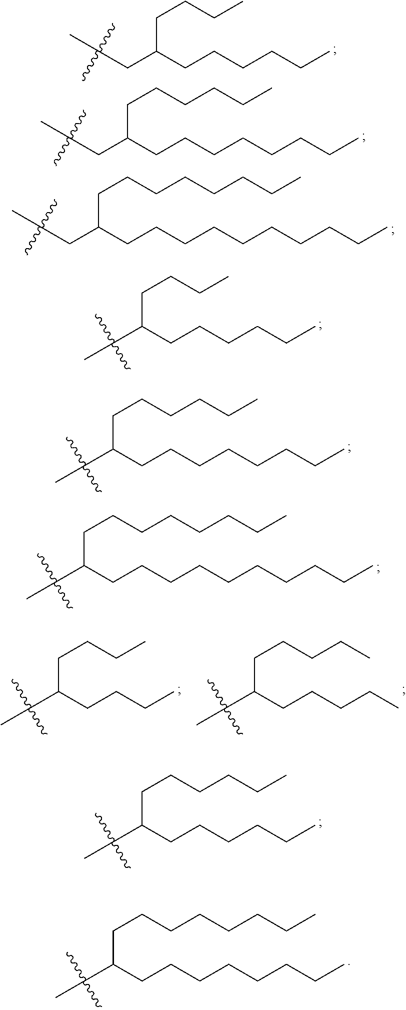

In some of the foregoing embodiments of Formula (III), R³ is OH, CN, —C(═O)OR⁴, —OC(═O)R⁴ or —NHC(═O)R⁴. In some embodiments, R⁴ is methyl or ethyl. In various different embodiments, the cationic lipid of Formula (III) has one of the structures set forth in the table below.

### Representative Compounds of Formula (III).

| No. | Structure |
|---|---|
| III-1 | 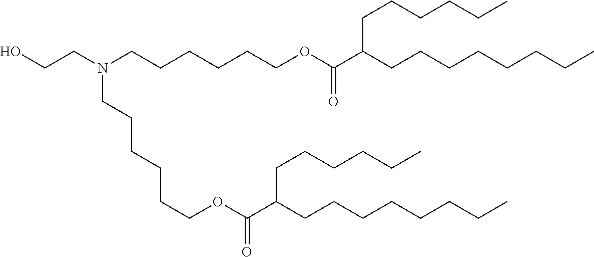 [SMILES: CCCCCCCCC(CCCCCC)C(=O)OCCCCCCN(CCO)CCCCCCOC(=O)C(CCCCCC)CCCCCCCC] |
| III-2 | 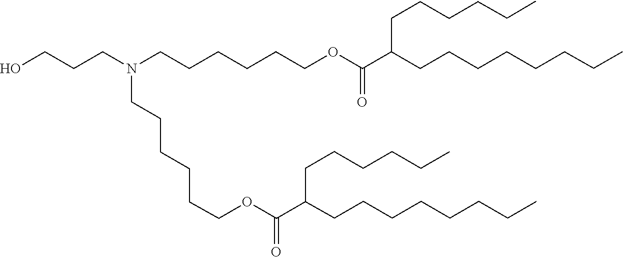 [SMILES: CCCCCCCCC(CCCCCC)C(=O)OCCCCCCN(CCCO)CCCCCCOC(=O)C(CCCCCC)CCCCCCCC] |
| III-3 | 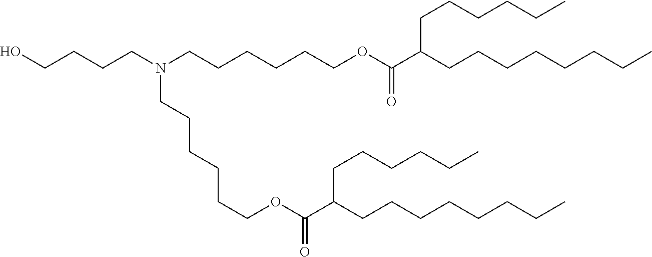 [SMILES: CCCCCCCCC(CCCCCC)C(=O)OCCCCCCN(CCCCO)CCCCCCOC(=O)C(CCCCCC)CCCCCCCC] |
| III-4 | 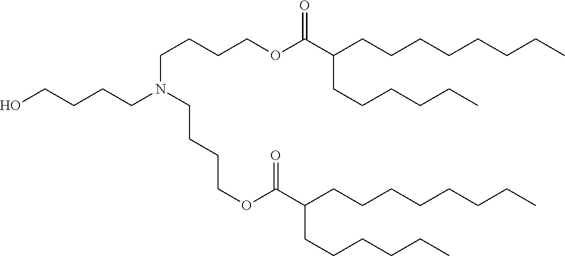 [SMILES: CCCCCCCCC(CCCCCC)C(=O)OCCCCN(CCCCO)CCCCOC(=O)C(CCCCCC)CCCCCCCC] |
| III-5 | 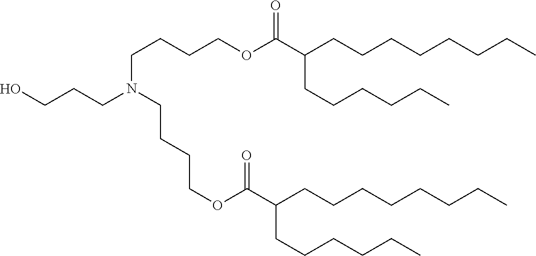 [SMILES: CCCCCCCCC(CCCCCC)C(=O)OCCCCN(CCCO)CCCCOC(=O)C(CCCCCC)CCCCCCCC] |
| III-6 | 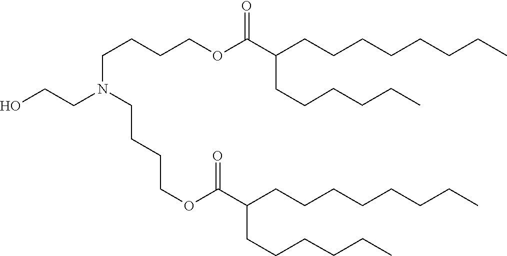 [SMILES: CCCCCCCCC(CCCCCC)C(=O)OCCCCN(CCO)CCCCOC(=O)C(CCCCCC)CCCCCCCC] |
| III-7 | 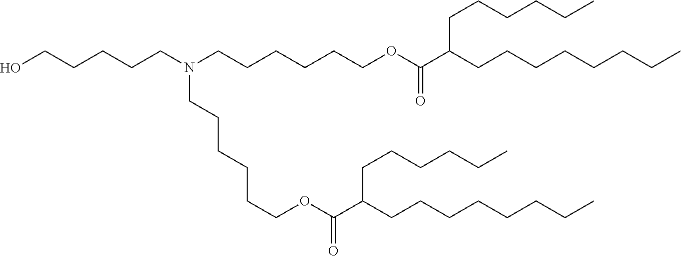 [SMILES: CCCCCCCCC(CCCCCC)C(=O)OCCCCCCN(CCCCCO)CCCCCCOC(=O)C(CCCCCC)CCCCCCCC] |
| III-8 | 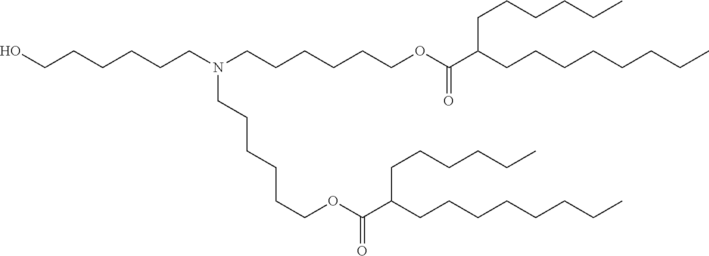 [SMILES: CCCCCCCCC(CCCCCC)C(=O)OCCCCCCN(CCCCCCO)CCCCCCOC(=O)C(CCCCCC)CCCCCCCC] |
| III-9 | 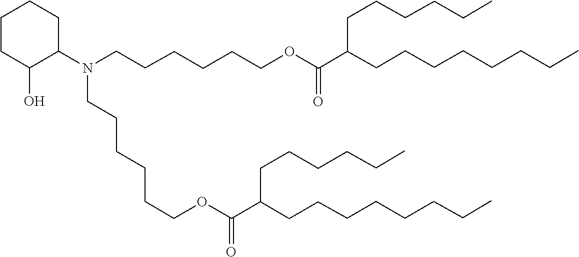 [SMILES: CCCCCCCCC(CCCCCC)C(=O)OCCCCCCN(CCCCCCOC(=O)C(CCCCCC)CCCCCCCC)C1CCCCC1O] |
| III-10 | 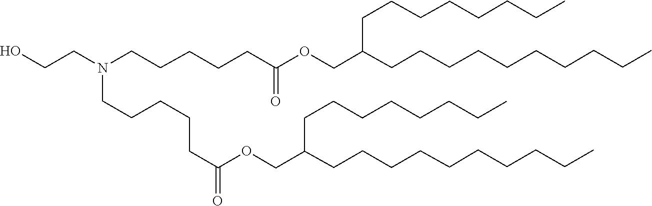 [SMILES: CCCCCCCCCCC(CCCCCCCC)COC(=O)CCCCCN(CCO)CCCCCC(=O)OCC(CCCCCCCC)CCCCCCCCCC] |
| III-11 | 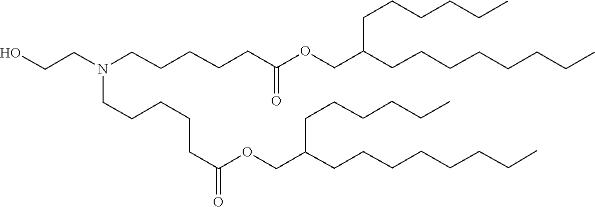 [SMILES: CCCCCCCCC(CCCCCC)COC(=O)CCCCCN(CCO)CCCCCC(=O)OCC(CCCCCC)CCCCCCCC] |
| III-12 | 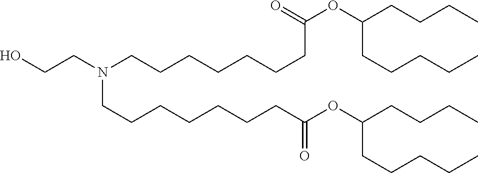 [SMILES: CCCCCC(CCCC)OC(=O)CCCCCCCN(CCO)CCCCCCCC(=O)OC(CCCC)CCCCC] |
| III-13 | 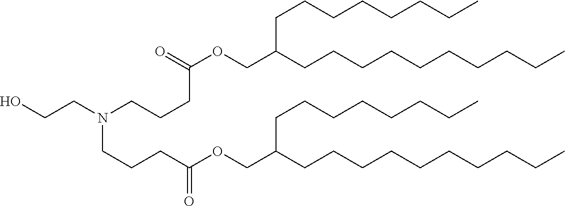 [SMILES: CCCCCCCCCCC(CCCCCCCC)COC(=O)CCCN(CCO)CCCC(=O)OCC(CCCCCCCC)CCCCCCCCCC] |
| III-14 | 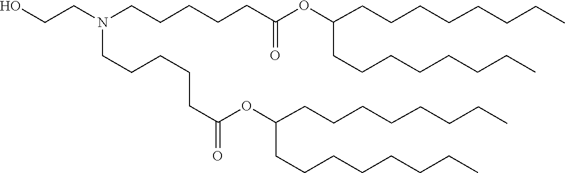 [SMILES: CCCCCCCCC(CCCCCCCC)OC(=O)CCCCCN(CCO)CCCCCC(=O)OC(CCCCCCCC)CCCCCCCC] |
| III-15 | 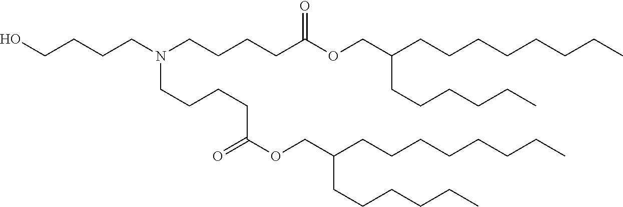 [SMILES: CCCCCCCCC(CCCCCC)COC(=O)CCCCN(CCCCO)CCCCC(=O)OCC(CCCCCC)CCCCCCCC] |
| III-16 | 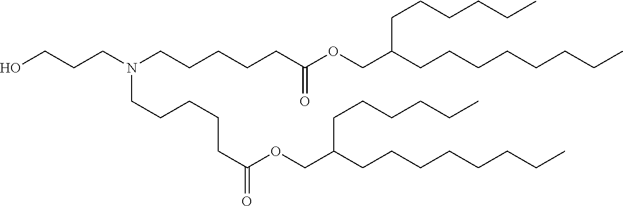 [SMILES: CCCCCCCCC(CCCCCC)COC(=O)CCCCCN(CCCO)CCCCCC(=O)OCC(CCCCCC)CCCCCCCC] |
| III-17 | 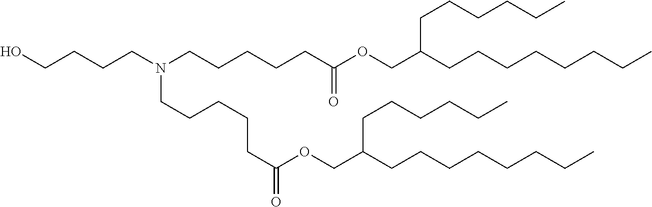 [SMILES: CCCCCCCCC(CCCCCC)COC(=O)CCCCCN(CCCCO)CCCCCC(=O)OCC(CCCCCC)CCCCCCCC] |
| III-18 | 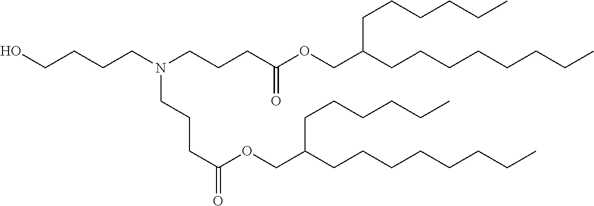 [SMILES: CCCCCCCCC(CCCCCC)COC(=O)CCCN(CCCCO)CCCC(=O)OCC(CCCCCC)CCCCCCCC] |
| III-19 | 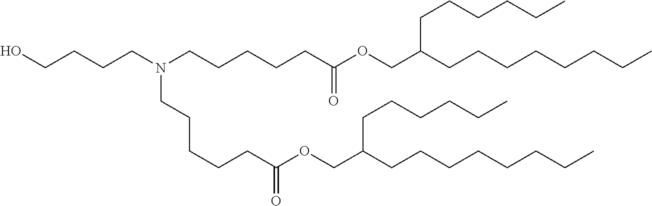 [SMILES: CCCCCCCCC(CCCCCC)COC(=O)CCCCCN(CCCCO)CCCCCC(=O)OCC(CCCCCC)CCCCCCCC] |
| III-20 | 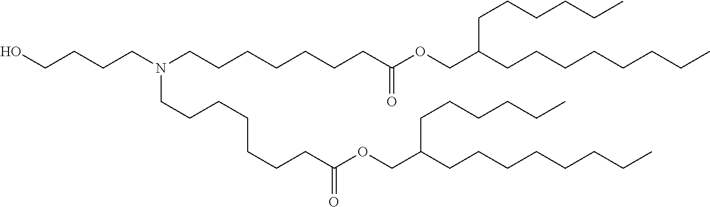 [SMILES: CCCCCCCCC(CCCCCC)COC(=O)CCCCCCCN(CCCCO)CCCCCCCC(=O)OCC(CCCCCC)CCCCCCCC] |
| III-21 | 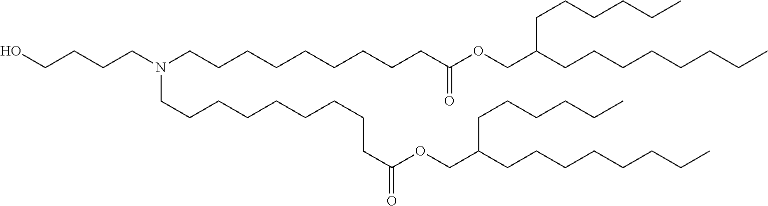 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 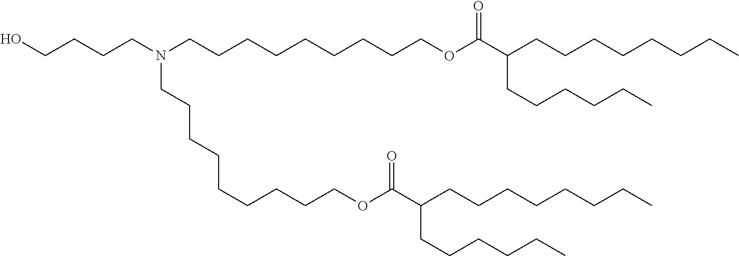 [SMILES: CCCCCCCCC(CCCCCC)C(=O)OCCCCCCCCCN(CCCCO)CCCCCCCCCOC(=O)C(CCCCCC)CCCCCCCC] |
| III-23 | 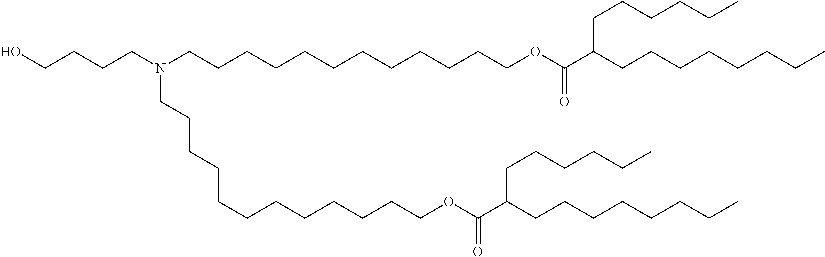 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 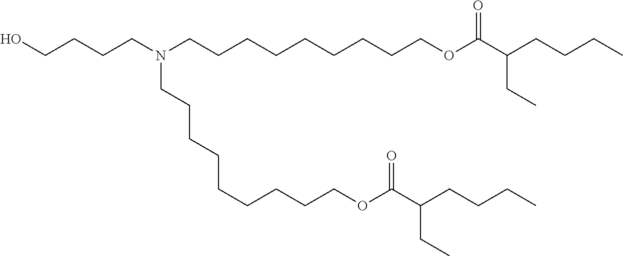 [SMILES: CCCCC(CC)C(=O)OCCCCCCCCCN(CCCCO)CCCCCCCCCOC(=O)C(CC)CCCC] |
| III-25 | 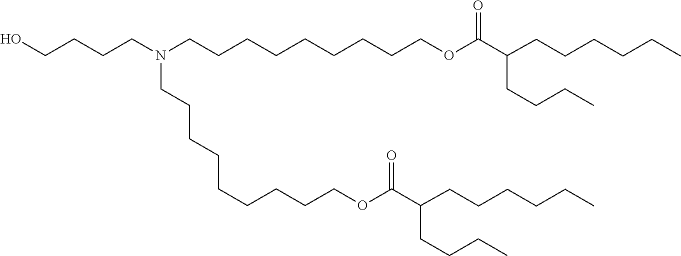 [SMILES: CCCCCCC(CCCC)C(=O)OCCCCCCCCCN(CCCCO)CCCCCCCCCOC(=O)C(CCCC)CCCCCC] |
| III-26 | 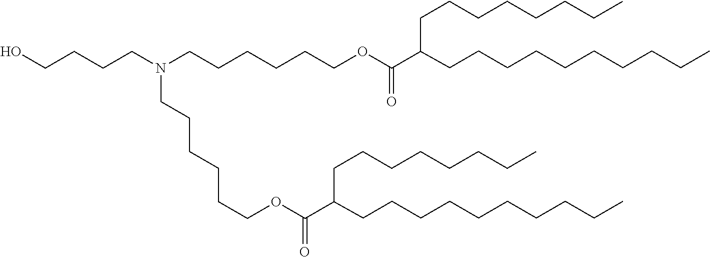 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 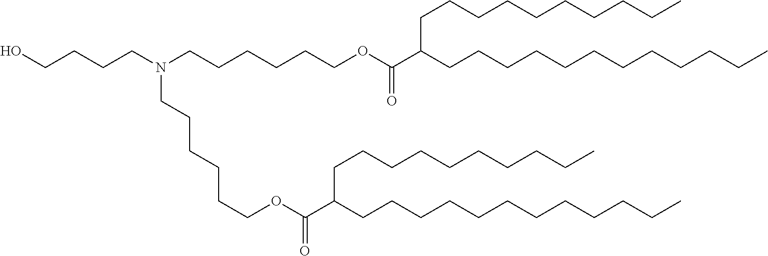 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 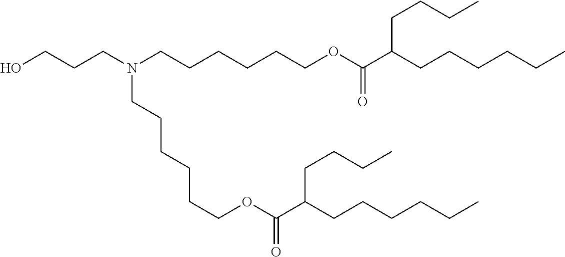 [SMILES: CCCCCCC(CCCC)C(=O)OCCCCCCN(CCCO)CCCCCCOC(=O)C(CCCC)CCCCCC] |
| III-29 | 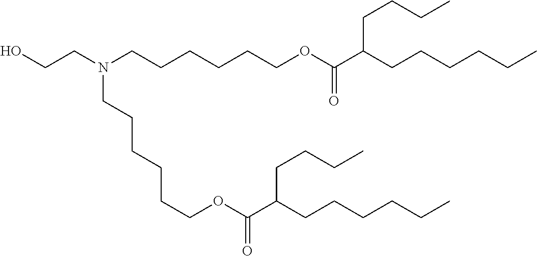 [SMILES: CCCCCCC(CCCC)C(=O)OCCCCCCN(CCO)CCCCCCOC(=O)C(CCCC)CCCCCC] |
| III-30 | 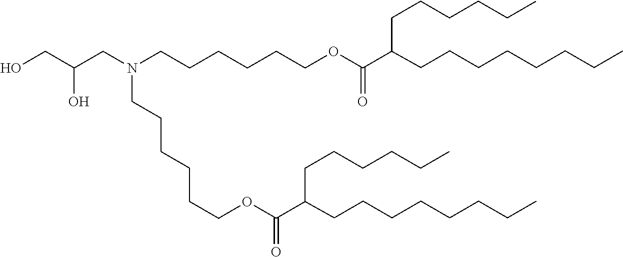 [SMILES: CCCCCCCCC(CCCCCC)C(=O)OCCCCCCN(CCCCCCOC(=O)C(CCCCCC)CCCCCCCC)CC(O)CO] |
| III-31 | 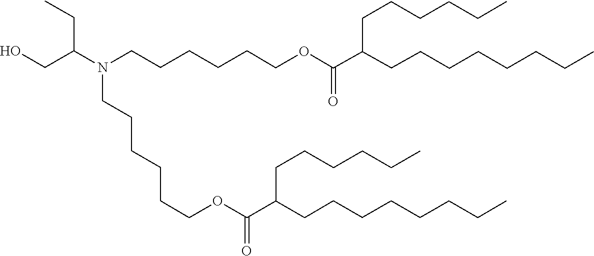 [SMILES: CCCCCCCCC(CCCCCC)C(=O)OCCCCCCN(CCCCCCOC(=O)C(CCCCCC)CCCCCCCC)C(CC)CO] |
| III-32 | 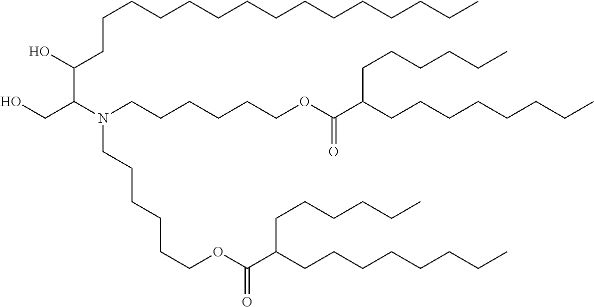 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 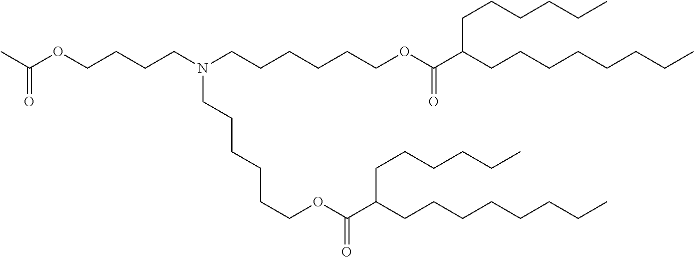 [SMILES: CCCCCCCCC(CCCCCC)C(=O)OCCCCCCN(CCCCCCOC(=O)C(CCCCCC)CCCCCCCC)CCCCOC(C)=O] |
| III-34 | 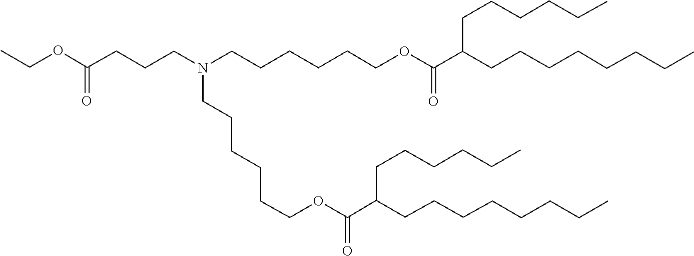 [SMILES: CCCCCCCCC(CCCCCC)C(=O)OCCCCCCN(CCCCCCOC(=O)C(CCCCCC)CCCCCCCC)CCCC(=O)OCC] |
| III-35 | 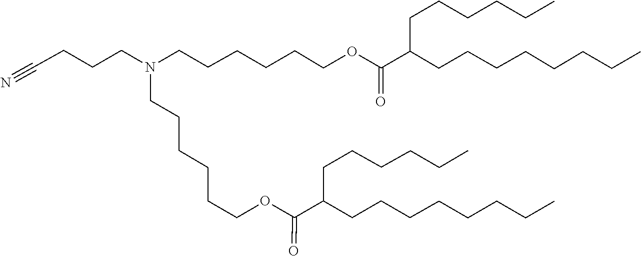 [SMILES: CCCCCCCCC(CCCCCC)C(=O)OCCCCCCN(CCCC#N)CCCCCCOC(=O)C(CCCCCC)CCCCCCCC] |
| III-36 | 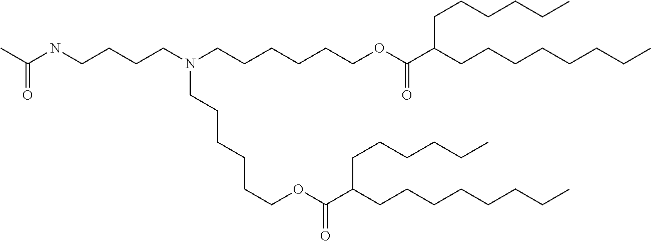 |

In some embodiments, an LNP comprises a lipid of Formula (III), RNA, a neutral lipid, a steroid and a pegylated lipid. In some embodiments, a lipid of Formula (III) is compound III-3. In some embodiments, a neutral lipid is DSPC. In some embodiments, a steroid is cholesterol. In some embodiments, a pegylated lipid is ALC-0159.

In some embodiments, the cationic lipid is present in the LNP in an amount from about 40 to about 50 mole percent. In some embodiments, the neutral lipid is present in the LNP in an amount from about 5 to about 15 mole percent. In some embodiments, the steroid is present in the LNP in an amount from about 35 to about 45 mole percent. In some embodiments, the pegylated lipid is present in the LNP in an amount from about 1 to about 10 mole percent.

In some embodiments, the LNP comprises compound III-3 in an amount from about 40 to about 50 mole percent, DSPC in an amount from about 5 to about 15 mole percent, cholesterol in an amount from about 35 to about 45 mole percent, and ALC-0159 in an amount from about 1 to about 10 mole percent.

In some embodiments, the LNP comprises compound III-3 in an amount of about 47.5 mole percent, DSPC in an amount of about 10 mole percent, cholesterol in an amount of about 40.7 mole percent, and ALC-0159 in an amount of about 1.8 mole percent. In various different embodiments, the cationic lipid has one of the structures set forth in the table below.

| No. | Structure |
|---|---|
| A | 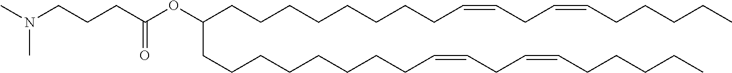 [SMILES: CCCCC/C=C\C/C=C\CCCCCCCCC(CCCCCCCC/C=C\C/C=C\CCCCC)OC(=O)CCCN(C)C] |
| B | 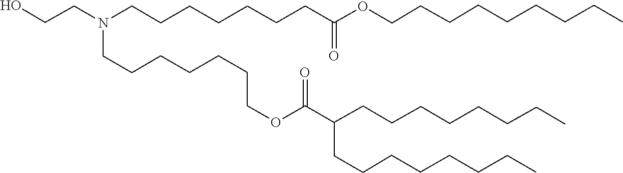 [SMILES: CCCCCCCCCOC(=O)CCCCCCCN(CCO)CCCCCCCOC(=O)C(CCCCCCCC)CCCCCCCC] |
| C | 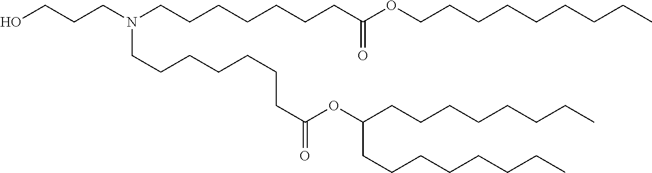 [SMILES: CCCCCCCCCOC(=O)CCCCCCCN(CCCO)CCCCCCCC(=O)OC(CCCCCCCC)CCCCCCCC] |
| D | 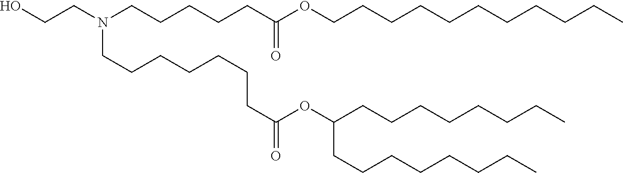 [SMILES: CCCCCCCCCCCOC(=O)CCCCCN(CCO)CCCCCCCC(=O)OC(CCCCCCCC)CCCCCCCC] |
| E | 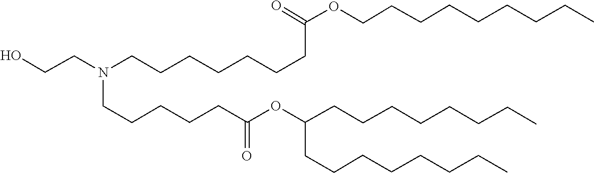 [SMILES: CCCCCCCCCOC(=O)CCCCCCCN(CCO)CCCCCC(=O)OC(CCCCCCCC)CCCCCCCC] |
| F | 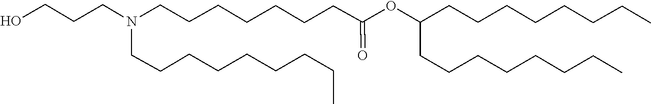 [SMILES: CCCCCCCCCN(CCCO)CCCCCCCC(=O)OC(CCCCCCCC)CCCCCCCC] |

In some embodiments, the LNP comprises a cationic lipid shown in the above table, e.g., a cationic lipid of Formula (B) or Formula (D), in particular a cationic lipid of Formula (D), RNA, a neutral lipid, a steroid and a pegylated lipid. In some embodiments, the neutral lipid is DSPC. In some embodiments, the steroid is cholesterol. In some embodiments, the pegylated lipid is DMG-PEG 2000.

In some embodiments, the LNP comprises a cationic lipid that is an ionizable lipid-like material (lipidoid). In some embodiments, the cationic lipid has the following structure:

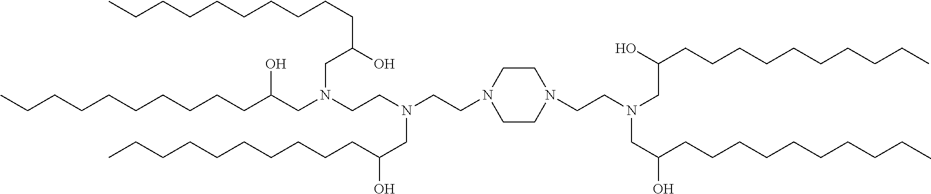 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

The N/P value is preferably at least about 4. In some embodiments, the N/P value ranges from 4 to 20, 4 to 12, 4 to 10, 4 to 8, or 5 to 7. In some embodiments, the N/P value is about 6.

LNP described herein may have an average diameter that in some embodiments ranges from about 30 nm to about 200 nm, or from about 60 nm to about 120 nm.

### Pharmaceutical Compositions

In some embodiments, a pharmaceutical composition comprises an RNA polynucleotide disclosed herein formulated as a particle. In some embodiments, a particle is or comprises a lipid nanoparticle (LNP) or a lipoplex (LPX) particle.

In some embodiments, an RNA polynucleotide disclosed herein may be administered in a pharmaceutical composition or a medicament and may be administered in the form of any suitable pharmaceutical composition.

In some embodiments, a pharmaceutical composition described herein is an immunogenic composition for inducing an immune response. For example, in some embodiments, an immunogenic composition is a vaccine.

In some embodiments, an RNA polynucleotide disclosed herein may be administered in a pharmaceutical composition which may comprise a pharmaceutically acceptable carrier and may optionally comprise one or more adjuvants, stabilizers etc. In some embodiments, a pharmaceutical composition is for therapeutic or prophylactic treatments.

The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cytokines, such as monokines, lymphokines, interleukins, chemokines. The cytokines may be IL1, IL2, IL3, IL4, IL5, IL6, IL7, IL8, IL9, IL10, IL12, IFNα, IFNγ, GM-CSF, LT-a. Further known adjuvants are aluminium hydroxide, Freund's adjuvant or oil such as Montanide® ISA51. Other suitable adjuvants for use in the present disclosure include lipopeptides, such as Pam3Cys. The pharmaceutical compositions according to the present disclosure are generally applied in a “pharmaceutically effective amount” and in “a pharmaceutically acceptable preparation”. The term “pharmaceutically acceptable” refers to the non-toxicity of a material which does not interact with the action of the active component of the pharmaceutical composition. The term “pharmaceutically effective amount” or “therapeutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

In some embodiments, a pharmaceutical composition disclosed herein may contain salts, buffers, preservatives, and optionally other therapeutic agents. In some embodiments, a pharmaceutical composition disclosed herein comprises one or more pharmaceutically acceptable carriers, diluents and/or excipients.

Suitable preservatives for use in a pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants. The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In some embodiments, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

In some embodiments, a pharmaceutical composition described herein may be administered intravenously, intraarterially, subcutaneously, intradermally or intramuscularly. In certain embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the pharmaceutical composition is formulated for intramuscular administration. In another embodiment, the pharmaceutical composition is formulated for systemic administration, e.g., for intravenous administration.

### Characterization

In some embodiments, an RNA polynucleotide disclosed herein is characterized in that, when assessed in an organism administered a composition or medical preparation comprising an RNA polynucleotide, elevated expression of a payload is observed relative to an appropriate reference comparator.

In some embodiments, an RNA polynucleotide disclosed herein is characterized in that, when assessed in an organism administered a composition or medical preparation comprising an RNA polynucleotide, increased duration of expression (e.g., prolonged expression) of a payload is observed relative to an appropriate reference comparator.

In some embodiments, an RNA polynucleotide disclosed herein is characterized in that, when assessed in an organism administered a composition or medical preparation comprising an RNA polynucleotide, decreased interaction with IFIT1 of an RNA polynucleotide is observed relative to an appropriate reference comparator.

In some embodiments, an RNA polynucleotide disclosed herein is characterized in that, when assessed in an organism administered a composition or medical preparation comprising an RNA polynucleotide, increased translation an RNA polynucleotide is observed relative to an appropriate reference comparator.

In some embodiments, a reference comparator comprises an organism administered an otherwise similar RNA polynucleotide without a cap described herein. In some embodiments, a reference comparator comprises an organism administered an otherwise similar RNA polynucleotide without a cap proximal sequence disclosed herein. In some embodiments, a reference comparator comprises an organism administered an otherwise similar RNA polynucleotide with a self-hybridizing sequence.

In some embodiments, an RNA polynucleotide disclosed herein is characterized in that, when assessed in an organism administered a composition or medical preparation comprising an RNA polynucleotide, elevated expression and increased duration of expression (e.g., prolonged expression) of a payload is observed relative to an appropriate reference comparator.

In some embodiments, elevated expression is determined at least 24 hours, at least 48 hours at least 72 hours, at least 96 hours or at least 120 hours after administration of a composition or medical preparation comprising an RNA polynucleotide. In some embodiments, elevated expression is determined at least 24 hours after administration of a composition or medical preparation comprising an RNA polynucleotide. In some embodiments, elevated expression is determined at least 48 hours after administration of a composition or medical preparation comprising an RNA polynucleotide. In some embodiments, elevated expression is determined at least 72 hours after administration of a composition or medical preparation comprising an RNA polynucleotide. In some embodiments, elevated expression is determined at least 96 hours after administration of a composition or medical preparation comprising an RNA polynucleotide. In some embodiments, elevated expression is determined at least 120 hours after administration of a composition or medical preparation comprising an RNA polynucleotide.

In some embodiments, elevated expression is determined at about 24-120 hours after administration of a composition or medical preparation comprising an RNA polynucleotide. In some embodiments, elevated expression is determined at about 24-110 hours, about 24-100 hours, about 24-90 hours, about 24-80 hours, about 24-70 hours, about 24-60 hours, about 24-50 hours, about 24-40 hours, about 24-30 hours, about 30-120 hours, about 40-120 hours, about 50-120 hours, about 60-120 hours, about 70-120 hours, about 80-120 hours, about 90-120 hours, about 100-120 hours, or about 110-120 hours after administration of a composition or medical preparation comprising an RNA polynucleotide.

In some embodiments, elevated expression of a payload is at least 2-fold to at least 10-fold. In some embodiments, elevated expression of a payload is at least 2-fold. In some embodiments, elevated expression of a payload is at least 3-fold. In some embodiments, elevated expression of a payload is at least 4-fold. In some embodiments, elevated expression of a payload is at least 6-fold. In some embodiments, elevated expression of a payload is at least 8-fold. In some embodiments, elevated expression of a payload is at least 10-fold.

In some embodiments, elevated expression of a payload is about 2-fold to about 50-fold. In some embodiments, elevated expression of a payload is about 2-fold to about 45-fold, about 2-fold to about 40-fold, about 2-fold to about 30-fold, about 2-fold to about 25-fold, about 2-fold to about 20-fold, about 2-fold to about 15-fold, about 2-fold to about 10-fold, about 2-fold to about 8-fold, about 2-fold to about 5-fold, about 5-fold to about 50-fold, about 10-fold to about 50-fold, about 15-fold to about 50-fold, about 20-fold to about 50-fold, about 25-fold to about 50-fold, about 30-fold to about 50-fold, about 40-fold to about 50-fold, or about 45-fold to about 50-fold.

In some embodiments, elevated expression (e.g., increased duration of expression) of a payload persists for at least 24 hours, at least 48 hours, at least 72 hours, at least 96 hours, or at least 120 hours after administration of a composition or a medical preparation comprising an RNA polynucleotide. In some embodiments, elevated expression of a payload persists for at least 24 hours after administration. In some embodiments, elevated expression of a payload persists for at least 48 hours after administration. In some embodiments, elevated expression of a payload persists for at least 72 hours after administration. In some embodiments, elevated expression of a payload persists for at least 96 hours after administration. In some embodiments, elevated expression of a payload persists for at least 120 hours after administration of a composition or medical preparation comprising an RNA polynucleotide.

In some embodiments, elevated expression of a payload persists for at about 24-120 hours after administration of a composition or medical preparation comprising an RNA polynucleotide. In some embodiments, elevated expression persists for about 24-110 hours, about 24-100 hours, about 24-90 hours, about 24-80 hours, about 24-70 hours, about 24-60 hours, about 24-50 hours, about 24-40 hours, about 24-30 hours, about 30-120 hours, about 40-120 hours, about 50-120 hours, about 60-120 hours, about 70-120 hours, about 80-120 hours, about 90-120 hours, about 100-120 hours, or about 110-120 hours after administration of a composition or medical preparation comprising an RNA polynucleotide.

### Uses

Disclosed herein, among other things, are methods of making and methods of using an RNA polynucleotide comprising a 5′cap; a 5′ UTR comprising a cap proximal structure; and a sequence encoding a payload.

In some embodiments, disclosed herein is a method of producing a polypeptide comprising a step of: providing an RNA polynucleotide that comprises a 5′ cap (e.g., as described herein), a cap proximal sequence that comprises positions +1, +2, +3, +4, and +5 of an RNA polynucleotide, and a sequence encoding a payload; wherein an RNA polynucleotide is characterized in that when assessed in an organism administered an RNA polynucleotide or a composition comprising the same, elevated expression and/or increased duration of expression of an payload is observed relative to an appropriate reference comparator.

In some embodiments, disclosed herein is a method comprising: administering to a subject, a pharmaceutical composition comprising an RNA polynucleotide formulated in a lipid nanoparticle (LNP) or a lipoplex (LPX) particle disclosed herein.

In some embodiments, disclosed herein is a method of inducing an immune response in a subject, comprising administering to a subject, a pharmaceutical composition comprising an RNA polynucleotide formulated in a lipid nanoparticle (LNP) or a lipoplex (LPX) particle disclosed herein.

In some embodiments, disclosed herein is a method of vaccination of a subject, comprising administering to a subject, a pharmaceutical composition comprising an RNA polynucleotide formulated in a lipid nanoparticle (LNP) or a lipoplex (LPX) particle disclosed herein.

In some embodiments, provided herein is a method of decreasing interaction with IFIT1 of an RNA polynucleotide that comprises a 5′ cap and a cap proximal sequence comprising positions +1, +2, +3, +4, and +5 of the RNA polynucleotide, the method comprising a step of: providing a variant of an RNA polynucleotide that differs from a parental RNA polynucleotide by substitution of one or more residues within the cap proximal sequence, and determining that interaction of a variant with IFIT1 is decreased relative to that of a parental RNA polynucleotide. In some embodiments, determining comprises administering the RNA polynucleotide or a composition comprising the same to a cell or an organism.

In some embodiments, disclosed herein is a method of increasing translatability of an RNA polynucleotide that comprises a 5′ cap, a cap proximal sequence that comprises positions +1, +2, +3, +4, and +5 of the RNA polynucleotide and a sequence encoding a payload, the method comprising a step of: providing a variant of an RNA polynucleotide that differs from a parental RNA polynucleotide by substitution of one or more residues within a cap proximal sequence; and determining that expression of a variant is increased relative to that of a parental RNA polynucleotide. In some embodiments, determining comprises administering the RNA polynucleotide or a composition comprising the same to a cell or an organism. In some embodiments, increased translatability is assessed by increased expression and/or a persistence of expression of the payload. In some embodiments, increased expression is determined at least 6 hours, at least 24 hours, at least 48 hours at least 72 hours, at least 96 hours or at least 120 hours after administering. In some embodiments, increase in expression is at least 2-fold to 10-fold. In some embodiments, increase in expression is about 2-fold to 50-fold. In some embodiments, elevated expression persists for at least 24 hours, at least 48 hours, at least 72 hours, at least 96 hours, or at least 120 hours after administration.

In some embodiments of any of the methods disclosed herein, an immune response is induced in a subject. In some embodiments of any of the methods disclosed herein, an immune response is a prophylactic immune response or a therapeutic immune response.

In some embodiments of any of the methods disclosed herein, a subject is a mammal.

In some embodiments of any of the methods disclosed herein, a subject is a human.

In some embodiments of any of the methods disclosed herein, a subject has a disease or disorder disclosed herein.

In some embodiments of any of the methods disclosed herein, vaccination generates an immune response to an agent. In some embodiments, an immune response is a prophylactic immune response.

In some embodiments of any of the methods disclosed herein, a subject has a disease or disorder disclosed herein.

In some embodiments of any of the methods disclosed herein, one dose of a pharmaceutical composition is administered.

In some embodiments of any of the methods disclosed herein, a plurality of doses of a pharmaceutical composition is administered.

In some embodiments of any of the methods disclosed herein, the method further comprises administration of one or more therapeutic agents. In some embodiments, one or more therapeutic agents are administered before, after, or concurrently with administration of a pharmaceutical composition comprising an RNA polynucleotide.

Also provided herein is a method of improving capping efficiency (e.g., percentage of capped transcripts in an in vitro transcription reaction) of RNA transcripts, the improvement that comprises including A or an analog thereof and U or an analog thereof at the +1 and +2 positions, respectively, of a transcription start site in a coding strand of a double-stranded DNA template for in vitro transcription. In some embodiments, a transcription start site may be AUA, AUC, AUG, or AUU. In some embodiments, such improvements can be observed independent of the identity of a 5′ UTR, capping method (e.g., enzymatic capping vs. co-transcriptional capping), cap structures (e.g., Cap0, Cap1, or Cap2), coding sequences, types of ribonucleotides (e.g., modified nucleotides vs. non-modified nucleotides), formulation (e.g., lipoplex vs. lipid nanoparticles) or combinations thereof.

Also provided herein in some embodiments, is a method of providing a framework for an RNA polynucleotide that comprises a 5′ cap, a cap proximal sequence, and a payload sequence, the method comprising a step of:
assessing at least two variants of an RNA polynucleotide, wherein:
   each variant includes a same 5′ cap and payload sequence; and
   the variants differ from one another at one or more specific residues of a cap proximal sequence;
wherein the assessing comprises determining expression levels and/or duration of expression of a payload; and
selecting at least one combination of 5′ cap and a cap proximal sequence that displays elevated expression relative to at least one other combination.

In some embodiments, assessing comprises administering an RNA construct or a composition comprising the same to a cell or an organism:

In some embodiments, elevated expression of a payload is detected at a time point at least 6 hours, at least 24 hours, at least 48 hours, at least 72 hours, at least 96 hours, or at least 120 hours after administering. In some embodiments, elevated expression is at least 2-fold to 10-fold. In some embodiments, elevated expression is about 2-fold to about 50-fold.

In some embodiments, elevated expression of a payload persists for at least 24 hours, at least 48 hours, at least 72 hours, at least 96 hours, or at least 120 hours after administering.

In some embodiments of any of the methods disclosed herein, an RNA polynucleotide comprises one or more features of an RNA polynucleotide provided herein.

In some embodiments of any of the methods disclosed herein, a composition comprising an RNA polynucleotide comprises a pharmaceutical composition provided herein.

### ENUMERATED EMBODIMENTS

1. A composition or medical preparation comprising an RNA polynucleotide comprising:
a 5′ cap; a cap proximal sequence comprising positions +1, +2, +3, +4, and +5 of the RNA polynucleotide; and a sequence encoding a payload, wherein:
(i) the 5′ cap is a trinucleotide cap structure comprises N₁pN₂, wherein N₁ is position +1 of the RNA polynucleotide and N₂ is position +2 of the RNA polynucleotide, and wherein
   N₁ is A or an analog thereof; and
   N₂ is U or an analog thereof; and
(ii) the cap proximal sequence comprises:
   N₁ and N₂ of the trinucleotide cap structure and a sequence comprising N₃N₄N₅ at positions +3, +4, and +5 respectively of the RNA polynucleotide, wherein N₃, N₄, and N₅ are each independently selected from: A, C, G, and U.

2. The composition or medical preparation of embodiment 1, wherein N₃ is A.

3. The composition or medical preparation of embodiment 1 or 2, wherein N₅ is U.

4. The composition or medical preparation of any one of embodiments 1-3, wherein N₄ is A.

5. The composition or medical preparation of any one of embodiments 1-3, wherein N₄ is C.

6. The composition or medical preparation of any one of embodiments 1-3, wherein N₄ is G.

7. The composition or medical preparation of any one of embodiments 1-3, wherein N₄ is U.

8. The composition or medical preparation of any one of embodiments 1-7, wherein the trinucleotide cap structure has a structure: G*N₁pN₂, wherein
G* comprises a structure of formula I:

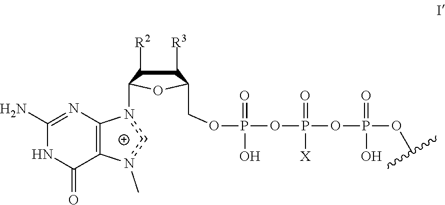

or a salt thereof,
wherein
each R² and R³ is —OH or —OCH₃; and
X is OH or SH (e.g., O⁻ or S⁻).

9. The composition or medical preparation of embodiment 8, wherein R² is —OH.

10. The composition or medical preparation of embodiment 8, wherein R² is —OCH₃.

11. The composition or medical preparation of any one of embodiments 8-10, wherein R³ is —OH.

12. The composition or medical preparation of any one of embodiments 8-10, wherein R³ is —OCH₃.

13. The composition or medical preparation of any one of embodiments 8-12, wherein X is OH (e.g., O⁻).

14. The composition or medical preparation of any one of embodiments 1-13, wherein the trinucleotide cap structure comprises a Cap1 structure.

15. The composition or medical preparation of any one of embodiments 1-14, wherein N₂ is of formula II:

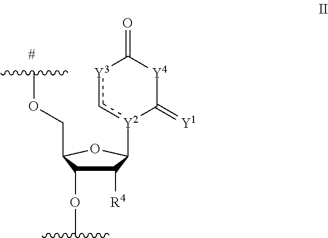

or a salt thereof, wherein:
each [symbol] is independently a single or double bond, as allowed by valency;
Y¹ is O or S;
Y² is N, C, or CH;
Y³ is N, NR^{a1}, CR^{a1}, or CHR^{a1};
Y⁴ is NR^{a2} or CHR^{a2};
each of R^{a1} or R^{a2} is independently hydrogen or C₁₋₆ aliphatic;
R⁴ is —OH or —OMe; and
#represents the point of attachment to p of N₁p.

16. The composition or medical preparation of embodiment 15, wherein N₂ is of formula IIa:

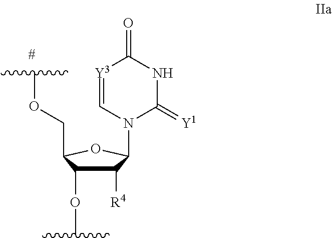

or a salt thereof.

17. The composition or medical preparation of embodiment 15, wherein N₂ is of formula IIb:

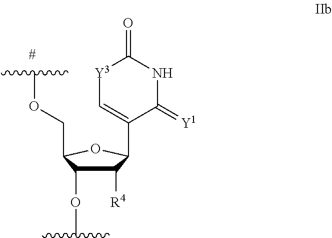

or a salt thereof.

18. The composition or medical preparation of any one of embodiments 15-17, wherein Y¹ is O.

19. The composition or medical preparation of any one of embodiments 15-17, wherein Y¹ is S.

20. The composition or medical preparation of embodiment 15 or 16, wherein Y³ is N.

21. The composition or medical preparation of embodiment 15 or 16, wherein Y³ is CR^{a1}.

22. The composition or medical preparation of embodiment 15 or 17, wherein Y³ is NR^{a1}.

23. The composition or medical preparation of embodiment 15 or 17, wherein Y³ is CHR^{a1}.

24. The composition or medical preparation of any one of embodiments 15-23, wherein each R^{a1} or R^{a2} is independently hydrogen or methyl.

25. The composition or medical preparation of any one of embodiments 15-24, wherein R⁴ is —OH.

26. The composition or medical preparation of any one of embodiments 15-24, wherein R⁴ is —OMe.

27. The composition or medical preparation of any one of embodiments 1-14, wherein N₂ is uridine, or a modified uridine (e.g., m1ψ, 2-thio-uridine, or 5-methyluridine).

28. The composition or medical preparation of any one of embodiments 1-14, wherein N₂ is:

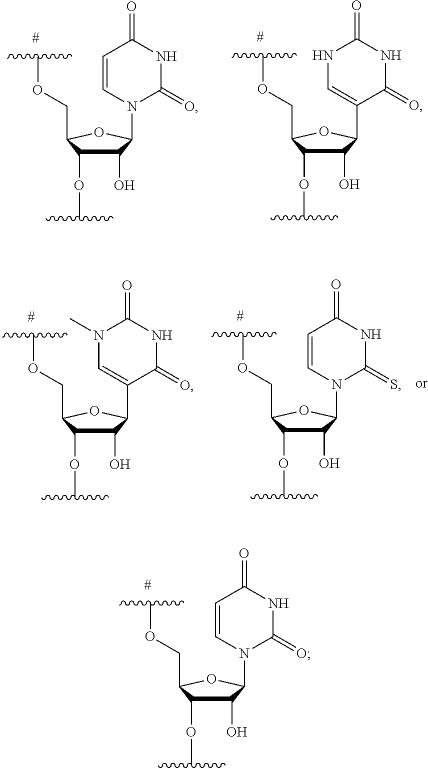

or a salt thereof;
wherein #represents the point of attachment to p of N₁p.

29. The composition or medical preparation of any one of embodiments 1-28, wherein N₁ is adenosine or a modified adenosine (e.g., 6-methyladenosine).

30. The composition or medical preparation of any one of embodiments 1-7, wherein the 5′ cap is (m^{7,2′-O})Gppp(m^{2′-O})A₁pU₂, (m^{7,3′-O})Gppp(m^{2′-O})A₁pU₂, (m^{7,2′-O})Gppp(m^{2′-O})A₁pΨ₂, (m^{7,3′-O})Gppp(m^{2′-O})A₁pΨ₂, (m^{7,2′-O})Gppp(m^{2′-O})A₁p(m¹)Ψ₂, (m^{7,3′-O})Gppp(m^{2′-O})A₁p(m¹)Ψ₂, (m^{7,2′-O})Gppp(m^{2′-O})A₁pS²U₂, (m^{7,3′-O})Gppp(m^{2′-O})A₁pS²U₂, (m^{7,2′-O})Gppp(m^{2′-O})A₁p(m⁵)U₂, or (m^{7,3′-O})Gppp(m^{2′-O})A₁p(m⁵)U₂.

31. The composition or medical preparation of any one of embodiments 1-7, wherein the 5′ cap is (m^{7,2′-O})Gppp(m₁^{6,2′-O})A₁pU₂, (m^{7,3′-O})Gppp(m^{6,2′-O})A₁pU₂, (m^{7,2′-O})Gppp(m^{6,2′-O})A₁pΨ₂, (m^{7,3′-O})Gppp(m^{6,2′-O})A₁pΨ₂, (m^{7,2′-O})Gppp(m^{6,2′-O})A₁p(m¹)Ψ₂, (m^{7,3′-O})Gppp(m^{6,2′-O})A₁p(m¹)Ψ₂, (m^{7,2′-O})Gppp(m^{6,2′-O})A₁pS²U₂, (m^{7,3′-O})Gppp(m^{6,2′-O})A₁pS²U₂, (m^{7,2′-O})Gppp(m^{6,2′-O})A₁p(m)U₂, or (m^{7,3′-O})Gppp(m^{6,2′-O})A₁p(m)U₂.

32. An in vitro transcription reaction comprising:
(i) a template DNA strand comprising a polynucleotide sequence complementary to an RNA polynucleotide sequence provided in any one of embodiments 1-31, wherein the template DNA strand comprises a sequence that is complementary to a AUA, AUC, AUG, or AUU transcription start site;
(ii) a polymerase;
(iii) ribonucleotides; and
(iv) a 5′ cap comprising N₁pN₂;
wherein N₁ is A or an analog thereof, and N₂ is U or an analog thereof;
wherein the sequence in the template strand that is complementary to AUA, AUC, AUG, or AUU is the start site of an RNA polymerase promoter.

33. The in vitro transcription reaction of embodiment 32, wherein the template DNA strand comprises a sequence that is complementary to a transcription start site comprising AUA.

34. The in vitro transcription reaction of embodiment 32, wherein the template DNA strand comprises a sequence that is complementary to a transcription start site comprising AUC.

35. The in vitro transcription reaction of embodiment 32, wherein the template DNA strand comprises a sequence that is complementary to a transcription start site comprising AUG.

36. The in vitro transcription reaction of embodiment 32, wherein the template DNA strand comprises a sequence that is complementary to a transcription start site comprising AUU.

37. The in vitro transcription reaction of any one of embodiments 32-36, wherein the template DNA strand comprises: a sequence encoding a 5′ UTR, a sequence encoding a payload, a sequence encoding a 3′ UTR, and a sequence encoding a polyA sequence.

38. The in vitro transcription reaction of any one of embodiments 32-37, wherein N₂ is uridine or a modified uridine (e.g., m1ψ, 2-thio-uridine, or 5-methyluridine).

39. An RNA polynucleotide produced from an in vitro transcription reaction provided in any one of embodiments 32-38.

40. A method of making a capped RNA polynucleotide comprising a 5′ cap comprising N₁pN₂; a cap proximal sequence comprising positions +1, +2, +3, +4, and +5 of the RNA polynucleotide; and a sequence encoding a payload, wherein:
the cap proximal sequence comprises N₁ and N₂ of the 5′ cap, and N₃, N₄, and N₅, wherein N₁ to N₅ correspond to positions +1, +2, +3, +4, and +5 of the RNA polynucleotide, wherein N₁ is A or an analog thereof, N₂ is U or an analog thereof, and N₃, N₄, and N₅ are each independently chosen from: A, C, G, and U; and
wherein the method comprises transcribing a template DNA strand in the presence of the 5′ cap and an RNA polymerase, wherein the template DNA strand comprises an RNA polymerase promoter sequence and a sequence that is complementary to a transcription start site, wherein the sequence that is complementary to the transcription start site is the start site of the RNA polymerase promoter.

41. The method of embodiment 39, wherein N₁ is complementary to position +1 of the template DNA strand (corresponding to the first nucleotide of the transcription start site), and N₂ is complementary to position +2 of the template DNA strand (corresponding to the second nucleotide of the transcription start site).

42. The method of embodiment 39 or 40, wherein the RNA polymerase is T7 RNA polymerase.

43. The method of any one of embodiments 39-41, wherein N₂ is uridine or modified uridine (e.g., m1ψ, 2-thio-uridine, or 5-methyluridine).

44. A method of making a capped RNA polynucleotide comprising:
transcribing a DNA template strand in the presence of a 5′ cap, wherein the 5′ cap comprises the structure N₁pG₂,
wherein the DNA template strand comprises an RNA polymerase promoter sequence and a sequence that is complementary to a AUA, AUC, AUG, or AUU transcription start site;
wherein N₁ is A or an analog thereof, and N₂ is U or an analog thereof.

45. The method of embodiment 44, wherein N₂ is uridine or modified uridine (e.g., m1ψ, 2-thio-uridine, or 5-methyluridine).

46. A complex comprising a DNA template strand and a 5′ cap analog comprising a structure of N₁pN₂, wherein the DNA template strand comprises an RNA polymerase promoter sequence and a sequence that is complementary to a transcription start site;
wherein N₁ is A or an analog thereof, and N₂ is U or an analog thereof;
wherein N₁ interacts with the +1 position of the DNA template strand (corresponding to the first nucleotide of the transcription start site) and N₂ interacts with the +2 position of the DNA template strand (corresponding to the second nucleotide of the transcription start site); and
wherein the sequence in the template strand that is complementary to the transcription start site is the start site of an RNA polymerase promoter.

47 The complex of embodiment 46, wherein position +1 and position +2 of the DNA template strand are T and A, respectively.

48. The complex of embodiment 46 or 47, wherein the nucleotides of the cap interact with the nucleotides of the template DNA strand via canonical Watson-Crick base pairing.

49. The complex of any one of embodiments 46-48, wherein the RNA polymerase promoter sequence is a T7 RNA polymerase promoter sequence.

50. The complex of any one of embodiments 46-49, wherein the complex further comprises an RNA polymerase (e.g., a T7 RNA polymerase).

51. The complex of any one of embodiments 46-50, wherein N₂ is uridine or modified uridine (e.g., m1ψ, 2-thio-uridine, or 5-methyluridine).

52. A method of formulating a pharmaceutical composition, the method comprising combining a preparation comprising an RNA polynucleotide of any one of embodiments 1 to 31 with a preparation comprising lipids.

53. The method of embodiment 52, wherein the method comprises combining the preparation comprising RNA polynucleotide with the preparation comprising lipids to form lipid nanoparticles that encapsulate the RNA polynucleotide.

54. The method of embodiment 52, wherein the method comprises combining the preparation comprising RNA polynucleotide with the preparation comprising lipids to form RNA lipoplexes.

55. A compound of formula G*N₁pN₂, wherein:
G* is of formula I′:

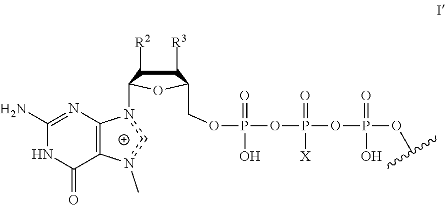

or a salt thereof, wherein:
   each R² and R³ is —OH or —OCH₃; and
   X is OH or SH (e.g., O— or S—);
p is a phosphate linker;
N₁ is A or an analog thereof; and
N₂ is U or an analog thereof.

56. The compound of embodiment 55, wherein R² is —OH.

57. The compound of embodiment 55, wherein R² is —OCH₃.

58. The compound of any one of embodiments 55-57, wherein R³ is —OH.

59. The compound of any one of embodiments 55-57, wherein R³ is —OCH₃.

60. The compound of any one of embodiments 55-59, wherein X is OH (e.g., O⁻).

61. The compound of any one of embodiments 55-60, wherein N₂ is of formula II′:

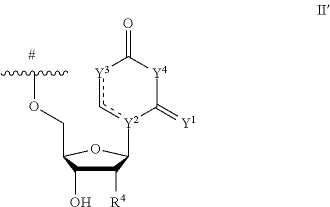

or a salt thereof, wherein:
each [symbol] is independently a single or double bond, as allowed by valency;
Y¹ is O or S;
Y² is N, C, or CH;
Y³ is N, NR^{a1}, CR^{a1}, or CHR^{a1};
Y⁴ is NR^{a2} or CHR^{a2};
each of R¹ or R^{a2} is independently hydrogen or C₁₋₆ aliphatic;
R⁴ is —OH or —OMe; and
#represents the point of attachment to p of N₁p.

62. The compound of embodiment 15, wherein N² is of formula IIa:

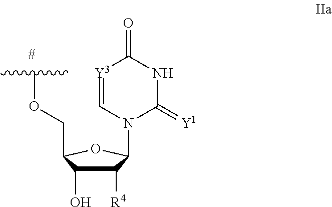

or a salt thereof.

63. The compound of embodiment 62, wherein N² is of formula IIb:

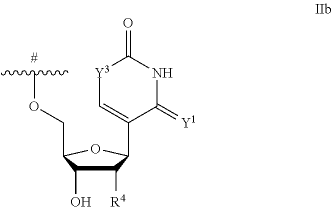

or a salt thereof.

64. The compound of any one of embodiments 61-63, wherein Y¹ is O.

65. The compound of any one of embodiments 61-63, wherein Y¹ is S.

66. The compound of embodiment 61 or 62, wherein Y³ is N.

67. The compound of embodiment 61 or 62, wherein Y³ is CR^{a1}.

68. The compound of embodiment 61 or 63, wherein Y³ is NR^{a1}.

69. The compound of embodiment 61 or 63, wherein Y³ is CHR^{a1}.

70. The compound of any one of embodiments 61-69, wherein each R^{a1} or R^{a2} is independently hydrogen or methyl.

71. The compound of any one of embodiments 61-70, wherein R⁴ is —OH.

72. The compound of any one of embodiments 61-70, wherein R⁴ is —OMe.

73. The compound of any one of embodiments 55-60, wherein N₂ is:

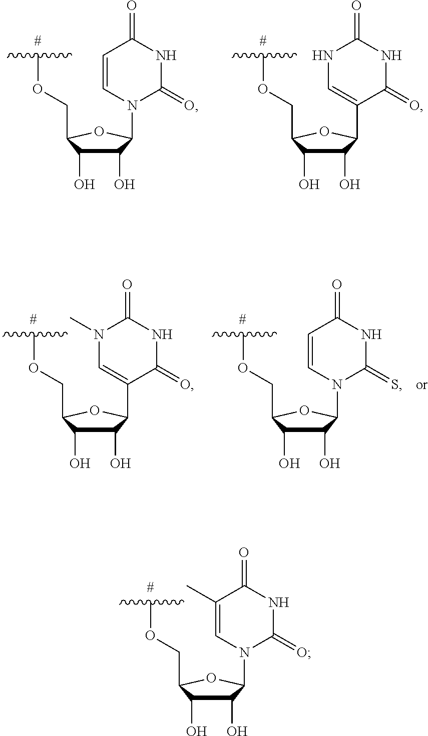

or a salt thereof;
wherein #represents the point of attachment to p of N₁p.

74. The compound of any one of embodiments 55-73, wherein N₁ is adenosine or 6-methyladenosine.

75. The compound of any one of embodiments 55-73, wherein N₁ is:

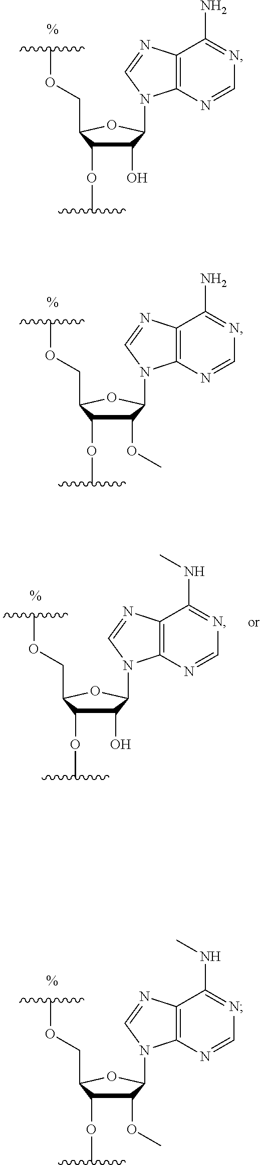

or a salt thereof,

76. The compound of any one of embodiments 55-75, wherein p is —P(═O)(OH)—, or a salt thereof.

77. The compound of embodiment 55, wherein the compound is (m^{7,2′-O})Gppp(m^{2′-O})A₁pU₂, (m^{7,3′-O})Gppp(m^{2′-O})A₁pU₂, (m^{7,2′-O})Gppp(m^{2′-O})A₁pΨ₂, (m^{7,3′-O})Gppp(m^{2′-O})A₁pΨ₂, (m^{7,2′-O})Gppp(m^{2′-O})A₁p(m¹)Ψ₂, (m^{7,3′-O})Gppp(m^{2′-O})A₁p(m¹)Ψ₂, (m^{7,2′-O})Gppp(m^{2′-O})A₁pS²U₂, (m^{7,3′-O})Gppp(m^{2′-O})A₁pS²U₂, (m^{7,2′-O})Gppp(m^{2′-O})A₁p(m⁵)U₂, or (m^{7,3′-O})Gppp(m^{2′-O})A₁p(m⁵)U₂, or a salt thereof.

78. The compound of embodiment 55, wherein the compound is (m^{7,2′-O})Gppp(m^{6,2′-O})A₁pU₂, (m^{7,3′-O})Gppp(m^{6,2′-O})A₁pU₂, (m^{7,2′-O})Gppp(m^{6,2′-O})A₁pΨ₂, (m^{7,3′-O})Gppp(m^{6,2′-O})A₁pΨ₂, (m^{7,2′-O})Gppp(m^{6,2′-O})A₁p(m¹)Ψ₂, (m^{7,3′-O})Gppp(m^{6,2′-O})A₁p(m¹)Ψ₂, (m^{7,2′-O})Gppp(m^{6,2′-O})A₁pS²U₂, (m^{7,3′-O})Gppp(m^{6,2′-O})A₁pS²U₂, (m^{7,2′-O})Gppp(m^{6,2′-O})A₁p(m⁵)U₂, or (m^{7,3′-O})Gppp(m^{6,2′-O})A₁p(m⁵)U₂, or a salt thereof.

79. The composition or medical preparation of embodiment 15 or 17, wherein Y³ is N.

80. A method of making a capped RNA polynucleotide comprising:
transcribing a DNA template strand in the presence of a 5′ cap, wherein the 5′ cap comprises the structure N₁pN₂,
wherein the DNA template strand comprises an RNA polymerase promoter sequence and a sequence that is complementary to a AUA, AUC, AUG, or AUU transcription start site;
wherein N₁ is A or an analog thereof, and N₂ is U or an analog thereof.

### EXEMPLIFICATION

### Example 1—Evaluation of (m₂^{7,3′-O})Gppp(m^{2′-O})ApU and (m^{7,3′-O})Gppp(m^{2′-O})A₁p(m¹)Ψ₂

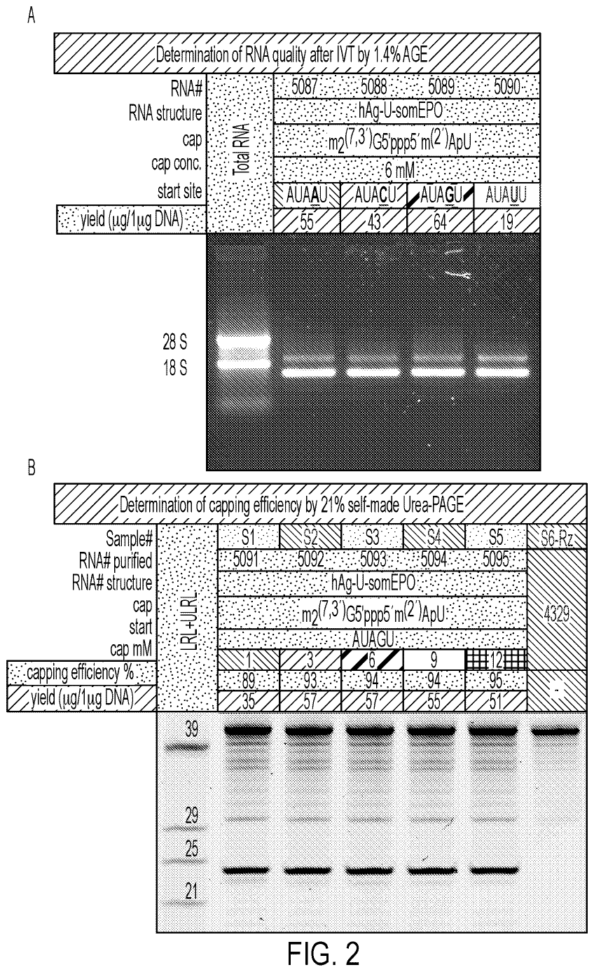

For templates, linearized plasmid encoding codon-optimized murine erythropoietin (EPO) were used. The mRNAs starting with AUAAU, AUACU, AUAGU or AUAUU were designed to contain the 5′ untranslated region (5′UTR) sequences of human α-globin (hAg) mRNA, an FI element as the 3′UTR, and an interrupted 100 nt-long 3′ poly(A) tail flanking the coding sequence. The MEGAscript T7 Transcription kit (Thermo Fisher Scientific, Waltham, MA, USA) was used for transcription, and UTP was kept or was replaced with N1-methylpseudouridine (m1Ψ) triphosphate (TriLink, San Diego, CA, USA). Capping of in vitro-transcribed mRNAs was performed co-transcriptionally using trinucleotide cap analogs (Cap 1 corresponds to compound I′-1 ((m₂^{7,3′-O})Gppp(m^{2′-O})ApU); Cap 2 corresponds to compound I′-6 ((m₂^{7,3′-O})Gppp(m^{2′-O})Ap(m¹)Ψ); CC114 corresponds to (m⁷)Gppp(m^{2′-O})ApU (TriLink, USA); CC413 corresponds to (m₂^{7,3′-O})Gppp(m^{2′-O})ApG (TriLink, USA). To obtain the desired transcripts generated with cap analogs, the initial GTP and UTP or m1ΨTP concentration in the transcription reaction was reduced from 7.5 mM to 1.5 mM and the 1.5 mL tubes were incubated at 37° C. for 30 min in a hybridization chamber. Sequential additions of 1.5 mM GTP and UTP or m1ΨTP were required to supplement the reaction at 30, 60, 90 and 120 min. incubated further at 37° C. for 30 min. To remove template DNA, Turbo DNase (Thermo Fisher Scientific, USA) was added to the reaction mix after the transcription reaction was completed and incubated for 15 min at 37° C. The synthesized mRNA was precipitated by adding a half volume of 8 M LiCl solution (Merck, Darmstadt, Germany) to the reaction mix and then pelleted by centrifugation. After dissolving in nuclease free water, mRNAs were cellulose-purified to remove double-stranded RNA contaminants, as described in Baiersdörfer, M., et al. (2019) Molecular therapy. Nucleic acids, 15, 26-35. The mRNA concentration and quality were measured on a NanoDrop™ 2000c spectrophotometer (Thermo Fisher Scientific, USA). Small aliquots of mRNA samples were stored in siliconized tubes at −20° C. These findings demonstrated that (m₂^{7,3′-O})Gppp(m^{2′-O})ApU paired with an AUAGU transcriptional start site generated EPO-encoding mRNAs (EPO mRNA) with the highest RNA yield (64 μg/Unit) (FIG. 2A).

In order to determine the capping efficiency of mRNAs, in vitro transcription reaction followed by a Ribozyme assay was performed in a cap analog concentration-dependent manner (1, 3, 6, 9 and 12 mM). The Ribozyme cleavage reaction contained 0.45 μM mRNA. A 3-fold molar excess of ribozyme over mRNA substrate was added in aqueous solution containing 30 mM HEPES and 150 mM NaCl. The ribozyme cleavage reaction was performed on a PCR machine utilizing the following program: 95° C. for 2 min, chill the mixture up to 37° C. by ramping rate of 0.1° C./sec, 37° C. for 5 min; after adding 30 mM MgCl₂ solution to each sample, the mixtures were maintained at 37° C. for 60 min followed by stopping the annealing at 80° C. for 2 min and transferring to ice for 5 min. Then, the short and long RNA fragments were separated using the RNA Clean & Concentrator-5 kit (Zymo Research Europe, Freiburg, Germany) according to the manufacturer's instructions. In this study, the following custom-designed hammerhead ribozyme specific for the hAg 5′UTR was used: 5′-UGU GGG CUG AUG AGG CCG UGA GGC CGA AAC CAG AAG AAU-3′ (SEQ ID NO: 44) (synthesized by Metabion International AG, Planegg, Germany). To detect the short fragments, the samples (30 ng) were resolved on a 21% (vol/vol) 19:1 acrylamide:bisacrylamide denaturing gel supplemented with 8 M urea (Merck, Germany). Before loading, the samples were denatured by incubation at 75° C. for 5 min in the presence of 2×RNA loading buffer (New England Biolabs, Germany). The gel was pre-run at 180 V for 60 min. When the pre-run was finished, the pockets were rinsed with 1×TBE buffer. Immediately afterwards, the samples were loaded, and the gel was run at 200 V constantly until the dye front reached the end of the gel. To identify the short, cleaved products, the gel was incubated with 1×TBE buffer containing 0.01% SYBR Gold nucleic acid stain (Thermo Fisher Scientific, USA) and the fluorescent signals were captured using a Gel Doc EZ Imager (Bio-Rad, Hercules, CA, USA). High yield is observed when the (m₂^{7,3′-O})Gppp(m^{2′-O})ApU was used in the range of 3-6 mM concentration (FIG. 2B), accordingly in further tests, the concentration of 4 mM was applied. The capping efficiency of (m₂^{7,3′-O})Gppp(m^{2′-O})ApU is close to 100% regardless of the concentration was used (FIG. 2B).

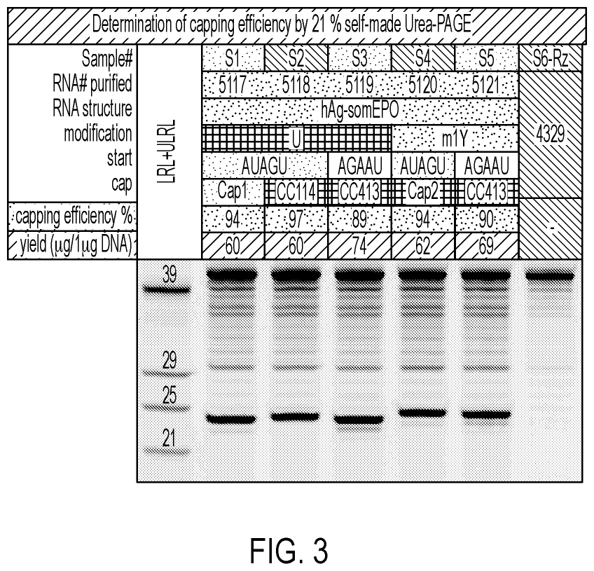

Using the optimized conditions, in vitro-transcribed (IVT) EPO mRNA containing U or m1Ψ was generated with trinucleotide cap analogs CC114, CC413, compound I′-1, or compound I′-6. High yield as well as capping efficiency is observed in terms of each tested mRNA regardless of cap analog. According to Ribozyme assay analysis, the capping efficiency of compound I′-1 and compound I′-6 is close to 100% and it is comparable to the commercially available CC114 and CC413 cap analogs (FIG. 3).

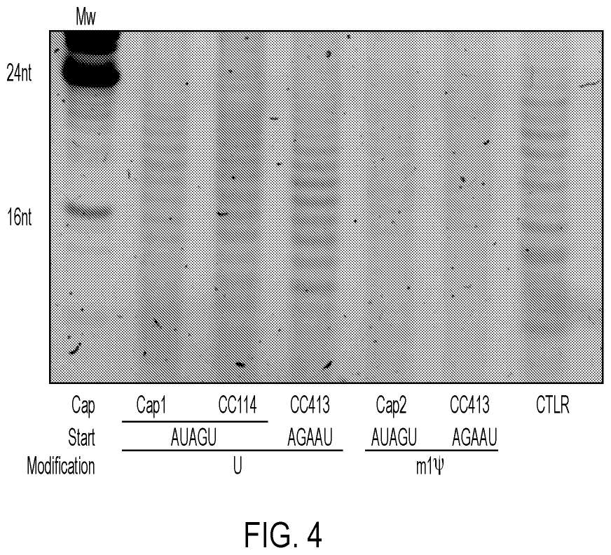

For detection of short byproducts, IVT mRNAs (1.5-2 μg) were resolved on a 21% (vol/vol) 19:1 acrylamide:bisacrylamide denaturing gel supplemented with 8 M urea (Merck, Germany). both When the pre-run was finished, the pockets were rinsed with 1×TBE buffer. Immediately afterwards, the samples were applied, and the gel was run at 180 V constantly until the dye front has reached the end of the gel. For identification of the short byproducts, the gel was incubated with 1×TBE buffer containing 0.01% SYBR Gold nucleic acid stain (Thermo Fisher Scientific, USA) and the fluorescent signals were captured using a Gel Doc EZ Imager (Bio-Rad, USA). Denaturing Urea Polyacrylamide Gel Electrophoresis showed minimal amount of short contaminants for compound I′-6 and CC413 for EPO m1Ψ-mRNAs, while for unmodified ones significant amount was observed independently of cap analogs (FIG. 4).

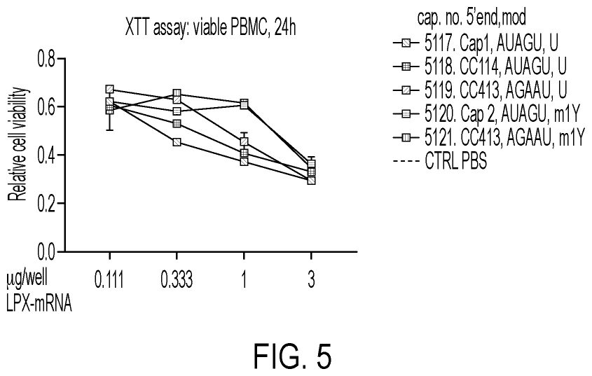

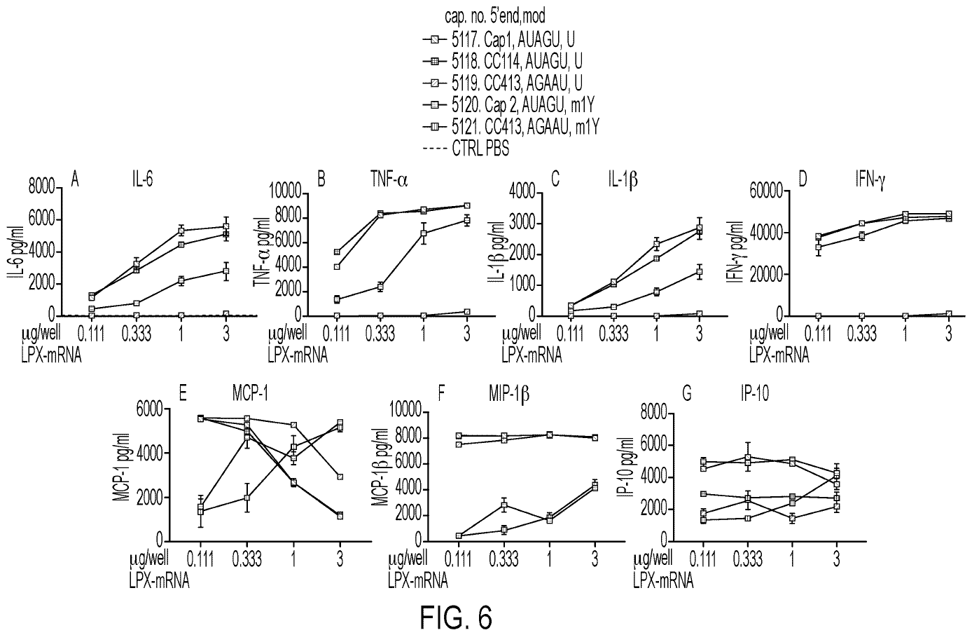

Human buffy coats from healthy individuals were obtained from the Faculty of Medicine of Johannes Gutenberg University, Mainz and used to isolate peripheral blood mononuclear cells (PBMCs) on a Ficoll-Paque™ PLUS (Cytiva, Marlborough, MA, USA) density gradient. In preparation for mRNA transfection, cryopreserved PBMCs were thawed and seeded into 96-well plates at a density of 5×10⁵ cells per well in 190 μL RPMI medium supplemented with 1% non-essential amino acids (NEAA), 1% sodium pyruvate and 10% Fetal Bovine Serum (Merck, Germany). Cells were maintained at 37° C. with 5% CO₂ until transfection with 0.5, 1.5, 5.0 and 15 μg/ml lipoplex-formulated EPO mRNA (LPX-RNA), as described in Kranz, L. M., et al. (2016) Nature, 534, 396-401. The complexed RNA (10 μl per well) was added in triplicates and supernatants were collected at 24 h after transfection to perform single cell cytotoxicity assay and to measure the cytokine/chemokine profile. To determine the cell viability and the production of selected cytokines/chemokines, supernatants from human PBMCs transfected with LPX-RNA were subjected to XTT cytotoxicity test using Cell Proliferation Kit II (Sigma-Aldrich) and cytokine/chemokine profile analysis using the Meso Scale Discovery V-PLEX Custom Human Biomarkers Proinflammatory and Chemokine Panel (Meso Scale Diagnostics—MSD, Rockville, MD, USA) according to the manufacturer's instructions, respectively. In regards of cytokine/chemokine profile analysis, a sample dilution of 1:5 (supernatant:MSD diluent) was used in each experiment. The levels of IL-6 (Interleukin 6), TNF-α (Tumor necrosis factor alpha), IL-1 (Interleukin 1 beta), IFN-γ (Interferon gamma), MCP-1 (Monocyte Chemoattractant Protein-1), MIP-10 (Macrophage inflammatory protein 1 beta) and IL-10 (Interleukin 10) were quantified 24 h after the mRNA transfection. These results demonstrated that no toxic effect on cell viability originating from compound I′-1 as well as compound I′-6 was observed (FIG. 5). No toxic effect on PBMCs up to 1 μg/well mRNA for m1Ψ-modified EPO mRNA (FIG. 5). Transfection of unmodified mRNAs leads to decrease in cellular viability starting at dose 1.5 μg/ml, however this effect does not depend on cap used but on mRNA modification (FIG. 5). Levels of each cytokine/chemokine showed zero to moderate increases in each sample measured (FIG. 6). Compound I′-6 was comparable to CC413 in regard to amount of cytokines/chemokines secreted by human PBMCs after transfection of EPO m1Ψ-mRNA (FIG. 6). This low immunogenicity can be directly attributed to the nucleoside modifications incorporated into the mRNA. To illustrate this, cells treated with lipid-complexed U-containing mRNA capped with compound I′-1, CC114 and CC413 induced much higher proinflammatory cytokine/chemokine responses even at their lowest dose (0.5 μg/ml) when compared to m1Ψ-modified mRNA (FIG. 6). In regard to cytokines/chemokines for unmodified mRNA, compound I′-1 is comparable to CC114 and leads to significantly higher cytokines/chemokines in comparison to CC413 (FIG. 6).

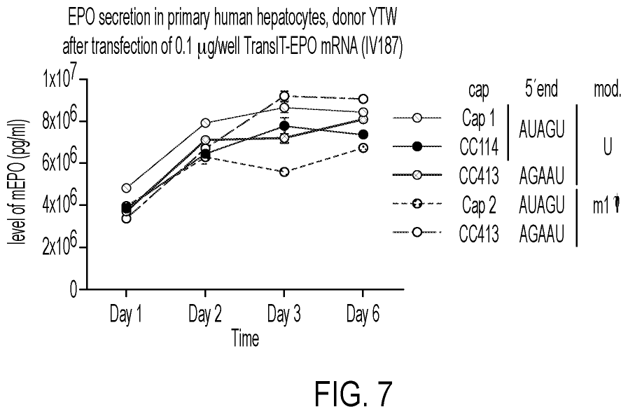

To measure the translational efficiency of EPO mRNA capped with compound I′-1, compound I′-6, CC114, and CC413 in vitro, human primary hepatocytes were seeded into 96-well plates at a density of 2.5×10⁴ cells per well and were transfected with mRNA samples (0.1 μg) complexed with TransIT reagent (Mirus Bio, Madison, WI, USA) in a final volume of 200 μL in InVitroGRO CP Medium (Sigma-Aldrich) supplemented with Thorpedo Antibiotic Mix (Sigma-Aldrich). To quantify EPO levels, the supernatant was collected at 1, 2, 3, 4, 5 and 6 day after transfection and EPO levels were analyzed by mouse Erythropoietin DuoSet ELISA kit (R&D Systems, Minneapolis, MN, USA) according to the manufacturer's instructions. Compound I′-1 resulted in higher translation compared to CC413 at each time points after transfection in a context of unmodified RNA (FIG. 7). However, compound I′-6 in m1Ψ-mRNA context leads to lower translation at later time points but in close to comparable translation to standard CC413 cap analog in human primary hepatocytes.

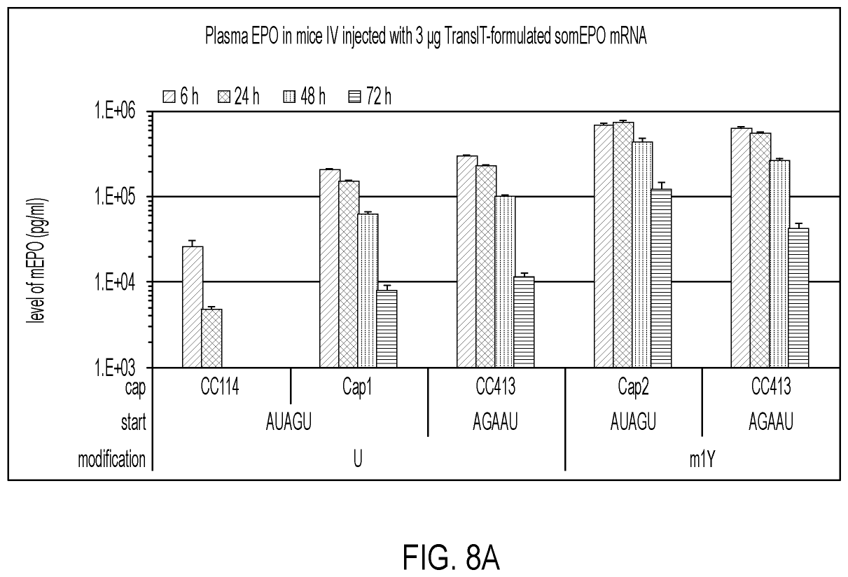

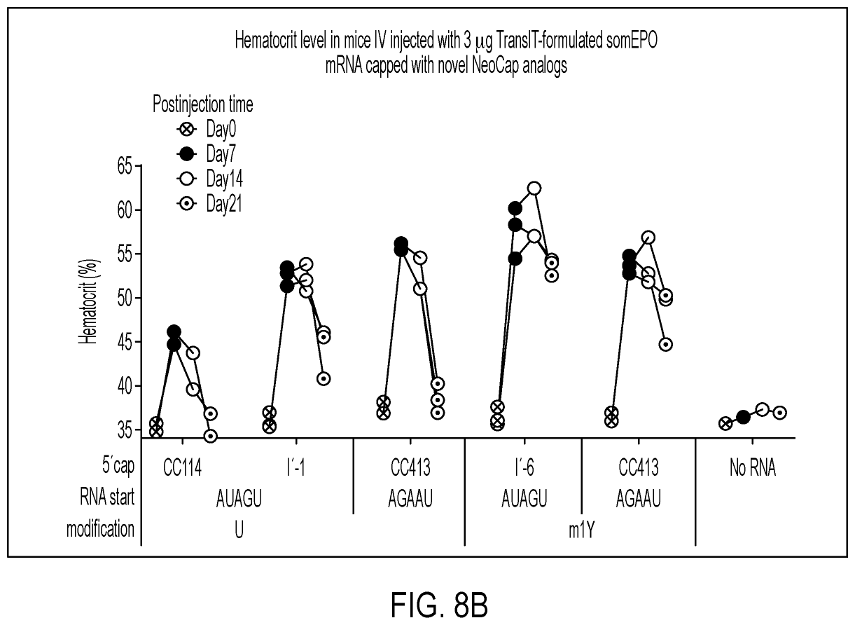

To confirm in vitro data, female BALB/c mice from Jackson Laboratory (Bar Harbor, ME, USA) at the age of eight to ten weeks were used for in vivo experiment in accordance with federal policies on animal research (Ethics approval number: G18-12-027). Mice (n=3/group) were injected intravenously (i.v.) with 3 μg TransIT-complexed (Mirus Bio) EPO mRNAs in a final volume of 200 μL Dulbecco's modified Eagle medium (DMEM). Mice used as controls were injected with TransIT-reagent diluted in DMEM but without RNA. Mice were injected with EPO mRNA complexed with TransIT-reagent and EPO levels in plasma were measured using ELISA (FIG. 8A). Hematocrit was measured from 18 μl of blood that was collected at the indicated times using centrifugation in Drummond microcaps glass capillary tubes (20 μl volume, Merck, Germany) as described (20) (FIG. 8B). After determination of the hematocrit, capillary tubes were snapped open, and the plasma was collected to measure EPO levels and analyzed for mouse Erythropoietin DuoSet ELISA kit (R&D Systems) according to the manufacturer's instructions. These results demonstrated again that mRNA capped with ARCA cap analog translated much better compared to those capped with non-ARCA version regardless of the cap initiators (FIG. 1, FIG. 8A, 8B). EPO RNA containing m1Ψ translated two-fold more than U-containing mRNA at each time points (FIG. 8A). Both compound I′-1 and compound I′-6 are suitable for translation of the encoded protein and each cap analog can be used for synthesizing of non-replicating functional mRNAs (FIG. 8A, 8B). Interestingly, compound I′-6 has a beneficial effect on translational efficiency of mRNA, meaning it showed 1.5-3 fold more translation compared to those capped with CC413 at 48 and 72 h after injection, respectively (FIG. 8A). This is confirmed by the fact that the 24-hour EPO values are higher than the 6-hour values, which is surprising after IV injection, and it never experienced before (FIG. 8A). The positive effect of compound I′-6 on the translational capacity of the mRNA is also expressed in the biological activities of the mRNA because hematocrit values remained very high level in mice injected with compound I′-6-capped EPO mRNA even at day 21 after injection (FIG. 8B). Hematocrit values started to decrease at D14 after injection of U-containing mRNA capped with CC114 and CC413, but it was not observed in regards to compound I′-1-capped mRNA (FIG. 8B). Hematocrit value in mice injected with compound I′-6-capped EPO mRNA at 21 days after administration is at the same level as the values of D7 and D14 in mice injected with CC413-capped mRNA (FIG. 8B).

In conclusion, the presented data demonstrated that m1Ψ-containing cap analog can be adapted to synthesis of non-replicating mRNA. These studies evaluated a unique, nucleoside modification-containing anti-reverse trinucleotide cap1 analog, compound I′-6, that was used to generate a functional mRNA with 5′ cap1 structure resulting in a long-term maintenance of the encoded protein. These data showed that an appropriate combination of initial sequence and compound I′-6 leads to a superior mRNA that significantly surpasses the translational capacity and biological activity of IVT mRNA capped with the commonly used trinucleotide cap analogs that has already been used successfully, e.g., in an mRNA-based vaccine against SARS-CoV-2.

### Example 2. Synthesis of 5′ Caps

### General Trinucleotide Cap1 Structure:

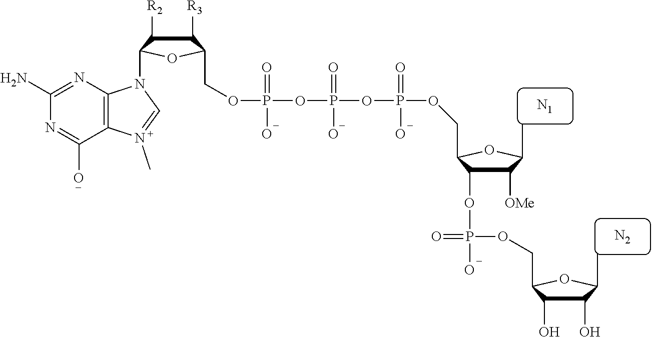

wherein:
R₂/R₃: OH/OMe
N₁ and N₂: A, U, G, C, Ψ, m⁶A, modified U, modified Ψ, any natural/unnatural nucleoside, any modified nucleoside
   General Trinucleotide Cap1 Structure Containing 2′-OMe-T Analogs at N₁ and Other Nucleosides at N₂:

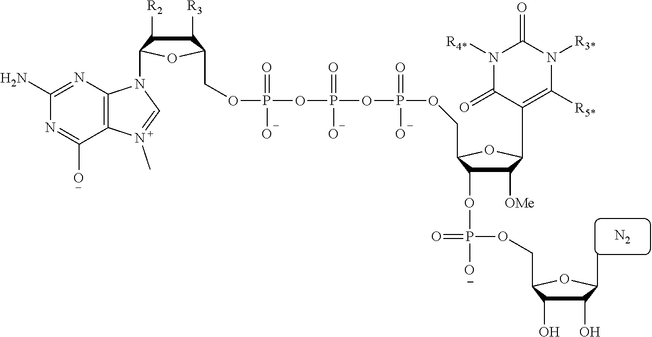

wherein:
R₂/R₃: OH/OMe
R_{3*}, R_{4*}, R_{5*}: H, Me, any alkyl, aryl, benzyl, naphthyl, vinyl, allyl, propargyl, carbocycles, heterocycles
N₂: A, G, m⁶A
   General Trinucleotide Cap1 Structure Containing 2′-OMe-A at N₁ and U/T Analogs at N₂:

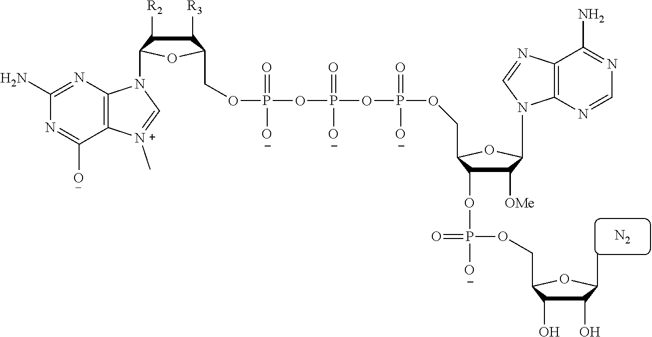

wherein:
R₂/R₃: OH/OMe
N₂: U, Ψ, modified U, modified Ψ

### General Synthetic Route of Different Cap Analogs

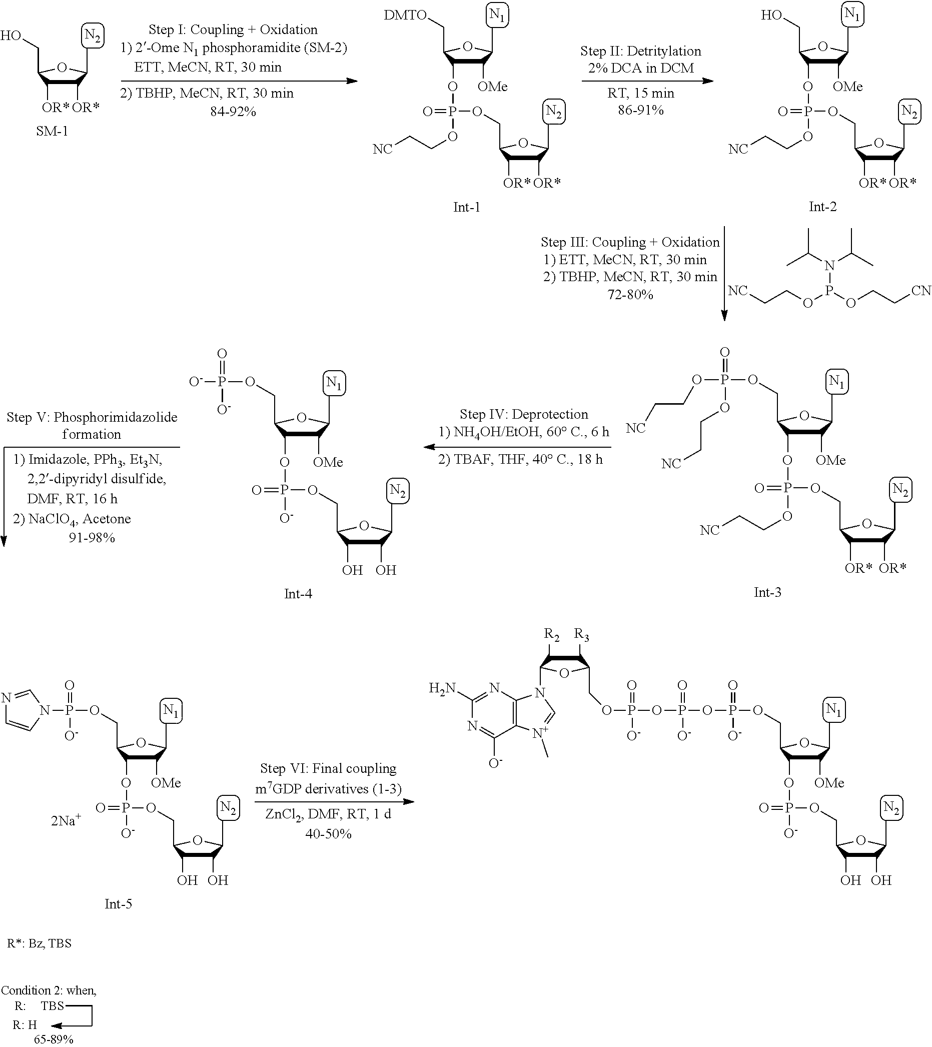

General Synthetic Route of Different Cap Analogs Containing 2′-OMe-A at N₁ and Various Ψ Derivatives at N₂

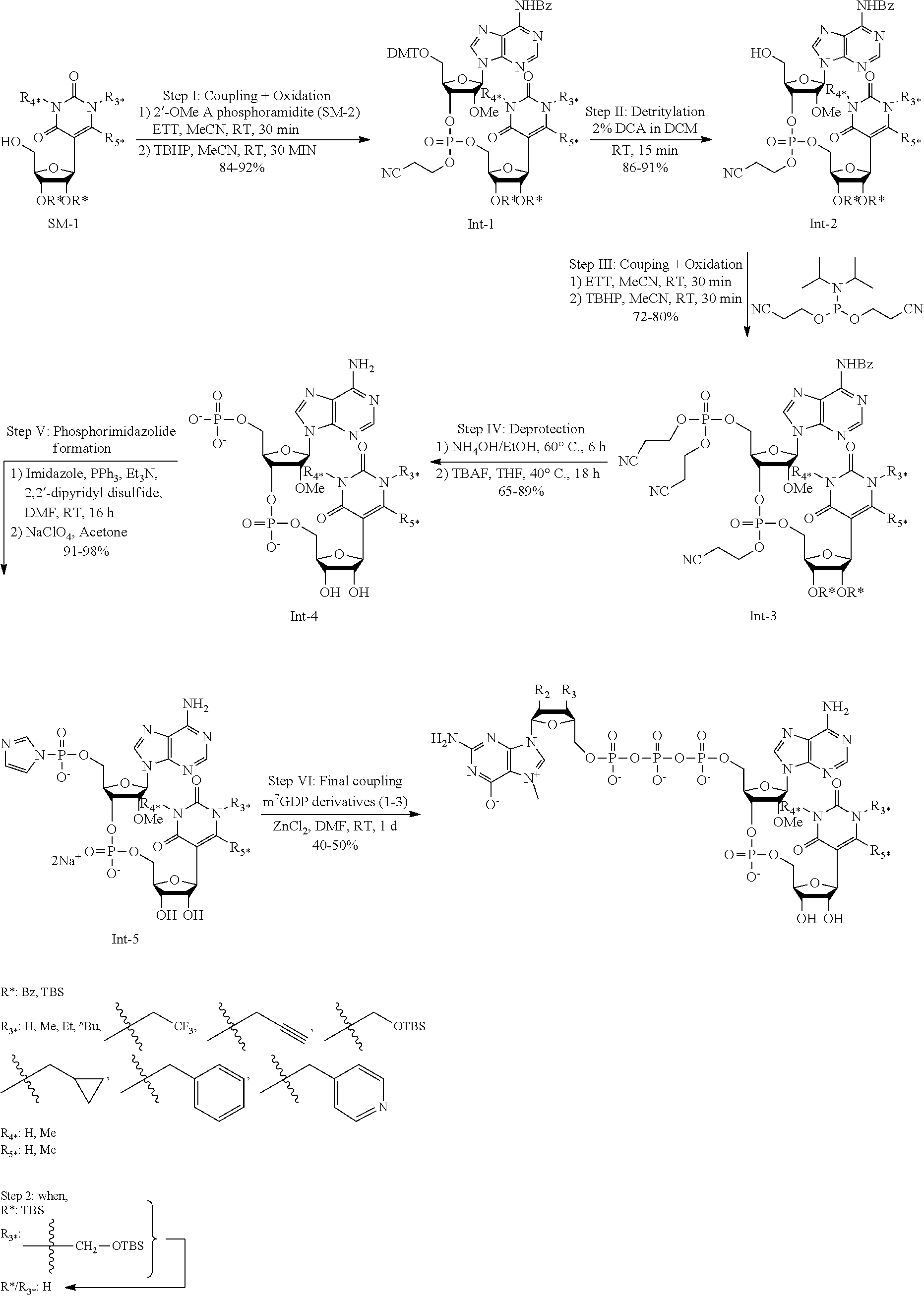

General Synthetic Route of Different m⁷GDP Derivatives (1-3)

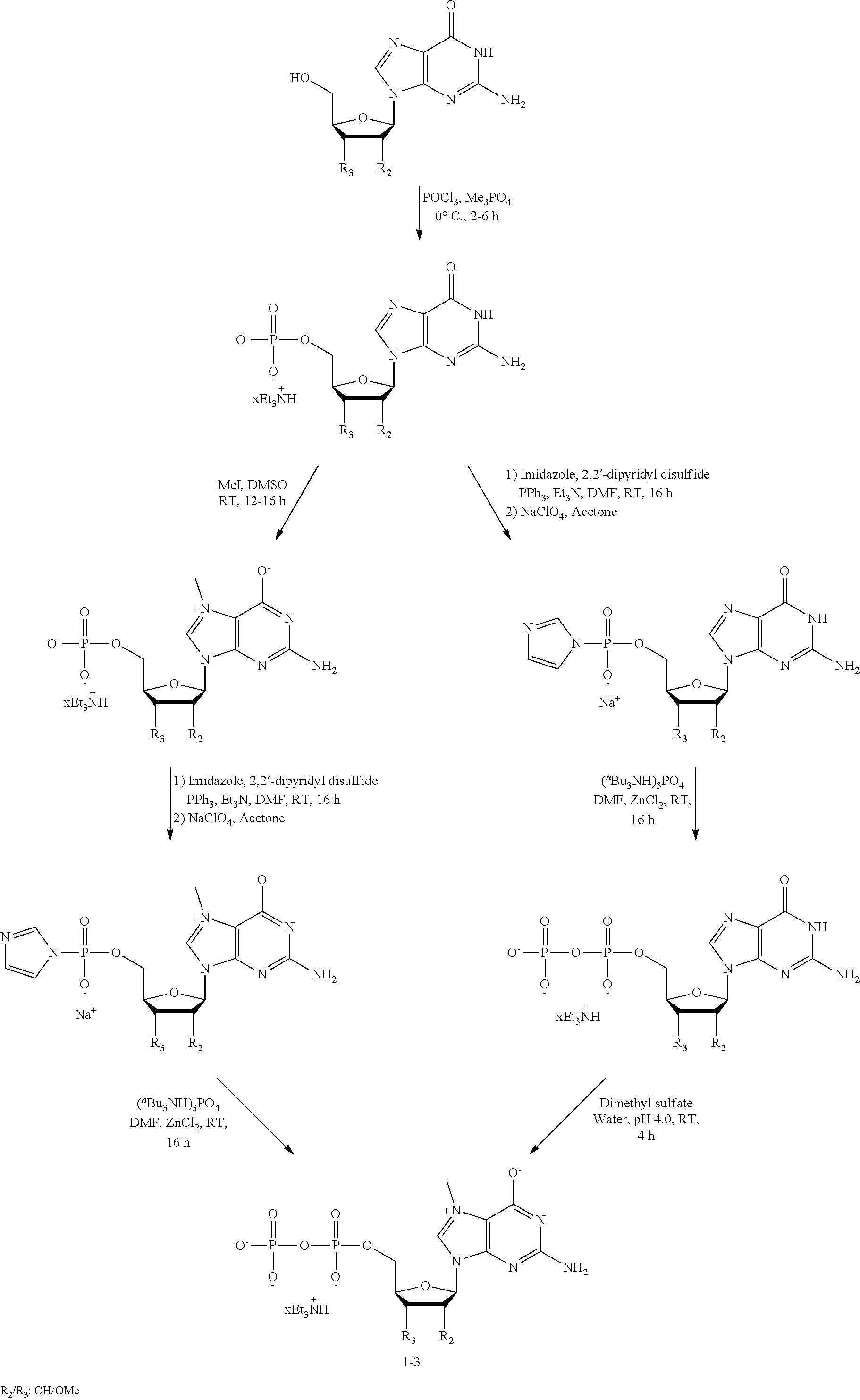

Synthesis of 7-Methyl-Guanosine 5′-Diphosphate Derivatives (m⁷GDP Derivatives, 1-3)

m⁷GDP derivative synthesis was performed according to modified published procedures (Patent US 2003/0194759 A1; Patent WO 2008/016473 A2; Patent WO2017053297A1; Bioorg. Med. Chem. Lett. 2007, 17, 5295.).

7-methylguanosine 5′-diphosphate (m⁷GDP, 1):

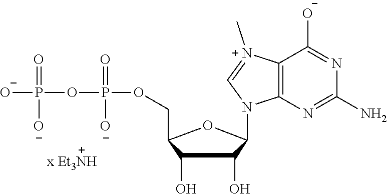

¹H NMR (300 MHz, D₂O): δ 6.04 (d, J=3.4 Hz, 1H), 4.66 (dd, J=4.8, 3.4 Hz, 1H), 4.50 (t, J=5.2 Hz, 1H), 4.42-4.28 (m, 2H), 4.20 (ddd, J=11.9, 5.3, 2.1 Hz, 1H), 4.10 (s, 3H), 3.19 (q, J=7.3 Hz, 22H), 1.26 (t, J=7.3 Hz, 33H).

³¹P NMR (121 MHz, D₂O): δ −10.40 (d, J=20.9 Hz), −11.38 (d, J=20.7 Hz).

MS (ESI⁻): Exact mass calculated for C₁₁H₁₆N₅O₁₁P₂ [M−H]⁻, 456.03. Found 456.01.

### 7-methyl-2′-O-methylguanosine 5′-diphosphate (m⁷GDP(2′-OMe), 2)

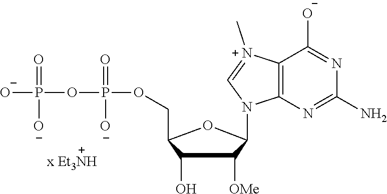

¹H NMR (300 MHz, D₂O): δ 6.21 (d, J=3.0 Hz, 1H), 4.69 (dd, J=6.1, 4.9 Hz, 1H), 4.43-4.34 (m, 3H), 4.31-4.22 (m, 1H), 4.17 (s, 3H), 3.65 (s, 3H), 3.25 (q, J=7.4 Hz, 9H, Et₃NH⁺), 1.33 (t, J=7.3 Hz, 14H, Et₃NH⁺).

³¹P NMR (121 MHz, D₂O): δ −8.98 (d, J=21.7 Hz), −11.03-−11.36 (m).

MS (ESI⁻): Exact mass calculated for C₁₂H₁₈N₅O₁₁P₂ [M−H]⁻, 470.05. Found 470.02.

### 7-methyl-3′-O-methylguanosine 5′-diphosphate (m⁷GDP(3′-OMe), 3)

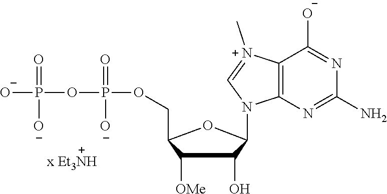

¹H NMR (300 MHz, D₂O): δ 6.11 (d, J=4.0 Hz, 1H), 4.90 (t, J=4.5 Hz, 1H), 4.54 (dq, J=5.1, 2.6 Hz, 1H), 4.39 (ddd, J=11.9, 4.5, 2.6 Hz, 1H), 4.30-4.19 (m, 2H), 4.18 (s, 3H), 3.55 (s, 3H), 3.25 (q, J=7.4 Hz, 8H, Et₃NH⁺), 1.33 (t, J=7.4 Hz, 12H, Et₃NH⁺).

³¹P NMR (121 MHz, D20: 6-8.95 (d, J=21.5 Hz), −11.20 (dq, J=21.2, 4.3 Hz).

MS (ESI⁻): Exact mass calculated for C₁₂H₁₈N₅O₁₁P₂ [M−H]⁻, 470.05. Found 470.09.

### General Scheme for the Synthesis of Different Cap Analogs

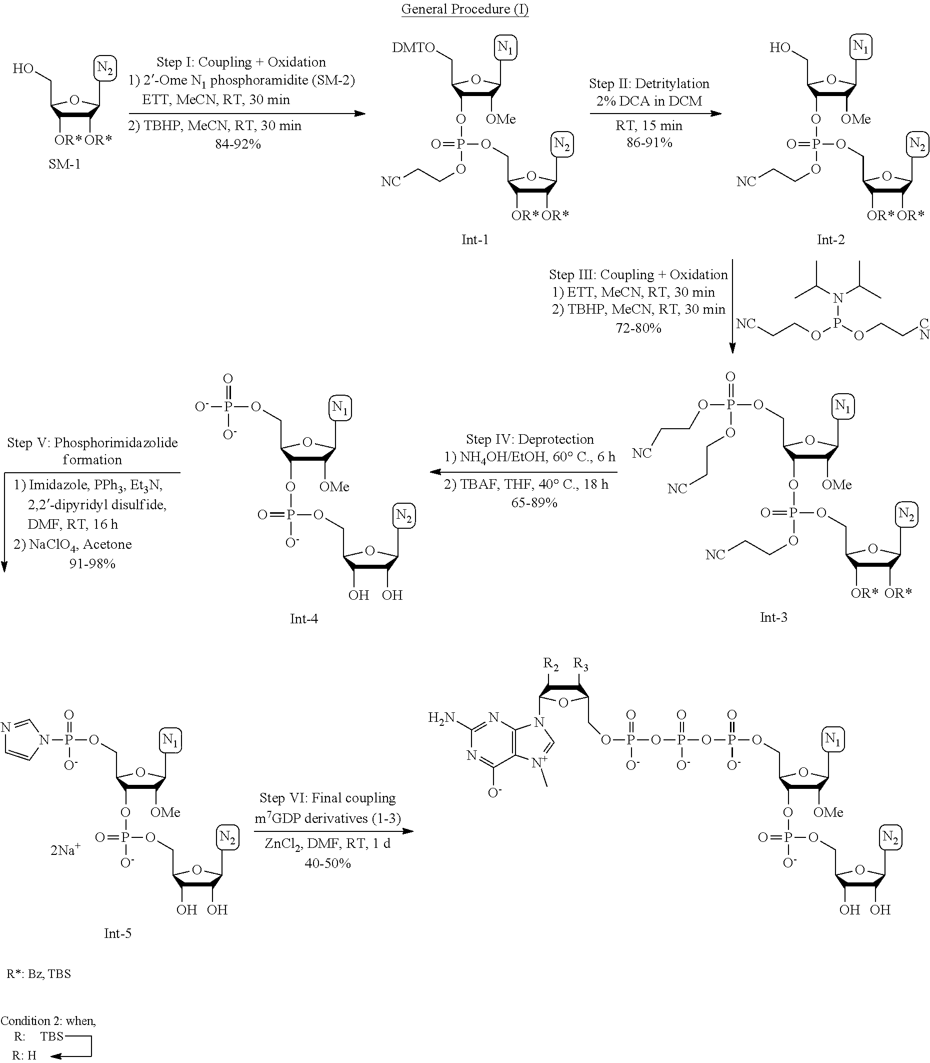

Cap synthesis involving multiple steps was performed according to modified published procedures. (Patent WO2017053297A1; WO2021162567A1, Nucleic Acids Res. 2020, 48, 1607.).

### Step I: Coupling+Oxidation

2′,3′-N-protected nucleoside derivative (SM-1, 5 mmol), 5-Ethylthio-1H-tetrazole (activator, 25 mmol) and 2′-OMe phosphoramidite derivative (SM-2, 5 mmol) were taken in a 100 mL Schlenk flask and 50 mL dry ACN was added. The solution was stirred for 30 min at RT. Later tert-Butyl hydroperoxide solution (to oxidize P(III) to P(V) state, 25 mmol) was added dropwise, solution turned yellow, stirred at RT for further 30 min. Solvent was evaporated and the residue was redissolved in DCM (300 mL), extracted with water (3×100 mL) and brine (100 mL). The organic phase was dried over anhydrous Na₂SO₄, filtered and the solvent was evaporated in vacuo, yellow sticky solid was obtained. Crude residue was purified by flash chromatography (0-5% MeOH/DCM) and coupling product (Int-1) was achieved as yellowish-white solid foam (yield 84-92%).

### Step II: Detritylation

Coupling product (Int-1, 4 mmol) was taken in a 250 mL Schlenk flask and 150 mL dry DCM was added, formed colorless solution. Then dichloroacetic acid (40 mmol) was added dropwise at RT over 5 min, the solution turned dark red. The reaction was stirred for 15 min at RT. TLC was performed to check complete deprotection of the DMT group. Later MeOH was added until faint red color persisted. Solvent was evaporated and the residue was dissolved in 300 mL DCM, again MeOH was added until only a faint red color persisted. The solution was extracted with saturated aqueous NaHCO₃ solution (3×100 mL), followed by extraction with brine (100 mL). The organic phase was dried over anhydrous Na₂SO₄, filtered and the solvent was evaporated in vacuo, yellow foam was obtained. Crude residue was purified by flash chromatography (0-10% MeOH/DCM) and compound (Int-2) was yielded as yellowish-white solid foam (yield 86-91%).

### Step III: Coupling+Oxidation

Compound (Int-2, 3 mmol), 5-Ethylthio-1H-tetrazole (activator, 15 mmol) were taken in a 100 mL Schlenk flask and 30 mL dry ACN was added. After that, 3-({[bis(propan-2-yl)amino](2-cyanoethoxy)phosphanyl}oxy)propanenitrile (4.5 mmol) was added dropwise. The solution was stirred for 30 min at RT. Later tert-Butyl hydroperoxide solution (15 mmol) was added dropwise, solution turned yellow, stirred at RT for further 30 min. Solvent was evaporated and the residue was redissolved in DCM (200 mL), extracted with water (3×50 mL) and brine (50 mL). The organic phase was dried over anhydrous Na₂SO₄, filtered and the solvent was evaporated in vacuo, yellow sticky residue was obtained. Crude residue was purified by flash chromatography (0-10% MeOH/DCM) and compound (Int-3) was obtained as yellowish-white solid foam (yield 72-80%).

### Step IV: Deprotection

Compound (Int-3, 2 mmol) was taken in an autoclave and 20 mL absolute EtOH was added, followed by the addition of 60 mL aq. NH₃ solution (32%). The reaction was stirred at 60° C. for 6 h, showed complete deprotection of the substrate in LCMS studies. The solution was concentrated in vacuo, redissolved in water/absolute ethanol (1:1, 50 mL) and evaporated under reduced pressure to obtain yellow residue.
when R*=Bz, the residue was purified directly by anion exchange chromatography, followed by reversed phase chromatography. Compound (pN₁(2′-OMe)pN₂, Int-4) was obtained as white solid foam as triethylammonium salt (yield 77-89%).
when R*=TBS, the residue was further treated with 1M TBAF in THF solution. Reaction was stirred at 40° C. for 18 h. Deprotection of the TBS group was monitored by LCMS studies. After complete deprotection, 100 mM TEAB (pH=7.5) was added and purified directly by anion exchange chromatography and reversed phase chromatography. Compound (pN₁(2′-OMe)pN₂, Int-4) was obtained as white solid foam as triethylammonium salt (yield 65-72%).

Anion exchange chromatography: XK 50/30 column filled with DEAE Sephadex A-25 (˜75 g resin, column volume ˜550 mL, Cytiva. Detection wavelengths: 220, 260 nm; Solvent systems: buffer A: 100 mM TEAB (pH=7.5); buffer B: 1.0 M TEAB (pH=7.5); Flow rate: 30 ml/min; Gradient: 0% B for 30 min, 0-80% B in 160 min, 80-100% B in 5 min, 100% B for 25 min). Product obtained after Anion exchange chromatography was further purified by reversed phase chromatography.

Reversed phase chromatography: Column: FP Select C18 330 g; Detection wavelengths: 220 nm, 260 nm; Solvent systems: solvent A: 100 mM TEAB (pH=7.5), solvent B: acetonitrile; Flow Rate: 60 mL/min; Gradient: 5% B for 6 min, 5-20% B in 24 min, 20-50% B in 5 min, 50% B for 5 min). Product containing fractions were collected and the solvents were evaporated and lyophilized.

### Step V: Phosphorimidazolide Formation

Compound (pN₁(2′-OMe)pN₂, Int-4, 1 mmol) was taken in a 100 mL RB flask and co-evaporated with dry DMF (2×15 mL). After that, 35 mL dry DMF was added. In another dry 50 mL Schlenk flask, triphenylphosphine (2 mmol), 2,2′-dipyridyldisulfide (2 mmol), and imidazole (2.5 mmol) were dissolved in 20 mL dry DMF and Et₃N (10 mmol). The solution was then dropwise added to the solution of dinucleotide derivative, turned to yellow solution. The reaction was stirred for 18 h at RT. The solution was then poured onto 1 L 2% anhydrous NaClO₄ in dry acetone (w/v) solution, white precipitate formation was observed. Filtration was performed and the residue was washed with 1 L dry acetone. The product was dried over anhydrous P₂O₅ under reduced pressure. Dinucleotide phosphorimidazolide (Im-pN₁(2′-OMe)pN₂, Int-5) was obtained as disodium salt (91-98%).

### Step VI: Final Coupling

7-methyl-guanosine 5′-diphosphate derivative (m⁷GDP derivative, 0.75 mmol) and dinucleotide phosphorimidazolide (Im-pN₁(2′-OMe)pN₂, Int-5, 0.9 mmol) were taken in a 50 mL RB flask and 15 mL dry DMF was added under argon atmosphere, formed suspension. Then anhydrous ZnCl₂ (7.5 mmol) was added under argon flow. The reaction mixture was stirred for 1 day at RT under argon atmosphere. Later the reaction mixture was added to aqueous Na₂EDTA solution (1.5 g in 75 mL water), and pH was adjusted to 7.0 using saturated sodium bicarbonate solution. The solution containing crude product was purified was purified directly by anion exchange chromatography, followed by reversed phase chromatography.

Anion exchange chromatography: XK 50/30 column filled with DEAE Sephadex A-25 (˜75 g resin, column volume ˜550 mL, Cytiva. Detection wavelengths: 220, 260 nm; Solvent systems: buffer A: 100 mM TEAB (pH=7.5), buffer B: 1.0 M TEAB (pH=7.5); Flow rate: 30 ml/min; Gradient: 0% B for 30 min, 0-80% B in 200 min, 80-100% B in 5 min, 100% B for 25 min). Product obtained after anion exchange chromatography was further purified by reversed phase chromatography.

Reversed phase chromatography: Column: Atlantis T3 OBD Prep Column, 100A, 5 μm, 50×100 mm, Waters; Detection wavelengths: 220 nm, 260 nm; Solvent systems: solvent A: 50 mM NH₄OAc in water (pH 7.0), solvent B: 100 mM NH₄OAc in water (pH 7.0)/ACN (1:1); Flow Rate: 90 mL/min; Gradient: 2-15% B in 45 min, 15-50% B in 5 min, 50% B for 5 min, 50-2% B in 2 min, 2% B for 10 min). Product containing fractions were collected and the solvents were evaporated and lyophilized.

The product after reversed phase chromatography was obtained as ammonium salt which was converted to sodium salt using 2% NaClO₄ in acetone (w/v) solution. Sodium salt of the desired cap analogs was obtained as white solid foam (yield 40-50%).

m⁷G(^{3′-OMe})pppA(2′-OMe)pU (I′-1):

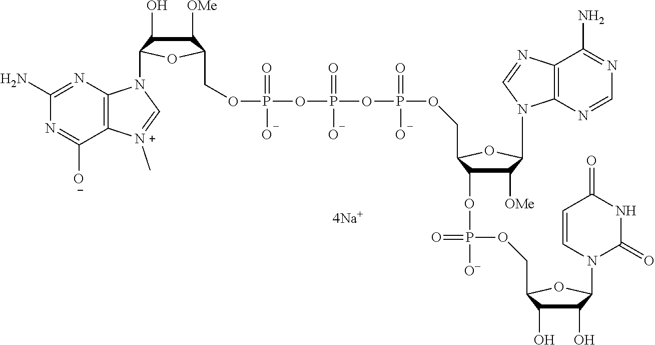

Compound I′-1 was synthesized according to the general procedure (I).

¹H NMR (300 MHz, D₂O): δ 9.15 (s, 1H), 8.60 (s, 1H), 8.34 (s, 1H), 7.89 (d, J=8.2 Hz, 1H), 6.16 (d, J=5.6 Hz, 1H), 5.93 (d, J=4.5 Hz, 1H), 5.86 (d, J=4.2 Hz, 1H), 5.82 (d, J=8.1 Hz, 1H), 4.98 (dt, J=8.2, 4.3 Hz, 1H), 4.73 (t, J=4.5 Hz, 1H), 4.63-4.56 (m, 1H), 4.50 (t, J=4.9 Hz, 1H), 4.46-4.11 (m, 11H), 4.06 (s, 3H), 3.51 (s, 3H), 3.48 (s, 3H).

³¹P NMR (121 MHz, D₂O): δ −1.01 (d, J=7.4 Hz, 1P), −11.23-−11.78 (m, 2P), −22.76 (t, J=18.5 Hz, 1P).

MS (ESI⁻): Exact mass calculated for C₃₂H₄₃N₁₂O₂₅P₄ [M−H]⁻, 1119.14. Found 1119.07.

m⁷G(2′-OMe)pppA(2′-OMe)pU (I′-2):

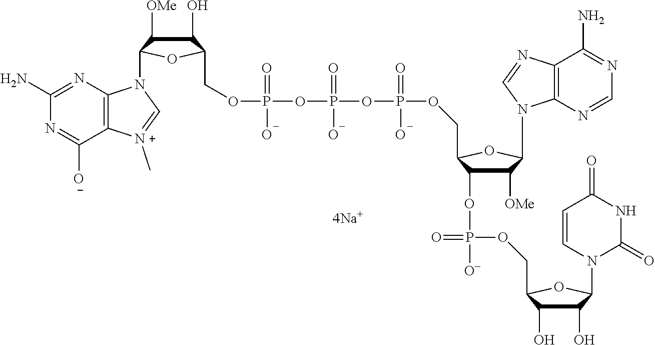

Compound I′-2 was synthesized according to the general procedure (I).

¹H NMR (300 MHz, D₂O): δ 9.11 (s, 1H), 8.56 (s, 1H), 8.32 (s, 1H), 7.93 (d, J=8.1 Hz, 1H), 6.16 (d, J=5.8 Hz, 1H), 5.99-5.95 (m, 2H), 5.87 (d, J=8.1 Hz, 1H), 5.00 (dt, J=8.0, 4.1 Hz, 1H), 4.63-4.51 (m, 3H), 4.48-4.16 (m, 11H), 4.10 (s, 3H), 3.60 (s, 3H), 3.54 (s, 3H).

³¹P NMR (121 MHz, D₂O): δ −1.00 (d, J=7.2 Hz, 1P), −11.22-−11.82 (m, 2P), −22.83 (t, J=18.4 Hz, 1P).

MS (ESI⁻): Exact mass calculated for C₃₂H₄₃N₁₂O₂₅P₄ [M−H]⁻, 1119.14. Found 1119.07.

m⁷G(^{3′-OMe})pppA(2′-OMe)pm³U (I′-11):

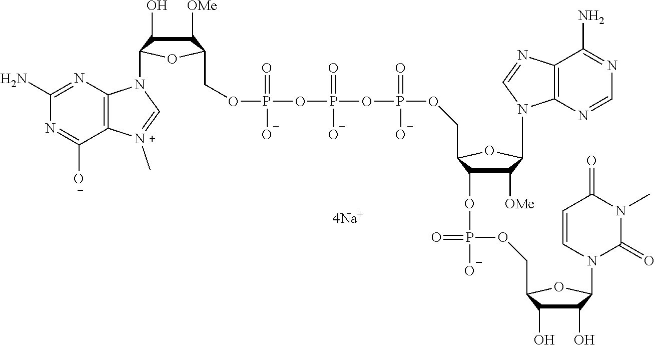

Compound I′-11 was synthesized according to the general procedure (I).

¹H NMR (300 MHz, D₂O): δ 8.46 (s, 1H), 8.19 (s, 1H), 7.88 (d, J=8.1 Hz, 1H), 6.10 (d, J=5.7 Hz, 1H), 5.98 (d, J=4.0 Hz, 1H), 5.89-5.84 (m, 2H), 4.97 (dt, J=8.2, 4.2 Hz, 1H), 4.71 (t, J=4.7 Hz, 1H), 4.62-4.57 (m, 1H), 4.50 (t, J=5.4 Hz, 1H), 4.46-4.17 (m, 10H), 4.13 (t, J=4.7 Hz, 1H), 4.08 (s, 3H), 3.54 (s, 3H), 3.51 (s, 3H), 3.30 (s, 3H).

³¹P NMR (121 MHz, D₂O): δ −1.05 (s, 1P), −11.55 (d, J=18.6 Hz, 2P), −22.92 (t, J=18.6 Hz, 1P).

MS (ESI⁻): Exact mass calculated for C₃₃H₄₅N₁₂O₂₅P₄ [M−H]⁻, 1133.16. Found 1133.19.

m⁷G(^{3′-OMe})pppA(2′-OMe)pm⁵U (I′-9):

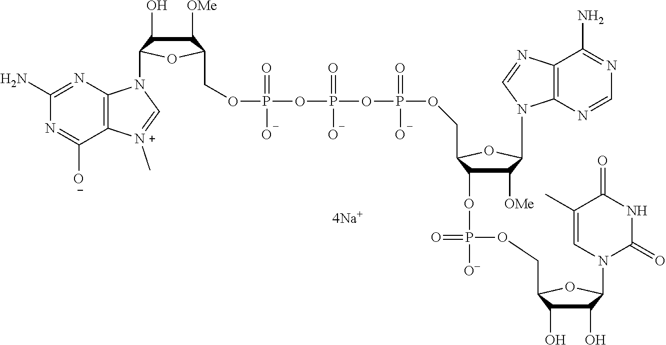

Compound I′-9 was synthesized according to the general procedure (I).

¹H NMR (300 MHz, D₂O): δ 9.20 (s, 1H), 8.65 (s, 1H), 8.38 (s, 1H), 7.75 (s, 1H), 6.21 (d, J=5.1 Hz, 1H), 5.97 (d, J=4.9 Hz, 1H), 5.92 (d, J=4.2 Hz, 1H), 5.00 (dt, J=8.5, 4.4 Hz, 1H), 4.65-4.60 (m, 1H), 4.55 (t, J=5.0 Hz, 1H), 4.49-4.15 (m, 11H), 4.11 (s, 3H), 3.57 (s, 3H), 3.53 (s, 3H), 1.87 (s, 3H).

³¹P NMR (121 MHz, D₂O): δ −1.25 (d, J=7.5 Hz, 1P), −11.54 (t, J=20.6 Hz, 2P), −22.76 (t, J=18.4 Hz, 1P).

MS (ESI⁻): Exact mass calculated for C₃₃H₄₅N₂O₂₅P₄ [M−H]⁻, 1133.16. Found 1133.12.

m⁷G(^{3′-OMe})pppA(2′-OMe)pmo⁵U (I′-12):

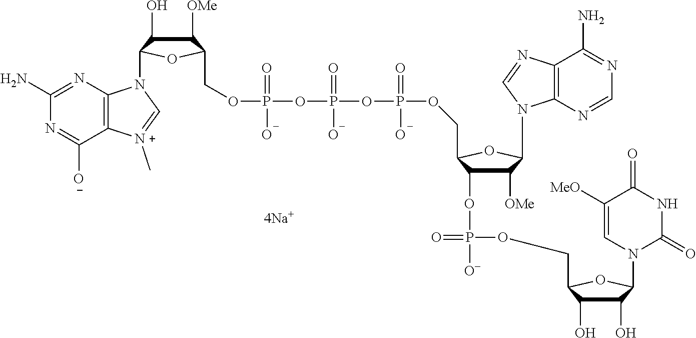

Compound I′-12 was synthesized according to the general procedure (I).

¹H NMR (300 MHz, D₂O): δ 9.11 (s, 1H), 8.52 (s, 1H), 8.28 (s, 1H), 7.34 (s, 1H), 6.15 (d, J=5.6 Hz, 1H), 6.07-6.02 (m, 1H), 5.90 (d, J=4.3 Hz, 1H), 5.01 (dt, J=8.0, 3.9 Hz, 1H), 4.74 (t, J=4.6 Hz, 1H), 4.63-4.59 (m, 1H), 4.56 (t, J=5.4 Hz, 1H), 4.48-4.19 (m, 10H), 4.15 (t, J=4.6 Hz, 1H), 4.09 (s, 3H), 3.80 (s, 3H), 3.52 (s, 3H), 3.51 (s, 3H).

³¹P NMR (121 MHz, D₂O): δ −1.24 (s, 1P), −11.34-−11.81 (m, 2P), −22.90 (t, J=18.5 Hz, 1P).

MS (ESI⁻): Exact mass calculated for C₃₃H₄₅N₁₂O₂₆P₄ [M−H]⁻, 1149.15. Found 1149.17.

m⁷GpppA(2′-OMe)pm¹Ψ (I′-13):

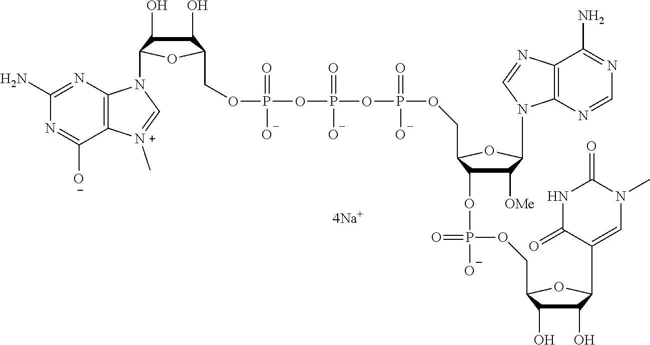

Compound I′-13 was synthesized according to the general procedure (I).

¹H NMR (300 MHz, D₂O): δ 9.18 (s, 1H), 8.65 (s, 1H), 8.36 (s, 1H), 7.75 (d, J=1.0 Hz, 1H), 6.20 (d, J=5.5 Hz, 1H), 5.94 (d, J=3.7 Hz, 1H), 5.00 (dt, J=8.2, 4.2 Hz, 1H), 4.66-4.57 (m, 3H), 4.52 (t, J=5.0 Hz, 1H), 4.47-4.37 (m, 3H), 4.36-4.11 (m, 7H), 4.09 (s, 3H), 3.55 (s, 3H), 3.38 (s, 3H).

³¹P NMR (121 MHz, D₂O): δ −0.72 (d, J=6.8 Hz, 1P), −11.44 (t, J=20.3 Hz, 2P), −22.73 (t, J=18.5 Hz, 1P).

MS (ESI⁻): Exact mass calculated for C₃₂H₄₃N₁₂O₂₅P₄ [M−H]⁻, 1119.14. Found 1119.27.

m⁷G(^{3′-OMe})pppA(2′-OMe)pm¹Ψ (I′-6):

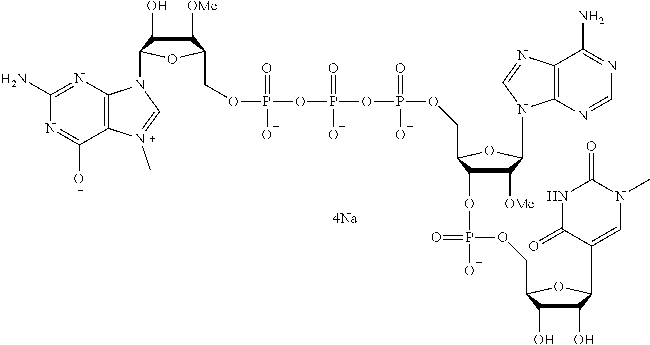

Compound I′-6 was synthesized according to the general procedure (I).

¹H NMR (300 MHz, D₂O): δ 9.08 (s, 1H), 8.57 (s, 1H), 8.27 (s, 1H), 7.63 (d, J=1.0 Hz, 1H), 6.10 (d, J=5.3 Hz, 1H), 5.77 (d, J=4.1 Hz, 1H), 4.87 (dq, J=9.3, 4.6 Hz, 1H), 4.66-4.61 (m, 2H), 4.57-4.44 (m, 2H), 4.36-4.28 (m, 3H), 4.24-3.98 (m, 8H), 3.96 (s, 3H), 3.43 (s, 3H), 3.39 (s, 3H), 3.25 (s, 3H).

³¹P NMR (121 MHz, D₂O): δ −1.82 (d, J=7.4 Hz, 1P), −12.61 (t, J=17.9 Hz, 2P), −23.86 (t, J=19.4 Hz, 1P).

MS (ESI⁻): Exact mass calculated for C₃₃H₄₅N₁₂O₂₅P₄ [M−H]⁻, 1133.16. Found 1133.18.

m⁷G(2′-OMe)pppA(2′-OMe)pm¹Ψ (I′-5):

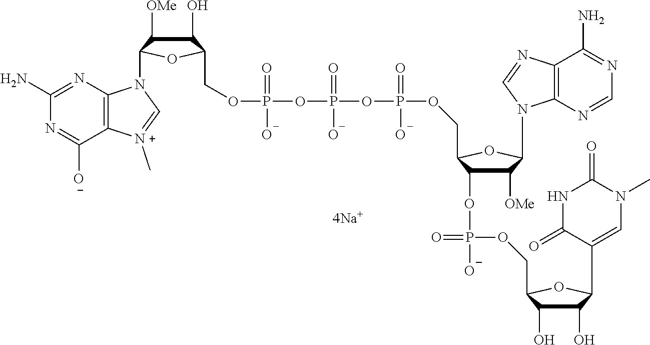

Compound I′-5 was synthesized according to the general procedure (I).

¹H NMR (300 MHz, D₂O): δ 9.15 (s, 1H), 8.63 (s, 1H), 8.35 (s, 1H), 7.75 (d, J=1.0 Hz, 1H), 6.18 (d, J=5.6 Hz, 1H), 5.98 (d, J=3.6 Hz, 1H), 5.00 (dt, J=8.2, 4.1 Hz, 1H), 4.68-4.56 (m, 3H), 4.48-4.38 (m, 2H), 4.37-4.12 (m, 9H), 4.10 (s, 3H), 3.60 (s, 3H), 3.55 (s, 3H), 3.38 (s, 3H).

³¹P NMR (121 MHz, D₂O): δ −0.74 (d, J=6.9 Hz, 1P), −11.50 (t, J=19.0 Hz, 2P), −22.78 (t, J=18.5 Hz, 1P).

MS (ESI⁻): Exact mass calculated for C₃₃H₄₅N₁₂O₂₅P₄ [M−H]⁻, 1133.16. Found 1133.18.

m⁷G(^{3′-OMe})pppA(2′-OMe)pΨ (I′-3):

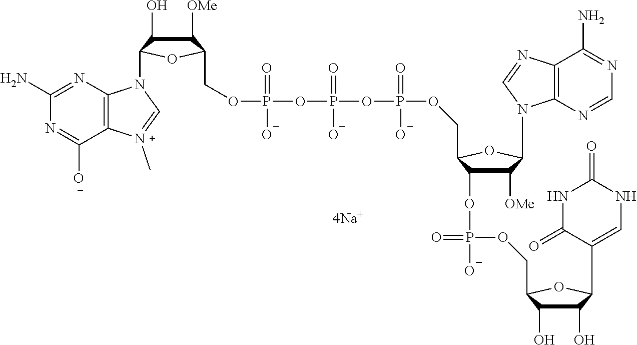

Compound I′-3 was synthesized according to the general procedure (I).

¹H NMR (300 MHz, D₂O): δ 9.19 (s, 1H), 8.62 (s, 1H), 8.35 (s, 1H), 7.60 (s, 1H), 6.21 (d, J=4.8 Hz, 1H), 5.92 (d, J=4.3 Hz, 1H), 4.96 (dt, J=8.7, 4.6 Hz, 1H), 4.77-4.73 (m, 2H), 4.67-4.58 (m, 2H), 4.47-4.39 (m, 3H), 4.34-4.14 (m, 8H), 4.11 (s, 3H), 3.59 (s, 3H), 3.52 (s, 3H).

³¹P NMR (121 MHz, D₂O): δ −0.70 (d, J=6.7 Hz, 1P), −11.33-−11.74 (m, 2P), −22.79 (t, J=18.5 Hz, 1P).

MS (ESI⁻): Exact mass calculated for C₃₂H₄₃N₁₂O₂₅P₄ [M−H]⁻, 1119.14. Found 1119.04.

m⁷G(^{3′-OMe})pppA(2′-OMe)pm³Ψ (I′-14):

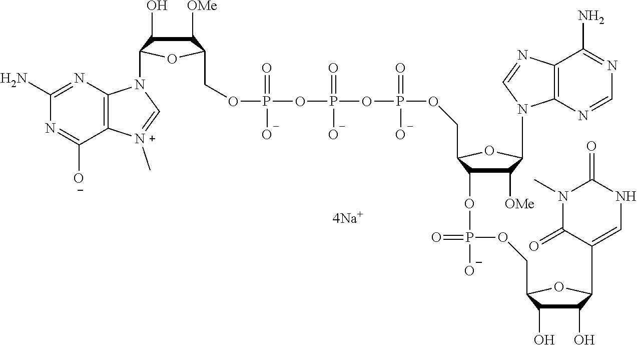

Compound I′-14 was synthesized according to the general procedure (I).

¹H NMR (300 MHz, D₂O): δ 9.24 (s, 1H), 8.70 (s, 1H), 8.41 (s, 1H), 7.60 (d, J=1.1 Hz, 1H), 6.24 (d, J=4.9 Hz, 1H), 5.93 (d, J=4.3 Hz, 1H), 4.98 (dt, J=8.6, 4.5 Hz, 1H), 4.66-4.61 (m, 2H), 4.50-4.39 (m, 3H), 4.38-4.13 (m, 8H), 4.12 (s, 3H), 3.60 (s, 3H), 3.53 (s, 3H), 3.27 (s, 3H).

³¹P NMR (121 MHz, D₂O): δ −0.70 (d, J=6.5 Hz, 1P), −11.27-−11.74 (m, 2P), −22.75 (t, J=18.4 Hz, 1P).

MS (ESI⁻): Exact mass calculated for C₃₃H₄₅N₁₂O₂₅P₄ [M−H]⁻, 1133.16. Found 1133.22.

m⁷G(^{3′-OMe})pppA(2′-OMe)ptfet¹Ψ (I′-15):

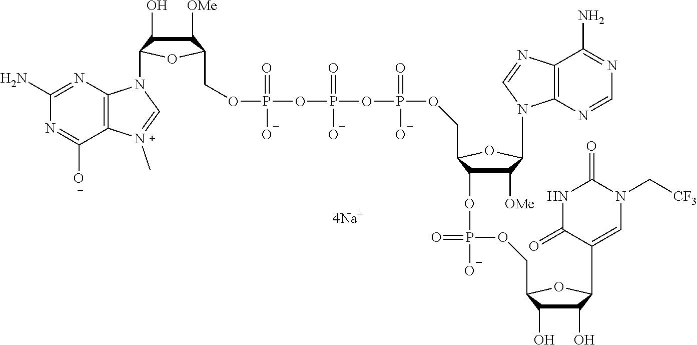

Compound I′-15 was synthesized according to the general procedure (I).

¹H NMR (300 MHz, D₂O): δ 9.10 (s, 1H), 8.54 (s, 1H), 8.29 (s, 1H), 7.86 (s, 1H), 6.15 (d, J=6.3 Hz, 1H), 5.89 (d, J=4.3 Hz, 1H), 5.01 (dt, J=8.0, 4.1 Hz, 1H), 4.83 (d, J=3.2 Hz, 1H), 4.74 (t, J=4.7 Hz, 1H), 4.69-4.52 (m, 4H), 4.47-4.12 (m, 11H), 4.10 (s, 3H), 3.52 (s, 3H), 3.51 (s, 3H).

³¹P NMR (121 MHz, D₂O): δ −0.81 (s, 1P), −11.54 (d, J=18.6 Hz, 2P), −22.89 (t, J=18.5 Hz, 1P).

MS (ESI⁻): Exact mass calculated for C₃₄H₄₄F₃N₁₂O₂₅P₄ [M−H]⁻, 1201.14. Found 1201.05.

m⁷G(^{3′-OMe})pppA(2′-OMe)p(ppg)¹Ψ (I′-16):

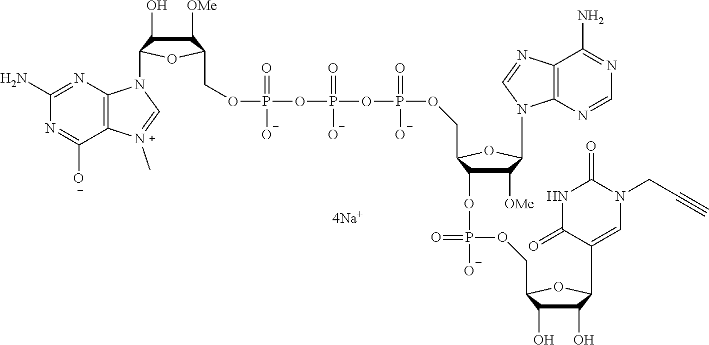

Compound I′-16 was synthesized according to the general procedure (I).

¹H NMR (300 MHz, D₂O): δ 8.48 (s, 1H), 8.21 (s, 1H), 7.84 (s, 1H), 6.12 (d, J=6.2 Hz, 1H), 5.86 (d, J=4.3 Hz, 1H), 5.00 (dt, J=7.9, 4.5 Hz, 1H), 4.70 (t, J=4.6 Hz, 1H), 4.66-4.61 (m, 1H), 4.60-4.54 (m, 3H), 4.45-4.10 (m, 12H), 4.09 (s, 3H), 3.51 (s, 6H), 2.85 (t, J=2.5 Hz, 1H).

³¹P NMR (121 MHz, D₂O): δ −0.73 (d, J=7.2 Hz, 1P), −11.26-−11.78 (m, 2P), −22.88 (t, J=18.6 Hz, 1P).

MS (ESI⁻): Exact mass calculated for C₃₅H₄₅N₁₂O₂₅P₄ [M−H]⁻, 1157.16. Found 1157.12.

m⁷G(^{3′-OMe})pppA(2′-OMe)pbn¹Ψ (I′-17):

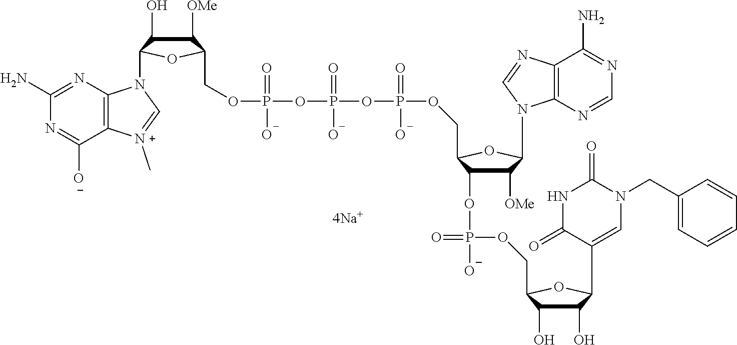

Compound I′-17 was synthesized according to the general procedure (I).

¹H NMR (300 MHz, D₂O): δ 9.13 (s, 1H), 8.53 (s, 1H), 8.24 (s, 1H), 7.90 (s, 1H), 7.35-7.18 (m, 5H), 6.12 (d, J=6.5 Hz, 1H), 5.90 (d, J=4.6 Hz, 1H), 5.08-5.00 (m, 1H), 4.96-4.80 (m, 3H), 4.76-4.67 (m, 2H), 4.56 (t, J=5.6 Hz, 1H), 4.48-4.12 (m, 11H), 4.09 (s, 3H), 3.52 (s, 3H), 3.45 (s, 3H).

³¹P NMR (121 MHz, D₂O): δ −0.86 (s, 1P), −11.34-−11.79 (m, 2P), −22.86 (t, J=18.6 Hz, 1P).

MS (ESI⁻): Exact mass calculated for C₃₉H₄₉N₁₂O₂₅P₄ [M−H]⁻, 1209.19. Found 1209.18.

m⁷G(^{3′-OMe})pppA(2′-OMe)pcpm¹Ψ (I′-18):

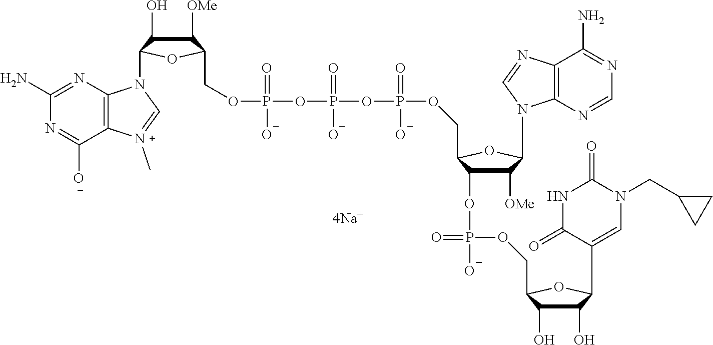

Compound I′-18 was synthesized according to the general procedure (I).

¹H NMR (300 MHz, D₂O): δ 8.49 (s, 1H), 8.22 (s, 1H), 7.82 (d, J=1.0 Hz, 1H), 6.12 (d, J=6.4 Hz, 1H), 5.87 (d, J=4.4 Hz, 1H), 5.00 (ddd, J=7.8, 4.8, 2.9 Hz, 1H), 4.85-4.80 (m, 1H), 4.72 (t, J=4.7 Hz, 1H), 4.66-4.60 (m, 1H), 4.56 (ddd, J=6.5, 4.9, 1.4 Hz, 1H), 4.46-4.10 (m, 11H), 4.09 (s, 3H), 3.66 (h, J=7.0 Hz, 2H), 3.52 (s, 3H), 3.50 (s, 3H), 1.17 (ddt, J=10.3, 7.7, 3.8 Hz, 1H), 0.63-0.54 (m, 2H), 0.40-0.32 (m, 2H).

³¹P NMR (121 MHz, D₂O): δ −0.80 (d, J=6.4 Hz, 1P), −11.53 (dt, J=18.7, 6.7 Hz, 2P), −22.90 (t, J=18.7 Hz, 1P).

MS (ESI⁻): Exact mass calculated for C₃₆H₄₉N₁₂O₂₅P₄ [M−H]⁻, 1173.19. Found 1173.07.

m⁷G(^{3′-OMe})pppA(2′-OMe)p(4-pm)¹Ψ (I′-19):

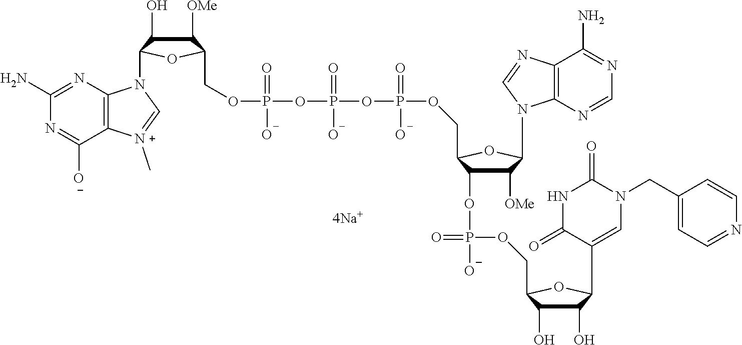

Compound I′-19 was synthesized according to the general procedure (I).

¹H NMR (300 MHz, D₂O): δ 8.53-8.49 (m, 2H), 8.48 (s, 1H), 8.20 (s, 1H), 7.91 (d, J=1.2 Hz, 1H), 7.45-7.40 (m, 2H), 6.08 (d, J=7.0 Hz, 1H), 5.91 (d, J=4.4 Hz, 1H), 5.08 (s, 2H), 5.01 (ddd, J=7.2, 4.7, 2.2 Hz, 1H), 4.90-4.84 (m, 1H), 4.77-4.74 (m, 1H), 4.71-4.66 (m, 1H), 4.58 (ddd, J=6.8, 4.8, 1.7 Hz, 1H), 4.50-4.38 (m, 2H), 4.32-4.11 (m, 9H), 4.09 (s, 3H), 3.52 (s, 3H), 3.45 (s, 3H).

³¹P NMR (121 MHz, D₂O): δ −0.86 (d, J=6.7 Hz, 1P), −11.30-−11.83 (m, 2P), −22.92 (t, J=18.7 Hz, 1P).

MS (ESI⁻): Exact mass calculated for C₃₈H₄₈N₁₃O₂₅P₄ [M−H]⁻, 1210.18. Found 1210.16.

### Example 3. Extended Translation of Non-Replicating mRNA by Novel Cap Analogs Containing Nucleoside Modification

### Background

One of the most defining characteristic features of mRNAs is a multifunctional cap at the five-prime end (5′) that was first discovered in the 1970s (1). For several cellular processes including nuclear transport (2), mRNA splicing (3,4), regulation of mRNA decay (5), and robust translation (6) mRNA requires a functional 5′ cap structure. Naturally occurring eukaryotic mRNA possesses a 7-methylguanosine (m7G) cap linked to the mRNA via a 5′ to 5′ triphosphate bridge resulting in what is termed as the Cap0 structure (m7GpppN). In most eukaryotic and some viral mRNA, further modifications can occur at the 2′-hydroxy-group (2′-OH) in the first and subsequent nucleotides, thereby producing Cap1 or Cap2 structures, respectively. Cap0-mRNA cannot be translated as efficiently as cap1 mRNA, where the role of 2′-O-met in the penultimate position at the mRNA 5′ end is determinant (7).

The use of co-transcriptional cap analogs offers a cost-effective and time-saving process for imitating the natural 5′ cap structure of endogenous mRNA. Dinucleotide cap analogs for capping of in vitro-transcribed (IVT) self-amplifying RNA (saRNA) and conventional messenger RNA (mRNA) are adequate substitutes for enzymatic capping and still widely used because of their many advantages including resistance to human decapping enzymes and to interferon-induced proteins with tetratricopeptide repeats (IFITs), which inhibit cap0-dependent translation (8,9). However, the main shortcoming is that GTP competes with the dinucleotide cap analogs during transcription, leading to poor capping efficiency and hence weak translational capacity. Nevertheless, cap1 analogs are commercially available that can be incorporated into both IVT saRNA (CleanCap AU, #N7-114, TriLink BioTechnologies) and IVT mRNA (CleanCap 3′OMe AG, #N7-413, TriLink BioTechnologies) in the correct orientation to produce cap1 mRNA with a high capping efficiency, all in a rapid co-transcriptional reaction (10). The most appealing feature is that this trinucleotide cap1 analog requires an A initiator, avoiding the slippage of RNA polymerases on the DNA template strand as opposed to those containing a G triplet as a transcriptional start site (11). Enzymatic capping can also yield cap1 mRNA, but it is time-consuming since it requires an extra purification step. Moreover, it demands a heating step to improve the accessibility of structured 5′ ends, thereby risking RNA degradation. In addition to this, the 5′ cap of enzymatically capped mRNA is not modified at the C2′ or C3′ position and it is well known that anti reverse cap analog (ARCA)-capped mRNA possesses higher translation efficiency compared to conventional cap analogs (12,13). There is also a variant of the CleanCap AG cap1 analog with a modification at the C3′ position of 7-methylguanosine (CleanCap AG 3′ OMe) which played an important role in the progress of immunotherapeutic vaccination strategy against SARS-CoV-2 (14). It is also well known that incorporation of nucleoside modifications including N1-methylpseudouridine (m1Ψ) (15) or N6-methyladenine (m6A) (16) into IVT mRNAs increases biological stability and thereby enhances the durability of the encoded protein compared to unmodified RNAs. Due to the fact that saRNA cannot contain modified nucleosides, its use is primarily limited to preventive vaccines against infectious diseases (17) in contrast to non-replicating mRNAs. The latter also have great potential in research areas such as gene editing, protein replacement therapy, where the reduction and elimination of immunomodulation is indispensable for reaching the appropriate therapeutic goal.

The advantages of using various modified nucleosides are indisputable in the mRNA research field. Nevertheless, the effects of cap analogs containing modified nucleosides on quality, translational efficiency and biological activity or immunogenicity of a long mRNA that encodes a potentially therapeutic protein is not understood. To answer this question, trinucleotide cap1 analogs containing various nucleoside modifications (i.e., compounds of formula I′) were synthesized and tested in vitro and in vivo using IVT mRNAs encoding murine erythropoietin. Our findings on the biological activity and immunogenicity of mRNAs capped with I′-6 (m₂^{7,3′O})Gppp(m^{2′O})Ap(m1)Ψ) cap analog suggest that this co-transcriptional trinucleotide cap analog containing modified nucleoside is a promising alternative to current capping strategies in mRNA vaccines and especially in RNA-based therapeutics.

### Evaluating the Impact of I′-6 Cap Analog Containing N1-Methylpseudouridine

For templates, linearized plasmid encoding codon-optimized murine erythropoietin (EPO) was used. The mRNAs starting with AUAGU was designed to contain the 5′ untranslated region (5′UTR) sequences of human α-globin (hAg) mRNA, an FI element as the 3′UTR, and an interrupted 100 nt-long 3′ poly(A) tail flanking the coding sequence. The MEGAscript T7 Transcription kit (Thermo Fisher Scientific, Waltham, MA, USA) was used for transcription, and UTP was kept or was replaced with N1-methylpseudouridine (m1Ψ) triphosphate (TriLink, San Diego, CA, USA). Capping of in vitro-transcribed mRNAs was performed co-transcriptionally using 4 mM of self-designed trinucleotide cap analogs which either contained a nucleoside modification (I′-3, I′-5, I′-6, I′-9, and I′-11 to I′-19) or not (I′-1 or I′-2) and commercially available reference cap analogs (CC114, CC413). To obtain the desired transcripts generated with these cap analogs, the initial GTP and UTP or m1ΨTP concentration in the transcription reaction was reduced from 7.5 mM to 1.5 mM and the 1.5 mL tubes were incubated at 37° C. for 30 min in a hybridization chamber. Sequential additions of 1.5 mM GTP and UTP or m1ΨTP were required to supplement the reaction at 30, 60, 90 and 120 min and incubated further at 37° C. for 30 min. To remove template DNA, Turbo DNase (Thermo Fisher Scientific, USA) was added to the reaction mix after the transcription reaction was completed and incubated for 15 min at 37° C. The synthesized mRNA was precipitated by adding a half volume of 8 M LiCl solution (Merck, Darmstadt, Germany) to the reaction mix and then pelleted by centrifugation. After dissolving in nuclease free water, mRNAs were cellulose-purified to remove double-stranded RNA contaminants, as described (18). The mRNA concentration and quality were measured on a NanoDrop™ 2000c spectrophotometer (Thermo Fisher Scientific, USA). Small aliquots of mRNA samples were stored in RNase-free tubes at −20° C.

In order to determine the capping efficiency of mRNAs capped with m1Ψ-containing I′-6 and cap analogs that do not contain uridine modifications (I′-1, CC413, and CC114), in vitro transcription reaction followed by a Ribozyme assay was performed. The Ribozyme cleavage reaction contained 0.45 μM mRNA. A 3-fold molar excess of ribozyme over mRNA substrate was added in an aqueous solution containing 30 mM HEPES and 150 mM NaCl. The ribozyme cleavage reaction was performed on a PCR machine utilizing the following program: 95° C. for 2 min, chill the mixture up to 37° C. by ramping rate of 0.1° C./sec, 37° C. for 5 min; after adding 30 mM MgCl₂ solution to each sample, the mixtures were maintained at 37° C. for 60 min followed by stopping the annealing at 80° C. for 2 min and transferring to ice for 5 min. Then, the short and long RNA fragments were separated using the RNA Clean & Concentrator-5 kit (Zymo Research Europe, Freiburg, Germany) according to the manufacturer's instructions. In our study, the following custom-designed hammerhead ribozyme specific for the hAg 5′UTR was used: 5′-UGU GGG CUG AUG AGG CCG UGA GGC CGA AAC CAG AAG AAU-3′ (synthesized by Metabion International AG, Planegg, Germany). To detect the short fragments, the samples (30 ng) were resolved on a 21% (vol/vol) 19:1 acrylamide:bisacrylamide denaturing gel supplemented with 8 M urea (Merck, Germany). Before loading the samples denatured by incubation at 75° C. for 5 min in the presence of 2×RNA loading buffer (New England Biolabs, Germany), the gel was pre-run at 180 V for 60 min. When the pre-run was finished, the pockets were rinsed with 1×TBE buffer. Immediately afterwards, the samples were loaded and the gel was run at 200 V constantly until the dye front reached the end of the gel. To identify the short, cleaved products, the gel was incubated with 1×TBE buffer containing 0.01% SYBR Gold nucleic acid stain (Thermo Fisher Scientific, USA) and the fluorescent signals were captured using a Gel Doc EZ Imager (Bio-Rad, Hercules, CA, USA). High capping efficiency is observed in terms of each tested mRNA regardless of cap analog (FIG. 3). According to Ribozyme assay analysis, the capping efficiency of novel m1Ψ-modified cap1 analog I′-6 is close to 100% and is comparable to the reference cap analogs (I′-1, CC413, and CC114) (FIG. 3).

For detection of short byproducts, IVT mRNAs (1.5-2 μg) were resolved on a 21% (vol/vol) 19:1 acrylamide:bisacrylamide denaturing gel supplemented with 8 M urea (Merck, Germany). Before loading the mRNA samples denatured by incubation at 75° C. for 10 min in the presence of 2×RNA loading buffer (New England Biolabs, Germany), the gel was pre-run at 180 V for 60 min. When the pre-run was finished, the pockets were rinsed with 1×TBE buffer. Immediately afterwards the samples were applied, and the gel was run at 180 V constantly until the dye front has reached the end of the gel. For identification of the short byproducts, the gel was incubated with 1×TBE buffer containing 0.01% SYBR Gold nucleic acid stain (Thermo Fisher Scientific, USA) and the fluorescent signals were captured using a Gel Doc EZ Imager (Bio-Rad, USA). Denaturing Urea Polyacrylamide Gel Electrophoresis showed minimal amount of short contaminants for I′-6 similar to CC413, while for unmodified mRNA samples a significant amount of contaminants was observed independent of cap analogs (FIG. 3).

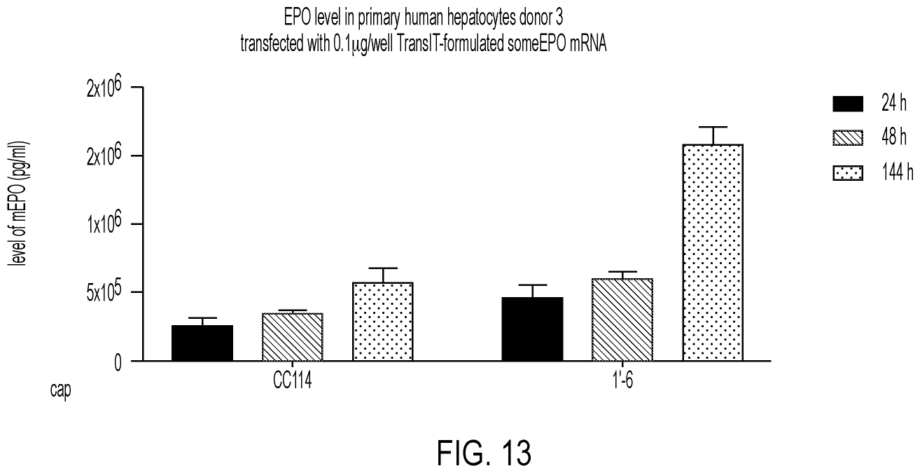

In each in vivo experiment in order to measure the translational efficiency and to determine the functionality of EPO mRNA capped with I′-1, I′-2, I′-3, I′-5, I′-6, I′-9, I′-12, I′-13, I′-16, CC114, and CC413 in vivo, female BALB/c mice from Jackson Laboratory (Bar Harbor, ME, USA) at the age of eight to ten weeks were used for in accordance with federal policies on animal research (Ethics approval number: G19-12-074). In each case, mice (n=3/group) were injected intravenously (i.v.) with 3 μg TransIT-complexed (Mirus Bio) EPO mRNAs in a final volume of 200 μL Dulbecco's modified Eagle medium (DMEM). Mice used as controls were injected with TransIT-reagent diluted in DMEM but without RNA. To measure hematocrits and EPO levels in the individual mice that were injected with EPO mRNA capped with I′-6, I′-1, CC114, and CC413 and complexed with TransIT-reagent, 18 μL of blood was collected at the indicated times (FIG. 8B) and centrifuged in Drummond microcaps glass capillary tubes (20 μl volume, Merck, Germany) as described (19). After determination of the hematocrit, capillary tubes were snapped open, and the plasma was collected to measure EPO levels and analyzed for mouse Erythropoietin DuoSet ELISA kit (R&D Systems) according to the manufacturer's instructions. First of all, these results demonstrated that mRNA capped with I′-6 (anti-reverse (ARCA) cap1 analog (m2(7,3′OMe)G(5′)ppp(5′)(2′OMe)Apm1Ψ)) outperformed each reference sample regardless of the cap initiators (FIG. 8A). EPO RNA containing m1Ψ modifications translated 2-3-fold, 5-fold, and 10-fold more than U-containing mRNAs at 6-24, 48 and 72 hours after injections, respectively. (FIG. 8A). I′-6 cap1 analog has a beneficial effect on long-term translational efficiency of m1Ψ-modified mRNA, meaning it showed 1.5-3-fold more plasma EPO level compared to those capped with CC413 at 48 and 72 h after injection, respectively (FIG. 8A). The positive effect of ARCA 3′-OMe I′-6 on the translational capacity of the mRNA is also expressed in the biological activities of the mRNA because hematocrit values remained very high level in mice injected with I′-6-capped EPO mRNA even at day 21 after injection (FIG. 8B). Hematocrit values started to decrease at D14 after injection of each mRNA capped with CC114 and CC413 but it was not observed in regards to I′-6-capped mRNA (FIG. 8B). Hematocrit value in mice injected with m1Ψ-mRNA capped with I′-6 at 21 days after administration is at the same level as the values of D7 and D14 in mice injected with CC413-capped mRNA (FIG. 8B). To address translation of I′-6-capped mRNA in human primary hepatocytes, EPO level was measured from supernatants transfected using 0.1 μg/well TransIT-formulated I′-6- or CC114-capped uRNA (FIG. 13). Increased secretion of EPO in human primary cells was detected at all three tested time points: 24 h, 48 h and 144 h (FIG. 13). These results suggest that cap1 analogs employing nucleoside modification (e.g., I′-6) are suitable for translation of the encoded protein and can be used for synthesizing non-replicating functional mRNAs.

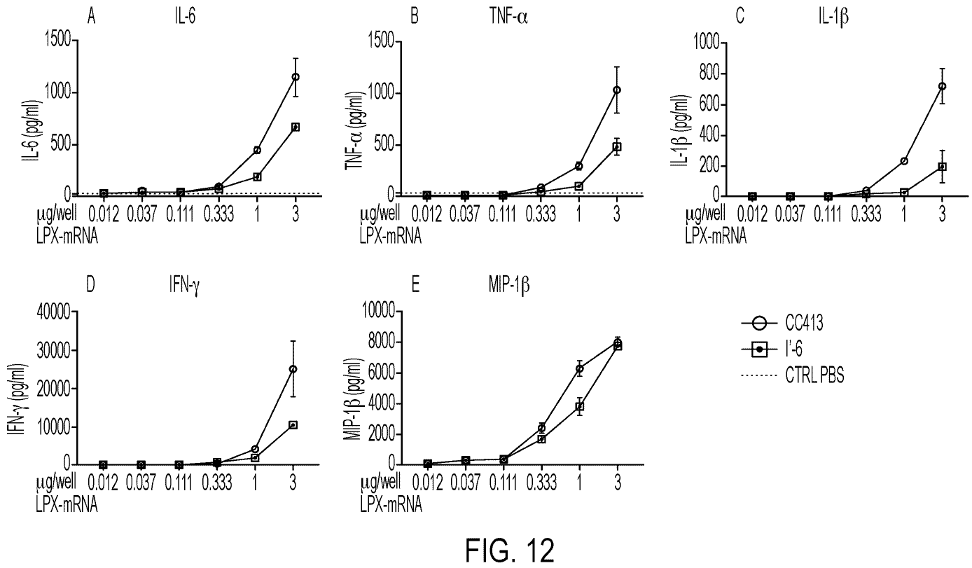

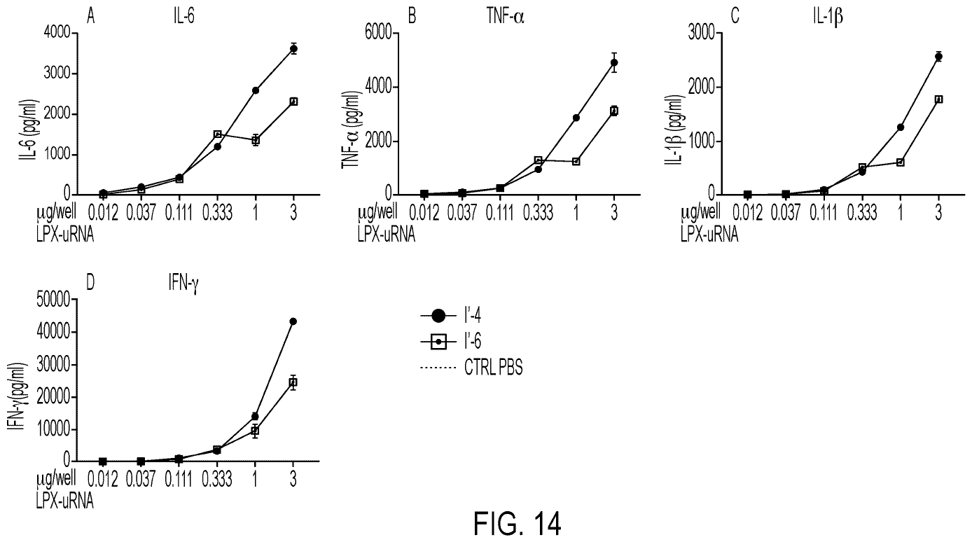

To test the effect of the cap types and mRNA 5′end on immunogenicity in human peripheral blood monocyte cells (PBMCs) we compared changes in levels of cytokines and chemokines after application of Lipoplex (LPX)-formulated EPO mRNAs. First, we compared the impact of m1Ψ-mRNA capped with I′-6 containing a UAGU 5′end vs. CC413 (AGAAU 5′end) (FIG. 12). Tested cytokines (IL-6, TNF-α, IL-1, IFN-γ) showed significant decrease when I′-6 capped mRNA was used after application of 1 and 3 μg/well, corresponding to 5 and 15 μg LPX-m1Ψ-mRNA/ml respectively. MIP-1β showed decrease when I′-6 was used after application of 0.333 and 1 μg/well, corresponding to 1.7 and 5 μg LPX-m1Ψ-mRNA/ml respectively. Second, we compared capped EPO uRNA with I′-6 or I′-1 (FIG. 14). In this case both mRNAs had the same TAGT 5′end. Still, I′-6 showed benefit leading to significantly lower cytokines (IL-6, TNF-α, IL-1β and IFN-γ) 24 h after application to human PBMCs. Thus, benefit of I′-6 in decreasing the immunogenicity and thus increasing safety was found when I′-6 was used.

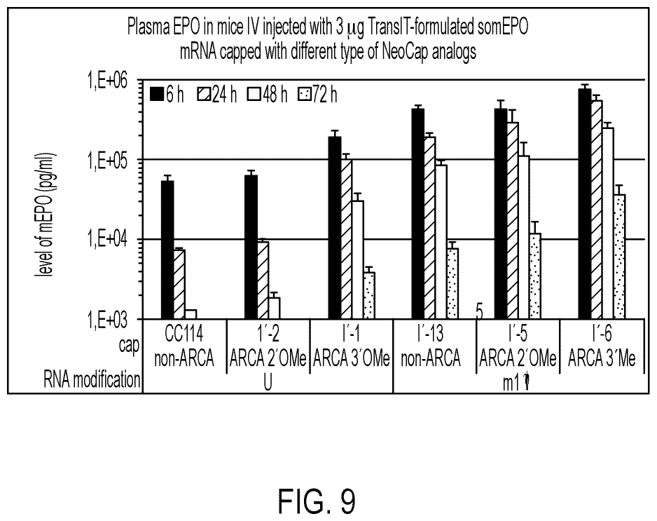

ARCA trinucleotide caps are not only much more effective than dinucleotide cap analogs and attributed to the correct orientation, but the additional methyl group on the m7G moiety might impact translation of the mRNA. To this end, we generated U-containing as well as m1Ψ-modified EPO RNA capped with cap analogs which belong to either the non-ARCA (I′-13) or ARCA (I′-6) classes. Moreover, the latter ones contain 2′-OMe (I′-5) or 3′-OMe (I′-6) at the ribose of the m7G moiety. Three μg of TransIT-complexed EPO mRNA capped with nucleoside modification-containing NeoCaps I′-13, I′-5 and I′-6 cap analogs were injected into mice intravenously. After that EPO level of plasma collected from mice was measured at 6, 24 and 48 hours after administration to compare the translational activity of mRNA capped with NeoCaps with U-containing or m1Ψ-modified mRNA capped with non-modified ARCA 3′OMe I′-1. EPO ELISA demonstrated that mRNA capped with m1Ψ-modified cap analogs, even non-ARCA I′-13 translated at least two-fold better at each tested time point compared to those capped with non-modified ARCA (3′-OMe I′-13), regardless of the mRNA modification (FIG. 9).

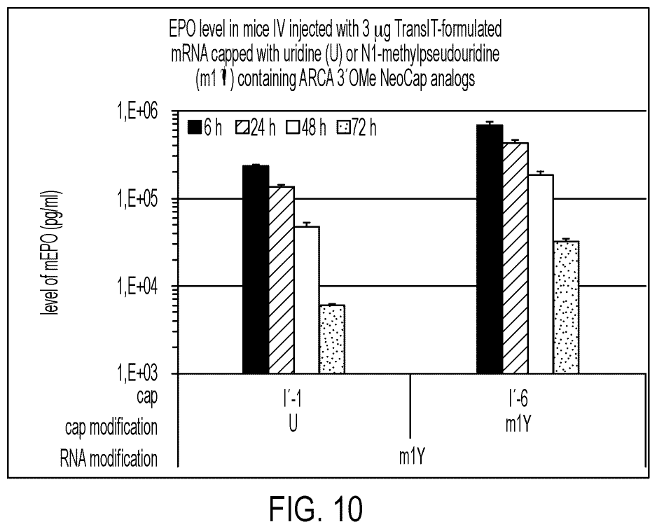

In order to investigate the importance of the presence of nucleoside modification (such as m1Ψ) in the cap analog on the long-term translational capacity of the m1Ψ-modified mRNA, two different combinations of cap analog were transcribed, one of which contained nucleoside modification (m1Ψ) at the N₂ position (I′-6), while the other did not (I′-1). Plasma EPO level of mice injected with 3 μg of each TransIT-complexed mRNA was determined using murine EPO-specific ELISA at 6, 24, 48 and 72 hours after intravenous administration. According to our in vivo result, the nucleoside modification, in this case m1Ψ, is determinant and very important to incorporate m1Ψ into the cap analog (I′-6) because the combination of m1Ψ-m1Ψtranslated 2-3-fold and 4-5 fold more than those capped with unmodified I′-1 6-24 hours and 48-72 hours after administration, respectively (FIG. 10). Taken together, these findings suggested that the incorporation of nucleoside modification into cap analogs has the beneficial effect on short and long-term translational activity of mRNA m1Ψ-modified ARCA 3′ OMe I′-6, which markedly stands out from other cap analogs.

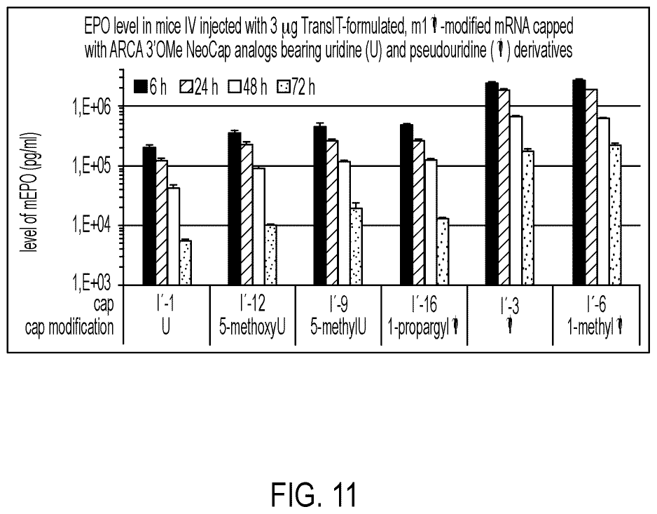

To test whether the incorporation of various uridine (U) or pseudouridine (Ψ) derivatives into the cap analogs improve the performance of IVT mRNA, EPO-encoding mRNA capped with cap analogs bearing N3-methyluridine (I′-11), N5-methyluridine (I′-9), N5-methoxyuridine (I′-12), N1-methylpseudouridine (I′-6), pseudouridine (I′-3), N3-methylpseudouridine I′-14), N1-trifluoroethylpseudouridine (I′-15), N1-propargylpseudouridine (I′-16), N1-benzylpseudouridine (I′-17), N1-cyclopropylmethyl-pseudouridine (I′-18) and N1-4-pyridylmethylpseudouridine (I′-19) were prepared using the same IVT conditions as described above. After purification of preselected mRNAs, 3 μg of each was complexed with TransIT-mRNA reagent and injected into Balb/c mice intravenously. At 6, 24, 48 and 72 hours after injection of mRNA capped with I′-9, I′-12, I′-6, I′-3, I′-16, and I′-1 into mice, murine EPO-specific ELISA was performed. For all cap analogs comprising a modified nucleoside at position N2, EPO levels were higher as compared to unmodified cap analog I′-1. EPO level in mice injected with mRNA capped with Ψ-containing cap analog (I′-3—m2(7,3′OMe)G(5′)ppp(5′)(2′OMe)ApΨ) is equal or slightly less to those bearing m1Ψ (I′-6—m2(7,3′OMe)G(5′)ppp(5′)(2′OMe)Apm1Ψ) (FIG. 11). Our finding showed that neither uridine (U) and its derivatives (5-methylU, 5-methoxyU) nor pseudouridine (Ψ) and its derivatives (1-propargylΨ) could improve the potency of N₁-methylpseudouridine (1-methylΨ)-containing cap (I′-6). I′-6 cap1 analog incorporated into m1Ψ-modified mRNA surpasses the performance of other cap analogs with other modification despite of the same capping efficiency.

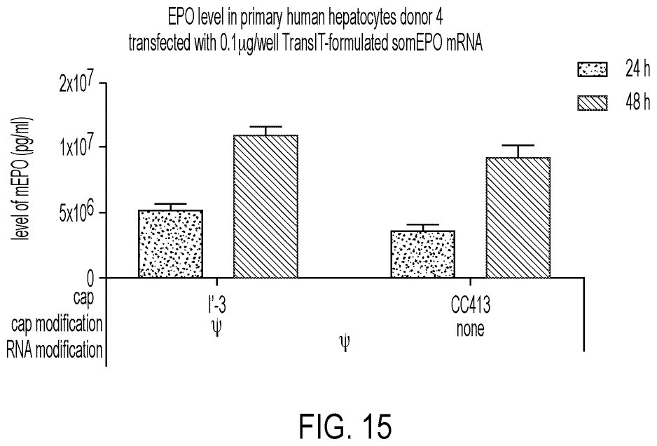

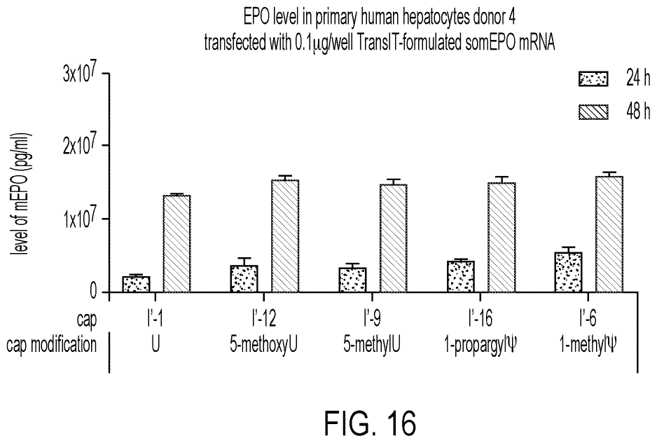

To test if various uridine (U) or pseudouridine (Ψ) derivatives of cap analogues can improve translation in human primary hepatocytes, we first measured EPO secretion after application of EPO-encoding Ψ-mRNA capped with compounds described herein analog bearing pseudouridine (I′-3) or CC413 (no modification). We found that level of EPO was higher at 24 h and 48 h when I′-3 was used compared to CC413 (FIG. 15). Comparison of EPO-encoding mRNA capped with cap1 analogs bearing N5-methyluridine (I′-9), N5-methoxyuridine (I′-12), N1-methylpseudouridine (I′-6) and N1-propargylpseudouridine (I′-16) showed increased level of secreted EPO at 24 h when I′-6 was used compared to other listed caps (FIG. 16). In addition, all tested modification bearing caps led to increase in EPO secretion at 24 h and 48 h when compared to the unmodified I′-1 (FIG. 16).

### CONCLUSION

In conclusion, the presented data demonstrated that m1Ψ-containing cap analog (I′-6—m2(7,3′OMe)G(5′)ppp(5′)(2′OMe)Apm1Ψ) bearing 3′-OMe at the ribose of the m7G moiety can be adapted to synthesis of non-replicating mRNA. We evaluated a unique, nucleoside modification-containing anti-reverse trinucleotide cap1 analog I′-6 that was used to generate a functional mRNA with 5′ cap1 structure resulting in a long-term maintenance of the encoded protein. We showed that using trinucleotide caps comprising a modified base at position N2, such as in particular I′-6 leads to a superior mRNA that significantly surpasses the translational capacity and biological activity of IVT mRNA capped with cap analogs that do not contain any nucleoside modifications (FIGS. 10, 11). Incorporation of naturally occurring modified nucleosides not only into mRNA but into cap analogs, due to their advantageous properties, may be appealing for other potential mRNA therapeutic applications, including protein replacement, cell therapy, and gene editing apart from the successfully used mRNA-based vaccines (14).

### REFERENCES

1. Furuichi, Y. (2015) Discovery of m(7)G-cap in eukaryotic mRNAs, Proceedings of the Japan Academy. Series B, Physical and biological sciences, 91, 394-409.
2. Izaurralde, E. and Mattaj, I. W. (1992) Transport of RNA between nucleus and cytoplasm, Seminars in cell biology, 3, 279-288.
3. Cooke, C. and Alwine, J. C. (1996) The cap and the 3′ splice site similarly affect polyadenylation efficiency, Molecular and cellular biology, 16, 2579-2584.
4. Izaurralde, E., Lewis, J., McGuigan, C., Jankowska, M., Darzynkiewicz, E. and Mattaj, I. W. (1994) A nuclear cap binding protein complex involved in pre-mRNA splicing, Cell, 78, 657-668.
5. Grudzien-Nogalska, E. and Kiledjian, M. (2017) New insights into decapping enzymes and selective mRNA decay, Wiley interdisciplinary reviews. RNA, 8. First published on Jul. 17, 2016.
6. Topisirovic, I., Svitkin, Y. V., Sonenberg, N. and Shatkin, A. J. (2011) Cap and cap-binding proteins in the control of gene expression, Wiley interdisciplinary reviews. RNA, 2, 277-298. First published on Oct. 28, 2010.
7. Diamond, M. S. (2014) IFIT1. A dual sensor and effector molecule that detects non-2′-O methylated viral RNA and inhibits its translation, Cytokine & growth factor reviews, 25, 543-550. First published on May 17, 2014.
8. Sahin, U., Derhovanessian, E., Miller, M., Kloke, B.-P., Simon, P., Lower, M., Bukur, V., Tadmor, A. D., Luxemburger, U. and Schrors, B. et al. (2017) Personalized RNA mutanome vaccines mobilize poly-specific therapeutic immunity against cancer, Nature, 547, 222-226.

First published on Jul. 5, 2017.
9. Wojtczak, B. A., Sikorski, P. J., Fac-Dabrowska, K., Nowicka, A., Warminski, M., Kubacka, D., Nowak, E., Nowotny, M., Kowalska, J. and Jemielity, J. (2018) 5′-Phosphorothiolate Dinucleotide Cap Analogues. Reagents for Messenger RNA Modification and Potent Small-Molecular Inhibitors of Decapping Enzymes, Journal of the American Chemical Society, 140, 5987-5999. First published on May 1, 2018.
10. Henderson, J. M., Ujita, A., Hill, E., Yousif-Rosales, S., Smith, C., Ko, N., McReynolds, T., Cabral, C. R., Escamilla-Powers, J. R. and Houston, M. E. (2021) Cap 1 Messenger RNA Synthesis with Co-transcriptional CleanCap Analog by In Vitro Transcription, Current protocols, 1, e39.
11. Imburgio, D., Rong, M., Ma, K. and McAllister, W. T. (2000) Studies of promoter recognition and start site selection by T7 RNA polymerase using a comprehensive collection of promoter variants, Biochemistry, 39, 10419-10430.
12. Stepinski, J., Waddell, C., Stolarski, R., Darzynkiewicz, E. and Rhoads, R. E. (2001) Synthesis and properties of mRNAs containing the novel “anti-reverse” cap analogs 7-methyl(3′—O-methyl)GpppG and 7-methyl (3′-deoxy)GpppG, RNA (New York, N.Y.), 7, 1486-1495.
13. Kuhn, A. N., Diken, M., Kreiter, S., Selmi, A., Kowalska, J., Jemielity, J., Darzynkiewicz, E., Huber, C., Tureci, O. and Sahin, U. (2010) Phosphorothioate cap analogs increase stability and translational efficiency of RNA vaccines in immature dendritic cells and induce superior immune responses in vivo, Gene therapy, 17, 961-971. First published on Apr. 22, 2010.
14. Sahin, U., Muik, A., Vogler, I., Derhovanessian, E., Kranz, L. M., Vormehr, M., Quandt, J., Bidmon, N., Ulges, A. and Baum, A. et al. (2021) BNT162b2 vaccine induces neutralizing antibodies and poly-specific T cells in humans, Nature, 595, 572-577. First published on May 27, 2021.
15. Karikó, K., Muramatsu, H., Welsh, F. A., Ludwig, J., Kato, H., Akira, S. and Weissman, D. (2008) Incorporation of pseudouridine into mRNA yields superior nonimmunogenic vector with increased translational capacity and biological stability, Molecular therapy: the journal of the American Society of Gene Therapy, 16, 1833-1840. First published on Sep. 16, 2008.
16. Gao, Y., Vasic, R., Song, Y., Teng, R., Liu, C., Gbyli, R., Biancon, G., Nelakanti, R., Lobben, K. and Kudo, E. et al. (2020) m6A Modification Prevents Formation of Endogenous Double-Stranded RNAs and Deleterious Innate Immune Responses during Hematopoietic Development, Immunity, 52, 1007-1021.e8. First published on Jun. 3, 2020.
17. Bloom, K., van den Berg, F. and Arbuthnot, P. (2021) Self-amplifying RNA vaccines for infectious diseases, Gene therapy, 28, 117-129. First published on Oct. 22, 2020.
18. Baiersdorfer, M., Boros, G., Muramatsu, H., Mahiny, A., Vlatkovic, I., Sahin, U. and Karikó, K. (2019) A Facile Method for the Removal of dsRNA Contaminant from In Vitro-Transcribed mRNA, Molecular therapy. Nucleic acids, 15, 26-35. First published on Feb. 27, 2019.
19. Mahiny, A. J. and Karikó, K. (2016) Measuring Hematocrit in Mice Injected with In Vitro-Transcribed Erythropoietin mRNA, Methods in molecular biology (Clifton, N. J.), 1428, 297-306.

### EQUIVALENTS

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

What is claimed is:

1. A trinucleotide cap G*N₁pN₂, or a salt thereof, wherein:
G* comprises a structure of formula I′: 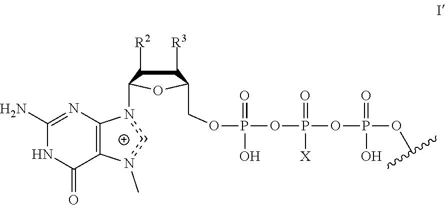
wherein:
each R² and R³ is independently —OH or —OCH₃; and
X is OH or SH;
N₁ is A or an analog thereof;
N₂ is U or an analog thereof; and
p is a group selected from phosphate (e.g., —P(═O)(OH)— or —P(═O)(O—)—) or thiophosphate (e.g., —P(═S)(OH)— or —P(═S)(O⁻)—).

2. The trinucleotide cap of claim 1, wherein R² is —OH and R³ is —OCH₃.

3. The trinucleotide cap of claim 1, wherein R² is —OCH₃ and R³ is —OH.

4. The trinucleotide cap of any one of claims 1-3, wherein X is OH or O⁻.

5. The trinucleotide cap of any one of claims 1-3, wherein X is SH or S⁻.

6. The trinucleotide cap of any one of claims 1-5, wherein N₁ is adenosine.

7. The trinucleotide cap of any one of claims 1-5, wherein N₁ is 6-methyladenosine.

8. The trinucleotide cap of any one of claims 1-5, wherein N₁ is 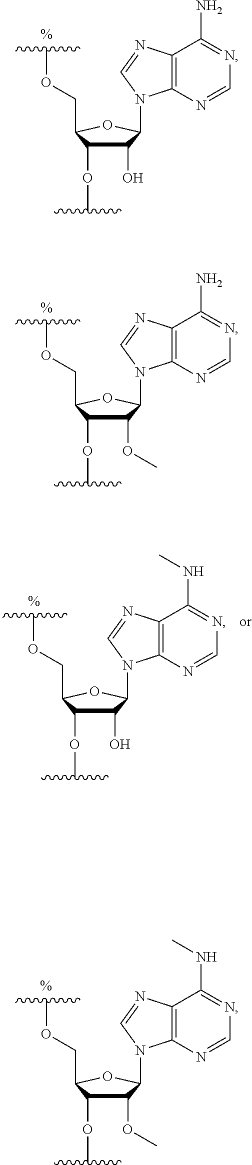
wherein % represents the point of attachment to G*.

9. The trinucleotide cap of any one of claims 1-8, wherein N₂ is a modified U.

10. The trinucleotide cap of any one of claims 1-8, wherein N₂ is of formula II′″: 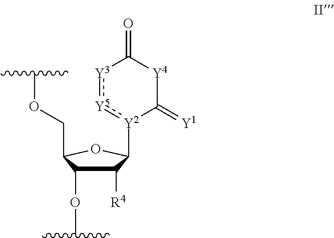
or a salt thereof, wherein:
each [symbol] is independently a single or double bond, as allowed by valency;
Y¹ is O or S;
Y² is N, C, or CH;
Y³ is N, NR^{a1}, CR^{a1}, or CHR^{a1};
Y⁴ is NR^{a2} or CHR^{a2};
Y⁵ is CR^{a3};
each of R¹, R^{a2} or R^{a3} is independently hydrogen, C₁₋₆ aliphatic, —CH₂R, or —O(C₁₋₄ alkyl);
R is C₁₋₄ aliphatic substituted with halogen, phenyl, a 3- to 6-membered saturated carbocyclic ring, or a 5- to 6-membered heteroaryl ring having 1-3 heteroatoms independently selected from nitrogen, oxygen, and sulfur;
R⁴ is —OH or —OMe; and
#represents the point of attachment to p of N₁p.

11. The trinucleotide cap of any one of claims 1-8, wherein N₂ is of formula II″: 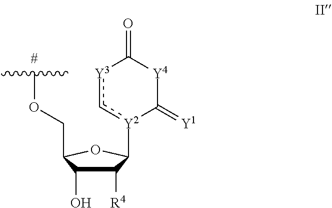
wherein:
each [symbol] is independently a single or double bond, as allowed by valency;
Y¹ is O or S;
Y² is N, C, or CH;
Y³ is N, NR^{a1}, CR^{a1}, or CHR^{a1};
Y⁴ is NR^{a2} or CHR^{a2};
each of R^{a1} or R^{a2} is independently hydrogen, C₁₋₆ aliphatic, —CH₂R, or —O(C₁₋₄ alkyl);
R is C₁₋₄ aliphatic substituted with halogen, phenyl, a 3- to 6-membered saturated carbocyclic ring, or a 5- to 6-membered heteroaryl ring having 1-3 heteroatoms independently selected from nitrogen, oxygen, and sulfur;
R⁴ is —OH or —OMe; and
#represents the point of attachment to p of N₁p.

12. The trinucleotide cap of claim 11, wherein N₂ is of formula IIa″:

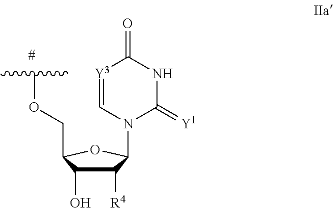

13. The trinucleotide cap of claim 11, wherein N₂ is of formula IIb″.

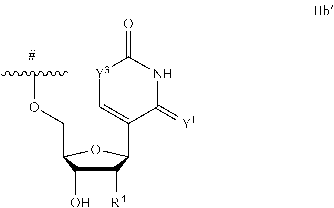

14. The trinucleotide cap of any one of claims 10-13, wherein Y¹ is O.

15. The trinucleotide cap of any one of claims 10-13, wherein Y¹ is S.

16. The trinucleotide cap of claim 12, wherein Y³ is CR^{a1}.

17. The trinucleotide cap of claim 13, wherein Y³ is NR^{a1}.

18. The trinucleotide cap of claim 16 or claim 17, wherein R^{a1} is hydrogen.

19. The trinucleotide cap of claim 16 or claim 17, wherein R^{a1} is C₁₋₆ aliphatic.

20. The trinucleotide cap of claim 19, wherein R^{a1} is methyl, ethyl, n-propyl, or isopropyl.

21. The trinucleotide cap of claim 20, wherein R^{a1} is methyl.

22. The trinucleotide cap of claim 16 or claim 17, wherein R^{a1} is —CH₂C≡CH.

23. The trinucleotide cap of claim 16, wherein R^{a1} is —O(C₁₋₄ alkyl).

24. The trinucleotide cap of claim 23, wherein R^{a1} is —OMe.

25. The trinucleotide cap of claim 16 or claim 17, wherein R^{a1} is —CH₂R.

26. The trinucleotide cap of claim 25, wherein R is C₁₋₄ aliphatic substituted with halogen.

27. The trinucleotide cap of claim 26, wherein R is C₁₋₂ aliphatic substituted with halogen.

28. The trinucleotide cap of claim 27, wherein R is —CF₃.

29. The trinucleotide cap of claim 25, wherein R is phenyl.

30. The trinucleotide cap of claim 25, wherein R is a 3- to 6-membered saturated carbocyclic ring.

31. The trinucleotide cap of claim 30, wherein R is a 3- to 4-membered saturated carbocyclic ring.

32. The trinucleotide cap of claim 31, wherein R is a 3-membered saturated carbocyclic ring.

33. The trinucleotide cap of claim 25, wherein R is a 5- to 6-membered heteroaryl ring having 1-3 heteroatoms independently selected from nitrogen, oxygen, and sulfur.

34. The trinucleotide cap of claim 33, wherein R is 4-pyridyl.

35. The trinucleotide cap of any one of claims 10-34, wherein R⁴ is —OH.

36. The trinucleotide cap of any one of claims 10-34, wherein R⁴ is —OMe.

37. The trinucleotide cap of any one of claims 1-8, wherein N₂ is selected from 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(τm5s2U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, 5-methyluridine (m⁵U), 1-methyl-pseudouridine (m1ψ), pseudouridine (ψ), 1-(2,2,2-trifluoroethyl)pseudouridine (tfet¹ψ), 1-propargylpseudouridine (ppg)¹ψ), 1-benzylpseudouridine (bn¹ψ), 1-(cyclopropylmethyl)pseudouridine (cpm¹ψ), and 1-(pyridin-4-ylmethyl)pseudouridine ((4-pm)¹ψ).

38. A trinucleotide cap selected from:

| Compound | |
|---|---|
| No. | Structure |
| I′-1 | 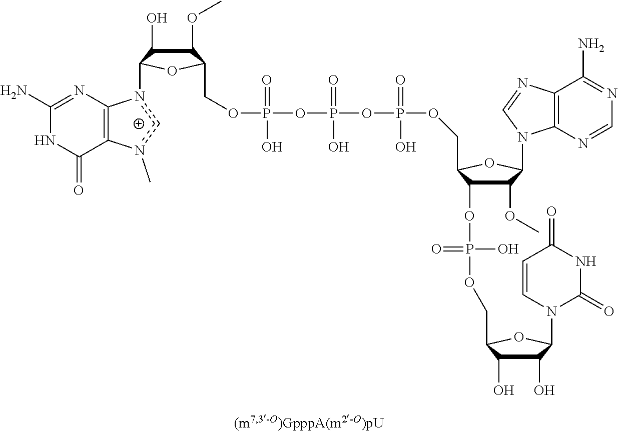 |
| I′-2 | 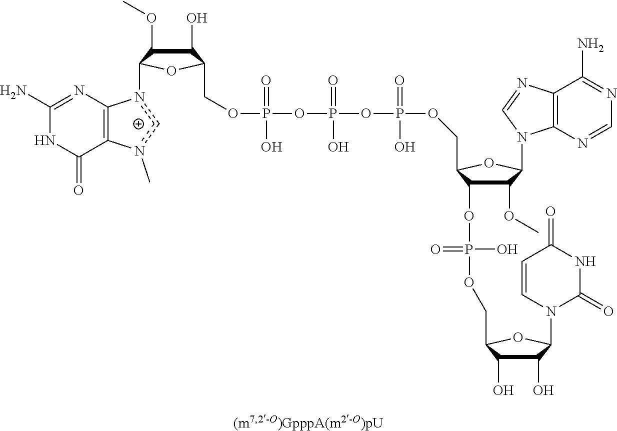 |
| I′-3 | 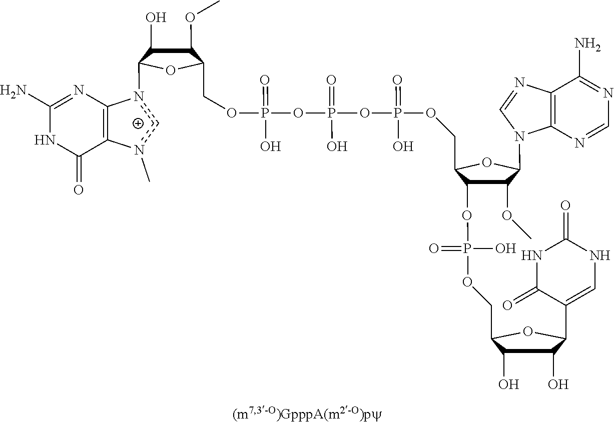 |
| I′-4 | 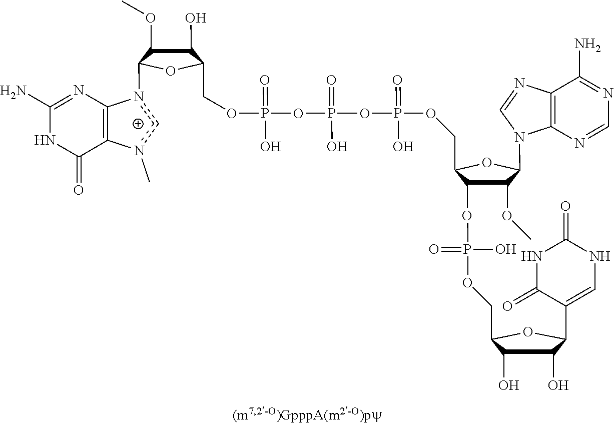 |
| I′-5 | 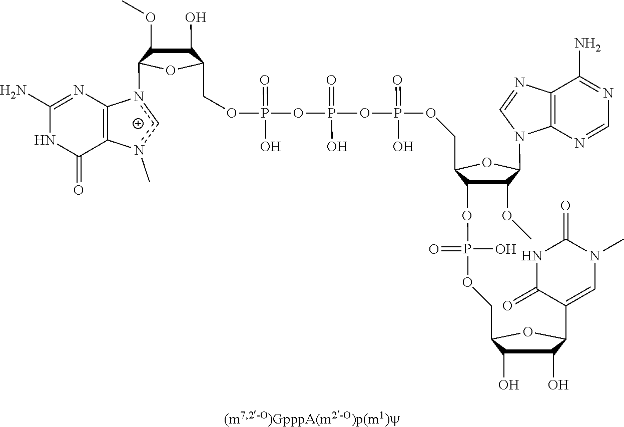 |
| I′-6 | 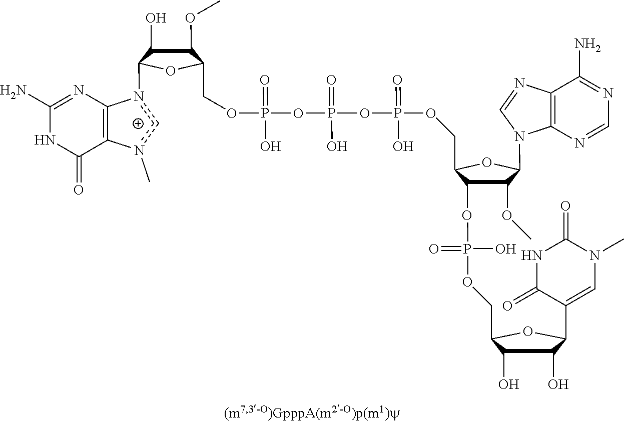 |
| I′-7 | 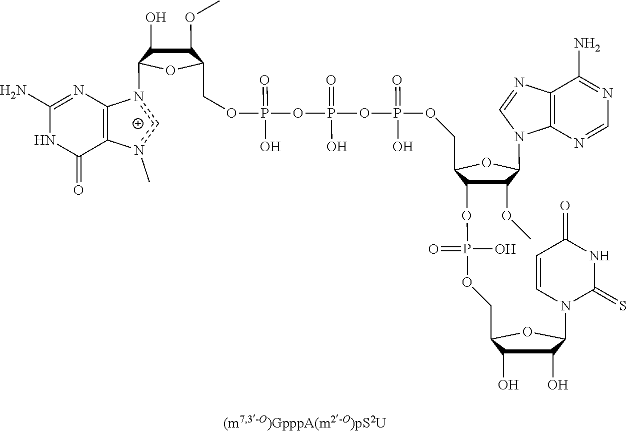 |
| I′-8 | 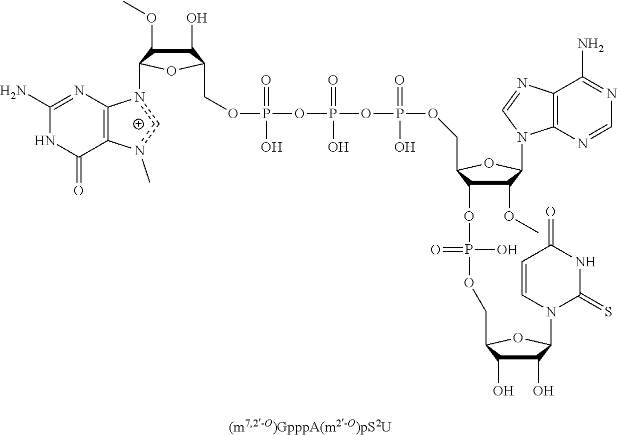 |
| I′-9 | 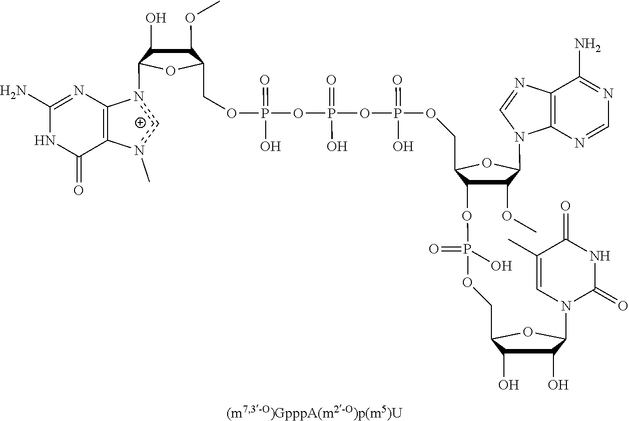 |
| I′-10 | 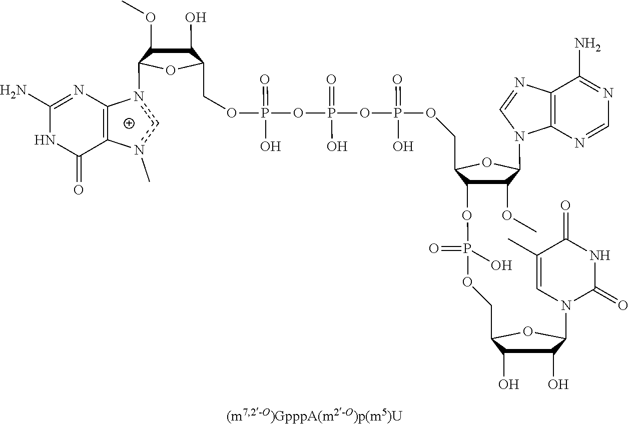 |
| I′-11 | 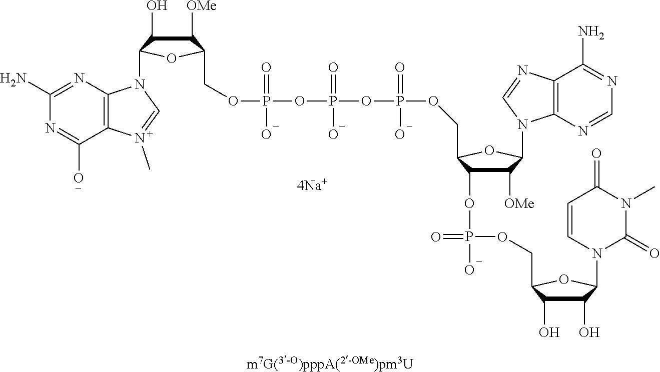 |
| I′-12 | 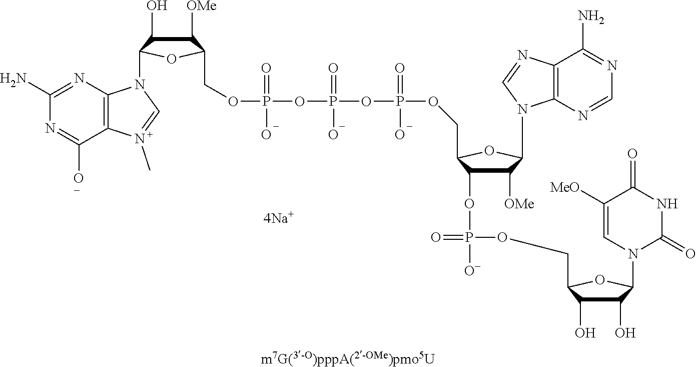 |
| I′-13 | 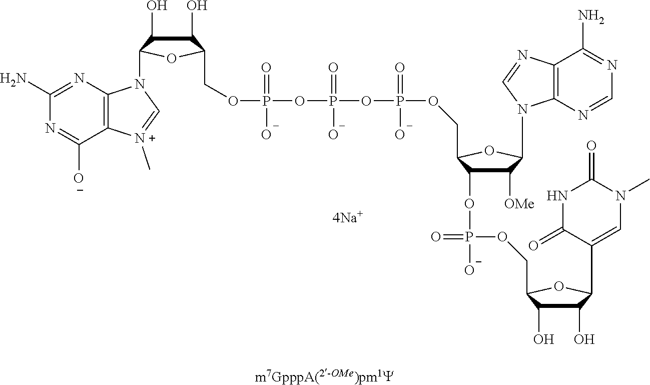 |
| I′-14 | 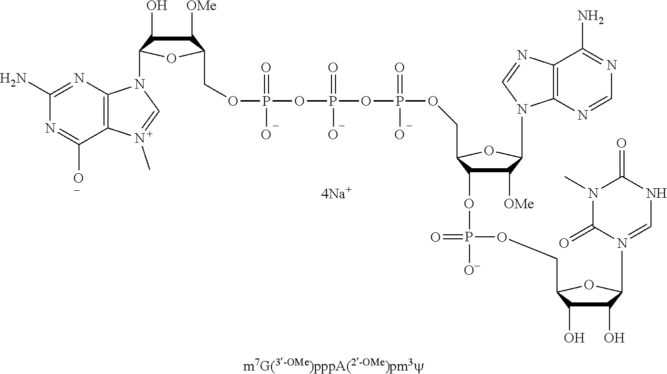 |
| I′-15 | 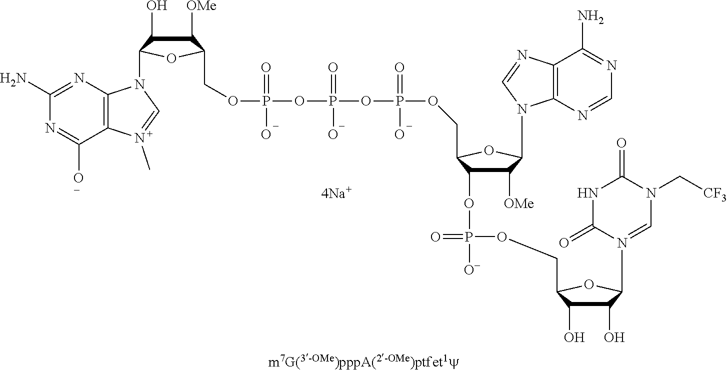 |
| I′-16 | 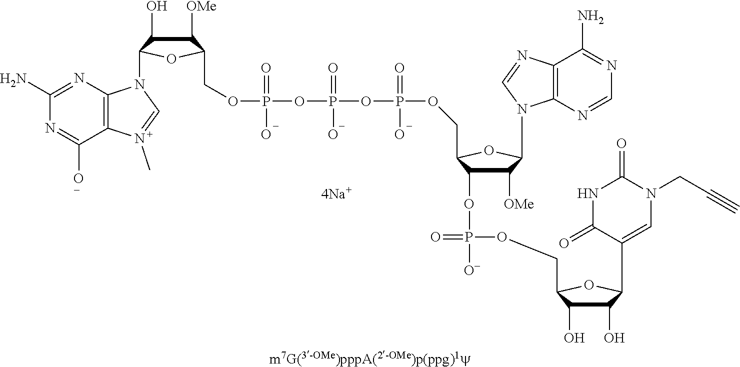 |
| I′-17 | 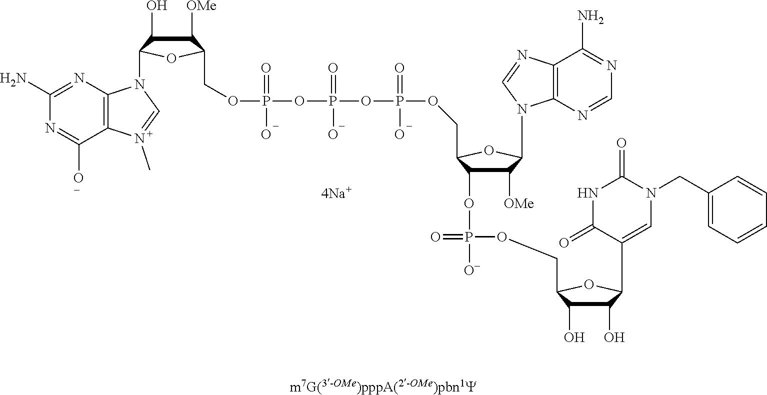 |
| I′-18 | 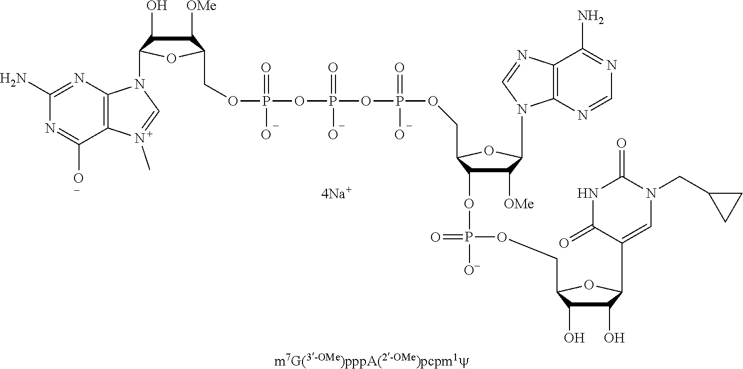 |
| I′-19 | 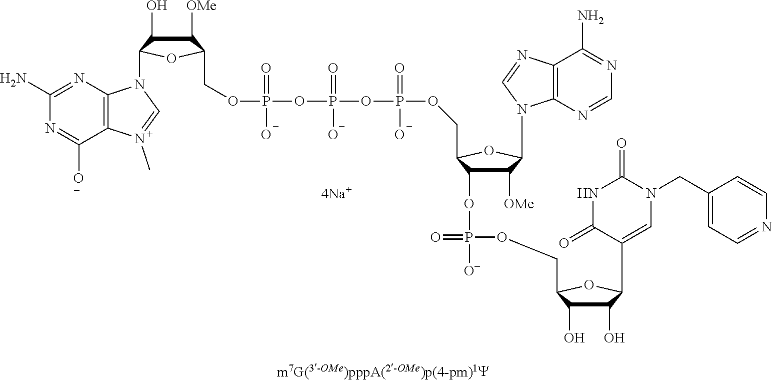 |

or a salt thereof.

39. A composition or medical preparation comprising a trinucleotide cap of any one of claims 1-38 or a salt thereof.
